(19) United States
(12) Patent Application Publication
(10) Pub. No.: US 2024/0016951 A1
(43) Pub. Date: Jan. 18, 2024

(54) COMPOSITIONS AND METHODS FOR TREATING ALLOGRAFT VASCULOPATHY, MOYAMOYA DISEASE, MOYAMOYA SYNDROME AND INTIMAL PROLIFERATION

(71) Applicants: Inozyme Pharma, Inc., Boston, MA (US); Westfälische Wilhelms-Universität Münster, Münster (DE)

(72) Inventors: Damon Banks, Boston, MA (US); Catherine A. Nester, Boston, MA (US); Edward Skolnik, Boston, MA (US); Markus Walz, Hoboken, NJ (US); Frank Rutsch, Münster (DE); Yvonne Nitschke, Münster (DE)

(21) Appl. No.: 18/148,888

(22) Filed: Dec. 30, 2022

Related U.S. Application Data
(63) Continuation of application No. PCT/US21/40356, filed on Jul. 2, 2021.
(60) Provisional application No. 63/047,877, filed on Jul. 2, 2020.
(60) Provisional application No. 63/047,848, filed on Jul. 2, 2020.
(60) Provisional application No. 63/047,865, filed on Jul. 2, 2020.
(60) Provisional application No. 63/047,793, filed on Jul. 2, 2020.

(51) Int. Cl. A61K 47/68; A61K 38/46; A61P 7/04

(52) U.S. Cl. CPC A61K 47/6815; A61K 38/465; A61P 7/04; C12Y 301/04001

(57) ABSTRACT

The present disclosure provides compositions and methods for treating allograft vasculopathy, for treating Moyamoya Diseases (MMD) and Moyamoya Syndrome (MMS), for treating inhibiting or preventing unwanted intimal proliferation in a subject by administering an ectonucleotide pyrophosphatase phosphodiesterase-1 (ENPP1) agent or an ectonucleotide pyrophosphatase phosphodiesterase-3 (ENPP3).

### CROSS REFERENCE

This U.S. patent application is a continuation of International Patent Application No. PCT/US2021/040356, filed Jul. 2, 2021, which claims priority to the following provisional applications, U.S. Application No. 63/047,793 filed on Jul. 2, 2020, U.S. Application No. 63/047,877 filed on Jul. 2, 2020, U.S. Application No. 63/047,865 filed on Jul. 2, 2020, and U.S. Application No. 63/047,848 filed on Jul. 2, 2020, the contents of each of which is herein incorporated by reference in its entirety.

### FIELD OF THE DISCLOSURE

The disclosure relates to compositions and methods of treating vascular diseases.

### SEQUENCE LISTING

This application contains a Sequence Listing which has been submitted electronically as a WIPO Standard ST.26 XML file via Patent Center, created on Dec. 30, 2022, is entitled “4427-10504.xml” and is 161,166 bytes in size. The sequence listing is incorporated herein by reference in its entirety.

### BACKGROUND

Myointimal proliferation or myointimal hyperplasia is a complex pathological process of the vascular system characterized by an abnormal proliferation of smooth muscle cells of the vascular wall. Proliferating smooth muscle cells migrate to the subendothelial area and form the hyperplastic lesion, which can cause stenosis and obstruction of the vascular lumen.

Cardiac Allograft Vasculopathy (CAV) is an accelerated fibroproliferative disorder affecting muscular vessels of the graft and is a leading cause of morbidity and mortality following cardiac transplantation. CAV is believed to be mediated by immunologic damage and infiltration of the endothelium, resulting in proliferation of vascular smooth muscle cells and subsequent luminal narrowing. Symptoms of CAV include progressive thickening of the arterial intima in both epicardial and intramyocardial arteries of the graft, often driven by immune-mediated vascular injury. CAV occurs in at least around a third (at varying severity) of all cardiac transplant recipients by three years post-transplant. CAV is often characterized by vascular smooth muscle cell proliferation, accumulation of inflammatory immune cells, and lipid deposition. CAV is a slow progressive disease but complications such as acute graft failure, arrhythmia, infarction, or cardiac death can often manifest without classic symptoms (such as angina) due to graft denervation.

Similar vasculopathy occurs in, and can severely limit long-term survival of, other solid organ allografts. Because such vasculopathies are difficult to treat, and can affect nearly all vessels of the allograft, they are associated with significant morbidity and mortality for allograft recipients and may require repeat transplantation. Therefore, effective therapies that may prevent or reduce the extent of such vasculopathies in solid organ allografts, such as cardiac allografts, are urgently needed.

Moyamoya is an occlusive cerebrovascular disorder first reported in 1957 in Japan and is characterized by stenosis of the supraclinoid portion of the internal carotid arteries (ICA) with the formation of an abnormal vascular network at the base of the brain. Moyamoya is a general term used to describe two different conditions affecting the intracranial internal carotid artery; moyamoya disease (MMD), a congenital disease causing bilateral arteriopathy which is more prominent among East Asian and Japanese children and adults, and Moyamoya syndrome (MMS), which is idiopathic, and typically seen among Caucasian adults ranging in age from 20 to 40 years. While there is no known genetic component in MMS, as there is in MMD, it is often associated with autoimmune disorders such as diabetes, lupus or rheumatoid arthritis. Treatment options for both MMD and MMS have involved daily aspirin use, lifestyle modifications to maximize cerebral perfusion, and surgical direct or indirect bypass to restore blood flow. Principally affecting women (70-85%) more than men (15-30%), moyamoya spans ethnicities, but is most prevalent in East Asians and Caucasians. Moyamoya disease (MMD) is prominent amongst the East Asian population presenting in both children and adults with a familial lineage. Moyamoya syndrome (MMS) is prominent amongst Caucasians in the 2^{nd}/3^{rd} decades of life, is idiopathic, and usually presents with co-morbidities (autoimmune diseases) Clinical literature often does not distinguish between those with MMD and MMS.

Chronic hemodialysis is a common treatment for patients suffering from poor kidney function. Such patients often undergo a surgical procedure in which an artificial arterio-venous fistula (AVF) is created usually in their non-dominant arm. The AVF provides a durable vascular access point for the hemodialysis process. A common complication with AVF is the occlusion of the AVF or vessels at or adjacent to the location of the AVF. Such occlusion can involve, for example, thromboses and intimal hyperplasia, and can result in permanent nerve damage or paralysis of the affected limb, if left untreated (see, e.g., Asif et al. (2006) Clin J Am Soc Nephrol. 1:332-339; Nath et al. (2003) Am J Pathol. 162:2079-90; and Stolic (2013) Med Pric Pract. 22(3):220-228).

### SUMMARY OF THE DISCLOSURE

In one aspect, the disclosure relates to a method for reducing and/or preventing allograft vasculopathy in a subject having an allograft, the method comprising: administering to the subject an effective amount of an ENPP1 agent or ENPP3 agent to thereby reduce and/or prevent allograft vasculopathy in said subject.

In another aspect, the disclosure relates to a method for preventing or ameliorating one or more symptoms associated with Moyamoya disease in a subject, the method comprising: administering to the subject an ENPP1 agent or ENPP3 agent in an amount sufficient to thereby prevent or ameliorate one or more symptoms associated with Moyamoya disease in the subject.

In another aspect, the disclosure relates to a method for inhibiting or preventing cerebral vascular occlusion in a subject who is expected to receive or who has received a surgical intervention as a treatment for Moyamoya disease, the method comprising: administering to the subject an ENPP1 agent or ENPP3 agent in an amount sufficient to thereby inhibit or prevent cerebral vascular occlusion in the subject.

In another aspect, the disclosure relates to a method for inhibiting or preventing unwanted vascular smooth muscle cell proliferation in a subject who is expected to receive or who has received a surgical intervention as a treatment for Moyamoya disease, the method comprising: administering to the subject an ENPP1 agent or ENPP3 agent in an amount sufficient to thereby inhibit or prevent unwanted vascular smooth muscle cell proliferation in the subject.

In another aspect, the disclosure also includes a method for inhibiting or slowing progression of Stage I Suzuki grade MMD to Stage II Suzuki grade MMD in a subject, the method comprising: administering to the subject an effective amount of an ENPP1 agent or an ENPP3 agent to thereby inhibit and/or slow progression of Stage I MMD to Stage II MMD in said subject.

In another aspect, the disclosure also includes a method for inhibiting or slowing progression of Stage I Suzuki grade MMD to Stage III Suzuki grade MMD in a subject, the method comprising: administering to the subject an effective amount of an ENPP1 agent or an ENPP3 agent to thereby inhibit and/or slow progression of Stage I MMD to Stage III MMD in said subject.

In yet another aspect, the disclosure relates to method for inhibiting or preventing cerebral vascular occlusion in a subject at risk for developing Moyamoya disease, the method comprising: administering to the subject an ENPP1 agent or ENPP3 agent in an amount sufficient to thereby inhibit or prevent cerebral vascular occlusion in the subject.

In yet another aspect, the disclosure relates to a method for inhibiting or preventing unwanted vascular smooth muscle cell proliferation in a subject at risk for developing Moyamoya disease, the method comprising: administering to the subject an ENPP1 agent or ENPP3 agent in an amount sufficient to thereby inhibit or prevent unwanted vascular smooth muscle cell proliferation in the subject.

In yet another aspect, the disclosure also relates to a method for treating a subject at risk for developing Moyamoya disease, the method comprising: administering to the subject an ENPP1 agent or ENPP3 agent in an amount sufficient to thereby treat the subject

In yet another aspect, the disclosure relates to a method for inhibiting or preventing cerebral vascular occlusion in a subject afflicted with Moyamoya disease, the method comprising: administering to the subject an ENPP1 agent or ENPP3 agent in an amount sufficient to thereby inhibit or prevent cerebral vascular occlusion in the subject. The disclosure relates to a method for inhibiting or preventing unwanted vascular smooth muscle cell proliferation in a subject afflicted with Moyamoya disease, the method comprising: administering to the subject an ENPP1 agent or ENPP3 agent in an amount sufficient to thereby inhibit or prevent unwanted cerebral vascular smooth muscle cell proliferation in the subject.

In yet another aspect, the disclosure relates to a method for treating a subject afflicted with Moyamoya disease, the method comprising: administering to the subject an ENPP1 agent or ENPP3 agent in an amount sufficient to thereby treat the subject.

In yet another aspect, the disclosure relates to a method for treating a subject having Moyamoya disease, the method comprising: administering to the subject an effective amount of an ENPP1 agent or ENPP3 agent to thereby treat said Moyamoya disease in said subject.

In yet another aspect, the disclosure relates to a method for treating a subject having Moyamoya syndrome, the method comprising: administering to the subject an effective amount of an ENPP1 agent or ENPP3 agent to thereby treat said Moyamoya syndrome in said subject.

In yet another aspect, the disclosure includes a method for reducing and/or preventing progression of vascular smooth muscle cell proliferation in a cerebral artery of a subject, the method comprising: administering to the subject an effective amount of an ENPP1 agent or ENPP3 agent to thereby reduce and/or prevent progression of said vascular smooth muscle cell proliferation in said cerebral artery of said subject. In some embodiments of any of the methods described herein, the subject has stage I, stage II or stage III, grade IV Suzuki grade MMD.

In yet another aspect, the disclosure also includes a method for inhibiting or slowing progression of Stage I Suzuki grade MMD to Stage II Suzuki grade MMD in a subject, the method comprising: administering to the subject an effective amount of an ENPP1 agent or an ENPP3 agent to thereby inhibit and/or slow progression of Stage I MMD to Stage II MMD in said subject.

In another aspect, the disclosure features a method for treating a subject having Moyamoya disease, the method comprising: administering to the subject an effective amount of an ENPP1 agent or an ENPP3 agent to thereby treat said peripheral artery disease in said subject.

In another aspect, the disclosure relates to a method for treating a subject having Moyamoya syndrome, the method comprising: administering to the subject an effective amount of an ENPP1 agent or an ENPP3 agent to thereby treat said Moyamoya syndrome in said subject.

In yet another aspect, the disclosure features a method for reducing and/or preventing progression of vascular smooth muscle cell proliferation in a cerebral artery of a subject having Moyamoya disease, the method comprising: administering to the subject an effective amount of an ENPP1 agent or ENPP3 agent to thereby reduce and/or prevent progression of said vascular smooth muscle cell proliferation in said cerebral artery of said subject.

The disclosure also relates to a method for reducing and/or preventing progression of vascular smooth muscle cell proliferation in a cerebral artery of a subject who undergoes surgery on said cerebral artery, the method comprising: administering to the subject an effective amount of an ENPP1 agent or ENPP3 agent to thereby reduce and/or prevent progression of vascular smooth muscle cell proliferation in said cerebral artery at a surgical site of said cerebral artery in said subject.

The disclosure also relates to a method for reducing and/or preventing progression of vascular smooth muscle cell proliferation in a cerebral artery of a subject who undergoes surgery on said cerebral artery, the method comprising: administering to the subject an effective amount of an ENPP1 agent or ENPP3 agent to thereby reduce and/or prevent progression of vascular smooth muscle cell proliferation in said cerebral artery at a surgical site of said cerebral artery in said subject.

In another aspect, the disclosure features a method for treating a subject having Moyamoya disease, the method comprising: administering to the subject an effective amount of an ENPP1 agent or an ENPP3 agent to thereby treat said peripheral artery disease in said subject. In another aspect, the disclosure relates to a method for treating a subject having Moyamoya syndrome, the method comprising: administering to the subject an effective amount of an ENPP1 agent or an ENPP3 agent to thereby treat said Moyamoya syndrome in said subject. In yet another aspect, the disclosure features a method for reducing and/or preventing progression of vascular smooth muscle cell proliferation in a cerebral artery of a subject having Moyamoya disease, the method comprising: administering to the subject an effective amount of an ENPP1 agent or an ENPP3 agent to thereby reduce and/or prevent progression of said vascular smooth muscle cell proliferation in said cerebral artery of said subject.

In another aspect, the disclosure also relates to a method for reducing and/or preventing progression of vascular smooth muscle cell proliferation in a subject's peripheral vessel at or around the site at which an arterio-venous dialysis shunt has been placed, the method comprising: administering to the subject an effective amount of an ENPP1 agent or an ENPP3 agent to thereby reduce and/or prevent progression of vascular smooth muscle cell proliferation in said peripheral vessel at or around the site the arterio-venous dialysis shunt has been placed.

In one aspect, the disclosure provides a method for reducing and/or preventing progression of vascular smooth muscle cell proliferation in a peripheral vessel of a subject who undergoes surgery on said peripheral vessel, the method comprising: administering to the subject an effective amount of an ENPP1 agent or an ENPP3 agent to thereby reduce and/or prevent progression of vascular smooth muscle cell proliferation in said peripheral vessel at a surgical site of said peripheral vessel in said subject, wherein the surgery comprises placement of an arterio-venous dialysis shunt.

In another aspect, the disclosure relates to a method for reducing and/or preventing progression of vascular smooth muscle cell proliferation in a peripheral vessel of a subject who requires surgery on said peripheral vessel, wherein the surgery comprises placement of an arterio-venous dialysis shunt, the method comprising: administering to the subject an effective amount of an ENPP1 agent or an ENPP3 agent to thereby reduce and/or prevent progression of vascular smooth muscle cell proliferation in said peripheral vessel at a surgical site of said peripheral vessel in said subject.

In another aspect, the disclosure also includes a method for reducing and/or preventing progression of vascular smooth muscle cell proliferation in a peripheral vessel of a subject who undergoes shunt placement in a peripheral vessel, the method comprising: administering to the subject an effective amount of an ENPP1 agent or an ENPP3 agent to thereby reduce and/or prevent progression of vascular smooth muscle cell proliferation in the peripheral vessel.

In another aspect, the disclosure features a method for reducing and/or preventing stenosis or restenosis in a peripheral vessel of a subject who undergoes shunt placement in the peripheral vessel, the method comprising: administering to the subject an effective amount of an ENPP1 agent or an ENPP3 agent to thereby reduce and/or prevent stenosis or restenosis in the peripheral vessel.

In another aspect, the disclosure relates to a method for reducing and/or preventing vasculopathy of an allografted vessel in a subject, the method comprising: administering to the subject an effective amount of: (i) an ENPP1 agent or ENPP3 agent and (ii) a complement inhibitor to thereby reduce and/or prevent vasculopathy of the allografted vessel in said subject. In some embodiments, the vessel is an artery. In some embodiments, the vessel is a vein.

In another aspect, the disclosure relates to a method for reducing and/or preventing vasculopathy of an allografted vessel in a subject, the method comprising: administering to the subject an effective amount of an ENPP1 agent or ENPP3 agent to thereby reduce and/or prevent vasculopathy of the allografted vessel in said subject. In some embodiments, the vessel is an artery. In some embodiments, the vessel is a vein.

In another aspect, the disclosure relates to a method for reducing and/or preventing vasculopathy of an allografted vessel in a subject, the method comprising: administering to the subject an effective amount of an ENPP1 agent or ENPP3 agent to thereby reduce and/or prevent vasculopathy of the allografted vessel in said subject. In some embodiments, the vessel is an artery. In some embodiments, the vessel is a vein. In some embodiments, the subject has received or is receiving a therapy comprising a complement inhibitor. In some embodiments, the methods comprise administering to the subject a complement inhibitor.

In another aspect, the disclosure relates to a method for reducing and/or preventing progression of vascular smooth muscle cell proliferation in an allografted vessel in a subject, the method comprising: administering to the subject an effective amount of: (i) an ENPP1 agent or ENPP3 agent and (ii) a complement inhibitor to thereby reduce and/or prevent progression of vascular smooth muscle cell proliferation in the allografted vessel in said subject. In some embodiments, the vessel is an artery. In some embodiments, the vessel is a vein.

In another aspect, the disclosure relates to a method for reducing and/or preventing allograft vasculopathy (for example, cardiac allograft vasculopathy) in a subject having an allograft, the method comprising: administering to the subject an effective amount of: (i) an ENPP1 agent or ENPP3 agent and (ii) a complement inhibitor to thereby reduce and/or prevent allograft vasculopathy in said subject.

In another aspect, the disclosure relates to a method for reducing and/or preventing progression of vascular smooth muscle cell proliferation in an allografted vessel in a subject, the method comprising: administering to the subject an effective amount of an ENPP1 agent or ENPP3 agent to thereby reduce and/or prevent progression of vascular smooth muscle cell proliferation in the allografted vessel in said subject. In some embodiments, the vessel is an artery. In some embodiments, the vessel is a vein.

In yet another aspect, the disclosure relates to a method for reducing and/or preventing allograft vasculopathy (for example, cardiac allograft vasculopathy) in a subject having an allograft and who has received or is receiving a therapy comprising a complement inhibitor, the method comprising: administering to the subject an effective amount of an ENPP1 agent or ENPP3 agent to thereby reduce and/or prevent allograft vasculopathy in said subject. In some embodiments, the methods further comprise administering the complement inhibitor to the subject.

In another aspect, the disclosure relates to a method for reducing and/or preventing allograft vasculopathy (for example, cardiac allograft vasculopathy) in a subject having an allograft and who has received or is receiving a therapy comprising an ENPP1 agent or ENPP3 agent, the method comprising: administering to the subject an effective amount of a complement inhibitor to thereby reduce and/or prevent allograft vasculopathy in said subject. In some embodiments, the methods further comprise administering the ENPP1 agent or ENPP3 agent to the subject.

In another aspect, the disclosure relates to a method for reducing and/or preventing progression of vascular smooth muscle cell proliferation in the vasculature of an allograft of a subject having said allograft, the method comprising administering to the subject an effective amount of: (i) an ENPP1 agent or an ENPP3 agent and (ii) a complement inhibitor to thereby reduce and/or prevent progression of said vascular smooth muscle cell proliferation in said vasculature of said allograft of said subject.

In another aspect, the disclosure relates to a method for reducing and/or preventing progression of vascular smooth muscle cell proliferation in the vasculature of an allograft of a subject having said allograft, wherein the subject has received or is receiving a therapy comprising a complement inhibitor, the method comprising administering to the subject an effective amount of an ENPP1 agent or an ENPP3 agent to thereby reduce and/or prevent progression of said vascular smooth muscle cell proliferation in said vasculature of said allograft of said subject. In some embodiments, the methods further comprise administering the complement inhibitor to the subject.

In another aspect, the disclosure relates to a method for reducing and/or preventing progression of vascular smooth muscle cell proliferation in the vasculature of an allograft of a subject having said allograft, wherein the subject has received or is receiving a therapy comprising an ENPP1 agent or an ENPP3 agent, the method comprising administering to the subject an effective amount of a complement inhibitor to thereby reduce and/or prevent progression of said vascular smooth muscle cell proliferation in said vasculature of said allograft of said subject. In some embodiments, the methods further comprise administering the ENPP1 agent or ENPP3 agent to the subject.

In yet another aspect, the disclosure also relates to a method for reducing and/or preventing progression of vascular smooth muscle cell proliferation in a solid organ transplant in a subject having a solid organ transplant and who undergoes surgery on said organ transplant, the method comprising administering to the subject an effective amount of: (i) an ENPP1 agent or an ENPP3 agent and (ii) a complement inhibitor to thereby reduce and/or prevent progression of vascular smooth muscle cell proliferation in said solid organ transplant of said subject.

In yet another aspect, the disclosure also features a method for delaying or preventing or for prophylaxis against failure of an allografted vessel in a subject having said allografted vessel, the method comprising: administering to the subject an effective amount of an ENPP1 agent or an ENPP3 agent to thereby delay, prevent or provide prophylaxis against failure of the allografted vessel in the subject. In some embodiments, the subject has received or is receiving a therapy comprising a complement inhibitor. In some embodiments, the methods comprise administering to the subject a complement inhibitor.

In yet another aspect, the disclosure also features a method for delaying or preventing or for prophylaxis against failure of an allografted vessel in a subject having said allografted vessel, the method comprising: administering to the subject an effective amount of an ENPP1 agent or an ENPP3 agent to thereby delay, prevent or provide prophylaxis against failure of the allografted vessel in the subject. In some embodiments, the subject has received or is receiving a therapy comprising a complement inhibitor. In some embodiments, the methods comprise administering to the subject a complement inhibitor.

In yet another aspect, the disclosure also features a method for delaying solid organ allograft failure in a subject having said solid organ allograft, the method comprising: administering to the subject an effective amount of an: (i) ENPP1 agent or an ENPP3 agent and (ii) a complement inhibitor to thereby delay solid organ allograft failure in the subject. In some embodiments, the allograft failure can be delayed for at least two months (e.g., at least six months, at least one year, at least two years, at least three years, at least five years, at least seven years, at least 10 years, or even more than 10 years).

In yet another aspect, the disclosure also features a method for delaying failure of an allografted vessel in a subject having said allografted vessel, the method comprising: administering to the subject an effective amount of an: (i) ENPP1 agent or an ENPP3 agent and (ii) a complement inhibitor to thereby delay failure of the allografted vessel in the subject. In some embodiments, the allograft failure can be delayed for at least two months (e.g., at least six months, at least one year, at least two years, at least three years, at least five years, at least seven years, at least 10 years, or even more than 10 years).

In yet another aspect, the disclosure also features a method for delaying solid organ allograft failure in a subject having said solid organ allograft, the method comprising: administering to the subject an effective amount of an ENPP1 agent or an ENPP3 agent to thereby delay solid organ allograft failure in the subject. In some embodiments, the allograft failure can be delayed for at least two months (e.g., at least six months, at least one year, at least two years, at least three years, at least five years, at least seven years, at least 10 years, or even more than 10 years). In some embodiments, the subject has received or is receiving a therapy comprising a complement inhibitor. In some embodiments, the methods comprise administering to the subject a complement inhibitor.

In another aspect, the disclosure relates to a method for reducing and/or preventing stenosis or restenosis in the vasculature of a solid organ allograft of a subject having a solid organ allograft, the method comprising: administering to the subject an effective amount of: (i) an ENPP1 agent or an ENPP3 agent and (ii) a complement inhibitor to thereby reduce and/or prevent stenosis or restenosis in said vasculature of said solid organ allograft.

In another aspect, the disclosure relates to a method for reducing and/or preventing stenosis or restenosis in the vasculature of a solid organ allograft of a subject having a solid organ allograft, the method comprising: administering to the subject an effective amount of an ENPP1 agent or an ENPP3 agent and a complement inhibitor to thereby reduce and/or prevent stenosis or restenosis in said vasculature of said solid organ allograft. In some embodiments, the subject has received or is receiving a therapy comprising a complement inhibitor. In some embodiments, the methods comprise administering to the subject a complement inhibitor.

In yet another aspect, the disclosure also features a method for delaying or preventing or as prophylaxis against solid organ allograft rejection in a subject having said solid organ allograft, the method comprising: administering to the subject an effective amount of an: (i) ENPP1 agent or an ENPP3 agent and (ii) a complement inhibitor to thereby delay or prevent solid organ allograft rejection in the subject.

In yet another aspect, the disclosure also features a method for delaying or preventing or as prophylaxis against solid organ allograft rejection in a subject having said solid organ allograft, wherein the subject is receiving or has received a therapy comprising an ENPP1 agent or an ENPP3 agent, the method comprising: administering to the subject an effective amount of a complement inhibitor to thereby delay or prevent solid organ allograft rejection in the subject. In some embodiments, the method can also include administering to the subject the ENPP1 agent or ENPP3 agent.

In yet another aspect, the disclosure also features a method for delaying or preventing or as prophylaxis against rejection of an allografted vessel in a subject having said allografted vessel, the method comprising: administering to the subject an effective amount of an: (i) ENPP1 agent or an ENPP3 agent and (ii) a complement inhibitor to thereby delay or prevent rejection of said vessel in the subject. In some embodiments, the vessel is an artery. In some embodiments, the vessel is a vein.

In yet another aspect, the disclosure also features a method for delaying or preventing or as prophylaxis against rejection of an allografted vessel in a subject having said allografted vessel, wherein the subject is receiving or has received a therapy comprising an ENPP1 agent or an ENPP3 agent, the method comprising: administering to the subject an effective amount of a complement inhibitor to thereby delay or prevent rejection of the allografted vessel in the subject. In some embodiments, the method can also include administering to the subject the ENPP1 agent or ENPP3 agent. In some embodiments, the vessel is an artery. In some embodiments, the vessel is a vein.

In another aspect, the disclosure relates to a method for reducing and/or preventing progression of vascular smooth muscle cell proliferation in the vasculature of an allograft of a subject having said allograft, the method comprising administering to the subject an effective amount of an ENPP1 agent to thereby reduce and/or prevent progression of said vascular smooth muscle cell proliferation in said vasculature of said allograft of said subject.

In another aspect, the disclosure relates to a method for reducing and/or preventing stenosis or restenosis in the vasculature of a solid organ allograft of a subject having a solid organ allograft, the method comprising: administering to the subject an effective amount of an ENPP1 agent or ENPP3 agent to thereby reduce and/or prevent stenosis or restenosis in said solid organ allograft

In another aspect, the disclosure relates to a method for prolonging the survival of a solid organ allograft in a subject having a solid organ allograft, the method comprising administering to said subject an ENPP1 agent or ENPP3 agent in an amount sufficient to thereby prolong survival of said solid organ allograft in said subject

In another aspect, the disclosure relates to a method for inhibiting or preventing vasculopathy in a solid organ allograft of a subject having a solid organ allograft, the method comprising administering to said subject an ENPP1 agent or ENPP3 agent in an amount sufficient to inhibit or prevent vasculopathy in the solid organ allograft.

In another aspect, the disclosure relates to a method for inhibiting or preventing vasculopathy of an allografted blood vessel in a subject having a blood vessel allograft, the method comprising administering to a subject an ENPP1 agent or ENPP3 agent in an amount sufficient to prevent or inhibit vasculopathy of said allografted vessel.

In another aspect, the disclosure relates to a method for inhibiting or preventing vascular smooth muscle cell proliferation in an allografted blood vessel in a subject having a blood vessel allograft, the method comprising administering to said subject an ENPP1 agent or ENPP3 agent in an amount sufficient to prevent or inhibit vascular smooth muscle cell proliferation in said allografted vessel

In another aspect, the disclosure relates to a method for prolonging the survival of an allografted blood vessel in a subject having a blood vessel allograft, the method comprising administering to said subject an ENPP1 agent or ENPP3 agent in an amount sufficient to thereby prolong survival of said allografted blood vessel.

In another aspect, the disclosure also relates to a method for reducing and/or preventing progression of vascular smooth muscle cell proliferation in a solid organ transplant in a subject having a solid organ transplant and who undergoes surgery on said organ transplant, the method comprising administering to the subject an effective amount of an ENPP1 agent to thereby reduce and/or prevent progression of vascular smooth muscle cell proliferation in said solid organ transplant of said subject.

In another aspect, the disclosure also features a method for preventing or for prophylaxis against solid organ allograft failure in a subject having said solid organ allograft, the method comprising: administering to the subject an effective amount of an ENPP1 agent or an ENPP3 agent to thereby prevent or provide prophylaxis against solid organ allograft failure in the subject.

In another aspect, the disclosure also features a method for delaying solid organ allograft failure in a subject having said solid organ allograft, the method comprising: administering to the subject an effective amount of an ENPP1 agent or an ENPP3 agent to thereby delay solid organ allograft failure in the subject. In some embodiments, the allograft failure can be delayed for at least two months (e.g., at least six months, at least one year, at least two years, at least three years, at least five years, at least seven years, at least 10 years, or even more than 10 years).

In another aspect, the disclosure relates to a method for reducing and/or preventing vasculopathy of an allografted vessel in a subject, the method comprising: administering to the subject an effective amount of: (i) an ENPP1 agent or ENPP3 agent and (ii) a complement inhibitor to thereby reduce and/or prevent vasculopathy of the allografted vessel in said subject. In some embodiments, the vessel is an artery. In some embodiments, the vessel is a vein.

In another aspect, the disclosure relates to a method for reducing and/or preventing vasculopathy of an allografted vessel in a subject, the method comprising: administering to the subject an effective amount of an ENPP1 agent or ENPP3 agent to thereby reduce and/or prevent vasculopathy of the allografted vessel in said subject. In some embodiments, the vessel is an artery. In some embodiments, the vessel is a vein.

In another aspect, the disclosure relates to a method for reducing and/or preventing vasculopathy of an allografted vessel in a subject, the method comprising: administering to the subject an effective amount of an ENPP1 agent or ENPP3 agent to thereby reduce and/or prevent vasculopathy of the allografted vessel in said subject. In some embodiments, the vessel is an artery. In some embodiments, the vessel is a vein. In some embodiments, the subject has received or is receiving a therapy comprising a complement inhibitor. In some embodiments, the methods comprise administering to the subject a complement inhibitor.

In another aspect, the disclosure relates to a method for reducing and/or preventing progression of vascular smooth muscle cell proliferation in an allografted vessel in a subject, the method comprising: administering to the subject an effective amount of an ENPP1 agent or ENPP3 agent to thereby reduce and/or prevent progression of vascular smooth muscle cell proliferation in the allografted vessel in said subject. In some embodiments, the vessel is an artery. In some embodiments, the vessel is a vein. In some embodiments, the subject has received or is receiving a therapy comprising a complement inhibitor. In some embodiments, the methods comprise administering to the subject a complement inhibitor.

In another aspect, the disclosure relates to a method for reducing and/or preventing allograft vasculopathy (for example, cardiac allograft vasculopathy) in a subject having an allograft, the method comprising: administering to the subject an effective amount of: (i) an ENPP1 agent or ENPP3 agent and (ii) a complement inhibitor to thereby reduce and/or prevent allograft vasculopathy in said subject.

In yet another aspect, the disclosure relates to a method for reducing and/or preventing allograft vasculopathy (for example, cardiac allograft vasculopathy) in a subject having an allograft and who has received or is receiving a therapy comprising a complement inhibitor, the method comprising: administering to the subject an effective amount of an ENPP1 agent or ENPP3 agent to thereby reduce and/or prevent allograft vasculopathy in said subject. In some embodiments, the methods further comprise administering the complement inhibitor to the subject.

In some embodiments of any of the methods described herein, the agent is administered prior to, during and/or after said surgery.

In some embodiments of any of the methods described herein, the agent is administered prior to, during and/or after shunt placement.

In some embodiments of any of the methods described herein, wherein the surgery and/or shunt placement further comprises introduction into the subject of a dialysis catheter.

In some embodiments, any of the methods described herein can comprise administering to the subject one or more of an anticoagulant, an antibiotic, and an antihypertensive.

In some embodiments, any of the methods described herein can comprise monitoring the subject for an occlusion of the shunt, such as a thrombosis.

In some embodiments, any of the methods described herein further include administering to the patient one or more immunosuppressants.

In some embodiments of any of the methods described herein, the ENPP1 agent comprises ENPP1 variants that retain enzymatic activity.

In some embodiments of any of the methods described herein, the ENPP3 agent comprises ENPP3 variants that retain enzymatic activity.

In some embodiments of any of the methods described herein, the subject is one who is receiving or who has received one or more of an anticoagulant, an antibiotic, and an antihypertensive.

In some embodiments of any of the methods described herein, the subject has received and/or is receiving an immunosuppressive therapy in conjunction with the solid organ allograft transplantation, such as one or more immunosuppressants.

In some embodiments of any of the methods described herein, the subject has received and/or is receiving in conjunction with the solid organ allograft transplantation one or more of a statin drug, a vasodialator, an anticoagulant (e.g., aspirin), and an immunosuppressant.

In some embodiments, any of the methods described herein further include administering to the patient one or more of a statin drug, a vasodialator, an anticoagulant (e.g., aspirin), and an immunosuppressant.

In some embodiments, any of the methods described herein further include performing revascularization surgery on the solid organ allograft.

In some embodiments of any of the methods described herein, the subject is expected to undergo, has undergone, or is undergoing revascularization surgery on the solid organ allograft.

In some embodiments, the revascularization surgery comprises angioplasty, a bypass graft, and/or a stent placement.

In some embodiments of any of the methods described herein, the agent is administered prior to, during and/or after said surgery.

In some embodiments of any of the methods described herein, the surgery comprises balloon angioplasty and/or placement of a stent.

In some embodiments, the methods described herein further comprise performing the surgery.

In some embodiments of any of the methods described herein, the ENPP1 agent comprises an ENPP1 polypeptide.

In some embodiments of any of the methods described herein, the ENPP1 agent comprises a nucleic acid encoding an ENPP1 polypeptide.

In some embodiments of any of the methods described herein, the ENPP1 agent comprises a viral vector comprising a nucleic acid encoding an ENPP1 polypeptide.

In some embodiments of any of the methods described herein, the ENPP1 polypeptide comprises the extracellular domain of ENPP1.

In some embodiments of any of the methods described herein, the ENPP1 polypeptide comprises the catalytic domain of ENPP1.

In some embodiments of any of the methods described herein, the ENPP1 polypeptide comprises amino acids 99 to 925 of SEQ ID NO:1.

In some embodiments of any of the methods described herein, the ENPP1 polypeptide comprises a heterologous protein.

In some embodiments of any of the methods described herein, the heterologous protein increases the circulating half-life of the ENPP1 polypeptide in mammal.

In some embodiments of any of the methods described herein, the heterologous protein is an Fc region of an immunoglobulin molecule.

In some embodiments of any of the methods described herein, the immunoglobulin molecule is an IgG1 molecule.

In some embodiments of any of the methods described herein, the heterologous protein is an albumin molecule.

In some embodiments of any of the methods described herein, the heterologous protein is carboxy-terminal to the ENPP1 polypeptide.

In some embodiments of any of the methods described herein, ENPP1 agent comprises a linker.

In some embodiments of any of the methods described herein, the linker separates the ENPP1 polypeptide and the heterologous protein.

In some embodiments of any of the methods described herein, the linker comprises the following amino acid sequence: (GGGGS)ₙ, wherein n is an integer from 1 to 10.

In some embodiments of any of the methods described herein, the ENPP1 agent is administered to the subject subcutaneously.

In some embodiments of any of the methods described herein, the ENPP1 agent is administered to the subject intravenously.

In some embodiments of any of the methods described herein, the subject: is a tobacco user, has hypertension, has elevated cholesterol or triglyceride levels, is a diabetic, has renal disease, or is obese.

In some embodiments of any of the methods described herein, the subject has stage I, stage II or stage III, Suzuki grade MMD. In another aspect, the disclosure features a method for inhibiting or slowing progression of Stage I Suzuki grade MMD peripheral artery disease to Stage III Suzuki grade MMD in a subject, the method comprising: administering to the subject an effective amount of an ENPP3 agent to thereby inhibit and/or slow progression of Stage I Suzuki grade MMD to Stage III Suzuki grade MMD in said subject.

In another aspect, the disclosure features a method for reducing and/or preventing progression of vascular smooth muscle cell proliferation in a cerebral artery of a subject who requires surgery on said cerebral artery, wherein the subject has Moyamoya disease, the method comprising: administering to the subject an effective amount of an ENPP1 agent or an ENPP3 agent to thereby reduce and/or prevent progression of vascular smooth muscle cell proliferation in said cerebral artery at a surgical site of said cerebral artery in said subject.

In some embodiments of any of the methods described herein, the cerebral artery is one or more of an external carotid artery (ECA), an internal carotid artery (ICA), a middle cerebral artery (MCA) and an anterior cerebral artery (ACA).

In some embodiments of any of the methods described herein, the ENPP3 agent is administered prior to, during and/or after stent placement.

In some embodiments of any of the methods described herein, the solid organ allograft is a cardiac allograft.

In some embodiments of any of the methods described herein, the solid organ allograft is a lung allograft, a liver allograft, or a kidney allograft.

In some embodiments of any of the methods described herein, the complement inhibitor is a complement component C5 inhibitor, such as an anti-05 antibody, e.g., eculizumab or ravulizumab-cwvz.

In some embodiments, the complement inhibitor is an inhibitor of complement component C1 (including C1s and C1q), C2, C3, C4, C5, C6, C7, C8, and/or C9, such as an antibody that binds to and inhibits the function of any one of such complement components.

In some embodiments, the complement inhibitor is compstatin or an analog thereof.

In some embodiments, the complement inhibitor is a C5a inhibitor, a C5aR inhibitor, a C3 inhibitor, a Factor D inhibitor, a Factor B inhibitor, a C4 inhibitor, a C1q inhibitor, a C1s inhibitor, or any combination thereof.

In some embodiments of any of the methods described herein, the complement inhibitor is a lectin pathway inhibitor, such as an anti-MASP2 antibody (e.g., OMS721).

In another aspect, the disclosure relates to a method for reducing and/or preventing stenosis or restenosis in the vasculature of a solid organ allograft of a subject having a solid organ allograft, the method comprising: administering to the subject an effective amount of an ENPP1 agent to thereby reduce and/or prevent stenosis or restenosis in said vasculature of said solid organ allograft.

In another aspect, the disclosure relates to a method for reducing and/or preventing vasculopathy of an allograft in a subject having allograft vasculopathy, the method comprising administering to the subject an effective amount of an ENPP3 agent to thereby treat said allograft vasculopathy in said subject.

In another aspect, the disclosure relates to a method for reducing and/or preventing progression of vascular smooth muscle cell proliferation in the vasculature of an allograft of a subject having said allograft, the method comprising administering to the subject an effective amount of an ENPP3 agent to thereby reduce and/or prevent progression of said vascular smooth muscle cell proliferation in said vasculature of said allograft of said subject.

In another aspect, the disclosure also relates to a method for reducing and/or preventing progression of vascular smooth muscle cell proliferation in a solid organ transplant in a subject having a solid organ transplant and who undergoes surgery on said organ transplant, the method comprising administering to the subject an effective amount of an ENPP3 agent to thereby reduce and/or prevent progression of vascular smooth muscle cell proliferation in said solid organ transplant of said subject.

In some embodiments of any of the methods described herein, the agent is administered prior to, during and/or after said surgery.

In some embodiments of any of the methods described herein, the surgery comprises balloon angioplasty and/or placement of a stent.

In some embodiments of any of the methods described herein, the subject does not have a deficiency of ENPP 1.

In some embodiments of any of the methods described herein, the ENPP3 agent comprises an ENPP3 polypeptide.

In some embodiments of any of the methods described herein, the ENPP3 agent comprises a nucleic acid encoding an ENPP3 polypeptide.

In some embodiments of any of the methods described herein, the ENPP3 agent comprises a viral vector comprising a nucleic acid encoding an ENPP3 polypeptide.

In some embodiments of any of the methods described herein, the ENPP3 polypeptide comprises the extracellular domain of ENPP3.

In some embodiments of any of the methods described herein, the ENPP3 polypeptide comprises the catalytic domain of ENPP3.

In some embodiments of any of the methods described herein, the ENPP3 polypeptide comprises amino acids 49 to 875 of SEQ ID NO:7

In some embodiments of any of the methods described herein, the ENPP3 polypeptide comprises a heterologous protein.

In some embodiments of any of the methods described herein, the heterologous protein increases the circulating half-life of the ENPP3 polypeptide in mammal.

In some embodiments of any of the methods described herein, the heterologous protein is an Fc region of an immunoglobulin molecule.

In some embodiments of any of the methods described herein, the immunoglobulin molecule is an IgG1 molecule.

In some embodiments of any of the methods described herein, the heterologous protein is an albumin molecule.

In some embodiments of any of the methods described herein, the heterologous protein is carboxy-terminal to the ENPP3 polypeptide.

In some embodiments of any of the methods described herein, the ENPP3 agent comprises a linker.

In some embodiments of any of the methods described herein, the linker separates the ENPP3 polypeptide and the heterologous protein.

In some embodiments of any of the methods described herein, the linker comprises the following amino acid sequence: (GGGGS)n, wherein n is an integer from 1 to 10.

In some embodiments of any of the methods described herein, the ENPP3 agent is administered to the subject subcutaneously.

In some embodiments of any of the methods described herein, the ENPP3 agent is administered to the subject intravenously.

In some embodiments of any of the methods described herein, the subject: is a tobacco user, has hypertension, has elevated cholesterol or triglyceride levels, is a diabetic, has renal disease, or is obese. In some embodiments of any of the methods describes herein, the subject has cerebral arterial occlusions.

In another aspect, the disclosure relates to a method for reducing and/or preventing stenosis or restenosis in the vasculature of a solid organ allograft of a subject having a solid organ allograft, the method comprising: administering to the subject an effective amount of an ENPP3 agent to thereby reduce and/or prevent stenosis or restenosis in said solid organ allograft.

Other features and advantages of the disclosure will be apparent from the following detailed description and claims.

### BRIEF DESCRIPTION OF THE DRAWINGS

FIG. 1 shows the schematic diagram of prophylactic treatment regimen of control and experimental mice prior and after transplant. The experimental mice are treated 7 days prior to aortic transplantation with ENPP1-Fc at an exemplary dosage of 10 mg/kg weight by subcutaneous injection every day. The control cohorts are injected with vehicle containing tris buffered saline, at pH 7.4. All mice are then dissected at 28 days after transplantation and the mice are approximately 10 weeks of age.

FIG. 2 shows a schematic diagram of heart transplant in mouse. It also shows morphometrical measurements of 5 μm sections of the transplanted aorta. The medial area, the intimal area and the intima/media ratio (I/M ratio) of each section are calculated.

FIG. 3 shows a schematic version of Porcine model of heterotopic heart transplantation. 3 (A) shows the donor heart is harvested after cardiac standstill achieved by using cold cardioplegic solution (Plegisol). 3(B) shows that the graft is maintained in the ice-saline slurry and prepared for implantation by creating an atrial septal defect and defunction the mitral valve to minimize left ventricular atrophy and intracavity thrombus formation. 3(C) shows the recipient's inferior vena cava (IVC) and the infrarenal aorta were isolated. 3(D) shows the graft heart is implanted by anastomosing the donor pulmonary artery to the recipient's IVC and the donor ascending aorta to the abdominal aorta of the recipient. Graft function was monitored by using (E) electrocardiography (ECG) and (F) echocardiography (UCG). Arrows indicate electrical spikes attributed to heterotopic cardiac allograft. (Hsu et al., Transplantation. 2018 December; 102(12): 2002-2011.)

FIG. 4 is a series of photographs of representative profunda artery images captured by angiography at day 14 and day 42 post stent implantation. The two control images illustrate a narrowing of the profunda due to intimal proliferation at day 42 relative to the morphology of the vessel at day 14. By contrast, in animals treated with ENPP1-Fc little visible change in profunda morphology was observed between day 14 and day 42. The upper and lower boundary of the stent within the vessel is identified in each photograph by rectangles.

FIG. 5 is a series of photographs of representative profunda artery images captured by Optical Coherence Tomography (OCT) at day 14 and day 42 post stent implantation. The two control images illustrate a pronounced intimal thickening within the profunda at day 42 relative to the morphology of the vessel at day 14. By contrast, in animals treated with ENPP1-Fc little visible intimal thickening was observed between day 14 and day 42. The extent of stenosis is highlighted in the day 42 photographs.

FIG. 6 is a bar graph depicting the percent area of stenosis at day 14 and day 42 in the profunda of pigs treated with ENPP1-Fc (Treatment) or given vehicle control (Control), as measured by OCT.

FIG. 7 shows the schematic diagram of prophylactic treatment regimen of control and experimental mice prior and after brain surgery to induce MMD. The experimental mice are treated 7 days prior to surgery with ENPP1-Fc at an exemplary dosage of 10 mg/kg weight by subcutaneous injection every day. The control cohorts are injected with vehicle containing tris buffered saline, at pH 7.4. All mice are then dissected at 28 days after transplantation and the mice are approximately 10 weeks of age.

FIG. 8 shows the process of creating MMD model by Internal Carotid Artery Stenosis. 8A) shows orientation of the mouse during the surgical procedure. Head (teeth), forepaws and tail are restrained, and incision is made in the midline of the neck (red dashed line). White box indicates region of images that follow. 8B) shows opening of the cervical region exposing the trachea, sternocleidomastoid (SCM) muscle and posterior belly of the digastric (PBD) muscle. 8C) shows suture (S1-2) placement retracting the SCM and PBD to expose the common, internal and external carotid (CCA, ICA, ECA) arteries. 8D) shows Identification of the occipital artery (OA), vagus nerve (VN) and ICA. 8E) shows suture ligation of the OA and dashed line showing cut to better expose the ICA. 8F) shows cut OA with ICA exposed and isolated using 6±0 suture. 8G) shows micro-coil placement on ICA deep to ECA (as seen in H). (Roberts et al., Internal carotid artery stenosis: A novel surgical model for moyamoya syndrome, PLoS One. 2018; 13(1): e0191312.)

FIG. 9 is a diagram of hemodialysis blood flow from a subject's arm, which contains a dialysis shunt, into a tube, past a pressure monitor, a blood pump, and a heparin pump, which prevents clotting. Blood flows past another pressure monitor before entering the dialyzer, or filter. Filtered blood continues past a venous pressure monitor, an air trap and air detector, and an air detector clamp, and returns to the subject's arm.

FIG. 10 is a view of an implantable shunt 2 positioned in the upper right chest area 100 of a subject. The implantable dialysis shunt 2 may also be implanted into other areas of the body, so long as it is implanted in reasonable proximity to a medium sized artery, typically between 6 and 8 mm, for use with the implantable dialysis shunt 2. The implantable dialysis shunt preferably comprises an arterial port 4 and a venous port 6 connected to each other in a single structure. In other embodiments, the ports 4, 6 may be separate structures which may include features to permit their attachment to each other. An arterial graft 12 generally extends through the arterial port 4 while a venous graft 18 extends from the venous port 6. During the implantation process, the arterial graft 12 is preferably connected at each of its ends to the sidewall of an artery 26 while the end of the venous graft 18 is connected to a vein 34. In other embodiments, the arterial graft 12 may be connected to the artery 26 by a pair of end-to-end anasomoses. Additionally, the venous graft 18 may take the form of a venous catheter which is inserted into the vein 34 such that it may enter the central venous system. Dialysis may be conducted by tapping the arterial port 4 with an arterial catheter 102 and the venous port with a venous catheter 104. Each of the arterial and venous catheters 102, 104 are connected to a dialysis machine.

### DETAILED DESCRIPTION

### Definitions

Unless defined otherwise, all technical and scientific terms used herein have the same meaning as commonly understood by one of ordinary skill in the art to which this disclosure belongs. Although any methods and materials similar or equivalent to those described herein can be used in practice or testing of the present disclosure, the preferred methods and materials are described.

For clarity, “NPP1” and “ENPP1” refer to the same protein and are used interchangeably herein. As used herein, the term “ENPP1 protein” or “ENPP1 polypeptide” refers to ectonucleotide pyrophosphatase/phosphodiesterase-1 protein encoded by the ENPP1 gene that is capable of cleaving ATP to generate PPi and also reduces ectopic calcification in soft tissue.

ENPP1 protein is a type II transmembrane glycoprotein and cleaves a variety of substrates, including phosphodiester bonds of nucleotides and nucleotide sugars and pyrophosphate bonds of nucleotides and nucleotide sugars. ENPP1 protein has a transmembrane domain and soluble extracellular domain. The extracellular domain is further subdivided into somatomedin B domain, catalytic domain and the nuclease domain. The sequence and structure of wild-type ENPP1 is described in detail in PCT Application Publication No. WO 2014/126965 to Braddock, et al., which is incorporated herein in its entirety by reference.

ENPP1 polypeptides as used herein encompasses polypeptides that exhibit ENPP1 enzymatic activity, mutants of ENPP1 that retain ENPP1 enzymatic activity, fragments of ENPP1 or variants of ENPP1 including deletion variants that exhibit ENPP1 enzymatic activity. ENPP1 enzymatic activity refers to the ability of the ENPP1 polypeptide to cleave Adenosine Triphosphate (ATP) into plasma pyrophosphate (PPi), as noted below.

ENPP3 polypeptides as used herein encompasses polypeptides that exhibit ATP cleavage enzymatic activity, mutants of ENPP3 that retain ATP cleavage enzymatic activity, fragments of ENPP3 or variants of ENPP3 including deletion variants that exhibit ATP cleavage enzymatic activity. ATP cleavage enzymatic activity refers to the ability of the ENPP3 polypeptide to cleave Adenosine Triphosphate (ATP) into plasma pyrophosphate (PPi), as noted below.

Some examples of ENPP1 and ENPP3 polypeptides, mutants, or mutant fragments thereof, have been previously disclosed in International PCT Application Publications No. WO/2014/126965—Braddock et al., WO/2016/187408-Braddock et al., WO/2017/087936-Braddock et al., and WO2018/027024-Braddock et al., all of which are incorporated by reference in their entireties herein.

Enzymatically active” with respect to an ENPP1 polypeptide or an ENPP3 polypeptide is defined as possessing ATP hydrolytic activity into AMP and PPi and/or AP3a hydrolysis to ADP and AMP. NPP1 and NPP3 readily hydrolyze ATP into AMP and PPi. The steady-state Michaelis-Menten enzymatic constants of NPP1 are determined using ATP as a substrate. NPP1 can be demonstrated to cleave ATP by HPLC analysis of the enzymatic reaction, and the identity of the substrates and products of the reaction are confirmed by using ATP, AMP, and ADP standards. The ATP substrate degrades over time in the presence of NPP1, with the accumulation of the enzymatic product AMP. Using varying concentrations of ATP substrate, the initial rate velocities for NPP1 are derived in the presence of ATP, and the data is fit to a curve to derive the enzymatic rate constants. At physiologic pH, the kinetic rate constants of NPP1 are Km=144 μM and kcatₜ=7.8 s⁻¹.

As used herein, the term “ENPP1 precursor protein” refers to ENPP1 with its signal peptide sequence at the ENPP1 N-terminus. Upon proteolysis, the signal sequence is cleaved from ENPP1 to provide the ENPP1 protein. Signal peptide sequences useful within the disclosure include, but are not limited to, Albumin signal sequence, Azurocidin signal sequence, ENPP1 signal peptide sequence, ENPP2 signal peptide sequence, ENPP7 signal peptide sequence, and/or ENPP5 signal peptide sequence.

As used herein, the term “ENPP3 precursor protein” refers to ENPP3 with its signal peptide sequence at the ENPP3 N-terminus. Upon proteolysis, the signal sequence is cleaved from ENPP3 to provide the ENPP3 protein. Signal peptide sequences useful within the disclosure include, but are not limited to, Albumin signal peptide sequence, Azurocidin signal peptide sequence, ENPP1 signal peptide sequence, ENPP2 signal peptide sequence, ENPP7 signal peptide sequence, and/or ENPP5 signal peptide sequence.

As used herein, the term “Azurocidin signal peptide sequence” refers to the signal peptide derived from human azurocidin. Azurocidin, also known as cationic antimicrobial protein CAP37 or heparin-binding protein (HBP), is a protein that in humans is encoded by the AZU1 gene. The nucleotide sequence encoding Azurocin signal peptide MTRLTVLALLAGLLASSRA (SEQ ID NO: 42) is fused with the nucleotide sequence of NPP1 or NPP3 gene which when encoded generates ENPP1 precursor protein or ENPP3 precursor protein. (Optimized signal peptides for the development of high expressing CHO cell lines, Kober et al., Biotechnol Bioeng. 2013 April; 110(4):1164-73)

As used herein, the term “ENPP1-Fc construct” refers to ENPP1 (e.g., the extracellular domain of ENPP1) recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “ENPP3-Fc construct” refers to ENPP3 recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “Fc” refers to a human IgG (immunoglobulin) Fc domain. Subtypes of IgG such as IgG1, IgG2, IgG3, and IgG4 are contemplated for use as Fc domains. The “Fc region or Fe polypeptide” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CHL CH2 and CH3 domains of the heavy chain and the CHL domain of the light chain.

As used herein the term “functional equivalent variant”, as used herein, relates to a polypeptide substantially homologous to the sequences of ENPP1 or ENPP3 (defined above) and that preserves the enzymatic and biological activities of ENPP1 or ENPP3, respectively. Methods for determining whether a variant preserves the biological activity of the native ENPP1 or ENPP3 are widely known to the skilled person and include any of the assays used in the experimental part of said application. Particularly, functionally equivalent variants of ENPP1 or ENPP3 delivered by viral vectors is encompassed by the present disclosure.

The functionally equivalent variants of ENPP1 or ENPP3 are polypeptides substantially homologous to the native ENPP1 or ENPP3 respectively. The expression “substantially homologous”, relates to a protein sequence when said protein sequence has a degree of identity with respect to the ENPP1 or ENPP3 sequences described above of at least 80%, at least 85%, at least 90%, at least 91%, at least 92%, at least 93%, at least 94%, at least 95%, at least 96%, at least 97%, at least 98% or at least 99% respectively and still retaining at least 50%, 55%, 60%, 70%, 80% or 90% activity of wild type ENPP1 or ENPP3 protein with respect to ATP cleavage.

The degree of identity between two polypeptides is determined using computer algorithms and methods that are widely known for the persons skilled in the art. The identity between two amino acid sequences is preferably determined by using the BLASTP algorithm (BLAST Manual, Altschul, S., et al., NCBI NLM NIH Bethesda, Md. 20894, Altschul, S., et al., J. Mol. Biol. 215: 403-410 (1990)), though other similar algorithms can also be used. BLAST and BLAST 2.0 are used, with the parameters described herein, to determine percent sequence identity. Software for performing BLAST analyses is publicly available through the National Center for Biotechnology Information.

Functionally equivalent variants of ENPP1 or ENPP3 may be obtained by replacing nucleotides within the polynucleotide accounting for codon preference in the host cell that is to be used to produce the ENPP1 or ENPP3 respectively. Such “codon optimization” can be determined via computer algorithms which incorporate codon frequency tables such as “Human high.cod” for codon preference as provided by the University of Wisconsin Package Version 9.0, Genetics Computer Group, Madison, Wis. The variants of ENPP1 or ENPP3 polypeptides are expected to retain at least 50%, 55%, 60%, 70%, 80% or 90% activity of wild type ENPP1 or ENPP3 protein with respect to ATP cleavage.

As used herein the term “ENPP1 fragment” refers to a fragment or a portion of ENPP1 protein or an active subsequence of the full-length NPP1 having at least an ENPP1 catalytic domain administered in protein form or in the form of a nucleic acid encoding the same.

As used herein, the term “ENPP1 agent” refers to ENPP1 polypeptide or fusion protein or ENPP1 fragment comprising at least catalytic domain capable of producing plasma pyrophosphate (Ppi) by cleavage of adenosine triphosphate (ATP) or a polynucleotide such as cDNA or RNA encoding ENPP1 fusion protein or ENPP1 fragment comprising at least catalytic domain capable of producing PPi by enzymatic cleavage of ATP or a vector such as a viral vector containing a polynucleotide encoding the same.

As used herein, the term “wild-type” refers to a gene or gene product isolated from a naturally occurring source. A wild-type gene is most frequently observed in a population and is thus arbitrarily designed the “normal” or “wild-type” form of the human NPP1 or NPP3 genes. In contrast, the term “functionally equivalent” refers to a NPP1 or NPP3 gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

As defined herein, the term “moiety” refers to a chemical component or biological molecule that can be covalently or non-covalently linked to ENPP1 or ENPP3 polypeptide and has the ability to confer a desired property to the protein to which it is attached. For example, the term moiety can refer to a bone targeting peptide such as polyaspartic acid or polyglutamic acid (of 4-20 consecutive asp or glu residues) or a molecule that extends the half-life of ENPP1 or ENPP3 polypeptide. Some other examples of moieties include Fc, albumin, transferrin, polyethylene glycol (PEG), homo-amino acid polymer (HAP), proline-alanine-serine polymer (PAS), elastin-like peptide (ELP), and gelatin-like protein (GLK).

As defined herein, the term “subject”, “individual” or “patient” refers to mammal preferably a human who does not possess a loss of function mutation in the NPP1 gene, such as those loss of function mutations that result in pathological calcification and pathological ossification diseases such as Generalized Arterial Calcification of Infancy (GACI), Autosomal Recessive Hypophosphatemic Rickets Type 2 (ARHR2), Infantile idiopathic arterial calcification (IIAC), Ossification of the Posterior Longitudinal Ligament (OPLL), hypophosphatemic rickets, osteoarthritis, calcification of atherosclerotic plaques, hereditary and non-hereditary forms of osteoarthritis, ankylosing spondylitis, hardening of the arteries occurring with aging, calciphylaxis resulting from end stage renal disease and progeria. Such a patient will have a normal level of NPP1 in serum which refers to the amount of NPP1 required to maintain a normal level of plasma pyrophosphate (PPi) in a healthy subject. A normal level of PPi corresponds to 2-3 μM.

As used herein the term “plasma pyrophosphate (PPi) levels” refers to the amount of pyrophosphate present in plasma of animals. In certain embodiments, animals include rat, mouse, cat, dog, human, cow and horse. There are several ways to measure PPi, one of which is by enzymatic assay using uridine-diphosphoglucose (UDPG) pyrophosphorylase (Lust & Seegmiller, 1976, Clin. Chim. Acta 66:241-249; Cheung & Suhadolnik, 1977, Anal. Biochem. 83:61-63) with modifications.

Typically, plasma PPi levels in healthy human subjects range from about 1 μm to about 3 μM, in some cases between 1-2 μm. Subjects who have defective ENPP1 expression tend to exhibit low ppi levels which range from at least 10% below normal levels, at least 20% below normal levels, at least 30% below normal levels, at least 40% below normal levels, at least 50% below normal levels, at least 60% below normal levels, at least 70% below normal levels, at least 80% below normal levels and combinations thereof. In patients afflicted with Generalized Arterial Calcification of Infancy (GACI), the ppi levels are found to be less than 1 μm and in some cases are below the level of detection. In patients afflicted with Pseudoxanthoma Elasticum (PXE), the ppi levels are below 0.5 μm. (Arterioscler Thromb Vasc Biol. 2014 September; 34(9):1985-9; Braddock et al., Nat Commun. 2015; 6: 10006.)

As used herein, the term “PPi” refers to inorganic pyrophosphate.

A “low level of PPi” refers to a condition in which the subject has at least 0.1%-0.99% less than 2%-5% of normal levels of plasma pyrophosphate (PPi). Normal levels of Plasma PPi in healthy human subjects are in the range of 1.8 to 2.6 μM.+/−0.1 μM (Arthritis and Rheumatism, Vol. 22, No. 8 (August 1979))

As used herein the term “non-surgical tissue injury” refers to injuries sustained to a tissue or blood vessel during a traumatic event including but not limited to physical altercations involving use of blunt force or sharp objects such as knife, mechanical injury such fall from elevation, workplace injury due to heavy machinery or vehicular injury such as car accidents.

As used herein, the term “myocardial infarction” refers to a permanent damage to the heart muscle that occurs due to the formation of plaques in the interior walls of the arteries resulting in reduced blood flow to the heart and injuring heart muscles because of lack of oxygen supply. The symptoms of MI include chest pain, which travels from left arm to neck, shortness of breath, sweating, nausea, vomiting, abnormal heart beating, anxiety, fatigue, weakness, stress, depression, and other factors.

As used herein, the term “moyamoya disease” or “moyamoya syndrome” refers to a steno-occlusive disease of the cerebral arteries, involving smooth muscle cell proliferation with intima hyperplasia causing arterial stenosis and occlusion around the circle of Willis. It involves development of new blood vessels resembling a “puff of smoke” (“moyamoya”) in the subcortical region. MMD occurs in children and adults with two peaks—at around age 5-10 and a second peak between the third and fifth decade of life. Common symptoms include headache or dizziness, weakness or paralysis in a limb or on one side of the body, problems with speech inability to speak or recall words, sensory or cognitive impairment, involuntary movements, seizures or loss of consciousness, vision problems, stroke, and cerebral hemorrhage. 80% of MMD cases are carriers of RNF213 and or R4810K mutations. Treatment options for both MMD and MMS involve daily aspirin use, lifestyle modifications to maximize cerebral perfusion, and surgical direct or indirect bypass to restore blood flow.

Diagnostic criteria for definitive MMD were revised to include patients with both bilateral and unilateral presentation of terminal carotid artery stenosis (ICA) with an abnormal vascular network at the base of the brain. Suzuki system of grading the patient population has been used for MMD. Definitive diagnosis of MMD requires catheter angiography in unilateral cases, whereas bilateral cases can be promptly diagnosed by either catheter angiography or magnetic resonance imaging/angiography (MRI/MRA).

As used herein, the phrase “cerebral vascular occlusion” refers to the temporary or permanent blockage of blood vessels in the brain. Restrictions in blood flow may occur from vessel narrowing (stenosis), clot formation (thrombosis), blockage (embolism) or blood vessel rupture (hemorrhage). Lack of sufficient blood flow (ischemia) affects brain tissue and may cause a stroke.

As used herein the term “Suzuki classification System” refers to classification system developed by Suzuki et al. (Suzuki J, Takaku A. Cerebrovascular “moyamoya” disease. Disease showing abnormal net-like vessels in base of brain. Arch Neurol. 1969; 20(3):288±99.). This classification system grades the clinical presentation of patients to four stages. The vast majority of patients will progress through some or all of the Suzuki stages, although progression may occur at different rates, and appears to occur more rapidly in children than in adolescents or adults. The system is solely based on conventional angiography and is as shown in table below.

| GRADE | SYMPTOMS |
|---|---|
| Stage I | Narrowing of the carotid fork; narrowed Internal |
| | carotid artery (ICA) bifurcation |
| Stage II | Initiation of the Moyamoya; dilated Anterior cerebral |
| | artery (ACA), Middle Cerebral artery (MCA) and narrowed |
| | ICA bifurcation with Moyamoya change |
| Stage III | Intensification of the Moyamoya, further increase in |
| | Moyamoya change of the ICA bifurcation and narrowed ACA |
| | and MCA |
| Stage IV | Minimization of the Moyamoya, Moyamoya change |
| | reducing with occlusive changes in ICA and tenuous ACA and |
| | MCA |
| Stage V | Reduction of the Moyamoya, further decrease in |
| | Moyamoya change with occlusion of ICA, ACA and MCA |
| Stage VI | Disappearance of the Moyamoya, ICA essentially |
| | disappeared with supply of brain from External Carotid artery |
| | (ECA) |

As used herein, the term “internal carotid artery (ICA)” refers to the artery that is located in the inner side of the neck and supplies blood to the brain and eyes.

As used herein, the term “external carotid artery (ECA)” refers to a major artery of the head and neck. It arises from the common carotid artery when it splits into the external and internal carotid artery. ECA supplies blood to face, scalp, skull, and meninges. As used herein, the term “anterior Cerebral Artery (ACA)” refers is an artery on the brain that supplies oxygenated blood to most midline portions of the frontal lobes and superior medial parietal lobes. A pair of anterior cerebral arteries arise from the internal carotid artery and are part of the circle of Willis.

As used herein, the term “medial cerebral artery (MCA)” refers to is one of the three major paired arteries that supply blood to the cerebrum. The MCA arises from the internal carotid and continues into the lateral sulcus where it then branches and projects to many parts of the lateral cerebral cortex. It also supplies blood to the anterior temporal lobes and the insular cortices.

As used herein, the term “conventional angiography” refers to Angiography or arteriography is a medical imaging technique used to visualize the inside, or lumen, of blood vessels and organs of the body, with particular interest in the arteries, veins, and the heart chambers. This is traditionally done by injecting a radio-opaque contrast agent into the blood vessel and imaging using X-ray based techniques such as fluoroscopy.

As used herein, the term “catheter angiography” refers to a medical procedure wherein a catheter, x-ray imaging guidance and an injection of contrast material to examine blood vessels in key areas of the body such as brain or heart for abnormalities such as aneurysms and disease such as atherosclerosis (plaque).

As used herein, the phrase “magnetic resonance angiography (MRA)” refers to a medical process wherein magnetic resonance imaging scanner is used to visualize the blockages in the blood vessels of critical regions such as brain, lungs and heart with aid of a contrast agent administered with an intravenous needle. It is non-invasive method to diagnose blockages or occlusions in the blood vessels.

As used herein, the term “subject who requires surgery” refers to a patient who is not ENPP1 deficient and has arterial occlusion in the peripheral arteries such as femoral, femoropopliteal or tibial-peroneal arteries.

As used herein, the term “site of surgery” refers to the region of the artery upon which a tissue injury has occurred either due to vascular trauma or accidental trauma.

As used herein, the term “brain calcification” (BC) refers to a nonspecific neuropathology wherein deposition of calcium and other mineral in blood vessel walls and tissue parenchyma occurs leading to neuronal death and gliosis. Brain calcification is” often associated with various chronic and acute brain disorders including Down's syndrome, Lewy body disease, Alzheimer's disease, Parkinson's disease, vascular dementia, brain tumors, and various endocrinologic conditions Calcification of heart tissue refers to accumulation of deposits of calcium (possibly including other minerals) in tissues of the heart, such as aorta tissue and coronary tissue.

As used herein with respect to use of a dialysis shunt, stenosis slows and reduces blood flow through an AV fistula, causing problems with the quality of dialysis treatment, prolonged bleeding after puncture, or pain in the fistula. Stenosis can also lead to a blocked or clotted access.

As used herein the term “scapel incision” refers to incision made in a tissue using a sharp object such as a scapel during surgical procedure. An incision is a cut made into the tissues of the body to expose the underlying tissue, bone, or organ so that a surgical procedure can be performed.

As used herein, the term “site of surgery” refers to the region of the artery upon which a tissue injury has occurred either due to vascular trauma or accidental trauma.

The term “arterio-venous shunt” or “AV shunt” or simply “shunt” refers to an implanted device which includes a tube to which an artery and vein is attached. A shunt connects the arterial and venous cannulas and provides a larger than normal volume of blood flow for effective hemodialysis. A shunt can be located in any part of the body, and is most often located in an arm, a leg or the chest area below the right collarbone.

As used herein, the term “coated shunt” refers to shunts that are capable of slowly eluting therapeutic compounds or polypeptides such as ENPP1 or ENPP3 to reduce the amount of vascular smooth muscle cell proliferation at the site of surgery, typically performed to remove blockage of the arteries.

As used herein, the term “hemodialysis” refers to a treatment that is required to compensate for abnormal kidney function, in which wastes and water are filtered out of blood and the filtered cleaner blood is returned to the body. Hemodialysis helps control blood pressure and balance important minerals, such as potassium, sodium, and calcium, in a subject's blood.

As used herein, the term “fistula” refers to an abnormal or surgically made passage between a hollow or tubular organ and the body surface, or between two hollow or tubular organs.

The term “stent” refers to a tubular support placed inside a blood vessel, canal, or duct to aid healing or relieve an obstruction.

The term “vessel” refers to a tubular structure carrying blood through the tissues and organs; a vein, artery, or capillary.

As used herein, the term “complement inhibitor” refer to a molecule (e.g., a protein (such as an antibody), a small molecule, or a peptide) that prevents or reduces activation and/or propagation of the complement cascade that results in the formation of C3a or signaling through the C3a receptor, C5a or signaling through the C5a receptor, or formation of terminal complement. Complement inhibitors are well known in the art and described in, e.g., Zipfel et al. (2019) Front Immunol 10:2166. See also, e.g., U.S. Pat. No. 5,679,345, the disclosure of which is incorporated by reference in its entirety.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

As defined herein, the phrase “medial area” is the area between lamina elastica externa and lamina elastica interna of an artery.

As defined herein, the phrase “intimal area” and said intimal area is the area between said lamina elastica interna and lumen of an artery.

As defined herein, the phrase “lamina elastica externa” refers to a layer of elastic connective tissue lying immediately outside the smooth muscle of the tunica media of an artery.

As defined herein, the phrase “lamina elastica interna” refers to a layer of elastic tissue that forms the outermost part of the tunica intima of blood vessels.

As defined herein, the phrase “lumen” refers to the interior of a vessel, such as the central space in an artery, vein or capillary through which blood flow occurs.

As defined herein, the phrase “vasculopathy” refers to disease of the vasculature. “Vasculature” refers to the arrangement of blood vessels in the body or in an organ, such as a solid organ transplant, or in a body part. A “blood vessel” refers to one or more of an artery, arteriole, capillary and vein in the body of a subject or of a solid organ allograft of a subject. “Vasculitis” refers to inflammation of veins, arteries, capillaries, or lymph vessels. A “vascularized graft” refers to a graft after the recipient vasculature has been connected with the vessels in the graft.

As defined herein, the phrase “cardiac allograft vasculopathy (CAV)” refers to a vascular complication of allograft or solid organ transplantation such as heart wherein the blood vessels supplying the transplanted heart gradually narrow and restrict its blood flow, subsequently leading to impairment of the heart muscle or sudden death. Diagnosis of CAV is by regular follow-up and monitoring of the transplanted organ such as heart for early signs of disease. This involves invasive diagnostics including coronary angiography and intravascular ultrasound, and non-invasive investigations including dobutamine stress echocardiography, positron emission tomography, computed tomographic angiography (CT angiography) and the levels of a variety of biomarkers such as C-reactive protein, serum brain natriuretic peptide, troponin and serum microRNA 628-5p.

As defined herein, “allograft” refers to the transplant of an organ or tissue from a donor to a recipient of the same species. Allografts account for many human organ and tissue transplants, including those from cadaveric, living related, and living unrelated donors.

As defined herein, a “solid organ allograft” refers to an allograft of a solid organ. A “solid organ” is an internal organ that has a firm tissue consistency and is neither hollow (such as the organs of the gastrointestinal tract) nor liquid (such as blood). A solid organ includes but is not limited to kidney, liver, cornea, intestines, heart, lung and pancreas.

As defined herein, the phrase “graft rejection” or “transplant rejection” refers to a condition wherein the transplanted organ or tissue is rejected by the recipient's immune system, which destroys the allograft and results in long-term loss of function in transplanted organs via fibrosis of the transplanted tissue blood vessels.

As defined herein, the phrase “prolonging the survival of an allograft” refers to the prevention of rejection of a transplanted donor organ or tissue by the recipient immune system and to improve the lifespan of the transplanted organ. Survival of an allograft may be prolonged by at least 12 months, 18 months, 2 years, 3 years, 4 years, 5 years, 8 years, 10 years or longer relative to allograft survival absent treatment.

As defined herein, the phrase “heart allograft” refers to a solid organ transplant involving a donor heart transplanted into a recipient or grafting of one or more donor arteries or veins into a recipient's heart. Graft rejection in heart allografts is commonly diagnosed by performing Endomyocardial biopsy.

As defined herein, the phrase “kidney allograft” refers to a solid organ transplant involving a donor kidney transplanted into a recipient or grafting of one or more donor arteries or veins into a recipient's kidney. Graft rejection in kidney allografts is commonly diagnosed by monitoring Urine protein levels such total protein-to-creatinine ratio, albumin-to-creatinine ratio, serum creatinine level and glomerular filtration rate.

As defined herein, the phrase “liver allograft” refers to a solid organ transplant involving a donor liver transplanted into a recipient or grafting of one or more donor arteries or veins into a recipient's liver. Graft rejection in liver allografts is diagnosed by monitoring Transaminase, bilirubin, and alkaline phosphatase levels.

As defined herein, the phrase “lung allograft” refers to refers to a solid organ transplant involving a donor lung transplanted into a recipient or grafting of one or more donor arteries or veins into a recipient's lung. Graft rejection in lung allografts is diagnosed by bronchoscopy with transbronchial biopsies and pulmonary function testing.

As defined herein, the phrase “allografted vessel” or “Allografted vasculature” refers to the grafting of one or more donor blood vessels such as artery, vein, capillary and/or arteriole into the recipient.

As defined herein, the phrase “allografted artery” refers to the grafting of one or more donor arteries into the recipient.

As defined herein, the phrase “allografted vein” refers to the grafting of one or more donor veins into the recipient.

As defined herein, the phrase “endomyocardial biopsy” refers to a procedure that percutaneously obtains small amounts of myocardial tissue for diagnostic, therapeutic, and research purposes. It is primarily used to (1) follow the transplanted heart for myocardial rejection; (2) diagnose specific inflammatory, infiltrative, or familial myocardial disorders; and (3) sample unknown myocardial masses.

As defined herein, the phrase “transbronchial lung biopsy” refers to a biopsy from the lung obtained by endoscopically-guided forceps, which is useful in evaluating lesions in the transplant distributed along bronchovascular bundles and in the central lung zones.

As defined herein, the phrase “surgery” refers to an invasive medical procedure that involves vascular interventions which result in tissue injury by scapel incision or radiofrequency ablation or cryoablation or laser ablation.

As defined herein, the phrase “tissue injury” refers to proliferation or onset of proliferation and migration of vascular smooth muscle eventually resulting in the thickening of arterial walls and decreased arterial lumen space resulting restenosis after percutaneous vascular interventions such as stenting or angioplasty.

As defined herein, the phrase “deficient for NPP1” or “ENPP 1 deficiency” refers to a reduction in an amount of NPP1 protein or in NPP1 activity relative to a normal serum level of NPP1 protein or normal activity of NPP1, wherein such a reduction results in a disease or disorder of pathological calcification and/or pathological ossification. Such pathological diseases include but are not limited to GACI and ARHR2. ENPP1 deficiency, as used herein, does not refer to small reductions in an amount of NPP1 protein and/or NPP1 activity that do not result in a disease or disorder of pathological calcification and/or pathological ossification.

As defined herein the phrase “restenosis” refers to recurrence of stenosis. Stenosis refers to the narrowing of a blood vessel, leading to restricted blood flow. Restenosis usually pertains to an artery or other large blood vessel that has become narrowed, received treatment to clear the blockage and subsequently become re-narrowed. Restenosis is commonly detected by using one or more of ultrasound, X-ray computed tomography (CT), nuclear imaging, optical imaging or contrast enhanced image or immunohistochemical detection. As defined herein the phrase “myointimal proliferation” refers to the proliferation of vascular smooth muscle cells that occurs at the tunica intima of an arterial wall of an individual.

As used herein, the phrase “reduce or prevent myointimal proliferation” refers to the ability of soluble NPP1 upon administration to reduce the level of proliferation vascular smooth muscle cells at the site of tissue injury thereby reducing the thickening of arterial walls and prevent the occurrence of or reduce the level of restenosis of the artery.

As used herein, the term “treatment” or “treating” is defined as the application or administration of soluble NPP1 (alone or in combination with another pharmaceutical agent), to a patient, or application or administration of a therapeutic agent to an isolated tissue or cell line from a patient (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder, or the potential to develop the disease or disorder. Such treatments may be specifically tailored or modified, based on knowledge obtained from the field of pharmacogenomics.

As used herein, the term “prevent” or “prevention” or “reduce” means no disorder or disease development if none had occurred, or no further disorder or disease development if there had already been development of the disorder or disease. Also considered is the ability of one to prevent some or all of the symptoms associated with the disorder or disease.

As used herein, the term “effective amount” refers to an amount of an agent (e.g., NPP1 fusion or NPP3 fusion polypeptides) which, as compared to a corresponding subject who has not received such an amount, sufficient to provide improvement of a condition, disorder, disease, or to provide a decrease in progression or advancement of a condition, disorder, or disease. An effective amount also may result in treating, healing, preventing or ameliorating a condition, disease, or disorder. The term also includes within its scope amounts effective to enhance normal physiological function. As used herein, the term “polypeptide” refers to a polymer composed of amino acid residues, related naturally occurring structural variants, and synthetic non-naturally occurring analogs thereof linked via peptide bonds.

As used here the term “Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

As used herein the term “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained; for example, phosphate-buffered saline (PBS)

As used herein, the term “pathological calcification” refers to the abnormal deposition of calcium salts in blood vessels, soft tissues, secretory and excretory passages of the body causing it to harden. There are two types, dystrophic calcification which occurs in dying and dead tissue and metastatic calcification which elevated extracellular levels of calcium (hypercalcemia), exceeding the homeostatic capacity of cells and tissues. Calcification can involve cells as well as extracellular matrix components such as collagen in basement membranes and elastic fibers in arterial walls. Some examples of tissues prone to calcification include: Gastric mucosa—the inner epithelial lining of the stomach, Kidneys and lungs, Cornea, heart valves, Systemic arteries and Pulmonary veins.

As used herein, the term “pathological ossification” refers to a pathological condition in which bone arises in tissues not in the osseous system and in connective tissues usually not manifesting osteogenic properties. Ossification is classified into three types depending on the nature of the tissue or organ being affected, endochondral ossification is ossification that occurs in and replaces cartilage. Intramembranous ossification is ossification of bone that occurs in and replaces connective tissue. Metaplastic ossification the development of bony substance in normally soft body structures; called also heterotrophic ossification.

As used herein, “reduction of calcification” is observed by using non-invasive methods like X-rays, micro CT and Mill. Reduction of calcification is also inferred by using radio imaging with 99mTc-pyrophosphate (99mPYP) uptake. The presence of calcifications in mice are evaluated via post-mortem by micro-computed tomography (CT) scans and histologic sections taken from the heart, aorta and kidneys with the use of dyes such as Hematoxylin and Eosin (H&E) and Alizarin red by following protocols established by Braddock et al. (Nature Communications volume 6, Article number: 10006 (2015))

As used herein the term “ectopic calcification” refers to a condition characterized by a pathologic deposition of calcium salts in tissues or bone growth in soft tissues.

As used herein the term “ectopic calcification of soft tissue” refers to inappropriate biomineralization, typically composed of calcium phosphate, hydroxyapatite, calcium oxalates and ocatacalcium phosphates occurring in soft tissues leading to loss of hardening of soft tissues. “Arterial calcification” refers to ectopic calcification that occurs in arteries and heart valves leading to hardening and or narrowing of arteries. Calcification in arteries is correlated with atherosclerotic plaque burden and increased risk of myocardial infarction, increased ischemic episodes in peripheral vascular disease, and increased risk of dissection following angioplasty.

As used herein, the term “venous calcification” refers to ectopic calcification that occurs in veins that reduces the elasticity of the veins and restricts blood flow which can then lead to increase in blood pressure and coronary defects

As used herein, the term “vascular calcification” refers to the pathological deposition of mineral in the vascular system. It has a variety of forms, including intimal calcification and medial calcification, but can also be found in the valves of the heart. Vascular calcification is associated with atherosclerosis, diabetes, certain heredity conditions, and kidney disease, especially CKD. Patients with vascular calcification are at higher risk for adverse cardiovascular events. Vascular calcification affects a wide variety of patients. Idiopathic infantile arterial calcification is a rare form of vascular calcification where the arteries of neonates calcify.

The terms “adeno-associated viral vector”, “AAV vector”, “adeno-associated virus”, “AAV virus”, “AAV virion”, “AAV viral particle” and “AA V particle”, as used interchangeably herein, refer to a viral particle composed of at least one AAV capsid protein (preferably by all of the capsid proteins of a particular AAV serotype) and an encapsidated recombinant viral genome. The particle comprises a recombinant viral genome having a heterologous polynucleotide comprising a sequence encoding human ENPP1 or human ENPP3 or a functionally equivalent variant thereof,) and a transcriptional regulatory region that at least comprises a promoter flanked by the AAV inverted terminal repeats. The particle is typically referred to as an “AAV vector particle” or “AAV vector”.

As used herein, the term “vector” means a nucleic acid molecule capable of transporting another nucleic acid to which it has been linked. In some embodiments, the vector is a plasmid, i.e., a circular double stranded DNA loop into which additional DNA segments may be ligated. In some embodiments, the vector is a viral vector, wherein additional nucleotide sequences may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) is integrated into the genome of a host cell upon introduction into the host cell, and thereby are replicated along with the host genome. Moreover, certain vectors (expression vectors) are capable of directing the expression of genes to which they are operatively linked.

As used herein, the term “recombinant host cell” (or simply “host cell”), as used herein, means a cell into which an exogenous nucleic acid and/or recombinant vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “recombinant viral genome”, as used herein, refers to an AAV genome in which at least one extraneous expression cassette polynucleotide is inserted into the naturally occurring AAV genome. The genome of the AAV according to the disclosure typically comprises the cis-acting 5′ and 3′ inverted terminal repeat sequences (ITRs) and an expression cassette.

The term “expression cassette”, as used herein, refers to a nucleic acid construct, generated recombinantly or synthetically, with a series of specified nucleic acid elements, which permit transcription of a particular nucleic acid in a target cell. The expression cassette of the recombinant viral genome of the AAV vector according to the disclosure comprises a transcriptional regulatory region operatively linked to a nucleotide sequence encoding ENPP1 or ENPP3 or a functionally equivalent variant thereof.

The term “transcriptional regulatory region”, as used herein, refers to a nucleic acid fragment capable of regulating the expression of one or more genes. The transcriptional regulatory region according to the disclosure includes a promoter and, optionally, an enhancer.

The term “promoter”, as used herein, refers to a nucleic acid fragment that functions to control the transcription of one or more polynucleotides, located upstream the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. Any kind of promoters may be used in the disclosure including inducible promoters, constitutive promoters and tissue-specific promoters.

The term “enhancer”, as used herein, refers to a DNA sequence element to which transcription factors bind to increase gene transcription. Examples of enhancers may be, without limitation, RSV enhancer, CMV enhancer, HCR enhancer, etc. In another embodiment, the enhancer is a liver-specific enhancer, more preferably a hepatic control region enhancer (HCR).

The term “operatively linked”, as used herein, refers to the functional relation and location of a promoter sequence with respect to a polynucleotide of interest (e.g. a promoter or enhancer is operably linked to a coding sequence if it affects the transcription of the sequence). Generally, a promoter operatively linked is contiguous to the sequence of interest. However, an enhancer does not have to be contiguous to the sequence of interest to control its expression. In another embodiment, the promoter and the nucleotide sequence encoding ENPP1 or ENPP3 or a functionally equivalent variant thereof.

The term “effective amount” refers to a nontoxic but sufficient amount of a viral vector encoding ENPP1 or ENPP3 to provide the desired biological result. That result may be reduction and/or alleviation of the signs, symptoms, or causes of a disease, or any other desired alteration of a biological system.

The term “Cap protein”, as used herein, refers to a polypeptide having at least one functional activity of a native AAV Cap protein (e.g. VP1, VP2, VP3). Examples of functional activities of Cap proteins include the ability to induce formation of a capsid, facilitate accumulation of single-stranded DNA, facilitate AAV DNA packaging into capsids (i.e. encapsidation), bind to cellular receptors, and facilitate entry of the virion into host cells. In principle, any Cap protein can be used in the context of the present disclosure.

The term “capsid”, as used herein, refers to the structure in which the viral genome is packaged. A capsid consists of several oligomeric structural subunits made of proteins. For instance, AAV have an icosahedral capsid formed by the interaction of three capsid proteins: VP1, VP2 and VP3.

The term “Rep protein”, as used herein, refers to a polypeptide having at least one functional activity of a native AAV Rep protein (e.g. Rep 40, 52, 68, 78). A “functional activity” of a Rep protein is any activity associated with the physiological function of the protein, including facilitating replication of DNA through recognition, binding and nicking of the AAV origin of DNA replication as well as DNA helicase activity.

The term “adeno-associated virus ITRs” or “AAV ITRs”, as used herein, refers to the inverted terminal repeats present at both ends of the DNA strand of the genome of an adeno-associated virus. The ITR sequences are required for efficient multiplication of the AAV genome. Another property of these sequences is their ability to form a hairpin. This characteristic contributes to its self-priming which allows the primase-independent synthesis of the second DNA strand. Procedures for modifying these ITR sequences are known in the art (Brown T, “Gene Cloning”, Chapman & Hall, London, G B, 1995; Watson R, et al., “Recombinant DNA”, 2^{nd} Ed. Scientific American Books, New York, N.Y., US, 1992; Alberts B, et al., “Molecular Biology of the Cell”, Garland Publishing Inc., New York, N.Y., US, 2008; Innis M, et al., Eds., “PCR Protocols. A Guide to Methods and Applications”, Academic Press Inc., San Diego, Calif., US, 1990; and Schleef M, Ed., “Plasmid for Therapy and Vaccination”, Wiley-VCH Verlag GmbH, Weinheim, Del., 2001).

The term “tissue-specific” promoter is only active in specific types of differentiated cells or tissues. Typically, the downstream gene in a tissue-specific promoter is one which is active to a much higher degree in the tissue(s) for which it is specific than in any other. In this case there may be little or substantially no activity of the promoter in any tissue other than the one(s) for which it is specific.

The term “inducible promoter”, as used herein, refers to a promoter that is physiologically or developmentally regulated, e.g. by the application of a chemical inducer. For example, it can be a tetracycline-inducible promoter, a mifepristone (RU-486)-inducible promoter and the like.

The term “constitutive promoter”, as used herein, refers to a promoter whose activity is maintained at a relatively constant level in all cells of an organism, or during most developmental stages, with little or no regard to cell environmental conditions. In another embodiment, the transcriptional regulatory region allows constitutive expression of ENPP1. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1a promoter (Boshart M, et al., Cell 1985; 41:521-530).

The term “polyadenylation signal”, as used herein, relates to a nucleic acid sequence that mediates the attachment of a polyadenine stretch to the 3′ terminus of the mRNA. Suitable polyadenylation signals include, without limitation, the SV40 early polyadenylation signal, the SV40 late polyadenylation signal, the HSV thymidine kinase polyadenylation signal, the protamine gene polyadenylation signal, the adenovirus 5 EIb polyadenylation signal, the bovine growth hormone polyadenylation signal, the human variant growth hormone polyadenylation signal and the like.

The term “signal peptide”, as used herein, refers to a sequence of amino acid residues (ranging in length from 10-30 residues) bound at the amino terminus of a nascent protein of interest during protein translation. The signal peptide is recognized by the signal recognition particle (SRP) and cleaved by the signal peptidase following transport at the endoplasmic reticulum. (Lodish et al., 2000, Molecular Cell Biology, 4th edition).

As used herein, the term “immune response” or “immune reaction” refers to the host's immune system to antigen in an invading (infecting) pathogenic organism, or to introduction or expression of foreign protein. The immune response is generally humoral and local; antibodies produced by B cells combine with antigen in an antigen-antibody complex to inactivate or neutralize antigen. Immune response is often observed when human proteins are injected into mouse model systems. Generally, the mouse model system is made immune tolerant by injecting immune suppressors prior to the introduction of a foreign antigen to ensure better viability.

As used herein, the term “immunesuppression” is a deliberate reduction of the activation or efficacy of the host immune system using immunesuppresant drugs to facilitate immune tolerance towards foreign antigens such as foreign proteins, organ transplants, bone marrow and tissue transplantation. Non limiting examples of immunosuppressant drugs include anti-CD4(GK1.5) antibody, Cyclophosphamide, Azathioprine (Imuran), Mycophenolate mofetil (Cellcept), Cyclosporine (Neoral, Sandimmune, Gengraf), Methotrexate (Rheumatrex), Leflunomide (Arava), Cyclophosphamide (Cytoxan) and Chlorambucil (Leukeran).

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from lto 6 should be considered to have specifically disclosed subranges such as from 1 to 3, from lto 4, from lto 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of Treatment

The present disclosure relates to administration of an ENPP1 or ENPP3 agent to treat PAD, which includes administering sNPP1 and sNPP3 polypeptides and fusion proteins thereof to a subject, and to administration of nucleic acids encoding such polypeptides. Sequences of such polypeptides include the following, without limitation.

Sequences

```
SEQ ID NO: 1-ENPP1 Amino Acid Sequence-Wild Type
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln AlaAla Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Val Leu Ser Leu
65 70 75 80
Val Leu Ser Val Cys Val Leu Thr Thr Ile Leu Gly Cys Ile Phe Gly
                                85 90 95
Leu Lys Pro Ser Cys Ala Lys Glu Val Lys Ser Cys Lys Gly Arg Cys
                        100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
          370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp
                915 920 925
SEQ ID No: 2-Azurocidin-ENPP1-FC
MTRLTVLALLAGLLASSRA**APSCAKEVKSCKGRCFERTFGNCRCDAACVELGNCCLDYQETCIEPEHI
WTCNKFRCGEKRLTRSLCACSDDCKDKGDCCINYSSVCQGEKSWVEEPCESINEPQCPAGFETPPTLLES
LDGFRAEYLHTWGGLLPVISKLKKCGTYTKNMRPVYPTKTFPNHYSIVTGLYPESHGIIDNKMYDPKMNA
SFSLKSKEKENPEWYKGEPIWVTAKYQGLKSGTFFWPGSDVEINGIFPDIYKMYNGSVPFEERILAVLQW
LQLPKDERPHFYTLYLEEPDSSGHSYGPVSSEVIKALQRVDGMVGMLMDGLKELNLHRCLNLILISDHGM
EQGSCKKYIYLNKYLGDVKNIKVIYGPAARLRPSDVPDKYYSENYEGIARNLSCREPNQHFKPYLKHFLP
KRLHFAKSDRIEPLTFYLDPQWQLALNPSERKYCGSGFHGSDNVFSNMQALFVGYGPGFKHGIEADTFEN
IEVYNLMCDLLNLTPAPNNGTHGSLNHLLKNPVYTPKHPKEVHPLVQCPFTRNPRDNLGCSCNPSILPIE
DFQTQFNLTVAEEKIIKHETLPYGRPRVLQKENTICLLSQHQFMSGYSQDILMPLWTSYTVDRNDSESTE
DFSNCLYQDFRIPLSPVHKCSFYKNNTKVSYGELSPPQLNKNSSGIYSEALLTTNIVPMYQSFQVIWRYF
HDTLLRKYAEERNGVNVVSGPVFDEDYDGRCDSLENLRQKRRVIRNQEILIPTHFFIVLTSCKDTSQTPL
HCENLDTLAFILPHRTDNSESCVHGKHDSSWVEELLMLHRARITDVEHITGLSFYQQRKEPVSDILKLKT
HLPTFSQEDLINDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYSKLTVDKSRWQ
QGNVFSCSVMHEALHNHYTQKSLSLSPGK
Single underline-Azurocidin signal sequence, Double underline-
Beginning and end of ENPP1 sequence, Bold residues-Fc sequence,
**indicates the cleavage point of the signal sequence.
SEQ ID No: 3-Azurocidin-ENPP1-Alb
MTRLTVLALLAGLLASSRA**APSCAKEVKSCKGRCFERTFGNCRCDAACVELGNCCLDYQETCIEPEHI
WTCNKFRCGEKRLTRSLCACSDDCKDKGDCCINYSSVCQGEKSWVEEPCESINEPQCPAGFETPPTLLES
LDGFRAEYLHTWGGLLPVISKLKKCGTYTKNMRPVYPTKTFPNHYSIVTGLYPESHGIIDNKMYDPKMNA
SFSLKSKEKENPEWYKGEPIWVTAKYQGLKSGTFFWPGSDVEINGIFPDIYKMYNGSVPFEERILAVLQW
LQLPKDERPHFYTLYLEEPDSSGHSYGPVSSEVIKALQRVDGMVGMLMDGLKELNLHRCLNLILISDHGM
EQGSCKKYIYLNKYLGDVKNIKVIYGPAARLRPSDVPDKYYSENYEGIARNLSCREPNQHFKPYLKHELP
KRLHFAKSDRIEPLTFYLDPQWQLALNPSERKYCGSGFHGSDNVFSNMQALFVGYGPGFKHGIEADTFEN
IEVYNLMCDLLNLTPAPNNGTHGSLNHLLKNPVYTPKHPKEVHPLVQCPFTRNPRDNLGCSCNPSILPIE
DFQTQFNLTVAEEKIIKHETLPYGRPRVLQKENTICLLSQHQFMSGYSQDILMPLWTSYTVDRNDSESTE
DESNCLYQDFRIPLSPVHKCSFYKNNTKVSYGELSPPQLNKNSSGIYSEALLTTNIVPMYQSFQVIWRYF
HDTLLRKYAEERNGVNVVSGPVEDEDYDGRCDSLENLRQKRRVIRNQEILIPTHFFIVLTSCKDTSQTPL
HCENLDTLAFILPHRTDNSESCVHGKHDSSWVEELLMLHRARITDVEHITGLSFYQQRKEPVSDILKLKT
HLPTFSQEDLINMKWVTFLLLLFVSGSAFSRGVFRREAHKSEIAHRYNDLGEQHFKGLVLIAFSQYLQKC
SYDEHAKLVQEVTDFAKTCVADESAANCDKSLHTLFGDKLCAIPNLRENYGELADCCTKQEPERNECFLQ
HKDDNPSLPPFERPEAEAMCTSFKENPTTFMGHYLHEVARRHPYFYAPELLYYAEQYNEILTQCCAEADK
ESCLTPKLDGVKEKALVSSVRQRMKCSSMQKFGERAFKAWAVARLSQTFPNADFAEITKLATDLTKVNKE
CCHGDLLECADDRAELAKYMCENQATISSKLQTCCDKPLLKKAHCLSEVEHDTMPADLPAIAADFVEDQE
VCKNYAEAKDVFLGTFLYEYSRRHPDYSVSLLLRLAKKYEATLEKCCAEANPPACYGTVLAEFQPLVEEP
KNLVKTNCDLYEKLGEYGFQNAILVRYTQKAPQVSTPTLVEAARNLGRVGTKCCTLPEDQRLPCVEDYLS
AILNRVCLLHEKTPVSEHVTKCCSGSLVERRPCFSALTVDETYVPKEFKAETFTFHSDICTLPEKEKQIK
KQTALAELVKHKPKATAEQLKTVMDDFAQFLDTCCKAADKDTCFSTEGPNLVTRCKDALARSWSHPQFEK
Single underline-Azurocidin signal sequence, Double underline-
Beginning and end of ENPP1 sequence, Bold residues-Albumin sequence,
**indicates the cleavage point of the signal sequence.
SEQ ID No: 4-Azurocidin-ENPP1
MTRLTVLALLAGLLASSRA**APSCAKEVKSCKGRCFERTFGNCRCDAACVELGNCCLDYQETCIEPEHI
WTCNKFRCGEKRLTRSLCACSDDCKDKGDCCINYSSVCQGEKSWVEEPCESINEPQCPAGFETPPTLLES
LDGFRAEYLHTWGGLLPVISKLKKCGTYTKNMRPVYPTKTFPNHYSIVTGLYPESHGIIDNKMYDPKMNA
SFSLKSKEKENPEWYKGEPIWVTAKYQGLKSGTFFWPGSDVEINGIFPDIYKMYNGSVPFEERILAVLQW
LQLPKDERPHFYTLYLEEPDSSGHSYGPVSSEVIKALQRVDGMVGMLMDGLKELNLHRCLNLILISDHGM
EQGSCKKYIYLNKYLGDVKNIKVIYGPAARLRPSDVPDKYYSFNYEGIARNLSCREPNQHFKPYLKHELP
KRLHFAKSDRIEPLTFYLDPQWQLALNPSERKYCGSGFHGSDNVFSNMQALFVGYGPGFKHGIEADTFEN
IEVYNLMCDLLNLTPAPNNGTHGSLNHLLKNPVYTPKHPKEVHPLVQCPFTRNPRDNLGCSCNPSILPIE
DFQTQFNLTVAEEKIIKHETLPYGRPRVLQKENTICLLSQHQFMSGYSQDILMPLWTSYTVDRNDSESTE
DFSNCLYQDFRIPLSPVHKCSFYKNNTKVSYGELSPPQLNKNSSGIYSEALLTTNIVPMYQSFQVIWRYF
HDTLLRKYAEERNGVNVVSGPVFDFDYDGRCDSLENLRQKRRVIRNQEILIPTHFFIVLTSCKDTSQTAP
SCAKEVKSCKGRCFERTEGNCRCDAACVELGNCCLDYQETCIEPEHIWTCNKERCGEKRLTRSLCACSDD
CKDKGDCCINYSSVCQGEKSWVEEPCESINEPQCPAGFETPPTLLESLDGFRAEYLHTWGGLLPVISKLK
KCGTYTKNMRPVYPTKTFPNHYSIVTGLYPESHGIIDNKMYDPKMNASESLKSKEKENPEWYKGEPIWVT
AKYQGLKSGTFFWPGSDVEINGIFPDIYKMYNGSVPFEERILAVLQWLQLPKDERPHFYTLYLEEPDSSG
HSYGPVSSEVIKALQRVDGMVGMLMDGLKELNLHRCLNLILISDHGMEQGSCKKYIYLNKYLGDVKNIKV
IYGPAARLRPSDVPDKYYSENYEGIARNLSCREPNQHFKPYLKHELPKRLHFAKSDRIEPLTFYLDPQWQ
LALNPSERKYCGSGFHGSDNVFSNMQALFVGYGPGFKHGIEADTFENIEVYNLMCDLLNLTPAPNNGTHG
SLNHLLKNPVYTPKHPKEVHPLVQCPFTRNPRDNLGCSCNPSILPIEDEQTQENLTVAEEKIIKHETLPY
GRPRVLQKENTICLLSQHQFMSGYSQDILMPLWTSYTVDRNDSESTEDESNCLYQDFRIPLSPVHKCSFY
KNNTKVSYGFLSPPQLNKNSSGIYSEALLTTNIVPMYQSFQVIWRYFHDTLLRKYAEERNGVNVVSGPVF
DEDYDGRCDSLENLRQKRRVIRNQEILIPTHFFIVLTSCKDTSQTPLHCENLDTLAFILPHRTDNSESCV
HGKHDSSWVEELLMLHRARITDVEHITGLSFYQQRKEPVSDILKLKTHLPTFSQED
Single underline-Azurocidin signal sequence, Double underline-
Beginning and end of ENPP1 sequence,
**indicates the cleavage pointof the signal sequence.
SEQ ID NO: 5-ENPP2 Amino Acid Sequence-Wild Type
Met Ala Arg Arg Ser Ser Phe Gln Ser Cys Gln Ile Ile Ser Leu Phe
1 5 10 15
Thr Phe Ala Val Gly Val Asn Ile Cys Leu Gly Phe Thr Ala His Arg
                        20 25 30
Ile Lys Arg Ala Glu Gly Trp Glu Glu Gly Pro Pro Thr Val Leu Ser
                35 40 45
Asp Ser Pro Trp Thr Asn Ile Ser Gly Ser Cys Lys Gly Arg Cys Phe
        50 55 60
Glu Leu Gln Glu Ala Gly Pro Pro Asp Cys Arg Cys Asp Asn Leu Cys
65 70 75 80
Lys Ser Tyr Thr Ser Cys Cys His Asp Phe Asp Glu Leu Cys Leu Lys
                                85 90 95
Thr Ala Arg Gly Trp Glu Cys Thr Lys Asp Arg Cys Gly Glu Val Arg
                        100 105 110
Asn Glu Glu Asn Ala Cys His Cys Ser Glu Asp Cys Leu Ala Arg Gly
                115 120 125
Asp Cys Cys Thr Asn Tyr Gln Val Val Cys Lys Gly Glu Ser His Trp
        130 135 140
Val Asp Asp Asp Cys Glu Glu Ile Lys Ala Ala Glu Cys Pro Ala Gly
145 150 155 160
Phe Val Arg Pro Pro Leu Ile Ile Phe Ser Val Asp Gly Phe Arg Ala
                                165 170 175
Ser Tyr Met Lys Lys Gly Ser Lys Val Met Pro Asn Ile Glu Lys Leu
                        180 185 190
Arg Ser Cys Gly Thr His Ser Pro Tyr Met Arg Pro Val Tyr Pro Thr
                195 200 205
Lys Thr Phe Pro Asn Leu Tyr Thr Leu Ala Thr Gly Leu Tyr Pro Glu
        210 215 220
Ser His Gly Ile Val Gly Asn Ser Met Tyr Asp Pro Val Phe Asp Ala
225 230 235 240
Thr Phe His Leu Arg Gly Arg Glu Lys Phe Asn His Arg Trp Trp Gly
                                245 250 255
Gly Gln Pro Leu Trp Ile Thr Ala Thr Lys Gln Gly Val Lys Ala Gly
                        260 265 270
Thr Phe Phe Trp Ser Val Val Ile Pro His Glu Arg Arg Ile Leu Thr
                275 280 285
Ile Leu Gln Trp Leu Thr Leu Pro Asp His Glu Arg Pro Ser Val Tyr
        290 295 300
Ala Phe Tyr Ser Glu Gln Pro Asp Phe Ser Gly His Lys Tyr Gly Pro
305 310 315 320
Phe Gly Pro Glu Met Thr Asn Pro Leu Arg Glu Ile Asp Lys Ile Val
                                325 330 335
Gly Gln Leu Met Asp Gly Leu Lys Gln Leu Lys Leu His Arg Cys Val
                        340 345 350
Asn Val Ile Phe Val Gly Asp His Gly Met Glu Asp Val Thr Cys Asp
                355 360 365
Arg Thr Glu Phe Leu Ser Asn Tyr Leu Thr Asn Val Asp Asp Ile Thr
        370 375 380
Leu Val Pro Gly Thr Leu Gly Arg Ile Arg Ser Lys Phe Ser Asn Asn
385 390 395 400
Ala Lys Tyr Asp Pro Lys Ala Ile Ile Ala Asn Leu Thr Cys Lys Lys
                                405 410 415
Pro Asp Gln His Phe Lys Pro Tyr Leu Lys Gln His Leu Pro Lys Arg
                        420 425 430
Leu His Tyr Ala Asn Asn Arg Arg Ile Glu Asp Ile His Leu Leu Val
                435 440 445
Glu Arg Arg Trp His Val Ala Arg Lys Pro Leu Asp Val Tyr Lys Lys
        450 455 460
Pro Ser Gly Lys Cys Phe Phe Gln Gly Asp His Gly Phe Asp Asn Lys
465 470 475 480
Val Asn Ser Met Gln Thr Val Phe Val Gly Tyr Gly Ser Thr Phe Lys
                                485 490 495
Tyr Lys Thr Lys Val Pro Pro Phe Glu Asn Ile Glu Leu Tyr Asn Val
                        500 505 510
Met Cys Asp Leu Leu Gly Leu Lys Pro Ala Pro Asn Asn Gly Thr His
                515 520 525
Gly Ser Leu Asn His Leu Leu Arg Thr Asn Thr Phe Arg Pro Thr Met
        530 535 540
Pro Glu Glu Val Thr Arg Pro Asn Tyr Pro Gly Ile Met Tyr Leu Gln
545 550 555 560
Ser Asp Phe Asp Leu Gly Cys Thr Cys Asp Asp Lys Val Glu Pro Lys
                                565 570 575
Asn Lys Leu Asp Glu Leu Asn Lys Arg Leu His Thr Lys Gly Ser Thr
                        580 585 590
Glu Ala Glu Thr Arg Lys Phe Arg Gly Ser Arg Asn Glu Asn Lys Glu
                595 600 605
Asn Ile Asn Gly Asn Phe Glu Pro Arg Lys Glu Arg His Leu Leu Tyr
        610 615 620
Gly Arg Pro Ala Val Leu Tyr Arg Thr Arg Tyr Asp Ile Leu Tyr His
625 630 635 640
Thr Asp Phe Glu Ser Gly Tyr Ser Glu Ile Phe Leu Met Pro Leu Trp
                                645 650 655
Thr Ser Tyr Thr Val Ser Lys Gln Ala Glu Val Ser Ser Val Pro Asp
                        660 665 670
His Leu Thr Ser Cys Val Arg Pro Asp Val Arg Val Ser Pro Ser Phe
                675 680 685
Ser Gln Asn Cys Leu Ala Tyr Lys Asn Asp Lys Gln Met Ser Tyr Gly
        690 695 700
Phe Leu Phe Pro Pro Tyr Leu Ser Ser Ser Pro Glu Ala Lys Tyr Asp
705 710 715 720
Ala Phe Leu Val Thr Asn Met Val Pro Met Tyr Pro Ala Phe Lys Arg
                                725 730 735
Val Trp Asn Tyr Phe Gln Arg Val Leu Val Lys Lys Tyr Ala Ser Glu
                        740 745 750
Arg Asn Gly Val Asn Val Ile Ser Gly Pro Ile Phe Asp Tyr Asp Tyr
                755 760 765
Asp Gly Leu His Asp Thr Glu Asp Lys Ile Lys Gln Tyr Val Glu Gly
        770 775 780
Ser Ser Ile Pro Val Pro Thr His Tyr Tyr Ser Ile Ile Thr Ser Cys
785 790 795 800
Leu Asp Phe Thr Gln Pro Ala Asp Lys Cys Asp Gly Pro Leu Ser Val
                                805 810 815
Ser Ser Phe Ile Leu Pro His Arg Pro Asp Asn Glu Glu Ser Cys Asn
                        820 825 830
Ser Ser Glu Asp Glu Ser Lys Trp Val Glu Glu Leu Met Lys Met His
                835 840 845
Thr Ala Arg Val Arg Asp Ile Glu His Leu Thr Ser Leu Asp Phe Phe
        850 855 860
Arg Lys Thr Ser Arg Ser Tyr Pro Glu Ile Leu Thr Leu Lys Thr Tyr
865 870 875 880
Leu His Thr Tyr Glu Ser Glu Ile
                                885
SEQ. ID NO: 6-Extracellular Domain of ENPP3:
Glu Lys Gln Gly Ser Cys Arg Lys Lys Cys Phe Asp Ala Ser Phe Arg
1 5 10 15
Gly Leu Glu Asn Cys Arg Cys Asp Val Ala Cys Lys Asp Arg Gly Asp
                        20 25 30
Cys Cys Trp Asp Phe Glu Asp Thr Cys Val Glu Ser Thr Arg Ile Trp
                35 40 45
Met Cys Asn Lys Phe Arg Cys Gly Glu Thr Arg Leu Glu Ala Ser Leu
        50 55 60
Cys Ser Cys Ser Asp Asp Cys Leu Gln Arg Lys Asp Cys Cys Ala Asp
65 70 75 80
Tyr Lys Ser Val Cys Gln Gly Glu Thr Ser Trp Leu Glu Glu Asn Cys
                                85 90 95
Asp Thr Ala Gln Gln Ser Gln Cys Pro Glu Gly Phe Asp LeuPro Pro
                        100 105 110
Val Ile Leu Phe Ser Met Asp Gly Phe Arg Ala Glu Tyr Leu Tyr Thr
                115 120 125
Trp Asp Thr Leu Met Pro Asn Ile Asn Lys Leu Lys Thr Cys Gly Ile
        130 135 140
His Ser Lys Tyr Met Arg Ala Met Tyr Pro Thr Lys Thr Phe Pro Asn
145 150 155 160
His Tyr Thr Ile Val Thr Gly Leu Tyr Pro Glu Ser His Gly Ile Ile
                                165 170 175
Asp Asn Asn Met Tyr Asp Val Asn Leu Asn Lys Asn Phe Ser Leu Ser
                        180 185 190
Ser Lys Glu Gln Asn Asn Pro Ala Trp Trp His Gly Gln Pro Met Trp
                195 200 205
Leu Thr Ala Met Tyr Gln Gly Leu Lys Ala Ala Thr Tyr Phe Trp Pro
        210 215 220
Gly Ser Glu Val Ala Ile Asn Gly Ser Phe Pro Ser Ile Tyr Met Pro
225 230 235 240
Tyr Asn Gly Ser Val Pro Phe Glu Glu Arg Ile Ser Thr Leu Leu Lys
                                245 250 255
Trp Leu Asp Leu Pro Lys Ala Glu Arg Pro Arg Phe Tyr Thr Met Tyr
                        260 265 270
Phe Glu Glu Pro Asp Ser Ser Gly His Ala Gly Gly Pro Val Ser Ala
                275 280 285
Arg Val Ile Lys Ala Leu Gln Val Val Asp His Ala Phe Gly Met Leu
        290 295 300
Met Glu Gly Leu Lys Gln Arg Asn Leu His Asn Cys Val Asn Ile Ile
305 310 315 320
Leu Leu Ala Asp His Gly Met Asp Gln Thr Tyr Cys Asn Lys Met Glu
                                325 330 335
Tyr Met Thr Asp Tyr Phe Pro Arg Ile Asn Phe Phe Tyr Met Tyr Glu
  340 340 345 350
Gly Pro Ala Pro Arg Ile Arg Ala His Asn Ile Pro His Asp Phe Phe
                355 360 365
Ser Phe Asn Ser Glu Glu Ile Val Arg Asn Leu Ser Cys Arg Lys Pro
        370 375 380
Asp Gln His Phe Lys Pro Tyr Leu Thr Pro Asp Leu Pro Lys Arg Leu
385 390 395 400
His Tyr Ala Lys Asn Val Arg Ile Asp Lys Val His Leu Phe Val Asp
                                405 410 415
Gln Gln Trp Leu Ala Val Arg Ser Lys Ser Asn Thr Asn Cys Gly Gly
                        420 425 430
Gly Asn His Gly Tyr Asn Asn Glu Phe Arg Ser Met Glu Ala Ile Phe
                435 440 445
Leu Ala His Gly Pro Ser Phe Lys Glu Lys Thr Glu Val Glu Pro Phe
        450 455 460
Glu Asn Ile Glu Val Tyr Asn Leu Met Cys Asp Leu Leu Arg Ile Gln
465 470 475 480
Pro Ala Pro Asn Asn Gly Thr His Gly Ser Leu Asn His Leu Leu Lys
                                485 490 495
Val Pro Phe Tyr Glu Pro Ser His Ala Glu Glu Val Ser Lys Phe Ser
                        500 505 510
Val Cys Gly Phe Ala Asn Pro Leu Pro Thr Glu Ser Leu Asp Cys Phe
                515 520 525
Cys Pro His Leu Gln Asn Ser Thr Gln Leu Glu Gln Val Asn Gln Met
        530 535 540
Leu Asn Leu Thr Gln Glu Glu Ile Thr Ala Thr Val Lys Val Asn Leu
545 550 555 560
Pro Phe Gly Arg Pro Arg Val Leu Gln Lys Asn Val Asp His Cys Leu
                                565 570 575
Leu Tyr His Arg Glu Tyr Val Ser Gly Phe Gly Lys Ala Met Arg Met
                        580 585 590
Pro Met Trp Ser Ser Tyr Thr Val Pro Gln Leu Gly Asp Thr Ser Pro
                595 600 605
Leu Pro Pro Thr Val Pro Asp Cys Leu Arg Ala Asp Val Arg Val Pro
        610 615 620
Pro Ser Glu Ser Gln Lys Cys Ser Phe Tyr Leu Ala Asp Lys Asn Ile
625 630 635 640
Thr His Gly Phe Leu Tyr Pro Pro Ala Ser Asn Arg Thr Ser Asp Ser
                                645 650 655
Gln Tyr Asp Ala Leu Ile Thr Ser Asn Leu Val Pro Met Tyr Glu Glu
                        660 665 670
Phe Arg Lys Met Trp Asp Tyr Phe His Ser Val Leu Leu Ile Lys His
                675 680 685
Ala Thr Glu Arg Asn Gly Val Asn Val Val Ser Gly Pro Ile Phe Asp
        690 695 700
Tyr Asn Tyr Asp Gly His Phe Asp Ala Pro Asp Glu Ile Thr Lys His
705 710 715 720
Leu Ala Asn Thr Asp Val Pro Ile Pro Thr His Tyr Phe Val Val Leu
                                725 730 735
Thr Ser Cys Lys Asn Lys Ser His Thr Pro Glu Asn Cys Pro Gly Trp
                        740 745 750
Leu Asp Val Leu Pro Phe Ile Ile Pro His Arg Pro Thr Asn Val Glu
                755 760 765
Ser Cys Pro Glu Gly Lys Pro Glu Ala Leu Trp Val Glu Glu Arg Phe
        770 775 780
Thr Ala His Ile Ala Arg Val Arg Asp Val Glu Leu Leu Thr Gly Leu
785 790 795 800
Asp Phe Tyr Gln Asp Lys Val Gln Pro Val Ser Glu Ile Leu Gln Leu
                                805 810 815
Lys Thr Tyr Leu Pro Thr Phe Glu Thr Thr Ile
                        820 825
SEQ. ID NO: 7-NPP3 Amino Acid Sequence:
Met Glu Ser Thr Leu Thr Leu Ala Thr Glu Gln Pro Val Lys Lys Asn
1 5 10 15
Thr Leu Lys Lys Tyr Lys Ile Ala Cys Ile Val Leu Leu Ala Leu Leu
                        20 25 30
Val Ile Met Ser Leu Gly Leu Gly Leu Gly Leu Gly Leu Arg Lys Leu
                35 40 45
Glu Lys Gln Gly Ser Cys Arg Lys Lys Cys Phe Asp Ala Ser Phe Arg
        50 55 60
Gly Leu Glu Asn Cys Arg Cys Asp Val Ala Cys Lys Asp Arg Gly Asp
65 70 75 80
Cys Cys Trp Asp Phe Glu Asp Thr Cys Val Glu Ser Thr Arg Ile Trp
                                85 90 95
Met Cys Asn Lys Phe Arg Cys Gly Glu Thr Arg Leu Glu Ala Ser Leu
                        100 105 110
Cys Ser Cys Ser Asp Asp Cys Leu Gln Arg Lys Asp Cys Cys Ala Asp
                115 120 125
Tyr Lys Ser Val Cys Gln Gly Glu Thr Ser Trp Leu Glu Glu Asn Cys
        130 135 140
Asp Thr Ala Gln Gln Ser Gln Cys Pro Glu Gly Phe Asp Leu Pro Pro
145 150 155 160
Val Ile Leu Phe Ser Met Asp Gly Phe Arg Ala Glu Tyr Leu Tyr Thr
                                165 170 175
Trp Asp Thr Leu Met Pro Asn Ile Asn Lys Leu Lys Thr Cys Gly Ile
                        180 185 190
His Ser Lys Tyr Met Arg Ala Met Tyr Pro Thr Lys Thr Phe Pro Asn
                195 200 205
His Tyr Thr Ile Val Thr Gly Leu Tyr Pro Glu Ser His Gly Ile Ile
        210 215 220
Asp Asn Asn Met Tyr Asp Val Asn Leu Asn Lys Asn Phe Ser Leu Ser
225 230 235 240
Ser Lys Glu Gln Asn Asn Pro Ala Trp Trp His Gly Gln Pro Met Trp
                                245 250 255
Leu Thr Ala Met Tyr Gln Gly Leu Lys Ala Ala Thr Tyr Phe Trp Pro
                        260 265 270
Gly Ser Glu Val Ala Ile Asn Gly Ser Phe Pro Ser Ile Tyr Met Pro
                275 280 285
Tyr Asn Gly Ser Val Pro Phe Glu Glu Arg Ile Ser Thr Leu Leu Lys
        290 295 300
Trp Leu Asp Leu Pro Lys Ala Glu Arg Pro Arg Phe Tyr Thr Met Tyr
305 310 315 320
Phe Glu Glu Pro Asp Ser Ser Gly His Ala Gly Gly Pro Val Ser Ala
                                325 330 335
Arg Val Ile Lys Ala Leu Gln Val Val Asp His Ala Phe Gly Met Leu
                        340 345 350
Met Glu Gly Leu Lys Gln Arg Asn Leu His Asn Cys Val Asn Ile Ile
                355 360 365
Leu Leu Ala Asp His Gly Met Asp Gln Thr Tyr Cys Asn Lys Met Glu
        370 375 380
Tyr Met Thr Asp Tyr Phe Pro Arg Ile Asn Phe Phe Tyr Met Tyr Glu
385 390 395 400
Gly Pro Ala Pro Arg Ile Arg Ala His Asn Ile Pro His Asp Phe Phe
                                405 410 415
Ser Phe Asn Ser Glu Glu Ile Val Arg Asn Leu Ser Cys Arg Lys Pro
                        420 425 430
Asp Gln His Phe Lys Pro Tyr Leu Thr Pro Asp Leu Pro Lys Arg Leu
                435 440 445
His Tyr Ala Lys Asn Val Arg Ile Asp Lys Val His Leu Phe Val Asp
        450 455 460
Gln Gln Trp Leu Ala Val Arg Ser Lys Ser Asn Thr Asn Cys Gly Gly
465 470 475 480
Gly Asn His Gly Tyr Asn Asn Glu Phe Arg Ser Met Glu Ala Ile Phe
                                485 490 495
Leu Ala His Gly Pro Ser Phe Lys Glu Lys Thr Glu Val Glu Pro Phe
                        500 505 510
Glu Asn Ile Glu Val Tyr Asn Leu Met Cys Asp Leu Leu Arg Ile Gln
                515 520 525
Pro Ala Pro Asn Asn Gly Thr His Gly Ser Leu Asn His Leu Leu Lys
        530 535 540
Val Pro Phe Tyr Glu Pro Ser His Ala Glu Glu Val Ser Lys Phe Ser
545 550 555 560
Val Cys Gly Phe Ala Asn Pro Leu Pro Thr Glu Ser Leu Asp Cys Phe
                                565 570 575
Cys Pro His Leu Gln Asn Ser Thr Gln Leu Glu Gln Val Asn Gln Met
                        580 585 590
Leu Asn Leu Thr Gln Glu Glu Ile Thr Ala Thr Val Lys Val Asn Leu
                595 600 605
Pro Phe Gly Arg Pro Arg Val Leu Gln Lys Asn Val Asp His Cys Leu
        610 615 620
Leu Tyr His Arg Glu Tyr Val Ser Gly Phe Gly Lys Ala Met Arg Met
625 630 635 640
Pro Met Trp Ser Ser Tyr Thr Val Pro Gln Leu Gly Asp Thr Ser Pro
                                645 650 655
Leu Pro Pro Thr Val Pro Asp Cys Leu Arg Ala Asp Val Arg Val Pro
                        660 665 670
Pro Ser Glu Ser Gln Lys Cys Ser Phe Tyr Leu Ala Asp Lys Asn Ile
                675 680 685
Thr His Gly Phe Leu Tyr Pro Pro Ala Ser Asn Arg Thr Ser Asp Ser
        690 695 700
Gln Tyr Asp Ala Leu Ile Thr Ser Asn Leu Val Pro Met Tyr Glu Glu
705 710 715 720
Phe Arg Lys Met Trp Asp Tyr Phe His Ser Val Leu Leu Ile Lys His
                                725 730 735
Ala Thr Glu Arg Asn Gly Val Asn Val Val Ser Gly Pro Ile Phe Asp
                        740 745 750
Tyr Asn Tyr Asp Gly His Phe Asp Ala Pro Asp Glu Ile Thr Lys His
                755 760 765
Leu Ala Asn Thr Asp Val Pro Ile Pro Thr His Tyr Phe Val Val Leu
        770 775 780
Thr Ser Cys Lys Asn Lys Ser His Thr Pro Glu Asn Cys Pro Gly Trp
785 790 795 800
Leu Asp Val Leu Pro Phe Ile Ile Pro His Arg Pro Thr Asn Val Glu
                                805 810 815
Ser Cys Pro Glu Gly Lys Pro Glu Ala Leu Trp Val Glu Glu Arg Phe
                        820 825 830
Thr Ala His Ile Ala Arg Val Arg Asp Val Glu Leu Leu Thr Gly Leu
                835 840 845
Asp Phe Tyr Gln Asp Lys Val Gln Pro Val Ser Glu Ile Leu Gln Leu
        850 855 860
Lys Thr Tyr Leu Pro Thr Phe Glu Thr Thr Ile
865 870 875
SEQ ID No: 8-Azurocidin-ENPP3-FC
          MTRLTVLALLAGLLASSRA**AKQGSCRKKCFDASFRGLENCRCDVACKDRGDCCWDFEDTCVES
TRIWMCNKFRCGETRLEASLCSCSDDCLQRKDCCADYKSVCQGETSWLEENCDTAQQSQCPEGFDLPPVI
LFSMDGFRAEYLYTWDTLMPNINKLKTCGIHSKYMRAMYPTKTFPNHYTIVTGLYPESHGIIDNNMYDVN
LNKNFSLSSKEQNNPAWWHGQPMNLTAMYQGLKAATYFWPGSEVAINGSEPSIYMPYNGSVPFEERISTL
LKWLDLPKAERPRFYTMYFEEPDSSGHAGGPVSARVIKALQVVDHAFGMLMEGLKQRNLHNCVNIILLAD
HGMDQTYCNKMEYMTDYFPRINFFYMYEGPAPRIRAHNIPHDFFSENSEEIVRNLSCRKPDQHFKPYLTP
DLPKRLHYAKNVRIDKVHLFVDQQWLAVRSKSNTNCGGGNHGYNNEFRSMEAIFLAHGPSFKEKTEVEPF
ENIEVYNLMCDLLRIQPAPNNGTHGSLNHLLKVPFYEPSHAEEVSKESVCGFANPLPTESLDCFCPHLQN
STQLEQVNQMLNLTQEEITATVKVNLPFGRPRVLQKNVDHCLLYHREYVSGFGKAMRMPMWSSYTVPQLG
DTSPLPPTVPDCLRADVRVPPSESQKCSFYLADKNITHGFLYPPASNRTSDSQYDALITSNLVPMYEEER
KMWDYFHSVLLIKHATERNGVNVVSGPIFDYNYDGHEDAPDEITKHLANTDVPIPTHYFVVLTSCKNKSH
TPENCPGWLDVLPFIIPHRPTNVESCPEGKPEALWVEERFTAHIARVRDVELLTGLDFYQDKVQPVSEIL
QLKTYLPTFETTIDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPVLDSDGSFFLYSKLTVDKSRW
QQGNVFSCSVMHEALHNHYTQKSLSLSPGK
Single underline-Azurocidin signal sequence, Double underline-
Beginning and end of ENPP3 sequence, Bold residues-Fc sequence,
**indicates the cleavage point of the signal sequence.
SEQ ID No: 9-Azurocidin-ENPP3-Albumin
          MTRLTVLALLAGLLASSRA**AKQGSCRKKCFDASFRGLENCRCDVACKDRGDCCWDFEDTCVES
TRIWMCNKFRCGETRLEASLCSCSDDCLQRKDCCADYKSVCQGETSWLEENCDTAQQSQCPEGEDLPPVI
LFSMDGFRAEYLYTWDTLMPNINKLKTCGIHSKYMRAMYPTKTFPNHYTIVTGLYPESHGIIDNNMYDVN
LNKNFSLSSKEQNNPAWWHGQPMNLTAMYQGLKAATYFWPGSEVAINGSFPSIYMPYNGSVPFEERISTL
LKWLDLPKAERPRFYTMYFEEPDSSGHAGGPVSARVIKALQVVDHAFGMLMEGLKQRNLHNCVNIILLAD
HGMDQTYCNKMEYMTDYFPRINFFYMYEGPAPRIRAHNIPHDFFSENSEEIVRNLSCRKPDQHFKPYLTP
DLPKRLHYAKNVRIDKVHLFVDQQWLAVRSKSNTNCGGGNHGYNNEFRSMEAIFLAHGPSFKEKTEVEPF
ENIEVYNLMCDLLRIQPAPNNGTHGSLNHLLKVPFYEPSHAEEVSKESVCGFANPLPTESLDCFCPHLQN
STQLEQVNQMLNLTQEEITATVKVNLPFGRPRVLQKNVDHCLLYHREYVSGFGKAMRMPMWSSYTVPQLG
DTSPLPPTVPDCLRADVRVPPSESQKCSFYLADKNITHGFLYPPASNRTSDSQYDALITSNLVPMYEEFR
KMWDYFHSVLLIKHATERNGVNVVSGPIFDYNYDGHEDAPDEITKHLANTDVPIPTHYFVVLTSCKNKSH
TPENCPGWLDVLPFIIPHRPTNVESCPEGKPEALWVEERFTAHIARVRDVELLTGLDFYQDKVQPVSEIL
QLKTYLPTFETTIMKWVTFLLLLFVSGSAFSRGVFRREAHKSEIAHRYNDLGEQHFKGLVLIAFSQYLQK
CSYDEHAKLVQEVTDFAKTCVADESAANCDKSLHTLFGDKLCAIPNLRENYGELADCCTKQEPERNECFL
QHKDDNPSLPPFERPEAEAMCTSFKENPTTFMGHYLHEVARRHPYFYAPELLYYAEQYNEILTQCCAEAD
KESCLTPKLDGVKEKALVSSVRQRMKCSSMQKFGERAFKAWAVARLSQTFPNADFAEITKLATDLTKVNK
ECCHGDLLECADDRAELAKYMCENQATISSKLQTCCDKPLLKKAHCLSEVEHDTMPADLPAIAADFVEDQ
EVCKNYAEAKDVFLGTFLYEYSRRHPDYSVSLLLRLAKKYEATLEKCCAEANPPACYGTVLAEFQPLVEE
PKNLVKTNCDLYEKLGEYGFQNAILVRYTQKAPQVSTPTLVEAARNLGRVGTKCCTLPEDQRLPCVEDYL
SAILNRVCLLHEKTPVSEHVTKCCSGSLVERRPCFSALTVDETYVPKEFKAETFTFHSDICTLPEKEKQI
KKQTALAELVKHKPKATAEQLKTVMDDFAQFLDTCCKAADKDTCFSTEGPNLVTRCKDALARSWSHPQFE
K
Single underline-Azurocidin signal sequence, Double underline-
Beginning and end of ENPP3 sequence, Bold residues- Albumin sequence,
**indicates the cleavage point of the signal sequence.
SEQ ID No: 10-Azurocidin-ENPP3
            MTRLTVLALLAGLLASSRA**AKQGSCRKKCFDASFRGLENCRCDVACKDRGDCCWDFEDTCVES
TRIWMCNKFRCGETRLEASLCSCSDDCLQRKDCCADYKSVCQGETSWLEENCDTAQQSQCPEGFDLPPVI
LFSMDGFRAEYLYTWDTLMPNINKLKTCGIHSKYMRAMYPTKTFPNHYTIVTGLYPESHGIIDNNMYDVN
LNKNFSLSSKEQNNPAWWHGQPMNLTAMYQGLKAATYFWPGSEVAINGSFPSIYMPYNGSVPFEERISTL
LKWLDLPKAERPRFYTMYFEEPDSSGHAGGPVSARVIKALQVVDHAFGMLMEGLKQRNLHNCVNIILLAD
HGMDQTYCNKMEYMTDYFPRINFFYMYEGPAPRIRAHNIPHDFFSENSEEIVRNLSCRKPDQHFKPYLTP
DLPKRLHYAKNVRIDKVHLFVDQQWLAVRSKSNTNCGGGNHGYNNEFRSMEAIFLAHGPSFKEKTEVEPF
ENIEVYNLMCDLLRIQPAPNNGTHGSLNHLLKVPFYEPSHAEEVSKESVCGFANPLPTESLDCFCPHLQN
STQLEQVNQMLNLTQEEITATVKVNLPFGRPRVLQKNVDHCLLYHREYVSGFGKAMRMPMWSSYTVPQLG
DTSPLPPTVPDCLRADVRVPPSESQKCSFYLADKNITHGFLYPPASNRTSDSQYDALITSNLVPMYEEER
KMWDYFHSVLLIKHATERNGVNVVSGPIFDYNYDGHFDAPDEITKHLANTDVPIPTHYFVVLTSCKNKSH
TPENCPGWLDVLPFIIPHRPTNVESCPEGKPEALWVEERFTAHIARVRDVELLTGLDFYQDKVQPVSEIL
QLKTYLPTFETTI
Single underline-Azurocidin signal sequence, Double underline-
Beginning and end of ENPP3 sequence, ** indicates the cleavage point
of the signal sequence.
SEQ. ID NO: 11-ENPP4 Amino Acid Sequence-Wild Type
Met Lys Leu Leu Val Ile Leu Leu Phe Ser Gly Leu Ile Thr Gly Phe
1 5 10 15
Arg Ser Asp Ser Ser Ser Ser Leu Pro Pro Lys Leu Leu Leu Val Ser
                        20 25 30
Phe Asp Gly Phe Arg Ala Asp Tyr Leu Lys Asn Tyr Glu Phe Pro His
                35 40 45
Leu Gln Asn Phe Ile Lys Glu Gly Val Leu Val Glu His Val Lys Asn
        50 55 60
Val Phe Ile Thr Lys Thr Phe Pro Asn His Tyr Ser Ile Val Thr Gly
65 70 75 80
Leu Tyr Glu Glu Ser His Gly Ile Val Ala Asn Ser Met Tyr Asp Ala
                                85 90 95
Val Thr Lys Lys His Phe Ser Asp Ser Asn Asp Lys Asp Pro Phe Trp
100 100 105 110
Trp Asn Glu Ala Val Pro Ile Trp Val Thr Asn Gln Leu Gln Glu Asn
                115 120 125
Arg Ser Ser Ala Ala Ala Met Trp Pro Gly Thr Asp Val Pro Ile His
        130 135 140
Asp Thr Ile Ser Ser Tyr Phe Met Asn Tyr Asn Ser Ser Val Ser Phe
145 150 155 160
Glu Glu Arg Leu Asn Asn Ile Thr Met Trp Leu Asn Asn Ser Asn Pro
                                165 170 175
Pro Val Thr Phe Ala Thr Leu Tyr Trp Glu Glu Pro Asp Ala Ser Gly
                        180 185 190
His Lys Tyr Gly Pro Glu Asp Lys Glu Asn Met Ser Arg Val Leu Lys
                195 200 205
Lys Ile Asp Asp Leu Ile Gly Asp Leu Val Gln Arg Leu Lys Met Leu
        210 215 220
Gly Leu Trp Glu Asn Leu Asn Val Ile Ile Thr Ser Asp His Gly Met
225 230 235 240
Thr Gln Cys Ser Gln Asp Arg Leu Ile Asn Leu Asp Ser Cys Ile Asp
                                245 250 255
His Ser Tyr Tyr Thr Leu Ile Asp Leu Ser Pro Val Ala Ala Ile Leu
                        260 265 270
Pro Lys Ile Asn Arg Thr Glu Val Tyr Asn Lys Leu Lys Asn Cys Ser
                275 280 285
Pro His Met Asn Val Tyr Leu Lys Glu Asp Ile Pro Asn Arg Phe Tyr
        290 295 300
Tyr Gln His Asn Asp Arg Ile Gln Pro Ile Ile Leu Val Ala Asp Glu
305 310 315 320
Gly Trp Thr Ile Val Leu Asn Glu Ser Ser Gln Lys Leu Gly Asp His
                                325 330 335
Gly Tyr Asp Asn Ser Leu Pro Ser Met His Pro Phe Leu Ala Ala His
                        340 345 350
Gly Pro Ala Phe His Lys Gly Tyr Lys His Ser Thr Ile Asn Ile Val
                355 360 365
Asp Ile Tyr Pro Met Met Cys His Ile Leu Gly Leu Lys Pro His Pro
        370 375 380
Asn Asn Gly Thr Phe Gly His Thr Lys Cys Leu Leu Val Asp Gln Trp
385 390 395 400
Cys Ile Asn Leu Pro Glu Ala Ile Ala Ile Val Ile Gly Ser Leu Leu
                                405 410 415
Val Leu Thr Met Leu Thr Cys Leu Ile Ile Ile Met Gln Asn Arg Leu
                        420 425 430
Ser Val Pro Arg Pro Phe Ser Arg Leu Gln Leu Gln Glu Asp Asp Asp
                435 440 445
Asp Pro Leu Ile Gly
        450
SEQ. ID NO: 12-ENPP51 Amino Acid Sequence
Met Thr Ser Lys Phe Leu Leu Val Ser Phe Ile Leu Ala Ala Leu Ser
1 5 10 15
Leu Ser Thr Thr Phe Ser Leu Gln**Pro Ser Cys Ala Lys Glu Val Lys
                        20 25 30
Ser Cys Lys Gly Arg Cys Phe Glu Arg Thr Phe Ser Asn Cys Arg Cys
                35 40 45
Asp Ala Ala Cys Val Ser Leu Gly Asn Cys Cys Leu Asp Phe Gln Glu
        50 55 60
Thr Cys Val Glu Pro Thr His Ile Trp Thr Cys Asn Lys Phe Arg Cys
65 70 75 80
Gly Glu Lys Arg Leu Ser Arg Phe Val Cys Ser Cys Ala Asp Asp Cys
                                85 90 95
Lys Thr His Asn Asp Cys Cys Ile Asn Tyr Ser Ser Val Cys Gln Asp
                        100 105 110
Lys Lys Ser Trp Val Glu Glu Thr Cys Glu Ser Ile Asp Thr Pro Glu
                115 120 125
Cys Pro Ala Glu Phe Glu Ser Pro Pro Thr Leu Leu Phe Ser Leu Asp
        130 135 140
Gly Phe Arg Ala Glu Tyr Leu His Thr Trp Gly Gly Leu Leu Pro Val
145 150 155 160
Ile Ser Lys Leu Lys Asn Cys Gly Thr Tyr Thr Lys Asn Met Arg Pro
                                165 170 175
Met Tyr Pro Thr Lys Thr Phe Pro Asn His Tyr Ser Ile Val Thr Gly
                        180 185 190
Leu Tyr Pro Glu Ser His Gly Ile Ile Asp Asn Lys Met Tyr Asp Pro
                195 200 205
Lys Met Asn Ala Ser Phe Ser Leu Lys Ser Lys Glu Lys Phe Asn Pro
        210 215 220
Leu Trp Tyr Lys Gly Gln Pro Ile Trp Val Thr Ala Asn His Gln Glu
225 230 235 240
Val Lys Ser Gly Thr Tyr Phe Trp Pro Gly Ser Asp Val Glu Ile Asp
                                245 250 255
Gly Ile Leu Pro Asp Ile Tyr Lys Val Tyr Asn Gly Ser Val Pro Phe
                        260 265 270
Glu Glu Arg Ile Leu Ala Val Leu Glu Trp Leu Gln Leu Pro Ser His
                275 280 285
Glu Arg Pro His Phe Tyr Thr Leu Tyr Leu Glu Glu Pro Asp Ser Ser
        290 295 300
Gly His Ser His Gly Pro Val Ser Ser Glu Val Ile Lys Ala Leu Gln
305 310 315 320
Lys Val Asp Arg Leu Val Gly Met Leu Met Asp Gly Leu Lys Asp Leu
                                325 330 335
Gly Leu Asp Lys Cys Leu Asn Leu Ile Leu Ile Ser Asp His Gly Met
                        340 345 350
Glu Gln Gly Ser Cys Lys Lys Tyr Val Tyr Leu Asn Lys Tyr Leu Gly
                355 360 365
Asp Val Asn Asn Val Lys Val Val Tyr Gly Pro Ala Ala Arg Leu Arg
        370 375 380
Pro Thr Asp Val Pro Glu Thr Tyr Tyr Ser Phe Asn Tyr Glu Ala Leu
385 390 395 400
Ala Lys Asn Leu Ser Cys Arg Glu Pro Asn Gln His Phe Arg Pro Tyr
                                405 410 415
Leu Lys Pro Phe Leu Pro Lys Arg Leu His Phe Ala Lys Ser Asp Arg
                        420 425 430
Ile Glu Pro Leu Thr Phe Tyr Leu Asp Pro Gln Trp Gln Leu Ala Leu
                435 440 445
Asn Pro Ser Glu Arg Lys Tyr Cys Gly Ser Gly Phe His Gly Ser Asp
        450 455 460
Asn Leu Phe Ser Asn Met Gln Ala Leu Phe Ile Gly Tyr Gly Pro Ala
465 470 475 480
Phe Lys His Gly Ala Glu Val Asp Ser Phe Glu Asn Ile Glu Val Tyr
                                485 490 495
Asn Leu Met Cys Asp Leu Leu Gly Leu Ile Pro Ala Pro Asn Asn Gly
                        500 505 510
Ser His Gly Ser Leu Asn His Leu Leu Lys Lys Pro Ile Tyr Asn Pro
                515 520 525
Ser His Pro Lys Glu Glu Gly Phe Leu Ser Gln Cys Pro Ile Lys Ser
        530 535 540
Thr Ser Asn Asp Leu Gly Cys Thr Cys Asp Pro Trp Ile Val Pro Ile
545 550 555 560
Lys Asp Phe Glu Lys Gln Leu Asn Leu Thr Thr Glu Asp Val Asp Asp
                                565 570 575
Ile Tyr His Met Thr Val Pro Tyr Gly Arg Pro Arg Ile Leu Leu Lys
                        580 585 590
Gln His Arg Val Cys Leu Leu Gln Gln Gln Gln Phe Leu Thr Gly Tyr
                595 600 605
Ser Leu Asp Leu Leu Met Pro Leu Trp Ala Ser Tyr Thr Phe Leu Ser
        610 615 620
Asn Asp Gln Phe Ser Arg Asp Asp Phe Ser Asn Cys Leu Tyr Gln Asp
625 630 635 640
Leu Arg Ile Pro Leu Ser Pro Val His Lys Cys Ser Tyr Tyr Lys Ser
                                645 650 655
Asn Ser Lys Leu Ser Tyr Gly Phe Leu Thr Pro Pro Arg Leu Asn Arg
                        660 665 670
Val Ser Asn His Ile Tyr Ser Glu Ala Leu Leu Thr Ser Asn Ile Val
                675 680 685
Pro Met Tyr Gln Ser Phe Gln Val Ile Trp His Tyr Leu His Asp Thr
        690 695 700
Leu Leu Gln Arg Tyr Ala His Glu Arg Asn Gly Ile Asn Val Val Ser
705 710 715 720
Gly Pro Val Phe Asp Phe Asp Tyr Asp Gly Arg Tyr Asp Ser Leu Glu
                                725 730 735
Ile Leu Lys Gln Asn Ser Arg Val Ile Arg Ser Gln Glu Ile Leu Ile
                        740 745 750
Pro Thr His Phe Phe Ile Val Leu Thr Ser Cys Lys Gln Leu Ser Glu
                755 760 765
Thr Pro Leu Glu Cys Ser Ala Leu Glu Ser Ser Ala Tyr Ile Leu Pro
        770 775 780
His Arg Pro Asp Asn Ile Glu Ser Cys Thr His Gly Lys Arg Glu Ser
785 790 795 800
Ser Trp Val Glu Glu Leu Leu Thr Leu His Arg Ala Arg Val Thr Asp
                                805 810 815
Val Glu Leu Ile Thr Gly Leu Ser Phe Tyr Gln Asp Arg Gln Glu Ser
                        820 825 830
Val Ser Glu Leu Leu Arg Leu Lys Thr His Leu Pro Ile Phe Ser Gln
                835 840 845
Gln Asp
        850
Singly underlined: signal peptide sequence; double-underlined:
beginning and end of NPP1; ** = cleavage position at the signal
peptide sequence
SEQ. ID NO: 13-ENPP51-ALB Amino Acid Sequence:
Met Thr Ser Lys Phe Leu Leu Val Ser Phe Ile Leu Ala Ala Leu Ser
1 5 10 15
Leu Ser Thr Thr Phe Ser Leu Gln**Pro Ser Cys Ala Lys Glu Val Lys
                        20 25 30
Ser Cys Lys Gly Arg Cys Phe Glu Arg Thr Phe Ser Asn Cys Arg Cys
                35 40 45
Asp Ala Ala Cys Val Ser Leu Gly Asn Cys Cys Leu Asp Phe Gln Glu
        50 55 60
Thr Cys Val Glu Pro Thr His Ile Trp Thr Cys Asn Lys Phe Arg Cys
65 70 75 80
Gly Glu Lys Arg Leu Ser Arg Phe Val Cys Ser Cys Ala Asp Asp Cys
                                85 90 95
Lys Thr His Asn Asp Cys Cys Ile Asn Tyr Ser Ser Val Cys Gln Asp
                        100 105 110
Lys Lys Ser Trp Val Glu Glu Thr Cys Glu Ser Ile Asp Thr Pro Glu
                115 120 125
Cys Pro Ala Glu Phe Glu Ser Pro Pro Thr Leu Leu Phe Ser Leu Asp
        130 135 140
Gly Phe Arg Ala Glu Tyr Leu His Thr Trp Gly Gly Leu Leu Pro Val
145 150 155 160
Ile Ser Lys Leu Lys Asn Cys Gly Thr Tyr Thr Lys Asn Met Arg Pro
                                165 170 175
Met Tyr Pro Thr Lys Thr Phe Pro Asn His Tyr Ser Ile Val Thr Gly
                        180 185 190
Leu Tyr Pro Glu Ser His Gly Ile Ile Asp Asn Lys Met Tyr Asp Pro
                195 200 205
Lys Met Asn Ala Ser Phe Ser Leu Lys Ser Lys Glu Lys Phe Asn Pro
        210 215 220
Leu Trp Tyr Lys Gly Gln Pro Ile Trp Val Thr Ala Asn His Gln Glu
225 230 235 240
Val Lys Ser Gly Thr Tyr Phe Trp Pro Gly Ser Asp Val Glu Ile Asp
                                245 250 255
Gly Ile Leu Pro Asp Ile Tyr Lys Val Tyr Asn Gly Ser Val Pro Phe
                        260 265 270
Glu Glu Arg Ile Leu Ala Val Leu Glu Trp Leu Gln Leu Pro Ser His
                275 280 285
Glu Arg Pro His Phe Tyr Thr Leu Tyr Leu Glu Glu Pro Asp Ser Ser
        290 295 300
Gly His Ser His Gly Pro Val Ser Ser Glu Val Ile Lys Ala Leu Gln
305 310 315 320
Lys Val Asp Arg Leu Val Gly Met Leu Met Asp Gly Leu Lys Asp Leu
                                325 330 335
Gly Leu Asp Lys Cys Leu Asn Leu Ile Leu Ile Ser Asp His Gly Met
                        340 345 350
Glu Gln Gly Ser Cys Lys Lys Tyr Val Tyr Leu Asn Lys Tyr Leu Gly
                355 360 365
Asp Val Asn Asn Val Lys Val Val Tyr Gly Pro Ala Ala Arg Leu Arg
        370 375 380
Pro Thr Asp Val Pro Glu Thr Tyr Tyr Ser Phe Asn Tyr Glu Ala Leu
385 390 395 400
Ala Lys Asn Leu Ser Cys Arg Glu Pro Asn Gln His Phe Arg Pro Tyr
                                405 410 415
Leu Lys Pro Phe Leu Pro Lys Arg Leu His Phe Ala Lys Ser Asp Arg
                        420 425 430
Ile Glu Pro Leu Thr Phe Tyr Leu Asp Pro Gln Trp Gln Leu Ala Leu
                435 440 445
Asn Pro Ser Glu Arg Lys Tyr Cys Gly Ser Gly Phe His Gly Ser Asp
        450 455 460
Asn Leu Phe Ser Asn Met Gln Ala Leu Phe Ile Gly Tyr Gly Pro Ala
465 470 475 480
Phe Lys His Gly Ala Glu Val Asp Ser Phe Glu Asn Ile Glu Val Tyr
                                485 490 495
Asn Leu Met Cys Asp Leu Leu Gly Leu Ile Pro Ala Pro Asn Asn Gly
                        500 505 510
Ser His Gly Ser Leu Asn His Leu Leu Lys Lys Pro Ile Tyr Asn Pro
                515 520 525
Ser His Pro Lys Glu Glu Gly Phe Leu Ser Gln Cys Pro Ile Lys Ser
        530 535 540
Thr Ser Asn Asp Leu Gly Cys Thr Cys Asp Pro Trp Ile Val Pro Ile
545 550 555 560
Lys Asp Phe Glu Lys Gln Leu Asn Leu Thr Thr Glu Asp Val Asp Asp
                                565 570 575
Ile Tyr His Met Thr Val Pro Tyr Gly Arg Pro Arg Ile Leu Leu Lys
                        580 585 590
Gln His Arg Val Cys Leu Leu Gln Gln Gln Gln Phe Leu Thr Gly Tyr
                595 600 605
Ser Leu Asp Leu Leu Met Pro Leu Trp Ala Ser Tyr Thr Phe Leu Ser
        610 615 620
Asn Asp Gln Phe Ser Arg Asp Asp Phe Ser Asn Cys Leu Tyr Gln Asp
625 630 635 640
Leu Arg Ile Pro Leu Ser Pro Val His Lys Cys Ser Tyr Tyr Lys Ser
                                645 650 655
Asn Ser Lys Leu Ser Tyr Gly Phe Leu Thr Pro Pro Arg Leu Asn Arg
                        660 665 670
Val Ser Asn His Ile Tyr Ser Glu Ala Leu Leu Thr Ser Asn Ile Val
                675 680 685
Pro Met Tyr Gln Ser Phe Gln Val Ile Trp His Tyr Leu His Asp Thr
        690 695 700
Leu Leu Gln Arg Tyr Ala His Glu Arg Asn Gly Ile Asn Val Val Ser
705 710 715 720
Gly Pro Val Phe Asp Phe Asp Tyr Asp Gly Arg Tyr Asp Ser Leu Glu
                                725 730 735
Ile Leu Lys Gln Asn Ser Arg Val Ile Arg Ser Gln Glu Ile Leu Ile
                        740 745 750
Pro Thr His Phe Phe Ile Val Leu Thr Ser Cys Lys Gln Leu Ser Glu
                755 760 765
Thr Pro Leu Glu Cys Ser Ala Leu Glu Ser Ser Ala Tyr Ile Leu Pro
        770 775 780
His Arg Pro Asp Asn Ile Glu Ser Cys Thr His Gly Lys Arg Glu Ser
785 790 795 800
Ser Trp Val Glu Glu Leu Leu Thr Leu His Arg Ala Arg Val Thr Asp
                                805 810 815
Val Glu Leu Ile Thr Gly Leu Ser Phe Tyr Gln Asp Arg Gln Glu Ser
                        820 825 830
Val Ser Glu Leu Leu Arg Leu Lys Thr His Leu Pro Ile Phe Ser Gln
                835 840 845
Glu Asp Gly Gly Ser Gly Gly Ser Met Lys Trp Val Thr Phe Leu Leu
        850 855 860
Leu Leu Phe Val Ser Gly Ser Ala Phe Ser Arg Gly Val Phe Arg Arg
865 870 875 880
Glu Ala His Lys Ser Glu Ile Ala His Arg Tyr Asn Asp Leu Gly Glu
                                885 890 895
Gln His Phe Lys Gly Leu Val Leu Ile Ala Phe Ser Gln Tyr Leu Gln
                        900 905 910
Lys Cys Ser Tyr Asp Glu His Ala Lys Leu Val Gln Glu Val Thr Asp
                915 920 925
Phe Ala Lys Thr Cys Val Ala Asp Glu Ser Ala Ala Asn Cys Asp Lys
        930 935 940
Ser Leu His Thr Leu Phe Gly Asp Lys Leu Cys Ala Ile Pro Asn Leu
945 950 955 960
Arg Glu Asn Tyr Gly Glu Leu Ala Asp Cys Cys Thr Lys Gln Glu Pro
                                965 970 975
Glu Arg Asn Glu Cys Phe Leu Gln His Lys Asp Asp Asn Pro Ser Leu
                        980 985 990
Pro Pro Phe Glu Arg Pro Glu Ala Glu Ala Met Cys Thr Ser Phe Lys
                995 1000 1005
Glu Asn Pro Thr Thr Phe Met Gly His Tyr Leu His Glu Val Ala
        1010 1015 1020
Arg Arg His Pro Tyr Phe Tyr Ala Pro Glu Leu Leu Tyr Tyr Ala
        1025 1030 1035
Glu Gln Tyr Asn Glu Ile Leu Thr Gln Cys Cys Ala Glu Ala Asp
        1040 1045 1050
Lys Glu Ser Cys Leu Thr Pro Lys Leu Asp Gly Val Lys Glu Lys
        1055 1060 1065
Ala Leu Val Ser Ser Val Arg Gln Arg Met Lys Cys Ser Ser Met
        1070 1075 1080
Gln Lys Phe Gly Glu Arg Ala Phe Lys Ala Trp Ala Val Ala Arg
        1085 1090 1095
Leu Ser Gln Thr Phe Pro Asn Ala Asp Phe Ala Glu Ile Thr Lys
        1100 1105 1110
Leu Ala Thr Asp Leu Thr Lys Val Asn Lys Glu Cys Cys His Gly
        1115 1120 1125
Asp Leu Leu Glu Cys Ala Asp Asp Arg Ala Glu Leu Ala Lys Tyr
        1130 1135 1140
Met Cys Glu Asn Gln Ala Thr Ile Ser Ser Lys Leu Gln Thr Cys
        1145 1150 1155
Cys Asp Lys Pro Leu Leu Lys Lys Ala His Cys Leu Ser Glu Val
        1160 1165 1170
Glu His Asp Thr Met Pro Ala Asp Leu Pro Ala Ile Ala Ala Asp
        1175 1180 1185
Phe Val Glu Asp Gln Glu Val Cys Lys Asn Tyr Ala Glu Ala Lys
        1190 1195 1200
Asp Val Phe Leu Gly Thr Phe Leu Tyr Glu Tyr Ser Arg Arg His
        1205 1210 1215
Pro Asp Tyr Ser Val Ser Leu Leu Leu Arg Leu Ala Lys Lys Tyr
        1220 1225 1230
Glu Ala Thr Leu Glu Lys Cys Cys Ala Glu Ala Asn Pro Pro Ala
        1235 1240 1245
Cys Tyr Gly Thr Val Leu Ala Glu Phe Gln Pro Leu Val Glu Glu
        1250 1255 1260
Pro Lys Asn Leu Val Lys Thr Asn Cys Asp Leu Tyr Glu Lys Leu
        1265 1270 1275
Gly Glu Tyr Gly Phe Gln Asn Ala Ile Leu Val Arg Tyr Thr Gln
        1280 1285 1290
Lys Ala Pro Gln Val Ser Thr Pro Thr Leu Val Glu Ala Ala Arg
        1295 1300 1305
Asn Leu Gly Arg Val Gly Thr Lys Cys Cys Thr Leu Pro Glu Asp
        1310 1315 1320
Gln Arg Leu Pro Cys Val Glu Asp Tyr Leu Ser Ala Ile Leu Asn
        1325 1330 1335
Arg Val Cys Leu Leu His Glu Lys Thr Pro Val Ser Glu His Val
        1340 1345 1350
Thr Lys Cys Cys Ser Gly Ser Leu Val Glu Arg Arg Pro Cys Phe
        1355
Ser Ala Leu Thr Val Asp Glu Thr Tyr Val Pro Lys Glu Phe Lys
        1370 1375 1380
Ala Glu Thr Phe Thr Phe His Ser Asp Ile Cys Thr Leu Pro Glu
        1385 1390 1395
Lys Glu Lys Gln Ile Lys Lys Gln Thr Ala Leu Ala Glu Leu Val
        1400 1405 1410
Lys His Lys Pro Lys Ala Thr Ala Glu Gln Leu Lys Thr Val Met
        1415 1420 1425
Asp Asp Phe Ala Gln Phe Leu Asp Thr Cys Cys Lys Ala Ala Asp
        1430 1435 1440
Lys Asp Thr Cys Phe Ser Thr Glu Gly Pro Asn Leu Val Thr Arg
        1445 1450 1455
Cys Lys Asp Ala Leu Ala Arg Ser Trp Ser His Pro Gln Phe Glu
        1460 1465 1470
Lys
Singly underlined: signal peptide sequence; double-underlined:
beginning and end of NPP1; ** = cleavage position at the signal
peptide sequence; bold residues indicate albumin sequence
SEQ. ID NO: 14-ENPP5-NPP3-Fc sequence
Met Thr Ser Lys Phe Leu Leu Val Ser Phe Ile Leu Ala Ala Leu Ser
1 5 10 15
Leu Ser Thr Thr Phe Ser**Lys Gln Gly Ser Cys Arg Lys Lys Cys Phe
                        20 25 30
Asp Ala Ser Phe Arg Gly Leu Glu Asn Cys Arg Cys Asp Val Ala Cys
                35 40 45
Lys Asp Arg Gly Asp Cys Cys Trp Asp Phe Glu Asp Thr Cys Val Glu
        50 55 60
Ser Thr Arg Ile Trp Met Cys Asn Lys Phe Arg Cys Gly Glu Arg Leu
65 70 75 80
Glu Ala Ser Leu Cys Ser Cys Ser Asp Asp Cys Leu Gln Arg Lys Asp
                                85 90 95
Cys Cys Ala Asp Tyr Lys Ser Val Cys Gln Gly Glu Thr Ser Trp Leu
                        100 105 110
Glu Glu Asn Cys Asp Thr Ala Gln Gln Ser Gln Cys Pro Glu Gly Phe
                115 120 125
Asp Leu Pro Pro Val Ile Leu Phe Ser Met Asp Gly Phe Arg Ala Glu
        130 135 140
Tyr Leu Tyr Thr Trp Asp Thr Leu Met Pro Asn Ile Asn Lys Leu Lys
145 150 155 160
Thr Cys Gly Ile His Ser Lys Tyr Met Arg Ala Met Tyr Pro Thr Lys
                                165 170 175
Thr Phe Pro Asn His Tyr Thr Ile Val Thr Gly Leu Tyr Pro Glu Ser
                        180 185 190
His Gly Ile Ile Asp Asn Asn Met Tyr Asp Val Asn Leu Asn Lys Asn
                195 200 205
Phe Ser Leu Ser Ser Lys Glu Gln Asn Asn Pro Ala Trp Trp His Gly
        210 215 220
Gln Pro Met Trp Leu Thr Ala Met Tyr Gln Gly Leu Lys Ala Ala Thr
225 230 235 240
Tyr Phe Trp Pro Gly Ser Glu Val Ala Ile Asn Gly Ser Phe Pro Ser
                                245 250 255
Ile Tyr Met Pro Tyr Asn Gly Ser Val Pro Phe Glu Glu Arg Ile Ser
                        260 265 270
Thr Leu Leu Lys Trp Leu Asp Leu Pro Lys Ala Glu Arg Pro Arg Phe
                275 280 285
Tyr Thr Met Tyr Phe Glu Glu Pro Asp Ser Ser Gly His Ala Gly Gly
        290 295 300
Pro Val Ser Ala Arg Val Ile Lys Ala Leu Gln Val Val Asp His Ala
305 310 315 320
Phe Gly Met Leu Met Glu Gly Leu Lys Gln Arg Asn Leu His Asn Cys
                                325 330 335
Val Asn Ile Ile Leu Leu Ala Asp His Gly Met Asp Gln Thr Tyr Cys
                        340 345 350
Asn Lys Met Glu Tyr Met Thr Asp Tyr Phe Pro Arg Ile Asn Phe Phe
                355 360 365
Tyr Met Tyr Glu Gly Pro Ala Pro Arg Ile Arg Ala His Asn Ile Pro
        370 375 380
His Asp Phe Phe Ser Phe Asn Ser Glu Glu Ile Val Arg Asn Leu Ser
385 390 395 400
Cys Arg Lys Pro Asp Gln His Phe Lys Pro Tyr Leu Thr Pro Asp Leu
                                405 410 415
Pro Lys Arg Leu His Tyr Ala Lys Asn Val Arg Ile Asp Lys Val His
                        420 425 430
Leu Phe Val Asp Gln Gln Trp Leu Ala Val Arg Ser Lys Ser Asn Thr
                435 440 445
Asn Cys Gly Gly Gly Asn His Gly Tyr Asn Asn Glu Phe Arg Ser Met
        450 455 460
Glu Ala Ile Phe Leu Ala His Gly Pro Ser Phe Lys Glu Lys Thr Glu
465 470 475 480
Val Glu Pro Phe Glu Asn Ile Glu Val Tyr Asn Leu Met Cys Asp Leu
                                485 490 495
Leu Arg Ile Gln Pro Ala Pro Asn Asn Gly Thr His Gly Ser Leu Asn
                        500 505 510
His Leu Leu Lys Val Pro Phe Tyr Glu Pro Ser His Ala Glu Glu Val
                515 520 525
Ser Lys Phe Ser Val Cys Gly Phe Ala Asn Pro Leu Pro Thr Glu Ser
        530 535 540
Leu Asp Cys Phe Cys Pro His Leu Gln Asn Ser Thr Gln Leu Glu Gln
545 550 555 560
Val Asn Gln Met Leu Asn Leu Thr Gln Glu Glu Ile Thr Ala Thr Val
                                565 570 575
Lys Val Asn Leu Pro Phe Gly Arg Pro Arg Val Leu Gln Lys Asn Val
                        580 585 590
Asp His Cys Leu Leu Tyr His Arg Glu Tyr Val Ser Gly Phe Gly Lys
                595 600 605
Ala Met Arg Met Pro Met Trp Ser Ser Tyr Thr Val Pro Gln Leu Gly
        610 615 620
Asp Thr Ser Pro Leu Pro Pro Thr Val Pro Asp Cys Leu Arg Ala Asp
625 630 635 640
Val Arg Val Pro Pro Ser Glu Ser Gln Lys Cys Ser Phe Tyr Leu Ala
                                645 650 655
Asp Lys Asn Ile Thr His Gly Phe Leu Tyr Pro Pro Ala Ser Asn Arg
                        660 665 670
Thr Ser Asp Ser Gln Tyr Asp Ala Leu Ile Thr Ser Asn Leu Val Pro
                675 680 685
Met Tyr Glu Glu Phe Arg Lys Met Trp Asp Tyr Phe His Ser Val Leu
        690 695 700
Leu Ile Lys His Ala Thr Glu Arg Asn Gly Val Asn Val Val Ser Gly
705 710 715 720
Pro Ile Phe Asp Tyr Asn Tyr Asp Gly His Phe Asp Ala Pro Asp Glu
                                725 730 735
Ile Thr Lys His Leu Ala Asn Thr Asp Val Pro Ile Pro Thr His Tyr
                        740 745 750
Phe Val Val Leu Thr Ser Cys Lys Asn Lys Ser His Thr Pro Glu Asn
                755 760 765
Cys Pro Gly Trp Leu Asp Val Leu Pro Phe Ile Ile Pro His Arg Pro
        770 775 780
Thr Asn Val Glu Ser Cys Pro Glu Gly Lys Pro Glu Ala Leu Trp Val
785 790 795 800
Glu Glu Arg Phe Thr Ala His Ile Ala Arg Val Arg Asp Val Glu Leu
                                805 810 815
Leu Thr Gly Leu Asp Phe Tyr Gln Asp Lys Val Gln Pro Val Ser Glu
                        820 825 830
Ile Leu Gln Leu Lys Thr Tyr Leu Pro Thr Phe Glu Thr Thr Ile Asp
                835 840 845
Lys Thr His Thr Cys Pro Pro Cys Pro Ala Pro Glu Leu Leu Gly Gly
        850 855 860
Pro Ser Val Phe Leu Phe Pro Pro Lys Pro Lys Asp Thr Leu Met Ile
865 870 875 880
Ser Arg Thr Pro Glu Val Thr Cys Val Val Val Asp Val Ser His Glu
                                885 890 895
Asp Pro Glu Val Lys Phe Asn Trp Tyr Val Asp Gly Val Glu Val His
                        900 905 910
Asn Ala Lys Thr Lys Pro Arg Glu Glu Gln Tyr Asn Ser Thr Tyr Arg
                915 920 925
Val Val Ser Val Leu Thr Val Leu His Gln Asp Trp Leu Asn Gly Lys
        930 935 940
Glu Tyr Lys Cys Lys Val Ser Asn Lys Ala Leu Pro Ala Pro Ile Glu
945 950 955 960
Lys Thr Ile Ser Lys Ala Lys Gly Gln Pro Arg Glu Pro Gln Val Tyr
                                965 970 975
Thr Leu Pro Pro Ser Arg Glu Glu Met Thr Lys Asn Gln Val Ser Leu
                        980 985 990
Thr Cys Leu Val Lys Gly Phe Tyr Pro Ser Asp Ile Ala Val Glu Trp
                995 1000 1005
Glu Ser Asn Gly Gln Pro Glu Asn Asn Tyr Lys Thr Thr Pro Pro
        1010 1015 1020
Val Leu Asp Ser Asp Gly Ser Phe Phe Leu Tyr Ser Lys Leu Thr
        1025 1030 1035
Val Asp Lys Ser Arg Trp Gln Gln Gly Asn Val Phe Ser Cys Ser
        1040 1045 1050
Val Met His Glu Ala Leu His Asn His Tyr Thr Gln Lys Ser Leu
        1055 1060 1065
Ser Leu Ser Pro Gly Lys
        1070
Singly underlined: signal peptide sequence; double-underlined:
beginning and end of NPP3; ** = cleavage position at the signal
peptide sequence; bold residues indicate albumin sequence
SEQ. ID NO: 15-ENPP5-NPP3-Albumin sequence
Met Thr Ser Lys Phe Leu Leu Val Ser Phe Ile Leu Ala Ala Leu Ser
1 5 10 15
Leu Ser Thr Thr Phe Ser**Lys Gln Gly Ser Cys Arg Lys Lys Cys Phe
                        20 25 30
Asp Ala Ser Phe Arg Gly Leu Glu Asn Cys Arg Cys Asp Val Ala Cys
              35 40 45
Lys Asp Arg Gly Asp Cys Cys Trp Asp Phe Glu Asp Thr Cys Val Glu
        50 55 60
Ser Thr Arg Ile Trp Met Cys Asn Lys Phe Arg Cys Gly Glu Arg Leu
65 70 75 80
Glu Ala Ser Leu Cys Ser Cys Ser Asp Asp Cys Leu Gln Arg Lys Asp
                                85 90 95
Cys Cys Ala Asp Tyr Lys Ser Val Cys Gln Gly Glu Thr Ser Trp Leu
                        100 105 110
Glu Glu Asn Cys Asp Thr Ala Gln Gln Ser Gln Cys Pro Glu Gly Phe
                115 120 125
Asp Leu Pro Pro Val Ile Leu Phe Ser Met Asp Gly Phe Arg Ala Glu
        130 135 140
Tyr Leu Tyr Thr Trp Asp Thr Leu Met Pro Asn Ile Asn Lys Leu Lys
145 150 155 160
Thr Cys Gly Ile His Ser Lys Tyr Met Arg Ala Met Tyr Pro Thr Lys
                                165 170 175
Thr Phe Pro Asn His Tyr Thr Ile Val Thr Gly Leu Tyr Pro Glu Ser
                      180 185 190
His Gly Ile Ile Asp Asn Asn Met Tyr Asp Val Asn Leu Asn Lys Asn
                195 200 205
Phe Ser Leu Ser Ser Lys Glu Gln Asn Asn Pro Ala Trp Trp His Gly
        210 215 220
Gln Pro Met Trp Leu Thr Ala Met Tyr Gln Gly Leu Lys Ala Ala Thr
225 230 235 240
Tyr Phe Trp Pro Gly Ser Glu Val Ala Ile Asn Gly Ser Phe Pro Ser
                                245 250 255
Ile Tyr Met Pro Tyr Asn Gly Ser Val Pro Phe Glu Glu Arg Ile Ser
                        260 265 270
Thr Leu Leu Lys Trp Leu Asp Leu Pro Lys Ala Glu Arg Pro Arg Phe
                275 280 285
Tyr Thr Met Tyr Phe Glu Glu Pro Asp Ser Ser Gly His Ala Gly Gly
        290 295 300
Pro Val Ser Ala Arg Val Ile Lys Ala Leu Gln Val Val Asp His Ala
305 310 315 320
Phe Gly Met Leu Met Glu Gly Leu Lys Gln Arg Asn Leu His Asn Cys
                                325 330 335
Val Asn Ile Ile Leu Leu Ala Asp His Gly Met Asp Gln Thr Tyr Cys
                        340 345 350
Asn Lys Met Glu Tyr Met Thr Asp Tyr Phe Pro Arg Ile Asn Phe Phe
                355 360 365
Tyr Met Tyr Glu Gly Pro Ala Pro Arg Ile Arg Ala His Asn Ile Pro
        370 375 380
His Asp Phe Phe Ser Phe Asn Ser Glu Glu Ile Val Arg Asn Leu Ser
385 390 395 400
Cys Arg Lys Pro Asp Gln His Phe Lys Pro Tyr Leu Thr Pro Asp Leu
                                405 410 415
Pro Lys Arg Leu His Tyr Ala Lys Asn Val Arg Ile Asp Lys Val His
                        420 425 430
Leu Phe Val Asp Gln Gln Trp Leu Ala Val Arg Ser Lys Ser Asn Thr
                435 440 445
Asn Cys Gly Gly Gly Asn His Gly Tyr Asn Asn Glu Phe Arg Ser Met
        450 455 460
Glu Ala Ile Phe Leu Ala His Gly Pro Ser Phe Lys Glu Lys Thr Glu
465 470 475 480
Val Glu Pro Phe Glu Asn Ile Glu Val Tyr Asn Leu Met Cys Asp Leu
                                485 490 495
Leu Arg Ile Gln Pro Ala Pro Asn Asn Gly Thr His Gly Ser Leu Asn
                        500 505 510
His Leu Leu Lys Val Pro Phe Tyr Glu Pro Ser His Ala Glu Glu Val
                515 520 525
Ser Lys Phe Ser Val Cys Gly Phe Ala Asn Pro Leu Pro Thr Glu Ser
        530 535 540
Leu Asp Cys Phe Cys Pro His Leu Gln Asn Ser Thr Gln Leu Glu Gln
545 550 555 560
Val Asn Gln Met Leu Asn Leu Thr Gln Glu Glu Ile Thr Ala Thr Val
                                565 570 575
Lys Val Asn Leu Pro Phe Gly Arg Pro Arg Val Leu Gln Lys Asn Val
                        580 585 590
Asp His Cys Leu Leu Tyr His Arg Glu Tyr Val Ser Gly Phe Gly Lys
                595 600 605
Ala Met Arg Met Pro Met Trp Ser Ser Tyr Thr Val Pro Gln Leu Gly
        610 615 620
Asp Thr Ser Pro Leu Pro Pro Thr Val Pro Asp Cys Leu Arg Ala Asp
625 630 635 640
Val Arg Val Pro Pro Ser Glu Ser Gln Lys Cys Ser Phe Tyr Leu Ala
                                645 650 655
Asp Lys Asn Ile Thr His Gly Phe Leu Tyr Pro Pro Ala Ser Asn Arg
                        660 665 670
Thr Ser Asp Ser Gln Tyr Asp Ala Leu Ile Thr Ser Asn Leu Val Pro
                675 680 685
Met Tyr Glu Glu Phe Arg Lys Met Trp Asp Tyr Phe His Ser Val Leu
        690 695 700
Leu Ile Lys His Ala Thr Glu Arg Asn Gly Val Asn Val Val Ser Gly
705 710 715 720
Pro Ile Phe Asp Tyr Asn Tyr Asp Gly His Phe Asp Ala Pro Asp Glu
                                725 730 735
Ile Thr Lys His Leu Ala Asn Thr Asp Val Pro Ile Pro Thr His Tyr
                        740 745 750
Phe Val Val Leu Thr Ser Cys Lys Asn Lys Ser His Thr Pro Glu Asn
                755 760 765
Cys Pro Gly Trp Leu Asp Val Leu Pro Phe Ile Ile Pro His Arg Pro
        770 775 780
Thr Asn Val Glu Ser Cys Pro Glu Gly Lys Pro Glu Ala Leu Trp Val
785 790 795 800
Glu Glu Arg Phe Thr Ala His Ile Ala Arg Val Arg Asp Val Glu Leu
                                805 810 815
Leu Thr Gly Leu Asp Phe Tyr Gln Asp Lys Val Gln Pro Val Ser Glu
                        820 825 830
Ile Leu Gln Leu Lys Thr Tyr Leu Pro Thr Phe Glu Thr Thr Ile Gly
                835 840 845
Gly Gly Ser Gly Gly Gly Gly Ser Gly Gly Gly Gly Ser Met Lys Trp
        850 855 860
Val Thr Phe Leu Leu Leu Leu Phe Val Ser Gly Ser Ala Phe Ser Arg
865 870 875 880
Gly Val Phe Arg Arg Glu Ala His Lys Ser Glu Ile Ala His Arg Tyr
                                885 890 895
Asn Asp Leu Gly Glu Gln His Phe Lys Gly Leu Val Leu Ile Ala Phe
                        900 905 910
Ser Gln Tyr Leu Gln Lys Cys Ser Tyr Asp Glu His Ala Lys Leu Val
                915 920 925
Gln Glu Val Thr Asp Phe Ala Lys Thr Cys Val Ala Asp Glu Ser Ala
        930 935 940
Ala Asn Cys Asp Lys Ser Leu His Thr Leu Phe Gly Asp Lys Leu Cys
945 950 955 960
Ala Ile Pro Asn Leu Arg Glu Asn Tyr Gly Glu Leu Ala Asp Cys Cys
                                965 970 975
Thr Lys Gln Glu Pro Glu Arg Asn Glu Cys Phe Leu Gln His Lys Asp
                        980 985 990
Asp Asn Pro Ser Leu Pro Pro Phe Glu Arg Pro Glu Ala Glu Ala Met
                995 1000 1005
Cys Thr Ser Phe Lys Glu Asn Pro Thr Thr Phe Met Gly His Tyr
        1010 1015 1020
Leu His Glu Val Ala Arg Arg His Pro Tyr Phe Tyr Ala Pro Glu
        1025 1030 1035
Leu Leu Tyr Tyr Ala Glu Gln Tyr Asn Glu Ile Leu Thr Gln Cys
        1040 1045 1050
Cys Ala Glu Ala Asp Lys Glu Ser Cys Leu Thr Pro Lys Leu Asp
        1055 1060 1065
Gly Val Lys Glu Lys Ala Leu Val Ser Ser Val Arg Gln Arg Met
        1070 1075 1080
Lys Cys Ser Ser Met Gln Lys Phe Gly Glu Arg Ala Phe Lys Ala
        1085 1090 1095
Trp Ala Val Ala Arg Leu Ser Gln Thr Phe Pro Asn Ala Asp Phe
        1100 1105 1110
Ala Glu Ile Thr Lys Leu Ala Thr Asp Leu Thr Lys Val Asn Lys
        1115 1120 1125
Glu Cys Cys His Gly Asp Leu Leu Glu Cys Ala Asp Asp Arg Ala
        1130 1135 1140
Glu Leu Ala Lys Tyr Met Cys Glu Asn Gln Ala Thr Ile Ser Ser
        1145 1150 1155
Lys Leu Gln Thr Cys Cys Asp Lys Pro Leu Leu Lys Lys Ala His
        1160 1165 1170
Cys Leu Ser Glu Val Glu His Asp Thr Met Pro Ala Asp Leu Pro
        1175 1180 1185
Ala Ile Ala Ala Asp Phe Val Glu Asp Gln Glu Val Cys Lys Asn
        1190 1195 1200
Tyr Ala Glu Ala Lys Asp Val Phe Leu Gly Thr Phe Leu Tyr Glu
        1205 1210 1215
Tyr Ser Arg Arg His Pro Asp Tyr Ser Val Ser Leu Leu Leu Arg
        1220 1225 1230
Leu Ala Lys Lys Tyr Glu Ala Thr Leu Glu Lys Cys Cys Ala Glu
        1235 1340 1245
Ala Asn Pro Pro Ala Cys Tyr Gly Thr Val Leu Ala Glu Phe Gln
        1250 1255 1260
Pro Leu Val Glu Glu Pro Lys Asn Leu Val Lys Thr Asn Cys Asp
        1265 1270 1275
Leu Tyr Glu Lys Leu Gly Glu Tyr Gly Phe Gln Asn Ala Ile Leu
        1280 1285 1290
Val Arg Tyr Thr Gln Lys Ala Pro Gln Val Ser Thr Pro Thr Leu
        1295 1300 1305
Val Glu Ala Ala Arg Asn Leu Gly Arg Val Gly Thr Lys Cys Cys
        1310 1315 1320
Thr Leu Pro Glu Asp Gln Arg Leu Pro Cys Val Glu Asp Tyr Leu
        1325 1330 1335
Ser Ala Ile Leu Asn Arg Val Cys Leu Leu His Glu Lys Thr Pro
        1340 1345 1350
Val Ser Glu His Val Thr Lys Cys Cys Ser Gly Ser Leu Val Glu
        1355 1360 1365
Arg Arg Pro Cys Phe Ser Ala Leu Thr Val Asp Glu Thr Tyr Val
        1370 1375 1380
Pro Lys Glu Phe Lys Ala Glu Thr Phe Thr Phe 1395 Ser Asp Ile
        1385 1390 1395
Cys Thr Leu Pro Glu Lys Glu Lys Gln Ile Lys Lys Gln Thr Ala
        1400 1405 1410
Leu Ala Glu Leu Val Lys His Lys Pro Lys Ala Thr Ala Glu Gln
        1415 1420 1425
Leu Lys Thr Val Met Asp Asp Phe Ala Gln Phe Leu Asp Thr Cys
        1430 1435 1440
Cys Lys Ala Ala Asp Lys Asp Thr Cys Phe Ser Thr Glu Gly Pro
        1445 1450 1455
Asn Leu Val Thr Arg Cys Lys Asp Ala Leu Ala
        1460 1465
Singly underlined: signal peptide sequence; double-underlined:
beginning and end of NPP3; ** = cleavage position at the signal
peptide sequence; bold residues indicate albumin sequence
SEQ. ID NO: 16-ENPP5 Protein Export Signal Sequence
Met Thr Ser Lys Phe Leu Leu Val Ser Phe Ile Leu Ala Ala Leu Ser
1 5 10 15
Leu Ser Thr Thr Phe Ser Xaa
                        20
SEQ. ID NO: 17-ENPP51-Fc
Met Thr Ser Lys Phe Leu Leu Val Ser Phe Ile Leu Ala Ala Leu Ser
1 5 10 15
Leu Ser Thr Thr Phe Ser**Gly Leu Lys Pro Ser Cys Ala Lys Glu Val
                        20 25 30
Lys Ser Cys Lys Gly Arg Cys Phe Glu Arg Thr Phe Gly Asn Cys Arg
                35 40 45
Cys Asp Ala Ala Cys Val Glu Leu Gly Asn Cys Cys Leu Asp Tyr Gln
        50 55 60
Glu Thr Cys Ile Glu Pro Glu His Ile Trp Thr Cys Asn Lys Phe Arg
65 70 75 80
Cys Gly Glu Lys Arg Leu Thr Arg Ser Leu Cys Ala Cys Ser Asp Asp
                                85 90 95
Cys Lys Asp Lys Gly Asp Cys Cys Ile Asn Tyr Ser Ser Val Cys Gln
                        100 105 110
Gly Glu Lys Ser Trp Val Glu Glu Pro Cys Glu Ser Ile Asn Glu Pro
                115 120 125
Gln Cys Pro Ala Gly Phe Glu Thr Pro Pro Thr Leu Leu Phe Ser Leu
        130 135 140
Asp Gly Phe Arg Ala Glu Tyr Leu His Thr Trp Gly Gly Leu Leu Pro
145 150 155 160
Val Ile Ser Lys Leu Lys Lys Cys Gly Thr Tyr Thr Lys Asn Met Arg
                                165 170 175
Pro Val Tyr Pro Thr Lys Thr Phe Pro Asn His Tyr Ser Ile Val Thr
                        180 185 190
Gly Leu Tyr Pro Glu Ser His Gly Ile Ile Asp Asn Lys Met Tyr Asp
                195 200 205
Pro Lys Met Asn Ala Ser Phe Ser Leu Lys Ser Lys Glu Lys Phe Asn
        210 215 220
Pro Glu Trp Tyr Lys Gly Glu Pro Ile Trp Val Thr Ala Lys Tyr Gln
225 230 235 240
Gly Leu Lys Ser Gly Thr Phe Phe Trp Pro Gly Ser Asp Val Glu Ile
                                245 250 255
Asn Gly Ile Phe Pro Asp Ile Tyr Lys Met Tyr Asn Gly Ser Val Pro
                        260 265 270
Phe Glu Glu Arg Ile Leu Ala Val Leu Gln Trp Leu Gln Leu Pro Lys
                275 280 285
Asp Glu Arg Pro His Phe Tyr Thr Leu Tyr Leu Glu Glu Pro Asp Ser
        290 295 300
Ser Gly His Ser Tyr Gly Pro Val Ser Ser Glu Val Ile Lys Ala Leu
305 310 315 320
Gln Arg Val Asp Gly Met Val Gly Met Leu Met Asp Gly Leu Lys Glu
                                325 330 335
Leu Asn Leu His Arg Cys Leu Asn Leu Ile Leu Ile Ser Asp His Gly
                        340 345 350
Met Glu Gln Gly Ser Cys Lys Lys Tyr Ile Tyr Leu Asn Lys Tyr Leu
                355 360 365
Gly Asp Val Lys Asn Ile Lys Val Ile Tyr Gly Pro Ala Ala Arg Leu
        370 375 380
Arg Pro Ser Asp Val Pro Asp Lys Tyr Tyr Ser Phe Asn Tyr Glu Gly
385 390 395 400
Ile Ala Arg Asn Leu Ser Cys Arg Glu Pro Asn Gln His Phe Lys Pro
                                405 410 415
Tyr Leu Lys His Phe Leu Pro Lys Arg Leu His Phe Ala Lys Ser Asp
                        420 425 430
Arg Ile Glu Pro Leu Thr Phe Tyr Leu Asp Pro Gln Trp Gln Leu Ala
                435 440 445
Leu Asn Pro Ser Glu Arg Lys Tyr Cys Gly Ser Gly Phe His Gly Ser
        450 455 460
Asp Asn Val Phe Ser Asn Met Gln Ala Leu Phe Val Gly Tyr Gly Pro
465 470 475 480
Gly Phe Lys His Gly Ile Glu Ala Asp Thr Phe Glu Asn Ile Glu Val
                                485 490 495
Tyr Asn Leu Met Cys Asp Leu Leu Asn Leu Thr Pro Ala Pro Asn Asn
                        500 505 510
Gly Thr His Gly Ser Leu Asn His Leu Leu Lys Asn Pro Val Tyr Thr
                515 520 525
Pro Lys His Pro Lys Glu Val His Pro Leu Val Gln Cys Pro Phe Thr
        530 535 540
Arg Asn Pro Arg Asp Asn Leu Gly Cys Ser Cys Asn Pro Ser Ile Leu
545 550 555 560
Pro Ile Glu Asp Phe Gln Thr Gln Phe Asn Leu Thr Val Ala Glu Glu
                                565 570 575
Lys Ile Ile Lys His Glu Thr Leu Pro Tyr Gly Arg Pro Arg Val Leu
                        580 585 590
Gln Lys Glu Asn Thr Ile Cys Leu Leu Ser Gln His Gln Phe Met Ser
                595 600 605
Gly Tyr Ser Gln Asp Ile Leu Met Pro Leu Trp Thr Ser Tyr Thr Val
        610 615 620
Asp Arg Asn Asp Ser Phe Ser Thr Glu Asp Phe Ser Asn Cys Leu Tyr
625 630 635 640
Gln Asp Phe Arg Ile Pro Leu Ser Pro Val His Lys Cys Ser Phe Tyr
                                645 650 655
Lys Asn Asn Thr Lys Val Ser Tyr Gly Phe Leu Ser Pro Pro Gln Leu
                        660 665 670
Asn Lys Asn Ser Ser Gly Ile Tyr Ser Glu Ala Leu Leu Thr Thr Asn
              675 680 685
Ile Val Pro Met Tyr Gln Ser Phe Gln Val Ile Trp Arg Tyr Phe His
        690 695 700
Asp Thr Leu Leu Arg Lys Tyr Ala Glu Glu Arg Asn Gly Val Asn Val
705 710 715 720
Val Ser Gly Pro Val Phe Asp Phe Asp Tyr Asp Gly Arg Cys Asp Ser
                                725 730 735
Leu Glu Asn Leu Arg Gln Lys Arg Arg Val Ile Arg Asn Gln Glu Ile
                        740 745 750
Leu Ile Pro Thr His Phe Phe Ile Val Leu Thr Ser Cys Lys Asp Thr
                755 760 765
Ser Gln Thr Pro Leu His Cys Glu Asn Leu Asp Thr Leu Ala Phe Ile
        770 775 780
Leu Pro His Arg Thr Asp Asn Ser Glu Ser Cys Val His Gly Lys His
785 790 795 800
Asp Ser Ser Trp Val Glu Glu Leu Leu Met Leu His Arg Ala Arg Ile
                                805 810 815
Thr Asp Val Glu His Ile Thr Gly Leu Ser Phe Tyr Gln Gln Arg Lys
                        820 825 830
Glu Pro Val Ser Asp Ile Leu Lys Leu Lys Thr His Leu Pro Thr Phe
                835 840 845
Ser Gln Glu Asp Asp Lys Thr His Thr Cys Pro Pro Cys Pro Ala Pro
        850 855 860
Glu Leu Leu Gly Gly Pro Ser Val Phe Leu Phe Pro Pro Lys Pro Lys
865 870 875 880
Asp Thr Leu Met Ile Ser Arg Thr Pro Glu Val Thr Cys Val Val Val
                                885 890 895
Asp Val Ser His Glu Asp Pro Glu Val Lys Phe Asn Trp Tyr Val Asp
                        900 905 910
Gly Val Glu Val His Asn Ala Lys Thr Lys Pro Arg Glu Glu Gln Tyr
                915 920 925
Asn Ser Thr Tyr Arg Val Val Ser Val Leu Thr Val Leu His Gln Asp
        930 935 940
Trp Leu Asn Gly Lys Glu Tyr Lys Cys Lys Val Ser Asn Lys Ala Leu
945 950 955 960
Pro Ala Pro Ile Glu Lys Thr Ile Ser Lys Ala Lys Gly Gln Pro Arg
                                965 970 975
Glu Pro Gln Val Tyr Thr Leu Pro Pro Ser Arg Glu Glu Met Thr Lys
                        980 985 990
Asn Gln Val Ser Leu Thr Cys Leu Val Lys Gly Phe Tyr Pro Ser Asp
                995 1000 1005
Ile Ala Val Glu Trp Glu Ser Asn Gly Gln Pro Glu Asn Asn Tyr
        1010 1015 1020
Lys Thr Thr Pro Pro Val Leu Asp Ser Asp Gly Ser Phe Phe Leu
        1025 1030 1035
Tyr Ser Lys Leu Thr Val Asp Lys Ser Arg Trp Gln Gln Gly Asn
        1040 1045 1050
Val Phe Ser Cys Ser Val Met His Glu Ala Leu His Asn His Tyr
        1055 1060 1065
Thr Gln Lys Ser Leu Ser Leu Ser Pro Gly Lys
        1070 1075
Singly underlined: signal peptide sequence; double-underlined:
beginning and end of NPP1; ** = cleavage position at the signal
peptide sequence; bold residues indicate Fc sequence
SEQ. ID NO: 18-ENPP71-Fc Amino Acid Sequence
Met Arg Gly Pro Ala Val Leu Leu Thr Val Ala Leu Ala Thr Leu Leu
1 5 10 15
Ala Pro Gly Ala Gly Ala**Gly Leu Lys Pro Ser Cys Ala Lys Glu Val
                        20 25 30
Lys Ser Cys Lys Gly Arg Cys Phe Glu Arg Thr Phe Gly Asn Cys Arg
                35 40 45
Cys Asp Ala Ala Cys Val Glu Leu Gly Asn Cys Cys Leu Asp Tyr Gln
        50 55 60
Glu Thr Cys Ile Glu Pro Glu His Ile Trp Thr Cys Asn Lys Phe Arg
65 70 75 80
Cys Gly Glu Lys Arg Leu Thr Arg Ser Leu Cys Ala Cys Ser Asp Asp
                                85 90 95
Cys Lys Asp Lys Gly Asp Cys Cys Ile Asn Tyr Ser Ser Val Cys Gln
                        100 105 110
Gly Glu Lys Ser Trp Val Glu Glu Pro Cys Glu Ser Ile Asn Glu Pro
                115 120 125
Gln Cys Pro Ala Gly Phe Glu Thr Pro Pro Thr Leu Leu Phe Ser Leu
        130 135 140
Asp Gly Phe Arg Ala Glu Tyr Leu His Thr Trp Gly Gly Leu Leu Pro
145 150 155 160
Val Ile Ser Lys Leu Lys Lys Cys Gly Thr Tyr Thr Lys Asn Met Arg
                                165 170 175
Pro Val Tyr Pro Thr Lys Thr Phe Pro Asn His Tyr Ser Ile Val Thr
                        180 185 190
Gly Leu Tyr Pro Glu Ser His Gly Ile Ile Asp Asn Lys Met Tyr Asp
                195 200 205
Pro Lys Met Asn Ala Ser Phe Ser Leu Lys Ser Lys Glu Lys Phe Asn
        210 215 220
Pro Glu Trp Tyr Lys Gly Glu Pro Ile Trp Val Thr Ala Lys Tyr Gln
225 230 235 240
Gly Leu Lys Ser Gly Thr Phe Phe Trp Pro Gly Ser Asp Val Glu Ile
                                245 250 255
Asn Gly Ile Phe Pro Asp Ile Tyr Lys Met Tyr Asn Gly Ser Val Pro
                        260 265 270
Phe Glu Glu Arg Ile Leu Ala Val Leu Gln Trp Leu Gln Leu Pro Lys
                275 280 285
Asp Glu Arg Pro His Phe Tyr Thr Leu Tyr Leu Glu Glu Pro Asp Ser
        290 295 300
Ser Gly His Ser Tyr Gly Pro Val Ser Ser Glu Val Ile Lys Ala Leu
305 310 315 320
Gln Arg Val Asp Gly Met Val Gly Met Leu Met Asp Gly Leu Lys Glu
                                325 330 335
Leu Asn Leu His Arg Cys Leu Asn Leu Ile Leu Ile Ser Asp His Gly
                        340 345 350
Met Glu Gln Gly Ser Cys Lys Lys Tyr Ile Tyr Leu Asn Lys Tyr Leu
                355 360 365
Gly Asp Val Lys Asn Ile Lys Val Ile Tyr Gly Pro Ala Ala Arg Leu
        370 375 380
Arg Pro Ser Asp Val Pro Asp Lys Tyr Tyr Ser Phe Asn Tyr Glu Gly
385 390 395 400
Ile Ala Arg Asn Leu Ser Cys Arg Glu Pro Asn Gln His Phe Lys Pro
                                405 410 415
Tyr Leu Lys His Phe Leu Pro Lys Arg Leu His Phe Ala Lys Ser Asp
                        420 425 430
Arg Ile Glu Pro Leu Thr Phe Tyr Leu Asp Pro Gln Trp Gln Leu Ala
                435 440 445
Leu Asn Pro Ser Glu Arg Lys Tyr Cys Gly Ser Gly Phe His Gly Ser
        450 455 460
Asp Asn Val Phe Ser Asn Met Gln Ala Leu Phe Val Gly Tyr Gly Pro
465 470 475 480
Gly Phe Lys His Gly Ile Glu Ala Asp Thr Phe Glu Asn Ile Glu Val
                                485 490 495
Tyr Asn Leu Met Cys Asp Leu Leu Asn Leu Thr Pro Ala Pro Asn Asn
                        500 505 510
Gly Thr His Gly Ser Leu Asn His Leu Leu Lys Asn Pro Val Tyr Thr
                515 520 525
Pro Lys His Pro Lys Glu Val His Pro Leu Val Gln Cys Pro Phe Thr
        530 535 540
Arg Asn Pro Arg Asp Asn Leu Gly Cys Ser Cys Asn Pro Ser Ile Leu
545 550 555 560
Pro Ile Glu Asp Phe Gln Thr Gln Phe Asn Leu Thr Val Ala Glu Glu
                                565 570 575
Lys Ile Ile Lys His Glu Thr Leu Pro Tyr Gly Arg Pro Arg Val Leu
                        580 585 590
Gln Lys Glu Asn Thr Ile Cys Leu Leu Ser Gln His Gln Phe Met Ser
                595 600 605
Gly Tyr Ser Gln Asp Ile Leu Met Pro Leu Trp Thr Ser Tyr Thr Val
        610 615 620
Asp Arg Asn Asp Ser Phe Ser Thr Glu Asp Phe Ser Asn Cys Leu Tyr
625 630 635 640
Gln Asp Phe Arg Ile Pro Leu Ser Pro Val His Lys Cys Ser Phe Tyr
                                645 650 655
Lys Asn Asn Thr Lys Val Ser Tyr Gly Phe Leu Ser Pro Pro Gln Leu
                        660 665 670
Asn Lys Asn Ser Ser Gly Ile Tyr Ser Glu Ala Leu Leu Thr Thr Asn
                675 680 685
Ile Val Pro Met Tyr Gln Ser Phe Gln Val Ile Trp Arg Tyr Phe His
        690 695 700
Asp Thr Leu Leu Arg Lys Tyr Ala Glu Glu Arg Asn Gly Val Asn Val
705 710 715 720
Val Ser Gly Pro Val Phe Asp Phe Asp Tyr Asp Gly Arg Cys Asp Ser
                                725 730 735
Leu Glu Asn Leu Arg Gln Lys Arg Arg Val Ile Arg Asn Gln Glu Ile
                        740 745 750
Leu Ile Pro Thr His Phe Phe Ile Val Leu Thr Ser Cys Lys Asp Thr
                755 760 765
Ser Gln Thr Pro Leu His Cys Glu Asn Leu Asp Thr Leu Ala Phe Ile
        770 775 780
Leu Pro His Arg Thr Asp Asn Ser Glu Ser Cys Val His Gly Lys His
785 790 795 800
Asp Ser Ser Trp Val Glu Glu Leu Leu Met Leu His Arg Ala Arg Ile
                                805 810 815
Thr Asp Val Glu His Ile Thr Gly Leu Ser Phe Tyr Gln Gln Arg Lys
                        820 825 830
Glu Pro Val Ser Asp Ile Leu Lys Leu Lys Thr His Leu Pro Thr Phe
                835 840 845
Ser Gln Glu Asp Leu Ile Asn Asp Lys Thr His Thr Cys Pro Pro Cys
        850 855 860
Pro Ala Pro Glu Leu Leu Gly Gly Pro Ser Val Phe Leu Phe Pro Pro
865 870 875 880
Lys Pro Lys Asp Thr Leu Met Ile Ser Arg Thr Pro Glu Val Thr Cys
                                885 890 895
Val Val Val Asp Val Ser His Glu Asp Pro Glu Val Lys Phe Asn Trp
                        900 905 910
Tyr Val Asp Gly Val Glu Val His Asn Ala Lys Thr Lys Pro Arg Glu
                915 920 925
Glu Gln Tyr Asn Ser Thr Tyr Arg Val Val Ser Val Leu Thr Val Leu
        930 935 940
His Gln Asp Trp Leu Asn Gly Lys Glu Tyr Lys Cys Lys Val Ser Asn
945 950 955 960
Lys Ala Leu Pro Ala Pro Ile Glu Lys Thr Ile Ser Lys Ala Lys Gly
                                965 970 975
Gln Pro Arg Glu Pro Gln Val Tyr Thr Leu Pro Pro Ser Arg Glu Glu
                        980 985 990
Met Thr Lys Asn Gln Val Ser Leu Thr Cys Leu Val Lys Gly Phe Tyr
                995 1000 1005
Pro Ser Asp Ile Ala Val Glu Trp Glu Ser Asn Gly Gln Pro Glu
        1010 1015 1020
Asn Asn Tyr Lys Thr Thr Pro Pro Val Leu Asp Ser Asp Gly Ser
        1025 1030 1035
Phe Phe Leu Tyr Ser Lys Leu Thr Val Asp Lys Ser Arg Trp Gln
        1040 1045 1050
Gln Gly Asn Val Phe Ser Cys Ser Val Met His Glu Ala Leu His
        1055 1060 1065
Asn His Tyr Thr Gln Lys Ser Leu Ser Leu Ser Pro Gly Lys
        1070 1075 1080
Singly underlined: signal peptide sequence; double-underlined:
beginning and end of NPP1; ** = cleavage position at the signal
peptide sequence; bold residues indicate Fc sequence
SEQ. ID NO: 19-ENPP71 (lacking NPP1 N-Terminus GLK) Amino Acid
Sequence:
Met Arg Gly Pro Ala Val Leu Leu Thr Val Ala Leu Ala Thr Leu Leu
1 5 10 15
Ala Pro Gly Ala Gly Ala**Pro Ser Cys Ala Lys Glu Val Lys Ser Cys
                        20 25 30
Lys Gly Arg Cys Phe Glu Arg Thr Phe Gly Asn Cys Arg Cys Asp Ala
                35 40 45
Ala Cys Val Glu Leu Gly Asn Cys Cys Leu Asp Tyr Gln Glu Thr Cys
        50 55 60
Ile Glu Pro Glu His Ile Trp Thr Cys Asn Lys Phe Arg Cys Gly Glu
65 70 75 80
Lys Arg Leu Thr Arg Ser Leu Cys Ala Cys Ser Asp Asp Cys Lys Asp
                                85 90 95
Lys Gly Asp Cys Cys Ile Asn Tyr Ser Ser Val Cys Gln Gly Glu Lys
                        100 105 110
Ser Trp Val Glu Glu Pro Cys Glu Ser Ile Asn Glu Pro Gln Cys Pro
                115 120 125
Ala Gly Phe Glu Thr Pro Pro Thr Leu Leu Phe Ser Leu Asp Gly Phe
        130 135 140
Arg Ala Glu Tyr Leu His Thr Trp Gly Gly Leu Leu Pro Val Ile Ser
145 150 155 160
Lys Leu Lys Lys Cys Gly Thr Tyr Thr Lys Asn Met Arg Pro Val Tyr
                                165 170 175
Pro Thr Lys Thr Phe Pro Asn His Tyr Ser Ile Val Thr Gly Leu Tyr
                        180 185 190
Pro Glu Ser His Gly Ile Ile Asp Asn Lys Met Tyr Asp Pro Lys Met
                195 200 205
Asn Ala Ser Phe Ser Leu Lys Ser Lys Glu Lys Phe Asn Pro Glu Trp
        210 215 220
Tyr Lys Gly Glu Pro Ile Trp Val Thr Ala Lys Tyr Gln Gly Leu Lys
225 230 235 240
Ser Gly Thr Phe Phe Trp Pro Gly Ser Asp Val Glu Ile Asn Gly Ile
                                245 250 255
Phe Pro Asp Ile Tyr Lys Met Tyr Asn Gly Ser Val Pro Phe Glu Glu
                        260 265 270
Arg Ile Leu Ala Val Leu Gln Trp Leu Gln Leu Pro Lys Asp Glu Arg
                275 280 285
Pro His Phe Tyr Thr Leu Tyr Leu Glu Glu Pro Asp Ser Ser Gly His
        290 295 300
Ser Tyr Gly Pro Val Ser Ser Glu Val Ile Lys Ala Leu Gln Arg Val
305 310 315 320
Asp Gly Met Val Gly Met Leu Met Asp Gly Leu Lys Glu Leu Asn Leu
                                325 330 335
His Arg Cys Leu Asn Leu Ile Leu Ile Ser Asp His Gly Met Glu Gln
                        340 345 350
Gly Ser Cys Lys Lys Tyr Ile Tyr Leu Asn Lys Tyr Leu Gly Asp Val
                355 360 365
Lys Asn Ile Lys Val Ile Tyr Gly Pro Ala Ala Arg Leu Arg Pro Ser
        370 375 380
Asp Val Pro Asp Lys Tyr Tyr Ser Phe Asn Tyr Glu Gly Ile Ala Arg
385 390 395 400
Asn Leu Ser Cys Arg Glu Pro Asn Gln His Phe Lys Pro Tyr Leu Lys
                                405 410 415
His Phe Leu Pro Lys Arg Leu His Phe Ala Lys Ser Asp Arg Ile Glu
                        420 425 430
Pro Leu Thr Phe Tyr Leu Asp Pro Gln Trp Gln Leu Ala Leu Asn Pro
                435 440 445
Ser Glu Arg Lys Tyr Cys Gly Ser Gly Phe His Gly Ser Asp Asn Val
        450 455 460
Phe Ser Asn Met Gln Ala Leu Phe Val Gly Tyr Gly Pro Gly Phe Lys
465 470 475 480
His Gly Ile Glu Ala Asp Thr Phe Glu Asn Ile Glu Val Tyr Asn Leu
                                485 490 495
Met Cys Asp Leu Leu Asn Leu Thr Pro Ala Pro Asn Asn Gly Thr His
                        500 505 510
Gly Ser Leu Asn His Leu Leu Lys Asn Pro Val Tyr Thr Pro Lys His
                515 520 525
Pro Lys Glu Val His Pro Leu Val Gln Cys Pro Phe Thr Arg Asn Pro
        530 535 540
Arg Asp Asn Leu Gly Cys Ser Cys Asn Pro Ser Ile Leu Pro Ile Glu
545 550 555 560
Asp Phe Gln Thr Gln Phe Asn Leu Thr Val Ala Glu Glu Lys Ile Ile
                                565 570 575
Lys His Glu Thr Leu Pro Tyr Gly Arg Pro Arg Val Leu Gln Lys Glu
                        580 585 590
Asn Thr Ile Cys Leu Leu Ser Gln His Gln Phe Met Ser Gly Tyr Ser
                595 600 605
Gln Asp Ile Leu Met Pro Leu Trp Thr Ser Tyr Thr Val Asp Arg Asn
        610 615 620
Asp Ser Phe Ser Thr Glu Asp Phe Ser Asn Cys Leu Tyr Gln Asp Phe
625 630 635 640
Arg Ile Pro Leu Ser Pro Val His Lys Cys Ser Phe Tyr Lys Asn Asn
                                645 650 655
Thr Lys Val Ser Tyr Gly Phe Leu Ser Pro Pro Gln Leu Asn Lys Asn
                        660 665 670
Ser Ser Gly Ile Tyr Ser Glu Ala Leu Leu Thr Thr Asn Ile Val Pro
                675 680 685
Met Tyr Gln Ser Phe Gln Val Ile Trp Arg Tyr Phe His Asp Thr Leu
        690 695 700
Leu Arg Lys Tyr Ala Glu Glu Arg Asn Gly Val Asn Val Val Ser Gly
705 710 715 720
Pro Val Phe Asp Phe Asp Tyr Asp Gly Arg Cys Asp Ser Leu Glu Asn
                                725 730 735
Leu Arg Gln Lys Arg Arg Val Ile Arg Asn Gln Glu Ile Leu Ile Pro
                        740 745 750
Thr His Phe Phe Ile Val Leu Thr Ser Cys Lys Asp Thr Ser Gln Thr
                755 760 765
Pro Leu His Cys Glu Asn Leu Asp Thr Leu Ala Phe Ile Leu Pro His
        770 775 780
Arg Thr Asp Asn Ser Glu Ser Cys Val His Gly Lys His Asp Ser Ser
785 790 795 800
Trp Val Glu Glu Leu Leu Met Leu His Arg Ala Arg Ile Thr Asp Val
                                805 810 815
Glu His Ile Thr Gly Leu Ser Phe Tyr Gln Gln Arg Lys Glu Pro Val
                        820 825 830
Ser Asp Ile Leu Lys Leu Lys Thr His Leu Pro Thr Phe SerGln Glu
                835 840 845
Asp
Singly underlined: signal peptide sequence; double-underlined:
beginning and end of NPP1; ** = cleavage position at the signal
peptide sequence
SEQ. ID NO: 20-ENPP71 (lacking NPP1 N-Terminus GLK)-Fc Amino
Acid Sequence:
Met Arg Gly Pro Ala Val Leu Leu Thr Val Ala Leu Ala Thr Leu Leu
1 5 10 15
Ala Pro Gly Ala Gly Ala**Pro Ser Cys Ala Lys Glu Val Lys Ser Cys
                        20 25 30
Lys Gly Arg Cys Phe Glu Arg Thr Phe Gly Asn Cys Arg Cys Asp Ala
                35 40 45
Ala Cys Val Glu Leu Gly Asn Cys Cys Leu Asp Tyr Gln Glu Thr Cys
        50 55 60
Ile Glu Pro Glu His Ile Trp Thr Cys Asn Lys Phe Arg Cys Gly Glu
65 70 75 80
Lys Arg Leu Thr Arg Ser Leu Cys Ala Cys Ser Asp Asp Cys Lys Asp
                                85 90 95
Lys Gly Asp Cys Cys Ile Asn Tyr Ser Ser Val Cys Gln Gly Glu Lys
                        100 105 110
Ser Trp Val Glu Glu Pro Cys Glu Ser Ile Asn Glu Pro Gln Cys Pro
                115 120 125
Ala Gly Phe Glu Thr Pro Pro Thr Leu Leu Phe Ser Leu Asp Gly Phe
        130 135 140
Arg Ala Glu Tyr Leu His Thr Trp Gly Gly Leu Leu Pro Val Ile Ser
145 150 155 160
Lys Leu Lys Lys Cys Gly Thr Tyr Thr Lys Asn Met Arg Pro Val Tyr
                                165 170 175
Pro Thr Lys Thr Phe Pro Asn His Tyr Ser Ile Val Thr Gly Leu Tyr
                        180 185 190
Pro Glu Ser His Gly Ile Ile Asp Asn Lys Met Tyr Asp Pro Lys Met
                195 200 205
Asn Ala Ser Phe Ser Leu Lys Ser Lys Glu Lys Phe Asn Pro Glu Trp
        210 215 220
Tyr Lys Gly Glu Pro Ile Trp Val Thr Ala Lys Tyr Gln Gly Leu Lys
225 230 235 240
Ser Gly Thr Phe Phe Trp Pro Gly Ser Asp Val Glu Ile Asn Gly Ile
                                245 250 255
Phe Pro Asp Ile Tyr Lys Met Tyr Asn Gly Ser Val Pro Phe Glu Glu
                        260 265 270
Arg Ile Leu Ala Val Leu Gln Trp Leu Gln Leu Pro Lys Asp Glu Arg
                275 280 285
Pro His Phe Tyr Thr Leu Tyr Leu Glu Glu Pro Asp Ser Ser Gly His
        290 295 300
Ser Tyr Gly Pro Val Ser Ser Glu Val Ile Lys Ala Leu Gln Arg Val
305 310 315 320
Asp Gly Met Val Gly Met Leu Met Asp Gly Leu Lys Glu Leu Asn Leu
                                325 330 335
His Arg Cys Leu Asn Leu Ile Leu Ile Ser Asp His Gly Met Glu Gln
                        340 345 350
Gly Ser Cys Lys Lys Tyr Ile Tyr Leu Asn Lys Tyr Leu Gly Asp Val
                355 360 365
Lys Asn Ile Lys Val Ile Tyr Gly Pro Ala Ala Arg Leu Arg Pro Ser
        370 375 380
Asp Val Pro Asp Lys Tyr Tyr Ser Phe Asn Tyr Glu Gly Ile Ala Arg
385 390 395 400
Asn Leu Ser Cys Arg Glu Pro Asn Gln His Phe Lys Pro Tyr Leu Lys
                                405 410 415
His Phe Leu Pro Lys Arg Leu His Phe Ala Lys Ser Asp Arg Ile Glu
                        420 425 430
Pro Leu Thr Phe Tyr Leu Asp Pro Gln Trp Gln Leu Ala Leu Asn Pro
                435 440 445
Ser Glu Arg Lys Tyr Cys Gly Ser Gly Phe His Gly Ser Asp Asn Val
        450 455 460
Phe Ser Asn Met Gln Ala Leu Phe Val Gly Tyr Gly Pro Gly Phe Lys
465 470 475 480
His Gly Ile Glu Ala Asp Thr Phe Glu Asn Ile Glu Val Tyr Asn Leu
                                485 490 495
Met Cys Asp Leu Leu Asn Leu Thr Pro Ala Pro Asn Asn Gly Thr His
                        500 505 510
Gly Ser Leu Asn His Leu Leu Lys Asn Pro Val Tyr Thr Pro Lys His
                515 520 525
Pro Lys Glu Val His Pro Leu Val Gln Cys Pro Phe Thr Arg Asn Pro
        530 535 540
Arg Asp Asn Leu Gly Cys Ser Cys Asn Pro Ser Ile Leu Pro Ile Glu
545 550 555 560
Asp Phe Gln Thr Gln Phe Asn Leu Thr Val Ala Glu Glu Lys Ile Ile
                                565 570 575
Lys His Glu Thr Leu Pro Tyr Gly Arg Pro Arg Val Leu Gln Lys Glu
                        580 585 590
Asn Thr Ile Cys Leu Leu Ser Gln His Gln Phe Met Ser Gly Tyr Ser
                595 600 605
Gln Asp Ile Leu Met Pro Leu Trp Thr Ser Tyr Thr Val Asp Arg Asn
        610 615 620
Asp Ser Phe Ser Thr Glu Asp Phe Ser Asn Cys Leu Tyr Gln Asp Phe
625 630 635 640
Arg Ile Pro Leu Ser Pro Val His Lys Cys Ser Phe Tyr Lys Asn Asn
                                645 650 655
Thr Lys Val Ser Tyr Gly Phe Leu Ser Pro Pro Gln Leu Asn Lys Asn
                        660 665 670
Ser Ser Gly Ile Tyr Ser Glu Ala Leu Leu Thr Thr Asn Ile Val Pro
                675 680 685
Met Tyr Gln Ser Phe Gln Val Ile Trp Arg Tyr Phe His Asp Thr Leu
        690 695 700
Leu Arg Lys Tyr Ala Glu Glu Arg Asn Gly Val Asn Val Val Ser Gly
705 710 715 720
Pro Val Phe Asp Phe Asp Tyr Asp Gly Arg Cys Asp Ser Leu Glu Asn
                                725 730 735
Leu Arg Gln Lys Arg Arg Val Ile Arg Asn Gln Glu Ile Leu Ile Pro
                        740 745 750
Thr His Phe Phe Ile Val Leu Thr Ser Cys Lys Asp Thr Ser Gln Thr
                755 760 765
Pro Leu His Cys Glu Asn Leu Asp Thr Leu Ala Phe Ile Leu Pro His
        770 775 780
Arg Thr Asp Asn Ser Glu Ser Cys Val His Gly Lys His Asp Ser Ser
785 790 795 800
Trp Val Glu Glu Leu Leu Met Leu His Arg Ala Arg Ile Thr Asp Val
                                805 810 815
Glu His Ile Thr Gly Leu Ser Phe Tyr Gln Gln Arg Lys Glu Pro Val
                        820 825 830
Ser Asp Ile Leu Lys Leu Lys Thr His Leu Pro Thr Phe Ser Gln Glu
                835 840 845
Asp Leu Ile Asn Asp Lys Thr His Thr Cys Pro Pro Cys Pro Ala Pro
        850 855 860
Glu Leu Leu Gly Gly Pro Ser Val Phe Leu Phe Pro Pro Lys Pro Lys
865 870 875 880
Asp Thr Leu Met Ile Ser Arg Thr Pro Glu Val Thr Cys Val Val Val
                                885 890 895
Asp Val Ser His Glu Asp Pro Glu Val Lys Phe Asn Trp Tyr Val Asp
                        900 905 910
Gly Val Glu Val His Asn Ala Lys Thr Lys Pro Arg Glu Glu Gln Tyr
                915 920 925
Asn Ser Thr Tyr Arg Val Val Ser Val Leu Thr Val Leu His Gln Asp
        930 935 940
Trp Leu Asn Gly Lys Glu Tyr Lys Cys Lys Val Ser Asn Lys Ala Leu
945 950 955 960
Pro Ala Pro Ile Glu Lys Thr Ile Ser Lys Ala Lys Gly Gln Pro Arg
                                965 970 975
Glu Pro Gln Val Tyr Thr Leu Pro Pro Ser Arg Glu Glu Met Thr Lys
                        980 985 990
Asn Gln Val Ser Leu Thr Cys Leu Val Lys Gly Phe Tyr Pro Ser Asp
                995 1000 1005
Ile Ala Val Glu Trp Glu Ser Asn Gly Gln Pro Glu Asn Asn Tyr
        1010 1015 1020
Lys Thr Thr Pro Pro Val Leu Asp Ser Asp Gly Ser Phe Phe Leu
        1025 1030 1035
Tyr Ser Lys Leu Thr Val Asp Lys Ser Arg Trp Gln Gln Gly Asn
        1040 1045 1050
Val Phe Ser Cys Ser Val Met His Glu Ala Leu His Asn His Tyr
        1055 1060 1065
Thr Gln Lys Ser Leu Ser Leu Ser Pro Gly Lys
        1070 1075
Singly underlined: signal peptide sequence; double-underlined:
beginning and end of NPP1; ** = cleavage position at the signal
peptide sequence; bold residues indicate Fc sequence
SEQ. ID NO: 21-ENPP71 (lacking NPP1 N-Terminus GLK)-ALB Amino
Acid Sequence
Met Arg Gly Pro Ala Val Leu Leu Thr Val Ala Leu Ala Thr Leu Leu
1 5 10 15
Ala Pro Gly Ala Gly Ala**Pro Ser Cys Ala Lys Glu Val Lys Ser Cys
                        20 25 30
Lys Gly Arg Cys Phe Glu Arg Thr Phe Gly Asn Cys Arg Cys Asp Ala
                35 40 45
Ala Cys Val Glu Leu Gly Asn Cys Cys Leu Asp Tyr Gln Glu Thr Cys
        50 55 60
Ile Glu Pro Glu His Ile Trp Thr Cys Asn Lys Phe Arg Cys Gly Glu
65 70 75 80
Lys Arg Leu Thr Arg Ser Leu Cys Ala Cys Ser Asp Asp Cys Lys Asp
                                85 90 95
Lys Gly Asp Cys Cys Ile Asn Tyr Ser Ser Val Cys Gln Gly Glu Lys
                        100 105 110
Ser Trp Val Glu Glu Pro Cys Glu Ser Ile Asn Glu Pro Gln Cys Pro
                115 120 125
Ala Gly Phe Glu Thr Pro Pro Thr Leu Leu Phe Ser Leu Asp Gly Phe
        130 135 140
Arg Ala Glu Tyr Leu His Thr Trp Gly Gly Leu Leu Pro Val Ile Ser
145 150 155 160
Lys Leu Lys Lys Cys Gly Thr Tyr Thr Lys Asn Met Arg Pro Val Tyr
                                165 170 175
Pro Thr Lys Thr Phe Pro Asn His Tyr Ser Ile Val Thr Gly Leu Tyr
                        180 185 190
Pro Glu Ser His Gly Ile Ile Asp Asn Lys Met Tyr Asp Pro Lys Met
                195 200 205
Asn Ala Ser Phe Ser Leu Lys Ser Lys Glu Lys Phe Asn Pro Glu Trp
        210 215 220
Tyr Lys Gly Glu Pro Ile Trp Val Thr Ala Lys Tyr Gln Gly Leu Lys
225 230 235 240
Ser Gly Thr Phe Phe Trp Pro Gly Ser Asp Val Glu Ile Asn Gly Ile
                                245 250 255
Phe Pro Asp Ile Tyr Lys Met Tyr Asn Gly Ser Val Pro Phe Glu Glu
                        260 265 270
Arg Ile Leu Ala Val Leu Gln Trp Leu Gln Leu Pro Lys Asp Glu Arg
                275 280 285
Pro His Phe Tyr Thr Leu Tyr Leu Glu Glu Pro Asp Ser Ser Gly His
        290 295 300
Ser Tyr Gly Pro Val Ser Ser Glu Val Ile Lys Ala Leu Gln Arg Val
305 310 315 320
Asp Gly Met Val Gly Met Leu Met Asp Gly Leu Lys Glu Leu Asn Leu
                                325 330 335
His Arg Cys Leu Asn Leu Ile Leu Ile Ser Asp His Gly Met Glu Gln
                        340 345 350
Gly Ser Cys Lys Lys Tyr Ile Tyr Leu Asn Lys Tyr Leu Gly Asp Val
                355 360 365
Lys Asn Ile Lys Val Ile Tyr Gly Pro Ala Ala Arg Leu Arg Pro Ser
        370 375 380
Asp Val Pro Asp Lys Tyr Tyr Ser Phe Asn Tyr Glu Gly Ile Ala Arg
385 390 395 400
Asn Leu Ser Cys Arg Glu Pro Asn Gln His Phe Lys Pro Tyr Leu Lys
                                405 410 415
His Phe Leu Pro Lys Arg Leu His Phe Ala Lys Ser Asp Arg Ile Glu
                        420 425 430
Pro Leu Thr Phe Tyr Leu Asp Pro Gln Trp Gln Leu Ala Leu Asn Pro
                435 440 445
Ser Glu Arg Lys Tyr Cys Gly Ser Gly Phe His Gly Ser Asp Asn Val
        450 455 460
Phe Ser Asn Met Gln Ala Leu Phe Val Gly Tyr Gly Pro Gly Phe Lys
465 470 475 480
His Gly Ile Glu Ala Asp Thr Phe Glu Asn Ile Glu Val Tyr Asn Leu
                                485 490 495
Met Cys Asp Leu Leu Asn Leu Thr Pro Ala Pro Asn Asn Gly Thr His
                        500 505 510
Gly Ser Leu Asn His Leu Leu Lys Asn Pro Val Tyr Thr Pro Lys His
                515 520 525
Pro Lys Glu Val His Pro Leu Val Gln Cys Pro Phe Thr Arg Asn Pro
        530 535 540
Arg Asp Asn Leu Gly Cys Ser Cys Asn Pro Ser Ile Leu Pro Ile Glu
545 550 555 560
Asp Phe Gln Thr Gln Phe Asn Leu Thr Val Ala Glu Glu Lys Ile Ile
                                565 570 575
Lys His Glu Thr Leu Pro Tyr Gly Arg Pro Arg Val Leu Gln Lys Glu
                        580 585 590
Asn Thr Ile Cys Leu Leu Ser Gln His Gln Phe Met Ser Gly Tyr Ser
                595 600 605
Gln Asp Ile Leu Met Pro Leu Trp Thr Ser Tyr Thr Val Asp Arg Asn
        610 615 620
Asp Ser Phe Ser Thr Glu Asp Phe Ser Asn Cys Leu Tyr Gln Asp Phe
625 630 635 640
Arg Ile Pro Leu Ser Pro Val His Lys Cys Ser Phe Tyr Lys Asn Asn
                                645 650 655
Thr Lys Val Ser Tyr Gly Phe Leu Ser Pro Pro Gln Leu Asn Lys Asn
                        660 665 670
Ser Ser Gly Ile Tyr Ser Glu Ala Leu Leu Thr Thr Asn Ile Val Pro
                675 680 685
Met Tyr Gln Ser Phe Gln Val Ile Trp Arg Tyr Phe His Asp Thr Leu
        690 695 700
Leu Arg Lys Tyr Ala Glu Glu Arg Asn Gly Val Asn Val Val Ser Gly
705 710 715 720
Pro Val Phe Asp Phe Asp Tyr Asp Gly Arg Cys Asp Ser Leu Glu Asn
                                725 730 735
Leu Arg Gln Lys Arg Arg Val Ile Arg Asn Gln Glu Ile Leu Ile Pro
                        740 745 750
Thr His Phe Phe Ile Val Leu Thr Ser Cys Lys Asp Thr Ser Gln Thr
                755 760 765
Pro Leu His Cys Glu Asn Leu Asp Thr Leu Ala Phe Ile Leu Pro His
        770 775 780
Arg Thr Asp Asn Ser Glu Ser Cys Val His Gly Lys His Asp Ser Ser
785 790 795 800
Trp Val Glu Glu Leu Leu Met Leu His Arg Ala Arg Ile Thr Asp Val
                                805 810 815
Glu His Ile Thr Gly Leu Ser Phe Tyr Gln Gln Arg Lys Glu Pro Val
                        820 825 830
Ser Asp Ile Leu Lys Leu Lys Thr His Leu Pro Thr Phe Ser Gln Glu
                835 840 845
Asp Arg Ser Gly Ser Gly Gly Ser Met Lys Trp Val Thr Phe Leu Leu
        850 855 860
Leu Leu Phe Val Ser Gly Ser Ala Phe Ser Arg Gly Val Phe Arg Arg
865 870 875 880
Glu Ala His Lys Ser Glu Ile Ala His Arg Tyr Asn Asp Leu Gly Glu
                                885 890 895
Gln His Phe Lys Gly Leu Val Leu Ile Ala Phe Ser Gln Tyr Leu Gln
                        900 905 910
Lys Cys Ser Tyr Asp Glu His Ala Lys Leu Val Gln Glu Val Thr Asp
                915 920 925
Phe Ala Lys Thr Cys Val Ala Asp Glu Ser Ala Ala Asn Cys Asp Lys
        930 935 940
Ser Leu His Thr Leu Phe Gly Asp Lys Leu Cys Ala Ile Pro Asn Leu
945 950 955 960
Arg Glu Asn Tyr Gly Glu Leu Ala Asp Cys Cys Thr Lys Gln Glu Pro
                                965 970 975
Glu Arg Asn Glu Cys Phe Leu Gln His Lys Asp Asp Asn Pro Ser Leu
                        980 985 990
Pro Pro Phe Glu Arg Pro Glu Ala Glu Ala Met Cys Thr Ser Phe Lys
                995 1000 1005
Glu Asn Pro Thr Thr Phe Met Gly His Tyr Leu His Glu Val Ala
        1010 1015 1020
Arg Arg His Pro Tyr Phe Tyr Ala Pro Glu Leu Leu Tyr Tyr Ala
        1025 1030 1035
Glu Gln Tyr Asn Glu Ile Leu Thr Gln Cys Cys Ala Glu Ala Asp
        1040 1045 1050
Lys Glu Ser Cys Leu Thr Pro Lys Leu Asp Gly Val Lys Glu Lys
        1055 1060 1065
Ala Leu Val Ser Ser Val Arg Gln Arg Met Lys Cys Ser Ser Met
        1070 1075 1080
Gln Lys Phe Gly Glu Arg Ala Phe Lys Ala Trp Ala Val Ala Arg
        1085 1090 1095
Leu Ser Gln Thr Phe Pro Asn Ala Asp Phe Ala Glu Ile Thr Lys
        1100 1105 1110
Leu Ala Thr Asp Leu Thr Lys Val Asn Lys Glu Cys Cys His Gly
        1115 1120 1125
Asp Leu Leu Glu Cys Ala Asp Asp Arg Ala Glu Leu Ala Lys Tyr
        1130 1135 1140
Met Cys Glu Asn Gln Ala Thr Ile Ser Ser Lys Leu Gln Thr Cys
        1145 1150 1155
Cys Asp Lys Pro Leu Leu Lys Lys Ala His Cys Leu Ser Glu Val
        1160 1165 1170
Glu His Asp Thr Met Pro Ala Asp Leu Pro Ala Ile Ala Ala Asp
        1175 1180 1185
Phe Val Glu Asp Gln Glu Val Cys Lys Asn Tyr Ala Glu Ala Lys
        1190 1195 1200
Asp Val Phe Leu Gly Thr Phe Leu Tyr Glu Tyr Ser Arg Arg His
        1205 1210 1215
Pro Asp Tyr Ser Val Ser Leu Leu Leu Arg Leu Ala Lys Lys Tyr
        1220 1225 1230
Glu Ala Thr Leu Glu Lys Cys Cys Ala Glu Ala Asn Pro Pro Ala
        1235 1340 1245
Cys Tyr Gly Thr Val Leu Ala Glu Phe Gln Pro Leu Val Glu Glu
        1250 1255 1260
Pro Lys Asn Leu Val Lys Thr Asn Cys Asp Leu Tyr Glu Lys Leu
        1265 1270 1275
Gly Glu Tyr Gly Phe Gln Asn Ala Ile Leu Val Arg Tyr Thr Gln
        1280 1285 1290
Lys Ala Pro Gln Val Ser Thr Pro Thr Leu Val Glu Ala Ala Arg
        1295 1300 1305
Asn Leu Gly Arg Val Gly Thr Lys Cys Cys Thr Leu Pro Glu Asp
        1310 1315 1320
Gln Arg Leu Pro Cys Val Glu Asp Tyr Leu Ser Ala Ile Leu Asn
        1325 1330 1335
Arg Val Cys Leu Leu His Glu Lys Thr Pro Val Ser Glu His Val
        1340 1345 1350
Thr Lys Cys Cys Ser Gly Ser Leu Val Glu Arg Arg Pro Cys Phe
        1355 1360 1365
Ser Ala Leu Thr Val Asp Glu Thr Tyr Val Pro Lys Glu Phe Lys
        1370 1375 1380
Ala Glu Thr Phe Thr Phe His Ser Asp Ile Cys Thr Leu Pro Glu
        1385 1390 1395
Lys Glu Lys Gln Ile Lys Lys Gln Thr Ala Leu Ala Glu Leu Val
        1400 1405 1410
Lys His Lys Pro Lys Ala Thr Ala Glu Gln Leu Lys Thr Val Met
        1415 1420 1425
Asp Asp Phe Ala Gln Phe Leu Asp Thr Cys Cys Lys Ala Ala Asp
        1430 1435 1440
Lys Asp Thr Cys Phe Ser Thr Glu Gly Pro Asn Leu Val Thr Arg
        1445 1450 1455
Cys Lys Asp Ala Leu Ala Arg Ser Trp Ser His Pro Gln Phe Glu
        1460 1465 1470
Singly underlined: signal peptide sequence; double-underlined:
beginning and end of NPP1; ** = cleavage position at the signal
peptide sequence; bold residues indicate albumin sequence
SEQ. ID NO: 22-ENPP7-NPP3-Fc sequence:
Met Arg Gly Pro Ala Val Leu Leu Thr Val Ala Leu Ala Thr Leu Leu
1 5 10 15
Ala Pro Gly Ala**Lys Gln Gly Ser Cys Arg Lys Lys Cys Phe Asp Ala
                        20 25 30
Ser Phe Arg Gly Leu Glu Asn Cys Arg Cys Asp Val Ala Cys Lys Asp
                35 40 45
Arg Gly Asp Cys Cys Trp Asp Phe Glu Asp Thr Cys Val Glu Ser Thr
        50 55 60
Arg Ile Trp Met Cys Asn Lys Phe Arg Cys Gly Glu Arg Leu Glu Ala
65 70 75 80
Ser Leu Cys Ser Cys Ser Asp Asp Cys Leu Gln Arg Lys Asp Cys Cys
                                85 90 95
Ala Asp Tyr Lys Ser Val Cys Gln Gly Glu Thr Ser Trp Leu Glu Glu
                        100 105 110
Asn Cys Asp Thr Ala Gln Gln Ser Gln Cys Pro Glu Gly Phe Asp Leu
                115 120 125
Pro Pro Val Ile Leu Phe Ser Met Asp Gly Phe Arg Ala Glu Tyr Leu
        130 135 140
Tyr Thr Trp Asp Thr Leu Met Pro Asn Ile Asn Lys Leu Lys Thr Cys
145 150 155 160
Gly Ile His Ser Lys Tyr Met Arg Ala Met Tyr Pro Thr Lys Thr Phe
                                165 170 175
Pro Asn His Tyr Thr Ile Val Thr Gly Leu Tyr Pro Glu Ser His Gly
                        180 185 190
Ile Ile Asp Asn Asn Met Tyr Asp Val Asn Leu Asn Lys Asn Phe Ser
                195 200 205
Leu Ser Ser Lys Glu Gln Asn Asn Pro Ala Trp Trp His Gly Gln Pro
        210 215 220
Met Trp Leu Thr Ala Met Tyr Gln Gly Leu Lys Ala Ala Thr Tyr Phe
225 230 235 240
Trp Pro Gly Ser Glu Val Ala Ile Asn Gly Ser Phe Pro Ser Ile Tyr
                                245 250 255
Met Pro Tyr Asn Gly Ser Val Pro Phe Glu Glu Arg Ile Ser Thr Leu
                        260 265 270
Leu Lys Trp Leu Asp Leu Pro Lys Ala Glu Arg Pro Arg Phe Tyr Thr
                275 280 285
Met Tyr Phe Glu Glu Pro Asp Ser Ser Gly His Ala Gly Gly Pro Val
        290 295 300
Ser Ala Arg Val Ile Lys Ala Leu Gln Val Val Asp His Ala Phe Gly
305 310 315 320
Met Leu Met Glu Gly Leu Lys Gln Arg Asn Leu His Asn Cys Val Asn
                                325 330 335
Ile Ile Leu Leu Ala Asp His Gly Met Asp Gln Thr Tyr Cys Asn Lys
                        340 345 350
Met Glu Tyr Met Thr Asp Tyr Phe Pro Arg Ile Asn Phe Phe Tyr Met
                355 360 365
Tyr Glu Gly Pro Ala Pro Arg Ile Arg Ala His Asn Ile Pro His Asp
        370 375 380
Phe Phe Ser Phe Asn Ser Glu Glu Ile Val Arg Asn Leu Ser Cys Arg
385 390 395 400
Lys Pro Asp Gln His Phe Lys Pro Tyr Leu Thr Pro Asp Leu Pro Lys
                                405 410 415
Arg Leu His Tyr Ala Lys Asn Val Arg Ile Asp Lys Val His Leu Phe
                        420 425 430
Val Asp Gln Gln Trp Leu Ala Val Arg Ser Lys Ser Asn Thr Asn Cys
                435 440 445
Gly Gly Gly Asn His Gly Tyr Asn Asn Glu Phe Arg Ser Met Glu Ala
        450 455 460
Ile Phe Leu Ala His Gly Pro Ser Phe Lys Glu Lys Thr Glu Val Glu
465 470 475 480
Pro Phe Glu Asn Ile Glu Val Tyr Asn Leu Met Cys Asp Leu Leu Arg
                                485 490 495
Ile Gln Pro Ala Pro Asn Asn Gly Thr His Gly Ser Leu Asn His Leu
                        500 505 510
Leu Lys Val Pro Phe Tyr Glu Pro Ser His Ala Glu Glu Val Ser Lys
                515 520 525
Phe Ser Val Cys Gly Phe Ala Asn Pro Leu Pro Thr Glu Ser Leu Asp
        530 535 540
Cys Phe Cys Pro His Leu Gln Asn Ser Thr Gln Leu Glu Gln Val Asn
545 550 555 560
Gln Met Leu Asn Leu Thr Gln Glu Glu Ile Thr Ala Thr Val Lys Val
                                565 570 575
Asn Leu Pro Phe Gly Arg Pro Arg Val Leu Gln Lys Asn Val Asp His
                        580 585 590
Cys Leu Leu Tyr His Arg Glu Tyr Val Ser Gly Phe Gly Lys Ala Met
                595 600 605
Arg Met Pro Met Trp Ser Ser Tyr Thr Val Pro Gln Leu Gly Asp Thr
        610 615 620
Ser Pro Leu Pro Pro Thr Val Pro Asp Cys Leu Arg Ala Asp Val Arg
625 630 635 640
Val Pro Pro Ser Glu Ser Gln Lys Cys Ser Phe Tyr Leu Ala Asp Lys
                                645 650 655
Asn Ile Thr His Gly Phe Leu Tyr Pro Pro Ala Ser Asn Arg Thr Ser
                        660 665 670
Asp Ser Gln Tyr Asp Ala Leu Ile Thr Ser Asn Leu Val Pro Met Tyr
                675 680 685
Glu Glu Phe Arg Lys Met Trp Asp Tyr Phe His Ser Val Leu Leu Ile
        690 695 700
Lys His Ala Thr Glu Arg Asn Gly Val Asn Val Val Ser Gly Pro Ile
705 710 715 720
Phe Asp Tyr Asn Tyr Asp Gly His Phe Asp Ala Pro Asp Glu Ile Thr
                                725 730 735
Lys His Leu Ala Asn Thr Asp Val Pro Ile Pro Thr His Tyr Phe Val
                        740 745 750
Val Leu Thr Ser Cys Lys Asn Lys Ser His Thr Pro Glu Asn Cys Pro
                755 760 765
Gly Trp Leu Asp Val Leu Pro Phe Ile Ile Pro His Arg Pro Thr Asn
        770 775 780
Val Glu Ser Cys Pro Glu Gly Lys Pro Glu Ala Leu Trp Val Glu Glu
785 790 795 800
Arg Phe Thr Ala His Ile Ala Arg Val Arg Asp Val Glu Leu Leu Thr
                                805 810 815
Gly Leu Asp Phe Tyr Gln Asp Lys Val Gln Pro Val Ser Glu Ile Leu
                        820 825 830
Gln Leu Lys Thr Tyr Leu Pro Thr Phe Glu Thr Thr Ile Asp Lys Thr
                835 840 845
His Thr Cys Pro Pro Cys Pro Ala Pro Glu Leu Leu Gly Gly Pro Ser
        850 855 860
Val Phe Leu Phe Pro Pro Lys Pro Lys Asp Thr Leu Met Ile Ser Arg
865 870 875 880
Thr Pro Glu Val Thr Cys Val Val Val Asp Val Ser His Glu Asp Pro
                                885 890 895
Glu Val Lys Phe Asn Trp Tyr Val Asp Gly Val Glu Val His Asn Ala
                        900 905 910
Lys Thr Lys Pro Arg Glu Glu Gln Tyr Asn Ser Thr Tyr Arg Val Val
                915 920 925
Ser Val Leu Thr Val Leu His Gln Asp Trp Leu Asn Gly Lys Glu Tyr
        930 935 940
Lys Cys Lys Val Ser Asn Lys Ala Leu Pro Ala Pro Ile Glu Lys Thr
945 950 955 960
Ile Ser Lys Ala Lys Gly Gln Pro Arg Glu Pro Gln Val Tyr Thr Leu
                                965 970 975
Pro Pro Ser Arg Glu Glu Met Thr Lys Asn Gln Val Ser Leu Thr Cys
                        980 985 990
Leu Val Lys Gly Phe Tyr Pro Ser Asp Ile Ala Val Glu Trp Glu Ser
                995 1000 1005
Asn Gly Gln Pro Glu Asn Asn Tyr Lys Thr Thr Pro Pro Val Leu
        1010 1015 1020
Asp Ser Asp Gly Ser Phe Phe Leu Tyr Ser Lys Leu Thr Val Asp
        1025 1030 1035
Lys Ser Arg Trp Gln Gln Gly Asn Val Phe Ser Cys Ser Val Met
        1040 1045 1050
His Glu Ala Leu His Asn His Tyr Thr Gln Lys Ser Leu Ser Leu
        1055 1060 1065
Ser Pro Gly Lys
        1070
Singly underlined: signal peptide sequence; double-underlined:
beginning and end of NPP3; ** = cleavage position at the signal
peptide sequence; bold residues indicate Fc sequence
SEQ. ID NO: 23-ENPP71-Albumin
Met Arg Gly Pro Ala Val Leu Leu Thr Val Ala Leu Ala Thr Leu Leu
1 5 10 15
Ala Pro Gly Ala Gly Leu Lys**Pro Ser Cys Ala Lys Glu Val Lys Ser
                        20 25 30
Cys Lys Gly Arg Cys Phe Glu Arg Thr Phe Gly Asn Cys Arg Cys Asp
                35 40 45
Ala Ala Cys Val Glu Leu Gly Asn Cys Cys Leu Asp Tyr Gln Glu Thr
        50 55 60
Cys Ile Glu Pro Glu His Ile Trp Thr Cys Asn Lys Phe Arg Cys Gly
65 70 75 80
Glu Lys Arg Leu Thr Arg Ser Leu Cys Ala Cys Ser Asp Asp Cys Lys
                                85 90 95
Asp Lys Gly Asp Cys Cys Ile Asn Tyr Ser Ser Val Cys Gln Gly Glu
                        100 105 110
Lys Ser Trp Val Glu Glu Pro Cys Glu Ser Ile Asn Glu Pro Gln Cys
                115 120 125
Pro Ala Gly Phe Glu Thr Pro Pro Thr Leu Leu Phe Ser Leu Asp Gly
        130 135 140
Phe Arg Ala Glu Tyr Leu His Thr Trp Gly Gly Leu Leu Pro Val Ile
145 150 155 160
Ser Lys Leu Lys Lys Cys Gly Thr Tyr Thr Lys Asn Met Arg Pro Val
                                165 170 175
Tyr Pro Thr Lys Thr Phe Pro Asn His Tyr Ser Ile Val Thr Gly Leu
                        180 185 190
Tyr Pro Glu Ser His Gly Ile Ile Asp Asn Lys Met Tyr Asp Pro Lys
                195 200 205
Met Asn Ala Ser Phe Ser Leu Lys Ser Lys Glu Lys Phe Asn Pro Glu
        210 215 220
Trp Tyr Lys Gly Glu Pro Ile Trp Val Thr Ala Lys Tyr Gln Gly Leu
225 230 235 240
Lys Ser Gly Thr Phe Phe Trp Pro Gly Ser Asp Val Glu Ile Asn Gly
                                245 250 255
Ile Phe Pro Asp Ile Tyr Lys Met Tyr Asn Gly Ser Val Pro Phe Glu
                        260 265 270
Glu Arg Ile Leu Ala Val Leu Gln Trp Leu Gln Leu Pro Lys Asp Glu
                275 280 285
Arg Pro His Phe Tyr Thr Leu Tyr Leu Glu Glu Pro Asp Ser Ser Gly
        290 295 300
His Ser Tyr Gly Pro Val Ser Ser Glu Val Ile Lys Ala Leu Gln Arg
305 310 315 320
Val Asp Gly Met Val Gly Met Leu Met Asp Gly Leu Lys Glu Leu Asn
                                325 330 335
Leu His Arg Cys Leu Asn Leu Ile Leu Ile Ser Asp His Gly Met Glu
                        340 345 350
Gln Gly Ser Cys Lys Lys Tyr Ile Tyr Leu Asn Lys Tyr Leu Gly Asp
                355 360 365
Val Lys Asn Ile Lys Val Ile Tyr Gly Pro Ala Ala Arg Leu Arg Pro
        370 375 380
Ser Asp Val Pro Asp Lys Tyr Tyr Ser Phe Asn Tyr Glu Gly Ile Ala
385 390 395 400
Arg Asn Leu Ser Cys Arg Glu Pro Asn Gln His Phe Lys Pro Tyr Leu
                                405 410 415
Lys His Phe Leu Pro Lys Arg Leu His Phe Ala Lys Ser Asp Arg Ile
                        420 425 430
Glu Pro Leu Thr Phe Tyr Leu Asp Pro Gln Trp Gln Leu Ala Leu Asn
                435 440 445
Pro Ser Glu Arg Lys Tyr Cys Gly Ser Gly Phe His Gly Ser Asp Asn
        450 455 460
Val Phe Ser Asn Met Gln Ala Leu Phe Val Gly Tyr Gly Pro Gly Phe
465 470 475 480
Lys His Gly Ile Glu Ala Asp Thr Phe Glu Asn Ile Glu Val Tyr Asn
                                485 490 495
Leu Met Cys Asp Leu Leu Asn Leu Thr Pro Ala Pro Asn Asn Gly Thr
                        500 505 510
His Gly Ser Leu Asn His Leu Leu Lys Asn Pro Val Tyr Thr Pro Lys
                515 520 525
His Pro Lys Glu Val His Pro Leu Val Gln Cys Pro Phe Thr Arg Asn
        530 535 540
Pro Arg Asp Asn Leu Gly Cys Ser Cys Asn Pro Ser Ile Leu Pro Ile
545 550 555 560
Glu Asp Phe Gln Thr Gln Phe Asn Leu Thr Val Ala Glu Glu Lys Ile
                                565 570 575
Ile Lys His Glu Thr Leu Pro Tyr Gly Arg Pro Arg Val Leu Gln Lys
                        580 585 590
Glu Asn Thr Ile Cys Leu Leu Ser Gln His Gln Phe Met Ser Gly Tyr
                595 600 605
Ser Gln Asp Ile Leu Met Pro Leu Trp Thr Ser Tyr Thr Val Asp Arg
        610 615 620
Asn Asp Ser Phe Ser Thr Glu Asp Phe Ser Asn Cys Leu Tyr Gln Asp
625 630 635 640
Phe Arg Ile Pro Leu Ser Pro Val His Lys Cys Ser Phe Tyr Lys Asn
                                645 650 655
Asn Thr Lys Val Ser Tyr Gly Phe Leu Ser Pro Pro Gln Leu Asn Lys
                        660 665 670
Asn Ser Ser Gly Ile Tyr Ser Glu Ala Leu Leu Thr Thr Asn Ile Val
                675 680 685
Pro Met Tyr Gln Ser Phe Gln Val Ile Trp Arg Tyr Phe His Asp Thr
        690 695 700
Leu Leu Arg Lys Tyr Ala Glu Glu Arg Asn Gly Val Asn Val Val Ser
705 710 715 720
Gly Pro Val Phe Asp Phe Asp Tyr Asp Gly Arg Cys Asp Ser Leu Glu
                                725 730 735
Asn Leu Arg Gln Lys Arg Arg Val Ile Arg Asn Gln Glu Ile Leu Ile
                        740 745 750
Pro Thr His Phe Phe Ile Val Leu Thr Ser Cys Lys Asp Thr Ser Gln
                755 760 765
Thr Pro Leu His Cys Glu Asn Leu Asp Thr Leu Ala Phe Ile Leu Pro
        770 775 780
His Arg Thr Asp Asn Ser Glu Ser Cys Val His Gly Lys His Asp Ser
785 790 795 800
Ser Trp Val Glu Glu Leu Leu Met Leu His Arg Ala Arg Ile Thr Asp
                                805 810 815
Val Glu His Ile Thr Gly Leu Ser Phe Tyr Gln Gln Arg Lys Glu Pro
                        820 825 830
Val Ser Asp Ile Leu Lys Leu Lys Thr His Leu Pro Thr Phe Ser Gln
                835 840 845
Glu Asp Gly Gly Ser Gly Gly Ser Met Lys Trp Val Thr Phe Leu Leu
        850 855 860
Leu Leu Phe Val Ser Gly Ser Ala Phe Ser Arg Gly Val Phe Arg Arg
865 870 875 880
Glu Ala His Lys Ser Glu Ile Ala His Arg Tyr Asn Asp Leu Gly Glu
                                885 890 895
Gln His Phe Lys Gly Leu Val Leu Ile Ala Phe Ser Gln Tyr Leu Gln
                        900 905 910
Lys Cys Ser Tyr Asp Glu His Ala Lys Leu Val Gln Glu Val Thr Asp
                915 920 925
Phe Ala Lys Thr Cys Val Ala Asp Glu Ser Ala Ala Asn Cys Asp Lys
        930 935 940
Ser Leu His Thr Leu Phe Gly Asp Lys Leu Cys Ala Ile Pro Asn Leu
945 950 955 960
Arg Glu Asn Tyr Gly Glu Leu Ala Asp Cys Cys Thr Lys Gln Glu Pro
                                965 970 975
Glu Arg Asn Glu Cys Phe Leu Gln His Lys Asp Asp Asn Pro Ser Leu
                        980 985 990
Pro Pro Phe Glu Arg Pro Glu Ala Glu Ala Met Cys Thr Ser Phe Lys
                995 1000 1005
Glu Asn Pro Thr Thr Phe Met Gly His Tyr Leu His Glu Val Ala
        1010 1015 1020
Arg Arg His Pro Tyr Phe Tyr Ala Pro Glu Leu Leu Tyr Tyr Ala
        1025 1030 1035
Glu Gln Tyr Asn Glu Ile Leu Thr Gln Cys Cys Ala Glu Ala Asp
        1040 1045 1050
Lys Glu Ser Cys Leu Thr Pro Lys Leu Asp Gly Val Lys Glu Lys
        1055 1060 1065
Ala Leu Val Ser Ser Val Arg Gln Arg Met Lys Cys Ser Ser Met
        1070 1075 1080
Gln Lys Phe Gly Glu Arg Ala Phe Lys Ala Trp Ala Val Ala Arg
        1085 1090 1095
Leu Ser Gln Thr Phe Pro Asn Ala Asp Phe Ala Glu Ile Thr Lys
        1100 1105 1110
Leu Ala Thr Asp Leu Thr Lys Val Asn Lys Glu Cys Cys His Gly
        1115 1120 1125
Asp Leu Leu Glu Cys Ala Asp Asp Arg Ala Glu Leu Ala Lys Tyr
        1130 1135 1140
Met Cys Glu Asn Gln Ala Thr Ile Ser Ser Lys Leu Gln Thr Cys
        1145 1150 1155
Cys Asp Lys Pro Leu Leu Lys Lys Ala His Cys Leu Ser Glu Val
        1160 1165 1170
Glu His Asp Thr Met Pro Ala Asp Leu Pro Ala Ile Ala Ala Asp
        1175 1180 1185
Phe Val Glu Asp Gln Glu Val Cys Lys Asn Tyr Ala Glu Ala Lys
        1190 1195 1200
Asp Val Phe Leu Gly Thr Phe Leu Tyr Glu Tyr Ser Arg Arg His
        1205 1210 1215
Pro Asp Tyr Ser Val Ser Leu Leu Leu Arg Leu Ala Lys Lys Tyr
        1220 1225 1230
Glu Ala Thr Leu Glu Lys Cys Cys Ala Glu Ala Asn Pro Pro Ala
        1235 1340 1245
Cys Tyr Gly Thr Val Leu Ala Glu Phe Gln Pro Leu Val Glu Glu
        1250 1255 1260
Pro Lys Asn Leu Val Lys Thr Asn Cys Asp Leu Tyr Glu Lys Leu
        1265 1270 1275
Gly Glu Tyr Gly Phe Gln Asn Ala Ile Leu Val Arg Tyr Thr Gln
        1280 1285 1290
Lys Ala Pro Gln Val Ser Thr Pro Thr Leu Val Glu Ala Ala Arg
        1295 1300 1305
Asn Leu Gly Arg Val Gly Thr Lys Cys Cys Thr Leu Pro Glu Asp
        1310 1315 1320
Gln Arg Leu Pro Cys Val Glu Asp Tyr Leu Ser Ala Ile Leu Asn
        1325 1330 1335
Arg Val Cys Leu Leu His Glu Lys Thr Pro Val Ser Glu His Val
        1340 1345 1350
Thr Lys Cys Cys Ser Gly Ser Leu Val Glu Arg Arg Pro Cys Phe
        1355 1360 1365
Ser Ala Leu Thr Val Asp Glu Thr Tyr Val Pro Lys Glu Phe Lys
        1370 1375 1380
Ala Glu Thr Phe Thr Phe His Ser Asp Ile Cys Thr Leu
        1385 1390 1395
Singly underlined: signal peptide sequence; double-underlined:
beginning and end of NPP3; ** = cleavage position at the signal
peptide sequence; bold residues indicate Fc sequence
SEQ. ID NO: 24-ENPP7-NPP3-Albumin
Met Arg Gly Pro Ala Val Leu Leu Thr Val Ala Leu Ala Thr Leu Leu
1 5 10 15
Ala Pro Gly Ala**Lys Gln Gly Ser Cys Arg Lys Lys Cys Phe Asp Ala
                        20 25 30
Ser Phe Arg Gly Leu Glu Asn Cys Arg Cys Asp Val Ala Cys Lys Asp
                35 40 45
Arg Gly Asp Cys Cys Trp Asp Phe Glu Asp Thr Cys Val Glu Ser Thr
        50 55 60
Arg Ile Trp Met Cys Asn Lys Phe Arg Cys Gly Glu Arg Leu Glu Ala
65 70 75 80
Ser Leu Cys Ser Cys Ser Asp Asp Cys Leu Gln Arg Lys Asp Cys Cys
                                85 90 95
Ala Asp Tyr Lys Ser Val Cys Gln Gly Glu Thr Ser Trp Leu Glu Glu
                        100 105 110
Asn Cys Asp Thr Ala Gln Gln Ser Gln Cys Pro Glu Gly Phe Asp Leu
                115 120 125
Pro Pro Val Ile Leu Phe Ser Met Asp Gly Phe Arg Ala Glu Tyr Leu
        130 135 140
Tyr Thr Trp Asp Thr Leu Met Pro Asn Ile Asn Lys Leu Lys Thr Cys
145 150 155 160
Gly Ile His Ser Lys Tyr Met Arg Ala Met Tyr Pro Thr Lys Thr Phe
                                165 170 175
Pro Asn His Tyr Thr Ile Val Thr Gly Leu Tyr Pro Glu Ser His Gly
                        180 185 190
Ile Ile Asp Asn Asn Met Tyr Asp Val Asn Leu Asn Lys Asn Phe Ser
                195 200 205
Leu Ser Ser Lys Glu Gln Asn Asn Pro Ala Trp Trp His Gly Gln Pro
        210 215 220
Met Trp Leu Thr Ala Met Tyr Gln Gly Leu Lys Ala Ala Thr Tyr Phe
225 230 235 240
Trp Pro Gly Ser Glu Val Ala Ile Asn Gly Ser Phe Pro Ser Ile Tyr
                                245 250 255
Met Pro Tyr Asn Gly Ser Val Pro Phe Glu Glu Arg Ile Ser Thr Leu
                        260 265 270
Leu Lys Trp Leu Asp Leu Pro Lys Ala Glu Arg Pro Arg Phe Tyr Thr
                275 280 285
Met Tyr Phe Glu Glu Pro Asp Ser Ser Gly His Ala Gly Gly Pro Val
        290 295 300
Ser Ala Arg Val Ile Lys Ala Leu Gln Val Val Asp His Ala Phe Gly
305 310 315 320
Met Leu Met Glu Gly Leu Lys Gln Arg Asn Leu His Asn Cys Val Asn
                                325 330 335
Ile Ile Leu Leu Ala Asp His Gly Met Asp Gln Thr Tyr Cys Asn Lys
                        340 345 350
Met Glu Tyr Met Thr Asp Tyr Phe Pro Arg Ile Asn Phe Phe Tyr Met
                355 360 365
Tyr Glu Gly Pro Ala Pro Arg Ile Arg Ala His Asn Ile Pro His Asp
        370 375 380
Phe Phe Ser Phe Asn Ser Glu Glu Ile Val Arg Asn Leu Ser Cys Arg
385 390 395 400
Lys Pro Asp Gln His Phe Lys Pro Tyr Leu Thr Pro Asp Leu Pro Lys
                                405 410 415
Arg Leu His Tyr Ala Lys Asn Val Arg Ile Asp Lys Val His Leu Phe
                        420 425 430
Val Asp Gln Gln Trp Leu Ala Val Arg Ser Lys Ser Asn Thr Asn Cys
                435 440 445
Gly Gly Gly Asn His Gly Tyr Asn Asn Glu Phe Arg Ser Met Glu Ala
        450 455 460
Ile Phe Leu Ala His Gly Pro Ser Phe Lys Glu Lys Thr Glu Val Glu
465 470 475 480
Pro Phe Glu Asn Ile Glu Val Tyr Asn Leu Met Cys Asp Leu Leu Arg
                                485 490 495
Ile Gln Pro Ala Pro Asn Asn Gly Thr His Gly Ser Leu Asn His Leu
                        500 505 510
Leu Lys Val Pro Phe Tyr Glu Pro Ser His Ala Glu Glu Val Ser Lys
                515 520 525
Phe Ser Val Cys Gly Phe Ala Asn Pro Leu Pro Thr Glu Ser Leu Asp
        530 535 540
Cys Phe Cys Pro His Leu Gln Asn Ser Thr Gln Leu Glu Gln Val Asn
545 550 555 560
Gln Met Leu Asn Leu Thr Gln Glu Glu Ile Thr Ala Thr Val Lys Val
                                565 570 575
Asn Leu Pro Phe Gly Arg Pro Arg Val Leu Gln Lys Asn Val Asp His
                        580 585 590
Cys Leu Leu Tyr His Arg Glu Tyr Val Ser Gly Phe Gly Lys Ala Met
                595 600 605
Arg Met Pro Met Trp Ser Ser Tyr Thr Val Pro Gln Leu Gly Asp Thr
        610 615 620
Ser Pro Leu Pro Pro Thr Val Pro Asp Cys Leu Arg Ala Asp Val Arg
625 630 635 640
Val Pro Pro Ser Glu Ser Gln Lys Cys Ser Phe Tyr Leu Ala Asp Lys
                                645 650 655
Asn Ile Thr His Gly Phe Leu Tyr Pro Pro Ala Ser Asn Arg Thr Ser
                        660 665 670
Asp Ser Gln Tyr Asp Ala Leu Ile Thr Ser Asn Leu Val Pro Met Tyr
                675 680 685
Glu Glu Phe Arg Lys Met Trp Asp Tyr Phe His Ser Val Leu Leu Ile
        690 695 700
Lys His Ala Thr Glu Arg Asn Gly Val Asn Val Val Ser Gly Pro Ile
705 710 715 720
Phe Asp Tyr Asn Tyr Asp Gly His Phe Asp Ala Pro Asp Glu Ile Thr
                                725 730 735
Lys His Leu Ala Asn Thr Asp Val Pro Ile Pro Thr His Tyr Phe Val
                        740 745 750
Val Leu Thr Ser Cys Lys Asn Lys Ser His Thr Pro Glu Asn Cys Pro
                755 760 765
Gly Trp Leu Asp Val Leu Pro Phe Ile Ile Pro His Arg Pro Thr Asn
        770 775 780
Val Glu Ser Cys Pro Glu Gly Lys Pro Glu Ala Leu Trp Val Glu Glu
785 790 795 800
Arg Phe Thr Ala His Ile Ala Arg Val Arg Asp Val Glu Leu Leu Thr
                                805 810 815
Gly Leu Asp Phe Tyr Gln Asp Lys Val Gln Pro Val Ser Glu Ile Leu
                        820 825 830
Gln Leu Lys Thr Tyr Leu Pro Thr Phe Glu Thr Thr Ile Gly Gly Gly
                835 840 845
Ser Gly Gly Gly Gly Ser Gly Gly Gly Gly Ser Met Lys Trp Val Thr
        850 855 860
Phe Leu Leu Leu Leu Phe Val Ser Gly Ser Ala Phe Ser Arg Gly Val
865 870 875 880
Phe Arg Arg Glu Ala His Lys Ser Glu Ile Ala His Arg Tyr Asn Asp
                                885 890 895
Leu Gly Glu Gln His Phe Lys Gly Leu Val Leu Ile Ala Phe Ser Gln
                        900 905 910
Tyr Leu Gln Lys Cys Ser Tyr Asp Glu His Ala Lys Leu Val Gln Glu
                915 920 925
Val Thr Asp Phe Ala Lys Thr Cys Val Ala Asp Glu Ser Ala Ala Asn
        930 935 940
Cys Asp Lys Ser Leu His Thr Leu Phe Gly Asp Lys Leu Cys Ala Ile
945 950 955 960
Pro Asn Leu Arg Glu Asn Tyr Gly Glu Leu Ala Asp Cys Cys Thr Lys
                                965 970 975
Gln Glu Pro Glu Arg Asn Glu Cys Phe Leu Gln His Lys Asp Asp Asn
                        980 985 990
Pro Ser Leu Pro Pro Phe Glu Arg Pro Glu Ala Glu Ala Met Cys Thr
                995 1000 10055
Ser Phe Lys Glu Asn Pro Thr Thr Phe Met Gly His Tyr Leu His
        1010 1015 1020
Glu Val Ala Arg Arg His Pro Tyr Phe Tyr Ala Pro Glu Leu Leu
        1025 1030 1035
Tyr Tyr Ala Glu Gln Tyr Asn Glu Ile Leu Thr Gln Cys Cys Ala
        1040 1045 1050
Glu Ala Asp Lys Glu Ser Cys Leu Thr Pro Lys Leu Asp Gly Val
        1055 1060 1065
Lys Glu Lys Ala Leu Val Ser Ser Val Arg Gln Arg Met Lys Cys
        1070 1075 1080
Ser Ser Met Gln Lys Phe Gly Glu Arg Ala Phe Lys Ala Trp Ala
        1085 1090 1095
Val Ala Arg Leu Ser Gln Thr Phe Pro Asn Ala Asp Phe Ala Glu
        1100 1105 1110
Ile Thr Lys Leu Ala Thr Asp Leu Thr Lys Val Asn Lys Glu Cys
        1115 1120 1125
Cys His Gly Asp Leu Leu Glu Cys Ala Asp Asp Arg Ala Glu Leu
        1130 1135 1140
Ala Lys Tyr Met Cys Glu Asn Gln Ala Thr Ile Ser Ser Lys Leu
        1145 1150 1155
Gln Thr Cys Cys Asp Lys Pro Leu Leu Lys Lys Ala His Cys Leu
        1160 1165 1170
Ser Glu Val Glu His Asp Thr Met Pro Ala Asp Leu Pro Ala Ile
        1175 1180 1185
Ala Ala Asp Phe Val Glu Asp Gln Glu Val Cys Lys Asn Tyr Ala
        1190 1195 1200
Glu Ala Lys Asp Val Phe Leu Gly Thr Phe Leu Tyr Glu Tyr Ser
        1205 1210 1215
Arg Arg His Pro Asp Tyr Ser Val Ser Leu Leu Leu Arg Leu Ala
        1220 1225 1230
Lys Lys Tyr Glu Ala Thr Leu Glu Lys Cys Cys Ala Glu Ala Asn
        1235 1340 1245
Pro Pro Ala Cys Tyr Gly Thr Val Leu Ala Glu Phe Gln Pro Leu
        1250 1255 1260
Val Glu Glu Pro Lys Asn Leu Val Lys Thr Asn Cys Asp Leu Tyr
        1265 1270 1275
Glu Lys Leu Gly Glu Tyr Gly Phe Gln Asn Ala Ile Leu Val Arg
        1280 1285 1290
Tyr Thr Gln Lys Ala Pro Gln Val Ser Thr Pro Thr Leu Val Glu
        1295 1300 1305
Ala Ala Arg Asn Leu Gly Arg Val Gly Thr Lys Cys Cys Thr Leu
        1310 1315 1320
Pro Glu Asp Gln Arg Leu Pro Cys Val Glu Asp Tyr Leu Ser Ala
        1325 1330 1335
Ile Leu Asn Arg Val Cys Leu Leu His Glu Lys Thr Pro Val Ser
        1340 1345 1350
Glu His Val Thr Lys Cys Cys Ser Gly Ser Leu Val Glu Arg Arg
        1355 1360 1365
Pro Cys Phe Ser Ala Leu Thr Val Asp Glu Thr Tyr Val Pro Lys
        1370 1375 1380
Glu Phe Lys Ala Glu Thr Phe Thr Phe His Ser Asp Ile Cys Thr
        1385 1390 1395
Leu Pro Glu Lys Glu Lys Gln Ile Lys Lys Gln Thr Ala Leu Ala
        1400 1405 1410
Glu Leu Val Lys His Lys Pro Lys Ala Thr Ala Glu Gln Leu Lys
        1415 1420 1425
Thr Val Met Asp Asp Phe Ala Gln Phe Leu Asp Thr Cys Cys Lys
        1430 1435 1440
Ala Ala Asp Lys Asp Thr Cys Phe Ser Thr Glu Gly Pro Asn Leu
        1445 1450 1455
Val Thr Arg Cys Lys Asp Ala Leu Ala
        1460 1465
Singly underlined: signal peptide sequence; double-underlined:
beginning and end of NPP3; ** = cleavage position at the signal
peptide sequence; bold residues indicate albumin sequence
SEQ. ID NO: 25-ENPP7-ENPP3-Albumin
Met Arg Gly Pro Ala Val Leu Leu Thr Val Ala Leu Ala Thr Leu Leu
1 5 10 15
Ala Pro Gly Ala**Lys Gln Gly Ser Cys Arg Lys Lys Cys Phe Asp Ala
                        20 25 30
Ser Phe Arg Gly Leu Glu Asn Cys Arg Cys Asp Val Ala Cys Lys Asp
                35 40 45
Arg Gly Asp Cys Cys Trp Asp Phe Glu Asp Thr Cys Val Glu Ser Thr
        50 55 60
Arg Ile Trp Met Cys Asn Lys Phe Arg Cys Gly Glu Arg Leu Glu Ala
65 70 75 80
Ser Leu Cys Ser Cys Ser Asp Asp Cys Leu Gln Arg Lys Asp Cys Cys
                                85 90 95
Ala Asp Tyr Lys Ser Val Cys Gln Gly Glu Thr Ser Trp Leu Glu Glu
                        100 105 110
Asn Cys Asp Thr Ala Gln Gln Ser Gln Cys Pro Glu Gly Phe Asp Leu
                115 120 125
Pro Pro Val Ile Leu Phe Ser Met Asp Gly Phe Arg Ala Glu Tyr Leu
        130 135 140
Tyr Thr Trp Asp Thr Leu Met Pro Asn Ile Asn Lys Leu Lys Thr Cys
145 150 155 160
Gly Ile His Ser Lys Tyr Met Arg Ala Met Tyr Pro Thr Lys Thr Phe
                                165 170 175
Pro Asn His Tyr Thr Ile Val Thr Gly Leu Tyr Pro Glu Ser His Gly
                        180 185 190
Ile Ile Asp Asn Asn Met Tyr Asp Val Asn Leu Asn Lys Asn Phe Ser
                195 200 205
Leu Ser Ser Lys Glu Gln Asn Asn Pro Ala Trp Trp His Gly Gln Pro
        210 215 220
Met Trp Leu Thr Ala Met Tyr Gln Gly Leu Lys Ala Ala Thr Tyr Phe
225 230 235 240
Trp Pro Gly Ser Glu Val Ala Ile Asn Gly Ser Phe Pro Ser Ile Tyr
                                245 250 255
Met Pro Tyr Asn Gly Ser Val Pro Phe Glu Glu Arg Ile Ser Thr Leu
                        260 265 270
Leu Lys Trp Leu Asp Leu Pro Lys Ala Glu Arg Pro Arg Phe Tyr Thr
                275 280 285
Met Tyr Phe Glu Glu Pro Asp Ser Ser Gly His Ala Gly Gly Pro Val
        290 295 300
Ser Ala Arg Val Ile Lys Ala Leu Gln Val Val Asp His Ala Phe Gly
305 310 315 320
Met Leu Met Glu Gly Leu Lys Gln Arg Asn Leu His Asn Cys Val Asn
                                325 330 335
Ile Ile Leu Leu Ala Asp His Gly Met Asp Gln Thr Tyr Cys Asn Lys
                        340 345 350
Met Glu Tyr Met Thr Asp Tyr Phe Pro Arg Ile Asn Phe Phe Tyr Met
                355 360 365
Tyr Glu Gly Pro Ala Pro Arg Ile Arg Ala His Asn Ile Pro His Asp
        370 375 380
Phe Phe Ser Phe Asn Ser Glu Glu Ile Val Arg Asn Leu Ser Cys Arg
385 390 395 400
Lys Pro Asp Gln His Phe Lys Pro Tyr Leu Thr Pro Asp Leu Pro Lys
                                405 410 415
Arg Leu His Tyr Ala Lys Asn Val Arg Ile Asp Lys Val His Leu Phe
                        420 425 430
Val Asp Gln Gln Trp Leu Ala Val Arg Ser Lys Ser Asn Thr Asn Cys
                435 440 445
Gly Gly Gly Asn His Gly Tyr Asn Asn Glu Phe Arg Ser Met Glu Ala
        450 455 460
Ile Phe Leu Ala His Gly Pro Ser Phe Lys Glu Lys Thr Glu Val Glu
465 470 475 480
Pro Phe Glu Asn Ile Glu Val Tyr Asn Leu Met Cys Asp Leu Leu Arg
                                485 490 495
Ile Gln Pro Ala Pro Asn Asn Gly Thr His Gly Ser Leu Asn His Leu
                        500 505 510
Leu Lys Val Pro Phe Tyr Glu Pro Ser His Ala Glu Glu Val Ser Lys
                515 520 525
Phe Ser Val Cys Gly Phe Ala Asn Pro Leu Pro Thr Glu Ser Leu Asp
        530 535 540
Cys Phe Cys Pro His Leu Gln Asn Ser Thr Gln Leu Glu Gln Val Asn
545 550 555 560
Gln Met Leu Asn Leu Thr Gln Glu Glu Ile Thr Ala Thr Val Lys Val
                                565 570 575
Asn Leu Pro Phe Gly Arg Pro Arg Val Leu Gln Lys Asn Val Asp His
                        580 585 590
Cys Leu Leu Tyr His Arg Glu Tyr Val Ser Gly Phe Gly Lys Ala Met
                595 600 605
Arg Met Pro Met Trp Ser Ser Tyr Thr Val Pro Gln Leu Gly Asp Thr
        610 615 620
Ser Pro Leu Pro Pro Thr Val Pro Asp Cys Leu Arg Ala Asp Val Arg
625 630 635 640
Val Pro Pro Ser Glu Ser Gln Lys Cys Ser Phe Tyr Leu Ala Asp Lys
                                645 650 655
Asn Ile Thr His Gly Phe Leu Tyr Pro Pro Ala Ser Asn Arg Thr Ser
                        660 665 670
Asp Ser Gln Tyr Asp Ala Leu Ile Thr Ser Asn Leu Val Pro Met Tyr
                675 680 685
Glu Glu Phe Arg Lys Met Trp Asp Tyr Phe His Ser Val Leu Leu Ile
        690 695 700
Lys His Ala Thr Glu Arg Asn Gly Val Asn Val Val Ser Gly Pro Ile
705 710 715 720
Phe Asp Tyr Asn Tyr Asp Gly His Phe Asp Ala Pro Asp Glu Ile Thr
                                725 730 735
Lys His Leu Ala Asn Thr Asp Val Pro Ile Pro Thr His Tyr Phe Val
                        740 745 750
Val Leu Thr Ser Cys Lys Asn Lys Ser His Thr Pro Glu Asn Cys Pro
                755 760 765
Gly Trp Leu Asp Val Leu Pro Phe Ile Ile Pro His Arg Pro Thr Asn
        770 775 780
Val Glu Ser Cys Pro Glu Gly Lys Pro Glu Ala Leu Trp Val Glu Glu
785 790 795 800
Arg Phe Thr Ala His Ile Ala Arg Val Arg Asp Val Glu Leu Leu Thr
                                805 810 815
Gly Leu Asp Phe Tyr Gln Asp Lys Val Gln Pro Val Ser Glu Ile Leu
                        820 825 830
Gln Leu Lys Thr Tyr Leu Pro Thr Phe Glu Thr Thr Ile Asp Lys Thr
                835 840 845
His Thr Cys Pro Pro Cys Pro Ala Pro Glu Leu Leu Gly Gly Pro Ser
        850 855 860
Val Phe Leu Phe Pro Pro Lys Pro Lys Asp Thr Leu Met Ile Ser Arg
865 870 875 880
Thr Pro Glu Val Thr Gly Gly Gly Ser Gly Gly Gly Gly Ser Gly Gly
                                885 890 895
Gly Gly Ser Met Lys Trp Val Thr Phe Leu Leu Leu Leu Phe Val Ser
                        900 905 910
Gly Ser Ala Phe Ser Arg Gly Val Phe Arg Arg Glu Ala His Lys Ser
                915 920 925
Glu Ile Ala His Arg Tyr Asn Asp Leu Gly Glu Gln His Phe Lys Gly
        930 935 940
Leu Val Leu Ile Ala Phe Ser Gln Tyr Leu Gln Lys Cys Ser Tyr Asp
945 950 955 960
Glu His Ala Lys Leu Val Gln Glu Val Thr Asp Phe Ala Lys Thr Cys
                                965 970 975
Val Ala Asp Glu Ser Ala Ala Asn Cys Asp Lys Ser Leu His Thr Leu
                        980 985 990
Phe Gly Asp Lys Leu Cys Ala Ile Pro Asn Leu Arg Glu Asn Tyr Gly
                995 1000 1005
Glu Leu Ala Asp Cys Cys Thr Lys Gln Glu Pro Glu ArgAsn Glu
        1010 1015 1020
Cys Phe Leu Gln His Lys Asp Asp Asn Pro Ser Leu Pro Pro Phe
        1025 1030 1035
Glu Arg Pro Glu Ala Glu Ala Met Cys Thr Ser Phe Lys Glu Asn
        1040 1045 1050
Pro Thr Thr Phe Met Gly His Tyr Leu His Glu Val Ala Arg Arg
        1055 1060 1065
His Pro Tyr Phe Tyr Ala Pro Glu Leu Leu Tyr Tyr Ala Glu Gln
        1070 1075 1080
Tyr Asn Glu Ile Leu Thr Gln Cys Cys Ala Glu Ala Asp Lys Glu
        1085 1090 1095
Ser Cys Leu Thr Pro Lys Leu Asp Gly Val Lys Glu Lys Ala Leu
        1100 1105 1110
Val Ser Ser Val Arg Gln Arg Met Lys Cys Ser Ser Met Gln Lys
        1115 1120 1125
Phe Gly Glu Arg Ala Phe Lys Ala Trp Ala Val Ala Arg Leu Ser
        1130 1135 1140
Gln Thr Phe Pro Asn Ala Asp Phe Ala Glu Ile Thr Lys Leu Ala
        1145 1150 1155
Thr Asp Leu Thr Lys Val Asn Lys Glu Cys Cys His Gly Asp Leu
        1160 1165 1170
Leu Glu Cys Ala Asp Asp Arg Ala Glu Leu Ala Lys Tyr Met Cys
        1175 1180 1185
Glu Asn Gln Ala Thr Ile Ser Ser Lys Leu Gln Thr Cys Cys Asp
        1190 1195 1200
Lys Pro Leu Leu Lys Lys Ala His Cys Leu Ser Glu Val Glu His
        1205 1210 1215
Asp Thr Met Pro Ala Asp Leu Pro Ala Ile Ala Ala Asp Phe Val
        1220 1225 1230
Glu Asp Gln Glu Val Cys Lys Asn Tyr Ala Glu Ala Lys Asp Val
        1235 1340 1245
Phe Leu Gly Thr Phe Leu Tyr Glu Tyr Ser Arg Arg His Pro Asp
        1250 1255 1260
Tyr Ser Val Ser Leu Leu Leu Arg Leu Ala Lys Lys Tyr Glu Ala
        1265 1270 1275
Thr Leu Glu Lys Cys Cys Ala Glu Ala Asn Pro ProAla Cys Tyr
        1280 1285 1290
Gly Thr Val Leu Ala Glu Phe Gln Pro Leu Val Glu Glu Pro Lys
        1295 1300 1305
Asn Leu Val Lys Thr Asn Cys Asp Leu Tyr Glu Lys Leu Gly Glu
        1310 1315 1320
Tyr Gly Phe Gln Asn Ala Ile Leu Val Arg Tyr Thr Gln Lys Ala
        1325 1330 1335
Pro Gln Val Ser Thr Pro Thr Leu Val Glu Ala Ala Arg Asn Leu
        1340 1345 1350
Gly Arg Val Gly Thr Lys Cys Cys Thr Leu Pro Glu Asp Gln Arg
        1355 1360 1365
Leu Pro Cys Val Glu Asp Tyr Leu Ser Ala Ile Leu Asn Arg Val
        1370 1375 1380
Cys Leu Leu His Glu Lys Thr Pro Val Ser Glu His Val Thr Lys
        1385 1390 1395
Cys Cys Ser Gly Ser Leu Val Glu Arg Arg Pro Cys Phe Ser Ala
        1400 1405 1410
Leu Thr Val Asp Glu Thr Tyr Val Pro Lys Glu Phe Lys Ala Glu
        1415 1420 1425
Thr Phe Thr Phe His Ser Asp Ile Cys Thr Leu Pro Glu Lys Glu
        1430 1435 1440
Lys Gln Ile Lys Lys Gln Thr Ala Leu Ala Glu Leu Val Lys His
        1445 1450 1455
Lys Pro Lys Ala Thr Ala Glu Gln Leu Lys Thr Val Met Asp Asp
        1460 1465 1470
Phe Ala Gln Phe Leu Asp Thr Cys Cys Lys Ala Ala Asp Lys Asp
        1475 1480 1485
Thr Cys Phe Ser Thr Glu Gly Pro Asn Leu Val Thr Arg Cys Lys
        1490 1495 1500
Asp Ala Leu Ala
        1505
Singly underlined: signal peptide sequence; double-underlined:
beginning and end of NPP3; ** = cleavage position at the signal
peptide sequence; bold residues indicate albumin sequence
SEQ. ID NO: 26-ENPP71 Amino Acid Sequence
Met Arg Gly Pro Ala Val Leu Leu Thr Val Ala Leu Ala Thr Leu Leu
1 5 10 15
Ala Pro Gly Ala Gly Ala**Gly Leu Lys Pro Ser Cys Ala Lys Glu Val
                        20 25 30
Lys Ser Cys Lys Gly Arg Cys Phe Glu Arg Thr Phe Gly Asn Cys Arg
                35 40 45
Cys Asp Ala Ala Cys Val Glu Leu Gly Asn Cys Cys Leu Asp Tyr Gln
        50 55 60
Glu Thr Cys Ile Glu Pro Glu His Ile Trp Thr Cys Asn Lys Phe Arg
65 70 75 80
Cys Gly Glu Lys Arg Leu Thr Arg Ser Leu Cys Ala Cys Ser Asp Asp
                                85 90 95
Cys Lys Asp Lys Gly Asp Cys Cys Ile Asn Tyr Ser Ser Val Cys Gln
                        100 105 110
Gly Glu Lys Ser Trp Val Glu Glu Pro Cys Glu Ser Ile Asn Glu Pro
                115 120 125
Gln Cys Pro Ala Gly Phe Glu Thr Pro Pro Thr Leu Leu Phe Ser Leu
        130 135 140
Asp Gly Phe Arg Ala Glu Tyr Leu His Thr Trp Gly Gly Leu Leu Pro
145 150 155 160
Val Ile Ser Lys Leu Lys Lys Cys Gly Thr Tyr Thr Lys Asn Met Arg
                                165 170 175
Pro Val Tyr Pro Thr Lys Thr Phe Pro Asn His Tyr Ser Ile Val Thr
                        180 185 190
Gly Leu Tyr Pro Glu Ser His Gly Ile Ile Asp Asn Lys Met Tyr Asp
                195 200 205
Pro Lys Met Asn Ala Ser Phe Ser Leu Lys Ser Lys Glu Lys Phe Asn
        210 215 220
Pro Glu Trp Tyr Lys Gly Glu Pro Ile Trp Val Thr Ala Lys Tyr Gln
225 230 235 240
Gly Leu Lys Ser Gly Thr Phe Phe Trp Pro Gly Ser Asp Val Glu Ile
                                245 250 255
Asn Gly Ile Phe Pro Asp Ile Tyr Lys Met Tyr Asn Gly Ser Val Pro
                        260 265 270
Phe Glu Glu Arg Ile Leu Ala Val Leu Gln Trp Leu Gln Leu Pro Lys
                275 280 285
Asp Glu Arg Pro His Phe Tyr Thr Leu Tyr Leu Glu Glu Pro Asp Ser
        290 295 300
Ser Gly His Ser Tyr Gly Pro Val Ser Ser Glu Val Ile Lys Ala Leu
305 310 315 320
Gln Arg Val Asp Gly Met Val Gly Met Leu Met Asp Gly Leu Lys Glu
                                325 330 335
Leu Asn Leu His Arg Cys Leu Asn Leu Ile Leu Ile Ser Asp His Gly
                        340 345 350
Met Glu Gln Gly Ser Cys Lys Lys Tyr Ile Tyr Leu Asn Lys Tyr Leu
                355 360 365
Gly Asp Val Lys Asn Ile Lys Val Ile Tyr Gly Pro Ala Ala Arg Leu
        370 375 380
Arg Pro Ser Asp Val Pro Asp Lys Tyr Tyr Ser Phe Asn Tyr Glu Gly
385 390 395 400
Ile Ala Arg Asn Leu Ser Cys Arg Glu Pro Asn Gln His Phe Lys Pro
                                405 410 415
Tyr Leu Lys His Phe Leu Pro Lys Arg Leu His Phe Ala Lys Ser Asp
                        420 425 430
Arg Ile Glu Pro Leu Thr Phe Tyr Leu Asp Pro Gln Trp Gln Leu Ala
                435 440 445
Leu Asn Pro Ser Glu Arg Lys Tyr Cys Gly Ser Gly Phe His Gly Ser
        450 455 460
Asp Asn Val Phe Ser Asn Met Gln Ala Leu Phe Val Gly Tyr Gly Pro
465 470 475 480
Gly Phe Lys His Gly Ile Glu Ala Asp Thr Phe Glu Asn Ile Glu Val
                                485 490 495
Tyr Asn Leu Met Cys Asp Leu Leu Asn Leu Thr Pro Ala Pro Asn Asn
                        500 505 510
Gly Thr His Gly Ser Leu Asn His Leu Leu Lys Asn Pro Val Tyr Thr
                515 520 525
Pro Lys His Pro Lys Glu Val His Pro Leu Val Gln Cys Pro Phe Thr
        530 535 540
Arg Asn Pro Arg Asp Asn Leu Gly Cys Ser Cys Asn Pro Ser Ile Leu
545 550 555 560
Pro Ile Glu Asp Phe Gln Thr Gln Phe Asn Leu Thr Val Ala Glu Glu
                                565 570 575
Lys Ile Ile Lys His Glu Thr Leu Pro Tyr Gly Arg Pro Arg Val Leu
                        580 585 590
Gln Lys Glu Asn Thr Ile Cys Leu Leu Ser Gln His Gln Phe Met Ser
                595 600 605
Gly Tyr Ser Gln Asp Ile Leu Met Pro Leu Trp Thr Ser Tyr Thr Val
        610 615 620
Asp Arg Asn Asp Ser Phe Ser Thr Glu Asp Phe Ser Asn Cys Leu Tyr
625 630 635 640
Gln Asp Phe Arg Ile Pro Leu Ser Pro Val His Lys Cys Ser Phe Tyr
                                645 650 655
Lys Asn Asn Thr Lys Val Ser Tyr Gly Phe Leu Ser Pro Pro Gln Leu
                        660 665 670
Asn Lys Asn Ser Ser Gly Ile Tyr Ser Glu Ala Leu Leu Thr Thr Asn
                675 680 685
Ile Val Pro Met Tyr Gln Ser Phe Gln Val Ile TrpArg Tyr Phe His
        690 695 700
Asp Thr Leu Leu Arg Lys Tyr Ala Glu Glu Arg Asn Gly Val Asn Val
705 710 715 720
Val Ser Gly Pro Val Phe Asp Phe Asp Tyr Asp Gly Arg Cys Asp Ser
                                725 730 735
Leu Glu Asn Leu Arg Gln Lys Arg Arg Val Ile Arg Asn Gln Glu Ile
                        740 745 750
Leu Ile Pro Thr His Phe Phe Ile Val Leu Thr Ser Cys Lys Asp Thr
                755 760 765
Ser Gln Thr Pro Leu His Cys Glu Asn Leu Asp ThrLeu Ala Phe Ile
        770 775 780
Leu Pro His Arg Thr Asp Asn Ser Glu Ser Cys Val His Gly Lys His
785 790 795 800
Asp Ser Ser Trp Val Glu Glu Leu Leu Met Leu His Arg Ala Arg Ile
                                805 810 815
Thr Asp Val Glu His Ile Thr Gly Leu Ser Phe Tyr Gln Gln Arg Lys
                        820 825 830
Glu Pro Val Ser Asp Ile Leu Lys Leu Lys Thr His Leu Pro Thr Phe
                835 840 845
Ser Gln Glu Asp
        850
Singly underlined: signal peptide sequence; double-underlined:
beginning and end of NPP1; ** = cleavage position at the signal
peptide sequence
SEQ. ID NO: 27-ENPP121 Amino Acid Sequence
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly**Phe Thr Ala Gly
                                85 90 95
Leu Lys Pro Ser Cys Ala Lys Glu Val Lys Ser Cys Lys Gly Arg Cys
                        100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
        370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp
                915 920 925
Singly underlined: signal peptide sequence; double-underlined:
beginning and end of NPP1; ** = cleavage position at the signal
peptide sequence
SEQ. ID. NO: 28-ENPP121-Fc Amino Acid Sequence
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly**Phe Thr Ala Gly
                                85 90 95
Leu Lys Pro Ser Cys Ala Lys Glu Val Lys Ser Cys Lys Gly Arg Cys
                        100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
        370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp Leu Ile Asn
                915 920 925
Asp Lys Thr His Thr Cys Pro Pro Cys Pro Ala Pro Glu Leu Leu Gly
        930 935 940
Gly Pro Ser Val Phe Leu Phe Pro Pro Lys Pro Lys Asp Thr Leu Met
945 950 955 960
Ile Ser Arg Thr Pro Glu Val Thr Cys Val Val Val Asp Val Ser His
                                965 970 975
Glu Asp Pro Glu Val Lys Phe Asn Trp Tyr Val Asp Gly Val Glu Val
                        980 985 990
His Asn Ala Lys Thr Lys Pro Arg Glu Glu Gln Tyr Asn Ser Thr Tyr
                995 1000 1005
Arg Val Val Ser Val Leu Thr Val Leu His Gln Asp Trp Leu Asn
        1010 1015 1020
Gly Lys Glu Tyr Lys Cys Lys Val Ser Asn Lys Ala Leu Pro Ala
        1025 1030 1035
Pro Ile Glu Lys Thr Ile Ser Lys Ala Lys Gly Gln Pro Arg Glu
        1040 1045 1050
Pro Gln Val Tyr Thr Leu Pro Pro Ser Arg Glu Glu Met Thr Lys
        1055 1060 1065
Asn Gln Val Ser Leu Thr Cys Leu Val Lys Gly Phe Tyr Pro Ser
        1070 1075 1080
Asp Ile Ala Val Glu Trp Glu Ser Asn Gly Gln Pro Glu Asn Asn
        1085 1090 1095
Tyr Lys Thr Thr Pro Pro Val Leu Asp Ser Asp Gly Ser Phe Phe
        1100 1105 1110
Leu Tyr Ser Lys Leu Thr Val Asp Lys Ser Arg Trp Gln Gln Gly
        1115 1120 1125
Asn Val Phe Ser Cys Ser Val Met His Glu Ala Leu His Asn His
        1130 1135 1140
Tyr Thr Gln Lys Ser Leu Ser Leu Ser Pro Gly Lys
        1145 1150 1155
Singly underlined: signal peptide sequence; double-underlined:
beginning and end of NPP1; ** = cleavage position at the signal
peptide sequence; bold residues indicate Fc sequence
SEQ. ID NO: 29-ENPP121-ALB Amino Acid Sequence:
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln AlaAla Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly**Phe Thr Ala Gly
                                85 90 95
Leu Lys Pro Ser Cys Ala Lys Glu Val Lys Ser Cys Lys Gly Arg Cys
                        100 105 110
Phe Glu Arg Thr Phe Gly Asn Cys Arg Cys Asp Ala Ala Cys Val Glu
                115 120 125
Leu Gly Asn Cys Cys Leu Asp Tyr Gln Glu Thr Cys Ile Glu Pro Glu
        130 135 140
His Ile Trp Thr Cys Asn Lys Phe Arg Cys Gly Glu Lys Arg Leu Thr
145 150 155 160
Arg Ser Leu Cys Ala Cys Ser Asp Asp Cys Lys Asp Lys Gly Asp Cys
                                165 170 175
Cys Ile Asn Tyr Ser Ser Val Cys Gln Gly Glu Lys Ser Trp Val Glu
                        180 185 190
Glu Pro Cys Glu Ser Ile Asn Glu Pro Gln Cys Pro Ala Gly Phe Glu
                195 200 205
Thr Pro Pro Thr Leu Leu Phe Ser Leu Asp Gly Phe Arg Ala Glu Tyr
        210 215 220
Leu His Thr Trp Gly Gly Leu Leu Pro Val Ile Ser Lys Leu Lys Lys
225 230 235 240
Cys Gly Thr Tyr Thr Lys Asn Met Arg Pro Val Tyr Pro Thr Lys Thr
                                245 250 255
Phe Pro Asn His Tyr Ser Ile Val Thr Gly Leu Tyr Pro Glu Ser His
                        260 265 270
Gly Ile Ile Asp Asn Lys Met Tyr Asp Pro Lys Met Asn Ala Ser Phe
                275 280 285
Ser Leu Lys Ser Lys Glu Lys Phe Asn Pro Glu Trp Tyr Lys Gly Glu
        290 295 300
Pro Ile Trp Val Thr Ala Lys Tyr Gln Gly Leu Lys Ser Gly Thr Phe
305 310 315 320
Phe Trp Pro Gly Ser Asp Val Glu Ile Asn Gly Ile Phe Pro Asp Ile
                                325 330 335
Tyr Lys Met Tyr Asn Gly Ser Val Pro Phe Glu Glu Arg Ile Leu Ala
                        340 345 350
Val Leu Gln Trp Leu Gln Leu Pro Lys Asp Glu Arg Pro His Phe Tyr
                355 360 365
Thr Leu Tyr Leu Glu Glu Pro Asp Ser Ser Gly His Ser Tyr Gly Pro
        370 375 380
Val Ser Ser Glu Val Ile Lys Ala Leu Gln Arg Val Asp Gly Met Val
385 390 395 400
Gly Met Leu Met Asp Gly Leu Lys Glu Leu Asn Leu His Arg Cys Leu
                                405 410 415
Asn Leu Ile Leu Ile Ser Asp His Gly Met Glu Gln Gly Ser Cys Lys
                        420 425 430
Lys Tyr Ile Tyr Leu Asn Lys Tyr Leu Gly Asp Val Lys Asn Ile Lys
                435 440 445
Val Ile Tyr Gly Pro Ala Ala Arg Leu Arg Pro Ser Asp Val Pro Asp
        450 455 460
Lys Tyr Tyr Ser Phe Asn Tyr Glu Gly Ile Ala Arg Asn Leu Ser Cys
465 470 475 480
Arg Glu Pro Asn Gln His Phe Lys Pro Tyr Leu Lys His Phe Leu Pro
                                485 490 495
Lys Arg Leu His Phe Ala Lys Ser Asp Arg Ile Glu Pro Leu Thr Phe
                        500 505 510
Tyr Leu Asp Pro Gln Trp Gln Leu Ala Leu Asn Pro Ser Glu Arg Lys
                515 520 525
Tyr Cys Gly Ser Gly Phe His Gly Ser Asp Asn Val Phe Ser Asn Met
        530 535 540
Gln Ala Leu Phe Val Gly Tyr Gly Pro Gly Phe Lys His Gly Ile Glu
545 550 555 560
Ala Asp Thr Phe Glu Asn Ile Glu Val Tyr Asn Leu Met Cys Asp Leu
                                565 570 575
Leu Asn Leu Thr Pro Ala Pro Asn Asn Gly Thr His Gly Ser Leu Asn
                        580 585 590
His Leu Leu Lys Asn Pro Val Tyr Thr Pro Lys His Pro Lys Glu Val
                595 600 605
His Pro Leu Val Gln Cys Pro Phe Thr Arg Asn Pro Arg Asp Asn Leu
        610 615 620
Gly Cys Ser Cys Asn Pro Ser Ile Leu Pro Ile Glu Asp Phe Gln Thr
625 630 635 640
Gln Phe Asn Leu Thr Val Ala Glu Glu Lys Ile Ile Lys His Glu Thr
                                645 650 655
Leu Pro Tyr Gly Arg Pro Arg Val Leu Gln Lys Glu Asn Thr Ile Cys
                        660 665 670
Leu Leu Ser Gln His Gln Phe Met Ser Gly Tyr Ser Gln Asp Ile Leu
                675 680 685
Met Pro Leu Trp Thr Ser Tyr Thr Val Asp Arg Asn Asp Ser Phe Ser
        690 695 700
Thr Glu Asp Phe Ser Asn Cys Leu Tyr Gln Asp Phe Arg Ile Pro Leu
705 710 715 720
Ser Pro Val His Lys Cys Ser Phe Tyr Lys Asn Asn Thr Lys Val Ser
                                725 730 735
Tyr Gly Phe Leu Ser Pro Pro Gln Leu Asn Lys Asn Ser Ser Gly Ile
                        740 745 750
Tyr Ser Glu Ala Leu Leu Thr Thr Asn Ile Val Pro Met Tyr Gln Ser
                755 760 765
Phe Gln Val Ile Trp Arg Tyr Phe His Asp Thr Leu Leu Arg Lys Tyr
        770 775 780
Ala Glu Glu Arg Asn Gly Val Asn Val Val Ser Gly Pro Val Phe Asp
785 790 795 800
Phe Asp Tyr Asp Gly Arg Cys Asp Ser Leu Glu Asn Leu Arg Gln Lys
                                805 810 815
Arg Arg Val Ile Arg Asn Gln Glu Ile Leu Ile Pro Thr His Phe Phe
                        820 825 830
Ile Val Leu Thr Ser Cys Lys Asp Thr Ser Gln Thr Pro Leu His Cys
                835 840 845
Glu Asn Leu Asp Thr Leu Ala Phe Ile Leu Pro His Arg Thr Asp Asn
        850 855 860
Ser Glu Ser Cys Val His Gly Lys His Asp Ser Ser Trp Val Glu Glu
865 870 875 880
Leu Leu Met Leu His Arg Ala Arg Ile Thr Asp Val Glu His Ile Thr
                                885 890 895
Gly Leu Ser Phe Tyr Gln Gln Arg Lys Glu Pro Val Ser Asp Ile Leu
                        900 905 910
Lys Leu Lys Thr His Leu Pro Thr Phe Ser Gln Glu Asp Arg Ser Gly
                915 920 925
Ser Gly Gly Ser Met Lys Trp Val Thr Phe Leu Leu Leu Leu Phe Val
        930 935 940
Ser Gly Ser Ala Phe Ser Arg Gly Val Phe Arg Arg Glu Ala His Lys
945 950 955 960
Ser Glu Ile Ala His Arg Tyr Asn Asp Leu Gly Glu Gln His Phe Lys
                                965 970 975
Gly Leu Val Leu Ile Ala Phe Ser Gln Tyr Leu Gln Lys Cys Ser Tyr
                        980 985 990
Asp Glu His Ala Lys Leu Val Gln Glu Val Thr Asp Phe Ala Lys Thr
                995 1000 1005
Cys Val Ala Asp Glu Ser Ala Ala Asn Cys Asp Lys Ser Leu His
        1010 1015 1020
Thr Leu Phe Gly Asp Lys Leu Cys Ala Ile Pro Asn Leu Arg Glu
        1025 1030 1035
Asn Tyr Gly Glu Leu Ala Asp Cys Cys Thr Lys Gln Glu Pro Glu
        1040 1045 1050
Arg Asn Glu Cys Phe Leu Gln His Lys Asp Asp Asn Pro Ser Leu
        1055 1060 1065
Pro Pro Phe Glu Arg Pro Glu Ala Glu Ala Met Cys Thr Ser Phe
        1070 1075 1080
Lys Glu Asn Pro Thr Thr Phe Met Gly His Tyr Leu His Glu Val
        1085 1090 1095
Ala Arg Arg His Pro Tyr Phe Tyr Ala Pro Glu Leu Leu Tyr Tyr
        1100 1105 1110
Ala Glu Gln Tyr Asn Glu Ile Leu Thr Gln Cys Cys Ala Glu Ala
        1115 1120 1125
Asp Lys Glu Ser Cys Leu Thr Pro Lys Leu Asp Gly Val Lys Glu
        1130 1135 1140
Lys Ala Leu Val Ser Ser Val Arg Gln Arg Met Lys Cys Ser Ser
        1145 1150 1155
Met Gln Lys Phe Gly Glu Arg Ala Phe Lys Ala Trp Ala Val Ala
        1160 1165 1170
Arg Leu Ser Gln Thr Phe Pro Asn Ala Asp Phe Ala Glu Ile Thr
        1175 1180 1185
Lys Leu Ala Thr Asp Leu Thr Lys Val Asn Lys Glu Cys Cys His
        1190 1195 1200
Gly Asp Leu Leu Glu Cys Ala Asp Asp Arg Ala Glu Leu Ala Lys
        1205 1210 1215
Tyr Met Cys Glu Asn Gln Ala Thr Ile Ser Ser Lys Leu Gln Thr
        1220 1225 1230
Cys Cys Asp Lys Pro Leu Leu Lys Lys Ala His Cys Leu Ser Glu
        1235 1340 1245
Val Glu His Asp Thr Met Pro Ala Asp Leu Pro Ala Ile Ala Ala
        1250 1255 1260
Asp Phe Val Glu Asp Gln Glu Val Cys Lys Asn Tyr Ala Glu Ala
        1265 1270 1275
Lys Asp Val Phe Leu Gly Thr Phe Leu Tyr Glu Tyr Ser Arg Arg
        1280 1285 1290
His Pro Asp Tyr Ser Val Ser Leu Leu Leu Arg Leu Ala Lys Lys
        1295 1300 1305
Tyr Glu Ala Thr Leu Glu Lys Cys Cys Ala Glu Ala Asn Pro Pro
        1310 1315 1320
Ala Cys Tyr Gly Thr Val Leu Ala Glu Phe Gln Pro Leu Val Glu
        1325 1330 1335
Glu Pro Lys Asn Leu Val Lys Thr Asn Cys Asp Leu Tyr Glu Lys
        1340 1345 1350
Leu Gly Glu Tyr Gly Phe Gln Asn Ala Ile Leu Val Arg Tyr Thr
        1355 1360 1365
Gln Lys Ala Pro Gln Val Ser Thr Pro Thr Leu Val Glu Ala Ala
        1370 1375 1380
Arg Asn Leu Gly Arg Val Gly Thr Lys Cys Cys Thr Leu Pro Glu
        1385 1390 1395
Asp Gln Arg Leu Pro Cys Val Glu Asp Tyr Leu Ser Ala Ile Leu
        1400 1405 1410
Asn Arg Val Cys Leu Leu His Glu Lys Thr Pro Val Ser Glu His
        1415 1420 1425
Val Thr Lys Cys Cys Ser Gly Ser Leu Val Glu Arg Arg Pro Cys
        1430 1435 1440
Phe Ser Ala Leu Thr Val Asp Glu Thr Tyr Val Pro Lys Glu Phe
        1445 1450 1455
Lys Ala Glu Thr Phe Thr Phe His Ser Asp Ile Cys Thr Leu Pro
        1460 1465 1470
Glu Lys Glu Lys Gln Ile Lys Lys Gln Thr Ala Leu Ala Glu Leu
        1475 1480 1485
Val Lys His Lys Pro Lys Ala Thr Ala Glu Gln Leu Lys Thr Val
        1490 1495 1500
Met Asp Asp Phe Ala Gln Phe Leu Asp Thr Cys Cys Lys Ala Ala
        1505 1510 1515
Asp Lys Asp Thr Cys Phe Ser Thr Glu Gly Pro Asn Leu Val Thr
        1520 1525 1530
Arg Cys Lys Asp Ala Leu Ala Arg Ser Trp Ser His Pro Gln Phe
        1535 1540 1545
Glu Lys
      1550
Singly underlined: signal peptide sequence; double-underlined:
beginning and end of NPP1; ** = cleavage position at the signal
peptide sequence; bold residues indicate albumin sequence
SEQ. ID NO: 30-ENPP121-NPP3-Fc sequence
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala**Lys
                                85 90 95
Gln Gly Ser Cys Arg Lys Lys Cys Phe Asp Ala Ser Phe Arg Gly Leu
                        100 105 110
Glu Asn Cys Arg Cys Asp Val Ala Cys Lys Asp Arg Gly Asp Cys Cys
                115 120 125
Trp Asp Phe Glu Asp Thr Cys Val Glu Ser Thr Arg Ile Trp Met Cys
        130 135 140
Asn Lys Phe Arg Cys Gly Glu Arg Leu Glu Ala Ser Leu Cys Ser Cys
145 150 155 160
Ser Asp Asp Cys Leu Gln Arg Lys Asp Cys Cys Ala Asp Tyr Lys Ser
                                165 170 175
Val Cys Gln Gly Glu Thr Ser Trp Leu Glu Glu Asn Cys Asp Thr Ala
                        180 185 190
Gln Gln Ser Gln Cys Pro Glu Gly Phe Asp Leu Pro Pro Val Ile Leu
                195 200 205
Phe Ser Met Asp Gly Phe Arg Ala Glu Tyr Leu Tyr Thr Trp Asp Thr
        210 215 220
Leu Met Pro Asn Ile Asn Lys Leu Lys Thr Cys Gly Ile His Ser Lys
225 230 235 240
Tyr Met Arg Ala Met Tyr Pro Thr Lys Thr Phe Pro Asn His Tyr Thr
                                245 250 255
Ile Val Thr Gly Leu Tyr Pro Glu Ser His Gly Ile Ile Asp Asn Asn
                        260 265 270
Met Tyr Asp Val Asn Leu Asn Lys Asn Phe Ser Leu Ser Ser Lys Glu
                275 280 285
Gln Asn Asn Pro Ala Trp Trp His Gly Gln Pro Met Trp Leu Thr Ala
        290 295 300
Met Tyr Gln Gly Leu Lys Ala Ala Thr Tyr Phe Trp Pro Gly Ser Glu
305 310 315 320
Val Ala Ile Asn Gly Ser Phe Pro Ser Ile Tyr Met Pro Tyr Asn Gly
                                325 330 335
Ser Val Pro Phe Glu Glu Arg Ile Ser Thr Leu Leu Lys Trp Leu Asp
                        340 345 350
Leu Pro Lys Ala Glu Arg Pro Arg Phe Tyr Thr Met Tyr Phe Glu Glu
                355 360 365
Pro Asp Ser Ser Gly His Ala Gly Gly Pro Val Ser Ala Arg Val Ile
        370 375 380
Lys Ala Leu Gln Val Val Asp His Ala Phe Gly Met Leu Met Glu Gly
385 390 395 400
Leu Lys Gln Arg Asn Leu His Asn Cys Val Asn Ile Ile Leu Leu Ala
                                405 410 415
Asp His Gly Met Asp Gln Thr Tyr Cys Asn Lys Met Glu Tyr Met Thr
                        420 425 430
Asp Tyr Phe Pro Arg Ile Asn Phe Phe Tyr Met Tyr Glu Gly Pro Ala
                435 440 445
Pro Arg Ile Arg Ala His Asn Ile Pro His Asp Phe Phe Ser Phe Asn
        450 455 460
Ser Glu Glu Ile Val Arg Asn Leu Ser Cys Arg Lys Pro Asp Gln His
465 470 475 480
Phe Lys Pro Tyr Leu Thr Pro Asp Leu Pro Lys Arg Leu His Tyr Ala
                                485 490 495
Lys Asn Val Arg Ile Asp Lys Val His Leu Phe Val Asp Gln Gln Trp
                        500 505 510
Leu Ala Val Arg Ser Lys Ser Asn Thr Asn Cys Gly Gly Gly Asn His
                515 520 525
Gly Tyr Asn Asn Glu Phe Arg Ser Met Glu Ala Ile Phe Leu Ala His
        530 535 540
Gly Pro Ser Phe Lys Glu Lys Thr Glu Val Glu Pro Phe Glu Asn Ile
545 550 555 560
Glu Val Tyr Asn Leu Met Cys Asp Leu Leu Arg Ile Gln Pro Ala Pro
                                565 570 575
Asn Asn Gly Thr His Gly Ser Leu Asn His Leu Leu Lys Val Pro Phe
                        580 585 590
Tyr Glu Pro Ser His Ala Glu Glu Val Ser Lys Phe Ser Val Cys Gly
                595 600 605
Phe Ala Asn Pro Leu Pro Thr Glu Ser Leu Asp Cys Phe Cys Pro His
        610 615 620
Leu Gln Asn Ser Thr Gln Leu Glu Gln Val Asn Gln Met Leu Asn Leu
625 630 635 640
Thr Gln Glu Glu Ile Thr Ala Thr Val Lys Val Asn Leu Pro Phe Gly
                                645 650 655
Arg Pro Arg Val Leu Gln Lys Asn Val Asp His Cys Leu Leu Tyr His
                        660 665 670
Arg Glu Tyr Val Ser Gly Phe Gly Lys Ala Met Arg Met Pro Met Trp
                675 680 685
Ser Ser Tyr Thr Val Pro Gln Leu Gly Asp Thr Ser Pro Leu Pro Pro
        690 695 700
Thr Val Pro Asp Cys Leu Arg Ala Asp Val Arg Val Pro Pro Ser Glu
705 710 715 720
Ser Gln Lys Cys Ser Phe Tyr Leu Ala Asp Lys Asn Ile Thr His Gly
                                725 730 735
Phe Leu Tyr Pro Pro Ala Ser Asn Arg Thr Ser Asp Ser Gln Tyr Asp
                        740 745 750
Ala Leu Ile Thr Ser Asn Leu Val Pro Met Tyr Glu Glu Phe Arg Lys
                755 760 765
Met Trp Asp Tyr Phe His Ser Val Leu Leu Ile Lys His Ala Thr Glu
        770 775 780
Arg Asn Gly Val Asn Val Val Ser Gly Pro Ile Phe Asp Tyr Asn Tyr
785 790 795 800
Asp Gly His Phe Asp Ala Pro Asp Glu Ile Thr Lys His Leu Ala Asn
                                805 810 815
Thr Asp Val Pro Ile Pro Thr His Tyr Phe Val Val Leu Thr Ser Cys
                        820 825 830
Lys Asn Lys Ser His Thr Pro Glu Asn Cys Pro Gly Trp Leu Asp Val
                835 840 845
Leu Pro Phe Ile Ile Pro His Arg Pro Thr Asn Val Glu Ser Cys Pro
        850 855 860
Glu Gly Lys Pro Glu Ala Leu Trp Val Glu Glu Arg Phe Thr Ala His
865 870 875 880
Ile Ala Arg Val Arg Asp Val Glu Leu Leu Thr Gly Leu Asp Phe Tyr
                                885 890 895
Gln Asp Lys Val Gln Pro Val Ser Glu Ile Leu Gln Leu Lys Thr Tyr
                        900 905 910
Leu Pro Thr Phe Glu Thr Thr Ile Asp Lys Thr His Thr Cys Pro Pro
                915 920 925
Cys Pro Ala Pro Glu Leu Leu Gly Gly Pro Ser Val Phe Leu Phe Pro
        930 935 940
Pro Lys Pro Lys Asp Thr Leu Met Ile Ser Arg Thr Pro Glu Val Thr
945 950 955 960
Cys Val Val Val Asp Val Ser His Glu Asp Pro Glu Val Lys Phe Asn
                                965 970 975
Trp Tyr Val Asp Gly Val Glu Val His Asn Ala Lys Thr Lys Pro Arg
                        980 985 990
Glu Glu Gln Tyr Asn Ser Thr Tyr Arg Val Val Ser Val Leu Thr Val
                995 1000 1005
Leu His Gln Asp Trp Leu Asn Gly Lys Glu Tyr Lys Cys Lys Val
        1010 1015 1020
Ser Asn Lys Ala Leu Pro Ala Pro Ile Glu Lys Thr Ile Ser Lys
        1025 1030 1035
Ala Lys Gly Gln Pro Arg Glu Pro Gln Val Tyr Thr Leu Pro Pro
        1040 1045 1050
Ser Arg Glu Glu Met Thr Lys Asn Gln Val Ser Leu Thr Cys Leu
        1055 1060 1065
Val Lys Gly Phe Tyr Pro Ser Asp Ile Ala Val Glu Trp Glu Ser
        1070 1075 1080
Asn Gly Gln Pro Glu Asn Asn Tyr Lys Thr Thr Pro Pro Val Leu
        1085 1090 1095
Asp Ser Asp Gly Ser Phe Phe Leu Tyr Ser Lys Leu Thr Val Asp
        1100 1105 1110
Lys Ser Arg Trp Gln Gln Gly Asn Val Phe Ser Cys Ser Val Met
        1115 1120 1125
His Glu Ala Leu His Asn His Tyr Thr Gln Lys Ser Leu Ser Leu
        1130 1135 1140
Ser Pro Gly Lys
        1145
Singly underlined: signal peptide sequence; double-underlined:
beginning and end of NPP1; ** = cleavage position at the signal
peptide sequence; bold residues indicate Fc sequence
SEQ. ID NO: 31-ENPP121-NPP3-Albumin sequence
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala**Lys
                                85 90 95
Gln Gly Ser Cys Arg Lys Lys Cys Phe Asp Ala Ser Phe Arg Gly Leu
                        100 105 110
Glu Asn Cys Arg Cys Asp Val Ala Cys Lys Asp Arg Gly Asp Cys Cys
                115 120 125
Trp Asp Phe Glu Asp Thr Cys Val Glu Ser Thr Arg Ile Trp Met Cys
        130 135 140
Asn Lys Phe Arg Cys Gly Glu Arg Leu Glu Ala Ser Leu Cys Ser Cys
145 150 155 160
Ser Asp Asp Cys Leu Gln Arg Lys Asp Cys Cys Ala Asp Tyr Lys Ser
                                165 170 175
Val Cys Gln Gly Glu Thr Ser Trp Leu Glu Glu Asn Cys Asp Thr Ala
                        180 185 190
Gln Gln Ser Gln Cys Pro Glu Gly Phe Asp Leu Pro Pro Val Ile Leu
                195 200 205
Phe Ser Met Asp Gly Phe Arg Ala Glu Tyr Leu Tyr Thr Trp Asp Thr
        210 215 220
Leu Met Pro Asn Ile Asn Lys Leu Lys Thr Cys Gly Ile His Ser Lys
225 230 235 240
Tyr Met Arg Ala Met Tyr Pro Thr Lys Thr Phe Pro Asn His Tyr Thr
                                245 250 255
Ile Val Thr Gly Leu Tyr Pro Glu Ser His Gly Ile Ile Asp Asn Asn
                        260 265 270
Met Tyr Asp Val Asn Leu Asn Lys Asn Phe Ser Leu Ser Ser Lys Glu
                275 280 285
Gln Asn Asn Pro Ala Trp Trp His Gly Gln Pro Met Trp Leu Thr Ala
        290 295 300
Met Tyr Gln Gly Leu Lys Ala Ala Thr Tyr Phe Trp Pro Gly Ser Glu
305 310 315 320
Val Ala Ile Asn Gly Ser Phe Pro Ser Ile Tyr Met Pro Tyr Asn Gly
                                325 330 335
Ser Val Pro Phe Glu Glu Arg Ile Ser Thr Leu Leu Lys Trp Leu Asp
                        340 345 350
Leu Pro Lys Ala Glu Arg Pro Arg Phe Tyr Thr Met Tyr Phe Glu Glu
                355 360 365
Pro Asp Ser Ser Gly His Ala Gly Gly Pro Val Ser Ala Arg Val Ile
        370 375 380
Lys Ala Leu Gln Val Val Asp His Ala Phe Gly Met Leu Met Glu Gly
385 390 395 400
Leu Lys Gln Arg Asn Leu His Asn Cys Val Asn Ile Ile Leu Leu Ala
                                405 410 415
Asp His Gly Met Asp Gln Thr Tyr Cys Asn Lys Met Glu Tyr Met Thr
                        420 425 430
Asp Tyr Phe Pro Arg Ile Asn Phe Phe Tyr Met Tyr Glu Gly Pro Ala
                435 440 445
Pro Arg Ile Arg Ala His Asn Ile Pro His Asp Phe Phe Ser Phe Asn
        450 455 460
Ser Glu Glu Ile Val Arg Asn Leu Ser Cys Arg Lys Pro Asp Gln His
465 470 475 480
Phe Lys Pro Tyr Leu Thr Pro Asp Leu Pro Lys Arg Leu His Tyr Ala
                                485 490 495
Lys Asn Val Arg Ile Asp Lys Val His Leu Phe Val Asp Gln Gln Trp
                        500 505 510
Leu Ala Val Arg Ser Lys Ser Asn Thr Asn Cys Gly Gly Gly Asn His
                515 520 525
Gly Tyr Asn Asn Glu Phe Arg Ser Met Glu Ala Ile Phe Leu Ala His
        530 535 540
Gly Pro Ser Phe Lys Glu Lys Thr Glu Val Glu Pro Phe Glu Asn Ile
545 550 555 560
Glu Val Tyr Asn Leu Met Cys Asp Leu Leu Arg Ile Gln Pro Ala Pro
                                565 570 575
Asn Asn Gly Thr His Gly Ser Leu Asn His Leu Leu Lys Val Pro Phe
                        580 585 590
Tyr Glu Pro Ser His Ala Glu Glu Val Ser Lys Phe Ser Val Cys Gly
                595 600 605
Phe Ala Asn Pro Leu Pro Thr Glu Ser Leu Asp Cys Phe Cys Pro His
        610 615 620
Leu Gln Asn Ser Thr Gln Leu Glu Gln Val Asn Gln Met Leu Asn Leu
625 630 635 640
Thr Gln Glu Glu Ile Thr Ala Thr Val Lys Val Asn Leu Pro Phe Gly
                                645 650 655
Arg Pro Arg Val Leu Gln Lys Asn Val Asp His Cys Leu Leu Tyr His
                        660 665 670
Arg Glu Tyr Val Ser Gly Phe Gly Lys Ala Met Arg Met Pro Met Trp
                675 680 685
Ser Ser Tyr Thr Val Pro Gln Leu Gly Asp Thr Ser Pro Leu Pro Pro
        690 695 700
Thr Val Pro Asp Cys Leu Arg Ala Asp Val Arg Val Pro Pro Ser Glu
705 710 715 720
Ser Gln Lys Cys Ser Phe Tyr Leu Ala Asp Lys Asn Ile Thr His Gly
                                725 730 735
Phe Leu Tyr Pro Pro Ala Ser Asn Arg Thr Ser Asp Ser Gln Tyr Asp
                        740 745 750
Ala Leu Ile Thr Ser Asn Leu Val Pro Met Tyr Glu Glu Phe Arg Lys
                755 760 765
Met Trp Asp Tyr Phe His Ser Val Leu Leu Ile Lys His Ala Thr Glu
        770 775 780
Arg Asn Gly Val Asn Val Val Ser Gly Pro Ile Phe Asp Tyr Asn Tyr
785 790 795 800
Asp Gly His Phe Asp Ala Pro Asp Glu Ile Thr Lys His Leu Ala Asn
                                805 810 815
Thr Asp Val Pro Ile Pro Thr His Tyr Phe Val Val Leu Thr Ser Cys
                        820 825 830
Lys Asn Lys Ser His Thr Pro Glu Asn Cys Pro Gly Trp Leu Asp Val
                835 840 845
Leu Pro Phe Ile Ile Pro His Arg Pro Thr Asn Val Glu Ser Cys Pro
        850 855 860
Glu Gly Lys Pro Glu Ala Leu Trp Val Glu Glu Arg Phe Thr Ala His
865 870 875 880
Ile Ala Arg Val Arg Asp Val Glu Leu Leu Thr Gly Leu Asp Phe Tyr
                                885 890 895
Gln Asp Lys Val Gln Pro Val Ser Glu Ile Leu Gln Leu Lys Thr Tyr
                        900 905 910
Leu Pro Thr Phe Glu Thr Thr Ile Gly Gly Gly Ser Gly Gly Gly Gly
                915 920 925
Ser Gly Gly Gly Gly Ser Met Lys Trp Val Thr Phe Leu Leu Leu Leu
        930 935 940
Phe Val Ser Gly Ser Ala Phe Ser Arg Gly Val Phe Arg Arg Glu Ala
945 950 955 960
His Lys Ser Glu Ile Ala His Arg Tyr Asn Asp Leu Gly Glu Gln His
                                965 970 975
Phe Lys Gly Leu Val Leu Ile Ala Phe Ser Gln Tyr Leu Gln Lys Cys
                        980 985 990
Ser Tyr Asp Glu His Ala Lys Leu Val Gln Glu Val Thr Asp Phe Ala
                995 1000 1005
Lys Thr Cys Val Ala Asp Glu Ser Ala Ala Asn Cys Asp Lys Ser
        1010 1015 1020
Leu His Thr Leu Phe Gly Asp Lys Leu Cys Ala Ile Pro Asn Leu
        1025 1030 1035
Arg Glu Asn Tyr Gly Glu Leu Ala Asp Cys Cys Thr Lys Gln Glu
        1040 1045 1050
Pro Glu Arg Asn Glu Cys Phe Leu Gln His Lys Asp Asp Asn Pro
        1055 1060 1065
Ser Leu Pro Pro Phe Glu Arg Pro Glu Ala Glu Ala Met Cys Thr
        1070 1075 1080
Ser Phe Lys Glu Asn Pro Thr Thr Phe Met Gly His Tyr Leu His
        1085 1090 1095
Glu Val Ala Arg Arg His Pro Tyr Phe Tyr Ala Pro Glu Leu Leu
        1100 1105 1110
Tyr Tyr Ala Glu Gln Tyr Asn Glu Ile Leu Thr Gln Cys Cys Ala
        1115 1120 1125
Glu Ala Asp Lys Glu Ser Cys Leu Thr Pro Lys Leu Asp Gly Val
        1130 1135 1140
Lys Glu Lys Ala Leu Val Ser Ser Val Arg Gln Arg Met Lys Cys
        1145 1150 1155
Ser Ser Met Gln Lys Phe Gly Glu Arg Ala Phe Lys Ala Trp Ala
        1160 1165 1170
Val Ala Arg Leu Ser Gln Thr Phe Pro Asn Ala Asp Phe Ala Glu
        1175 1180 1185
Ile Thr Lys Leu Ala Thr Asp Leu Thr Lys Val Asn Lys Glu Cys
        1190 1195 1200
Cys His Gly Asp Leu Leu Glu Cys Ala Asp Asp Arg Ala Glu Leu
        1205 1210 1215
Ala Lys Tyr Met Cys Glu Asn Gln Ala Thr Ile Ser Ser Lys Leu
        1220 1225 1230
Gln Thr Cys Cys Asp Lys Pro Leu Leu Lys Lys Ala His Cys Leu
        1235 1340 1245
Ser Glu Val Glu His Asp Thr Met Pro Ala Asp Leu Pro Ala Ile
        1250 1255 1260
Ala Ala Asp Phe Val Glu Asp Gln Glu Val Cys Lys Asn Tyr Ala
        1265 1270 1275
Glu Ala Lys Asp Val Phe Leu Gly Thr Phe Leu Tyr Glu Tyr Ser
        1280 1285 1290
Arg Arg His Pro Asp Tyr Ser Val Ser Leu Leu Leu Arg Leu Ala
        1295 1300 1305
Lys Lys Tyr Glu Ala Thr Leu Glu Lys Cys Cys Ala Glu Ala Asn
        1310 1315 1320
Pro Pro Ala Cys Tyr Gly Thr Val Leu Ala Glu Phe Gln Pro Leu
        1325 1330 1335
Val Glu Glu Pro Lys Asn Leu Val Lys Thr Asn Cys Asp Leu Tyr
        1340 1345 1350
Glu Lys Leu Gly Glu Tyr Gly Phe Gln Asn Ala Ile Leu Val Arg
        1355 1360 1365
Tyr Thr Gln Lys Ala Pro Gln Val Ser Thr Pro Thr Leu Val Glu
        1370 1375 1380
Ala Ala Arg Asn Leu Gly Arg Val Gly Thr Lys Cys Cys Thr Leu
        1385 1390 1395
Pro Glu Asp Gln Arg Leu Pro Cys Val Glu Asp Tyr Leu Ser Ala
        1400 1405 1410
Ile Leu Asn Arg Val Cys Leu Leu His Glu Lys Thr Pro Val Ser
        1415 1420 1425
Glu His Val Thr Lys Cys Cys Ser Gly Ser Leu Val Glu Arg Arg
        1430 1435 1440
Pro Cys Phe Ser Ala Leu Thr Val Asp Glu Thr Tyr Val Pro Lys
        1445 1450 1455
Glu Phe Lys Ala Glu Thr Phe Thr Phe His Ser Asp Ile Cys Thr
        1460 1465 1470
Leu Pro Glu Lys Glu Lys Gln Ile Lys Lys Gln Thr Ala Leu Ala
        1475 1480 1485
Glu Leu Val Lys His Lys Pro Lys Ala Thr Ala Glu Gln Leu Lys
        1490 1495 1500
Thr Val Met Asp Asp Phe Ala Gln Phe Leu Asp Thr Cys Cys Lys
        1505 1510 1515
Ala Ala Asp Lys Asp Thr Cys Phe Ser Thr Glu Gly Pro Asn Leu
        1520 1525 1530
Val Thr Arg Cys Lys Asp Ala Leu Ala
        1535 1540
Singly underlined: signal peptide sequence; double-underlined:
beginning and end of NPP3; ** = cleavage position at the signal
peptide sequence; bold residues indicate albumin sequence
SEQ. ID NO: 32-ENPP121GLK Protein Export Signal Sequence
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala Gly
                                85 90 95
Leu Lys
SEQ. ID NO: 33-Albumin Sequence
Gly Gly Gly Gly Ser Gly Gly Gly Gly Ser Gly Gly Gly Gly Ser Met
1 5 10 15
Lys Trp Val Thr Phe Leu Leu Leu Leu Phe Val Ser Gly Ser Ala Phe
                        20 25 30
Ser Arg Gly Val Phe Arg Arg Glu Ala His Lys Ser Glu Ile Ala His
                35 40 45
Arg Tyr Asn Asp Leu Gly Glu Gln His Phe Lys Gly Leu Val Leu Ile
        50 55 60
Ala Phe Ser Gln Tyr Leu Gln Lys Cys Ser Tyr Asp Glu His Ala Lys
65 70 75 80
Leu Val Gln Glu Val Thr Asp Phe Ala Lys Thr Cys Val Ala Asp Glu
                                85 90 95
Ser Ala Ala Asn Cys Asp Lys Ser Leu His Thr Leu Phe Gly Asp Lys
                        100 105 110
Leu Cys Ala Ile Pro Asn Leu Arg Glu Asn Tyr Gly Glu Leu Ala Asp
                115 120 125
Cys Cys Thr Lys Gln Glu Pro Glu Arg Asn Glu Cys Phe Leu Gln His
        130 135 140
Lys Asp Asp Asn Pro Ser Leu Pro Pro Phe Glu Arg Pro Glu Ala Glu
145 150 155 160
Ala Met Cys Thr Ser Phe Lys Glu Asn Pro Thr Thr Phe Met Gly His
                                165 170 175
Tyr Leu His Glu Val Ala Arg Arg His Pro Tyr Phe Tyr Ala Pro Glu
                        180 185 190
Leu Leu Tyr Tyr Ala Glu Gln Tyr Asn Glu Ile Leu Thr Gln Cys Cys
                195 200 205
Ala Glu Ala Asp Lys Glu Ser Cys Leu Thr Pro Lys Leu Asp Gly Val
        210 215 220
Lys Glu Lys Ala Leu Val Ser Ser Val Arg Gln Arg Met Lys Cys Ser
225 230 235 240
Ser Met Gln Lys Phe Gly Glu Arg Ala Phe Lys Ala Trp Ala Val Ala
                                245 250 255
Arg Leu Ser Gln Thr Phe Pro Asn Ala Asp Phe Ala Glu Ile Thr Lys
                        260 265 270
Leu Ala Thr Asp Leu Thr Lys Val Asn Lys Glu Cys Cys His Gly Asp
                275 280 285
Leu Leu Glu Cys Ala Asp Asp Arg Ala Glu Leu Ala Lys Tyr Met Cys
        290 295 300
Glu Asn Gln Ala Thr Ile Ser Ser Lys Leu Gln Thr Cys Cys Asp Lys
305 310 315 320
Pro Leu Leu Lys Lys Ala His Cys Leu Ser Glu Val Glu His Asp Thr
                                325 330 335
Met Pro Ala Asp Leu Pro Ala Ile Ala Ala Asp Phe Val Glu Asp Gln
                        340 345 350
Glu Val Cys Lys Asn Tyr Ala Glu Ala Lys Asp Val Phe Leu Gly Thr
                355 360 365
Phe Leu Tyr Glu Tyr Ser Arg Arg His Pro Asp Tyr Ser Val Ser Leu
        370 375 380
Leu Leu Arg Leu Ala Lys Lys Tyr Glu Ala Thr Leu Glu Lys Cys Cys
385 390 395 400
Ala Glu Ala Asn Pro Pro Ala Cys Tyr Gly Thr Val Leu Ala Glu Phe
                                405 410 415
Gln Pro Leu Val Glu Glu Pro Lys Asn Leu Val Lys Thr Asn Cys Asp
                        420 425 430
Leu Tyr Glu Lys Leu Gly Glu Tyr Gly Phe Gln Asn Ala Ile Leu Val
                435 440 445
Arg Tyr Thr Gln Lys Ala Pro Gln Val Ser Thr Pro Thr Leu Val Glu
        450 455 460
Ala Ala Arg Asn Leu Gly Arg Val Gly Thr Lys Cys Cys Thr Leu Pro
465 470 475 480
Glu Asp Gln Arg Leu Pro Cys Val Glu Asp Tyr Leu Ser Ala Ile Leu
                                485 490 495
Asn Arg Val Cys Leu Leu His Glu Lys Thr Pro Val Ser Glu His Val
                        500 505 510
Thr Lys Cys Cys Ser Gly Ser Leu Val Glu Arg Arg Pro Cys Phe Ser
                515 520 525
Ala Leu Thr Val Asp Glu Thr Tyr Val Pro Lys Glu Phe Lys Ala Glu
        530 535 540
Thr Phe Thr Phe His Ser Asp Ile Cys Thr Leu Pro Glu Lys Glu Lys
545 550 555 560
Gln Ile Lys Lys Gln Thr Ala Leu Ala Glu Leu Val Lys His Lys Pro
                                565 570 575
Lys Ala Thr Ala Glu Gln Leu Lys Thr Val Met Asp Asp Phe Ala Gln
                        580 585 590
Phe Leu Asp Thr Cys Cys Lys Ala Ala Asp Lys Asp Thr Cys Phe Ser
                595 600 605
Thr Glu Gly Pro Asn Leu Val Thr Arg Cys Lys Asp Ala Leu Ala
        610 615 620
SEQ. ID NO: 34-Human IgG Fc domain, Fc
Asp Lys Thr His Thr Cys Pro Pro Cys Pro Ala Pro Glu Leu Leu Gly
1 5 10 15
Gly Pro Ser Val Phe Leu Phe Pro Pro Lys Pro Lys Asp Thr Leu Met
                        20 25 30
Ile Ser Arg Thr Pro Glu Val Thr Cys Val Val Val Asp Val Ser His
                35 40 45
Glu Asp Pro Glu Val Lys Phe Asn Trp Tyr Val Asp Gly Val Glu Val
        50 55 60
His Asn Ala Lys Thr Lys Pro Arg Glu Glu Gln Tyr Asn Ser Thr Tyr
65 70 75 80
Arg Val Val Ser Val Leu Thr Val Leu His Gln Asp Trp Leu Asn Gly
                                85 90 95
Lys Glu Tyr Lys Cys Lys Val Ser Asn Lys Ala Leu Pro Ala Pro Ile
                        100 105 110
Glu Lys Thr Ile Ser Lys Ala Lys Gly Gln Pro Arg Glu Pro Gln Val
                115 120 125
Tyr Thr Leu Pro Pro Ser Arg Glu Glu Met Thr Lys Asn Gln Val Ser
        130 135 140
Leu Thr Cys Leu Val Lys Gly Phe Tyr Pro Ser Asp Ile Ala Val Glu
145 150 155 160
Trp Glu Ser Asn Gly Gln Pro Glu Asn Asn Tyr Lys Thr Thr Pro Pro
                                165 170 175
Val Leu Asp Ser Asp Gly Ser Phe Phe Leu Tyr Ser Lys Leu Thr Val
                        180 185 190
Asp Lys Ser Arg Trp Gln Gln Gly Asn Val Phe Ser Cys Ser Val Met
                195 200 205
His Glu Ala Leu His Asn His Tyr Thr Gln Lys Ser Leu Ser Leu Ser
        210 215 220
Pro Gly Lys
225
SEQ. ID NO: 35-Albumin Sequence
Met Lys Trp Val Thr Phe Leu Leu Leu Leu Phe Val Ser Gly Ser Ala
1 5 10 15
Phe Ser Arg Gly Val Phe Arg Arg Glu Ala His Lys Ser Glu Ile Ala
                        20 25 30
His Arg Tyr Asn Asp Leu Gly Glu Gln His Phe Lys Gly Leu Val Leu
                35 40 45
Ile Ala Phe Ser Gln Tyr Leu Gln Lys Cys Ser Tyr Asp Glu His Ala
        50 55 60
Lys Leu Val Gln Glu Val Thr Asp Phe Ala Lys Thr Cys Val Ala Asp
65 70 75 80
Glu Ser Ala Ala Asn Cys Asp Lys Ser Leu His Thr Leu Phe Gly Asp
                                85 90 95
Lys Leu Cys Ala Ile Pro Asn Leu Arg Glu Asn Tyr Gly Glu Leu Ala
                        100 105 110
Asp Cys Cys Thr Lys Gln Glu Pro Glu Arg Asn Glu Cys Phe Leu Gln
                115 120 125
His Lys Asp Asp Asn Pro Ser Leu Pro Pro Phe Glu Arg Pro Glu Ala
        130 135 140
Glu Ala Met Cys Thr Ser Phe Lys Glu Asn Pro Thr Thr Phe Met Gly
145 150 155 160
His Tyr Leu His Glu Val Ala Arg Arg His Pro Tyr Phe Tyr Ala Pro
                                165 170 175
Glu Leu Leu Tyr Tyr Ala Glu Gln Tyr Asn Glu Ile Leu Thr Gln Cys
                        180 185 190
Cys Ala Glu Ala Asp Lys Glu Ser Cys Leu Thr Pro Lys Leu Asp Gly
                195 200 205
Val Lys Glu Lys Ala Leu Val Ser Ser Val Arg Gln Arg Met Lys Cys
        210 215 220
Ser Ser Met Gln Lys Phe Gly Glu Arg Ala Phe Lys Ala Trp Ala Val
225 230 235 240
Ala Arg Leu Ser Gln Thr Phe Pro Asn Ala Asp Phe Ala Glu Ile Thr
                                245 250 255
Lys Leu Ala Thr Asp Leu Thr Lys Val Asn Lys Glu Cys Cys His Gly
                        260 265 270
Asp Leu Leu Glu Cys Ala Asp Asp Arg Ala Glu Leu Ala Lys Tyr Met
                275 280 285
Cys Glu Asn Gln Ala Thr Ile Ser Ser Lys Leu Gln Thr Cys Cys Asp
        290 295 300
Lys Pro Leu Leu Lys Lys Ala His Cys Leu Ser Glu Val Glu His Asp
305 310 315 320
Thr Met Pro Ala Asp Leu Pro Ala Ile Ala Ala Asp Phe Val Glu Asp
                                325 330 335
Gln Glu Val Cys Lys Asn Tyr Ala Glu Ala Lys Asp Val Phe Leu Gly
                        340 345 350
Thr Phe Leu Tyr Glu Tyr Ser Arg Arg His Pro Asp Tyr Ser Val Ser
                355 360 365
Leu Leu Leu Arg Leu Ala Lys Lys Tyr Glu Ala Thr Leu Glu Lys Cys
        370 375 380
Cys Ala Glu Ala Asn Pro Pro Ala Cys Tyr Gly Thr Val Leu Ala Glu
385 390 395 400
Phe Gln Pro Leu Val Glu Glu Pro Lys Asn Leu Val Lys Thr Asn Cys
                                405 410 415
Asp Leu Tyr Glu Lys Leu Gly Glu Tyr Gly Phe Gln Asn Ala Ile Leu
                        420 425 430
Val Arg Tyr Thr Gln Lys Ala Pro Gln Val Ser Thr Pro Thr Leu Val
                435 440 445
Glu Ala Ala Arg Asn Leu Gly Arg Val Gly Thr Lys Cys Cys Thr Leu
        450 455 460
Pro Glu Asp Gln Arg Leu Pro Cys Val Glu Asp Tyr Leu Ser Ala Ile
465 470 475 480
Leu Asn Arg Val Cys Leu Leu His Glu Lys Thr Pro Val Ser Glu His
                                485 490 495
Val Thr Lys Cys Cys Ser Gly Ser Leu Val Glu Arg Arg Pro Cys Phe
                        500 505 510
Ser Ala Leu Thr Val Asp Glu Thr Tyr Val Pro Lys Glu Phe Lys Ala
                515 520 525
Glu Thr Phe Thr Phe His Ser Asp Ile Cys Thr Leu Pro Glu Lys Glu
        530 535 540
Lys Gln Ile Lys Lys Gln Thr Ala Leu Ala Glu Leu Val Lys His Lys
545 550 555 560
Pro Lys Ala Thr Ala Glu Gln Leu Lys Thr Val Met Asp Asp Phe Ala
                                565 570 575
Gln Phe Leu Asp Thr Cys Cys Lys Ala Ala Asp Lys Asp Thr Cys Phe
                        580 585 590
Ser Thr Glu Gly Pro Asn Leu Val Thr Arg Cys Lys Asp Ala Leu Ala
                595 600 605
Arg Ser Trp Ser His Pro Gln Phe Glu Lys
        610 615
SEQ. ID NO: 36-ENPP2 Signal Peptide
Leu Phe Thr Phe Ala Val Gly Val Asn Ile Cys Leu Gly
1 5 10 15
Phe Thr Ala
SEQ. ID NO: 37-Signal Sequence ENPP7
Met Arg Gly Pro Ala Val Leu Leu Thr Val Ala Leu Ala Thr Leu Leu
1 5 10 15
Ala Pro Gly Ala
                        20
SEQ. ID NO: 38-Signal sequence ENPP7
Met Arg Gly Pro Ala Val Leu Leu Thr Val Ala Leu Ala Thr Leu Leu
1 5 10 15
Ala Pro Gly Ala Gly Ala
                        20
SEQ. ID NO: 39-Signal Sequence ENPP1-2-1
Met Glu Arg Asp Gly Cys Ala Gly Gly Gly Ser Arg Gly Gly Glu Gly
1 5 10 15
Gly Arg Ala Pro Arg Glu Gly Pro Ala Gly Asn Gly Arg Asp Arg Gly
                        20 25 30
Arg Ser His Ala Ala Glu Ala Pro Gly Asp Pro Gln Ala Ala Ala Ser
                35 40 45
Leu Leu Ala Pro Met Asp Val Gly Glu Glu Pro Leu Glu Lys Ala Ala
        50 55 60
Arg Ala Arg Thr Ala Lys Asp Pro Asn Thr Tyr Lys Ile Ile Ser Leu
65 70 75 80
Phe Thr Phe Ala Val Gly Val Asn Ile Cys Leu Gly Phe Thr Ala
                                85 90 95
SEQ. ID NO: 40-exENPP3
Leu Leu Val Ile Met Ser Leu Gly Leu Gly Leu Gly Leu Gly Leu Arg
1 5 10 15
Lys
SEQ. ID NO: 41-Signal Sequence ENPP5 :
Met Thr Ser Lys Phe Leu Leu Val Ser Phe Ile Leu Ala Ala Leu Ser
1 5 10 15
Leu Ser Thr Thr Phe Ser
                        20
SEQ ID NO: 42-Signal Sequence-Azurocidin
Met Thr Arg Leu Thr Val Leu Ala Leu Leu Ala Gly Leu Leu Ala Ser
Ser Arg Ala
SEQ. ID NO: 43-Linker
Asp Ser Ser
SEQ. ID NO: 44-Linker
Glu Ser Ser
SEQ. ID NO: 45-Linker
Arg Gln Gln
SEQ. ID NO: 46-Linker
Lys Arg
SEQ. ID NO: 47-Linker
(Arg)ₘ ; m = 0 − 15
SEQ. ID NO: 48-Linker
Asp Ser Ser Ser Glu Glu Lys Phe Leu Arg Arg Ile Gly Arg Phe Gly
SEQ. ID NO: 49-Linker
Glu Glu Glu Glu Glu Glu Glu Pro Arg Gly Asp Thr
1 5 10
SEQ. ID NO: 50-Linker
Ala Pro Trp His Leu Ser Ser Gln Tyr Ser Arg Thr
1 5 10
SEQ. ID NO: 51-Linker
Ser Thr Leu Pro Ile Pro His Glu Phe Ser Arg Glu
1 5 10
SEQ. ID NO: 52-Linker
Val Thr Lys His Leu Asn Gln Ile Ser Gln Ser Tyr
1 5 10
SEQ. ID NO: 53-Linker
(Glu)ₘ; m = 1 − 15
SEQ. ID NO: 54-Linker
Leu Ile Asn
SEQ. ID NO: 55-Linker
Gly Gly Ser Gly Gly Ser
1 5
SEQ. ID NO: 56-Linker
Arg Ser Gly Ser Gly Gly Ser
1 5
SEQ. ID NO: 57-Linker
(Asp)ₘ; m = 1 − 15
1
SEQ. ID NO: 58-Linker
Leu Val Ile Met Ser Leu Gly Leu Gly Leu Gly Leu Gly Leu Arg Lys
1 5 10 15
SEQ. ID NO: 59-Linker
Val Ile Met Ser Leu Gly Leu Gly Leu Gly Leu Gly Leu Arg Lys
1 5 10 15
SEQ. ID NO: 60-Linker
Ile Met Ser Leu Gly Leu Gly Leu Gly Leu Gly Leu Arg Lys
1 5 10
SEQ. ID NO: 61-Linker
Met Ser Leu Gly Leu Gly Leu Gly Leu Gly Leu Arg Lys
1 5 10
SEQ. ID NO: 62-Linker
Ser Leu Gly Leu Gly Leu Gly Leu Gly Leu Arg Lys
1 5 10
SEQ. ID NO: 63-Linker
Leu Gly Leu Gly Leu Gly Leu Gly Leu Arg Lys
1 5 10
SEQ. ID NO: 64-Linker
Gly Leu Gly Leu Gly Leu Gly Leu Arg Lys
1 5 10
SEQ. ID NO: 65-Linker
Leu Gly Leu Gly Leu Gly Leu Arg Lys
1 5
SEQ. ID NO: 66-Linker
Gly Leu Gly Leu Gly Leu Arg Lys
1 5
SEQ. ID NO: 67-Linker
Leu Gly Leu Gly Leu Arg Lys
1 5
SEQ. ID NO: 68-Linker
Gly Leu Gly Leu Arg Lys
1 5
SEQ. ID NO: 69-Linker
Leu Gly Leu Arg Lys
1 5
SEQ. ID NO: 70-Linker
Gly Leu Arg Lys
1
SEQ. ID NO: 71-Linker
Leu Arg Lys
1
SEQ. ID NO: 72-Linker
Arg Lys
1
SEQ. ID NO: 73-Linker
(Lys)ₘ; m = 0 − 15
1
SEQ. ID NO: 74 -Linker
Dₘ; m = 1 − 15
SEQ. ID NO: 75 -Linker
(GGGGS)ₙ; n = 1 − 10
SEQ. ID NO: 76-ENPP3 Nucleotide sequence
```

| | |
|---|---|
| atggaatcta cgttgacttt agcaacggaa caacctgtta agaagaacac tcttaagaaa | 60 |
| tataaaatag cttgcattgt tcttcttgct ttgctggtga tcatgtcact tggattaggc | 120 |
| ctggggcttg gactcaggaa actggaaaag caaggcagct gcaggaagaa gtgctttgat | 180 |
| gcatcattta gaggactgga gaactgccgg tgtgatgtgg catgtaaaga ccgaggtgat | 240 |
| tgctgctggg attttgaaga cacctgtgtg gaatcaactc gaatatggat gtgcaataaa | 300 |
| tttcgttgtg gagagaccag attagaggcc agcctttgct cttgttcaga tgactgtttg | 360 |
| cagaggaaag attgctgtgc tgactataag agtgtttgcc aaggagaaac ctcatggctg | 420 |
| gaagaaaact gtgacacagc ccagcagtct cagtgcccag aagggtttga cctgccacca | 480 |
| gttatcttgt tttctatgga tggatttaga gctgaatatt tatacacatg ggatacttta | 540 |
| atgccaaata tcaataaact gaaaacatgt ggaattcatt caaaatacat gagagctatg | 600 |
| tatcctacca aaaccttccc aaatcattac accattgtca cgggcttgta tccagagtca | 660 |
| catggcatca ttgacaataa tatgtatgat gtaaatctca acaagaattt ttcactttct | 720 |
| tcaaaggaac aaaataatcc agcctggtgg catgggcaac caatgtggct gacagcaatg | 780 |
| tatcaaggtt taaaagccgc tacctacttt tggcccggat cagaagtggc tataaatggc | 840 |
| tcctttcctt ccatatacat gccttacaac ggaagtgtcc catttgaaga gaggatttct | 900 |
| acactgttaa aatggctgga cctgcccaaa gctgaaagac ccaggtttta taccatgtat | 960 |
| tttgaagaac ctgattcctc tggacatgca ggtggaccag tcagtgccag agtaattaaa | 1020 |
| gccttacagg tagtagatca tgcttttggg atgttgatgg aaggcctgaa gcagcggaat | 1080 |
| ttgcacaact gtgtcaatat catccttctg gctgaccatg gaatggacca gacttattgt | 1140 |
| aacaagatgg aatacatgac tgattatttt cccagaataa acttcttcta catgtacgaa | 1200 |
| gggcctgccc cccgcatccg agctcataat atacctcatg acttttttag ttttaattct | 1260 |
| gaggaaattg ttagaaacct cagttgccga aaacctgatc agcatttcaa gccctatttg | 1320 |
| actcctgatt tgccaaagcg actgcactat gccaagaacg tcagaatcga caaagttcat | 1380 |
| ctctttgtgg atcaacagtg gctggctgtt aggagtaaat caaatacaaa ttgtggagga | 1440 |
| ggcaaccatg gttataacaa tgagtttagg agcatggagg ctatctttct ggcacatgga | 1500 |
| cccagtttta aagagaagac tgaagttgaa ccatttgaaa atattgaagt ctataaccta | 1560 |
| atgtgtgatc ttctacgcat tcaaccagca ccaaacaatg gaacccatgg tagtttaaac | 1620 |
| catcttctga aggtgccttt ttatgagcca tcccatgcag aggaggtgtc aaagttttct | 1680 |
| gtttgtggct ttgctaatcc attgcccaca gagtctcttg actgtttctg ccctcaccta | 1740 |
| caaaatagta ctcagctgga acaagtgaat cagatgctaa atctcaccca agaagaaata | 1800 |
| acagcaacag tgaaagtaaa tttgccattt gggaggccta gggtactgca gaagaacgtg | 1860 |
| gaccactgtc tcctttacca cagggaatat gtcagtggat ttggaaaagc tatgaggatg | 1920 |
| cccatgtgga gttcatacac agtcccccag ttgggagaca catcgcctct gcctcccact | 1980 |
| gtcccagact gtctgcgggc tgatgtcagg gttcctcctt ctgagagcca aaaatgttcc | 2040 |
| ttctatttag cagacaagaa tatcacccac ggcttcctct atcctcctgc cagcaataga | 2100 |
| acatcagata gccaatatga tgctttaatt actagcaatt tggtacctat gtatgaagaa | 2160 |
| ttcagaaaaa tgtgggacta cttccacagt gttcttctta taaaacatgc cacagaaaga | 2220 |
| aatggagtaa atgtggttag tggaccaata tttgattata attatgatgg ccattttgat | 2280 |
| gctccagatg aaattaccaa acatttagcc aacactgatg ttcccatccc aacacactac | 2340 |
| tttgtggtgc tgaccagttg taaaaacaag agccacacac cggaaaactg ccctgggtgg | 2400 |
| ctggatgtcc taccctttat catccctcac cgacctacca acgtggagag ctgtcctgaa | 2460 |
| ggtaaaccag aagctctttg ggttgaagaa agatttacag ctcacattgc ccgggtccgt | 2520 |
| gatgtagaac ttctcactgg gcttgacttc tatcaggata aagtgcagcc tgtctctgaa | 2580 |
| attttgcaac taaagacata tttaccaaca tttgaaacca ctatt | 2625 |

SEQ. ID NO: 77-ENPP1 Nucleotide sequence:

| | |
|---|---|
| atggaacggg acggctgtgc cggcggagga tcaagaggcg gagaaggcgg cagagcccct | 60 |
| agagaaggac ctgccggcaa cggcagagac agaggcagat ctcatgccgc cgaagcccct | 120 |
| ggcgatcctc aggctgctgc ttctctgctg gcccccatgg atgtgggcga ggaacctctg | 180 |
| gaaaaggccg ccagagccag aaccgccaag gaccccaaca cctacaaggt gctgagcctg | 240 |
| gtgctgtccg tgtgcgtgct gaccaccatc ctgggctgca tcttcggcct gaagcccagc | 300 |
| tgcgccaaag aagtgaagtc ctgcaagggc cggtgcttcg agcggacctt cggcaactgc | 360 |
| agatgcgacg ccgcctgtgt ggaactgggc aactgctgcc tggactacca ggaaacctgc | 420 |
| atcgagcccg agcacatctg gacctgcaac aagttcagat gcggcgagaa gcggctgacc | 480 |
| agatccctgt gtgcctgcag cgacgactgc aaggacaagg gcgactgctg catcaactac | 540 |
| agcagcgtgt gccagggcga gaagtcctgg gtggaagaac cctgcgagag catcaacgag | 600 |
| ccccagtgcc ctgccggctt cgagacacct cctaccctgc tgttcagcct ggacggcttt | 660 |
| cgggccgagt acctgcacac atggggaggc ctgctgcccg tgatcagcaa gctgaagaag | 720 |
| tgcggcacct acaccaagaa catgcggccc gtgtacccca ccaagacctt ccccaaccac | 780 |
| tactccatcg tgaccggcct gtaccccgag agccacggca tcatcgacaa caagatgtac | 840 |
| gaccccaaga tgaacgccag cttcagcctg aagtccaaag agaagttcaa ccccgagtgg | 900 |
| tataagggcg agcccatctg ggtcaccgcc aagtaccagg gcctgaaaag cggcacattc | 960 |
| ttttggcccg gcagcgacgt ggaaatcaac ggcatcttcc ccgacatcta taagatgtac | 1020 |
| aacggcagcg tgcccttcga ggaacggatc ctggctgtgc tgcagtggct gcagctgccc | 1080 |
| aaggatgagc ggccccactt ctacaccctg tacctggaag aacctgacag cagcggccac | 1140 |
| agctacggcc ctgtgtccag cgaagtgatc aaggccctgc agcgggtgga cggcatggtg | 1200 |
| ggaatgctga tggacggcct gaaagagctg aacctgcaca gatgcctgaa cctgatcctg | 1260 |
| atcagcgacc acggcatgga acagggatcc tgcaagaagt acatctacct gaacaagtac | 1320 |
| ctgggcgacg tgaagaacat caaagtgatc tacggcccag ccgccagact gaggcctagc | 1380 |
| gacgtgcccg acaagtacta cagcttcaac tacgagggaa tcgcccggaa cctgagctgc | 1440 |
| agagagccca accagcactt caagccctac ctgaagcact tcctgcccaa gcggctgcac | 1500 |
| ttcgccaaga gcgacagaat cgagcccctg accttctacc tggaccccca gtggcagctg | 1560 |
| gccctgaatc ccagcgagag aaagtactgc ggcagcggct tccacggctc cgacaacgtg | 1620 |
| ttcagcaaca tgcaggccct gttcgtgggc tacggacccg gctttaagca cggcatcgag | 1680 |
| gccgacacct tcgagaacat cgaggtgtac aatctgatgt gcgacctgct gaatctgacc | 1740 |
| cctgccccca acaatggcac ccacggcagc ctgaaccatc tgctgaagaa ccccgtgtac | 1800 |
| acccctaagc accccaaaga ggtgcacccc ctggtgcagt gccccttcac cagaaacccc | 1860 |
| agagacaacc tgggctgtag ctgcaacccc agcatcctgc ccatcgagga cttccagacc | 1920 |
| cagttcaacc tgaccgtggc cgaggaaaag atcatcaagc acgagacact gccctacggc | 1980 |
| agaccccggg tgctgcagaa agagaacacc atctgcctgc tgagccagca ccagttcatg | 2040 |
| agcggctact cccaggacat cctgatgccc ctgtggacca gctacaccgt ggaccggaac | 2100 |
| gacagcttct ccaccgagga tttcagcaac tgcctgtacc aggatttccg gatccccctg | 2160 |
| agccccgtgc acaagtgcag cttctacaag aacaacacca aggtgtccta cggcttcctg | 2220 |
| agccctcccc agctgaacaa gaacagctcc ggcatctaca gcgaggccct gctgactacc | 2280 |
| aacatcgtgc ccatgtacca gagcttccaa gtgatctggc ggtacttcca cgacaccctg | 2340 |
| ctgcggaagt acgccgaaga acggaacggc gtgaacgtgg tgtccggccc agtgttcgac | 2400 |
| ttcgactacg acggcagatg tgacagcctg gaaaatctgc ggcagaaaag aagagtgatc | 2460 |
| cggaaccagg aaattctgat ccctacccac ttctttatcg tgctgacaag ctgcaaggat | 2520 |
| accagccaga cccccctgca ctgcgagaac ctggataccc tggccttcat cctgcctcac | 2580 |
| cggaccgaca acagcgagag ctgtgtgcac ggcaagcacg acagctcttg ggtggaagaa | 2640 |
| ctgctgatgc tgcaccgggc cagaatcacc gatgtggaac acatcaccgg cctgagcttt | 2700 |
| taccagcagc ggaaagaacc cgtgtccgat atcctgaagc tgaaaaccca tctgcccacc | 2760 |
| ttcagccagg aagat | 2775 |

SEQ ID NO: 78-Azurocidin-ENPP1-FC Nucleotide sequence
ggtaccgccaccatgacaagactgacagtgctggctctgctggccggactgttggcctcttctagagctg
ctccttcctgcgccaaagaagtgaagtcctgcaagggcagatgcttcgagcggaccttcggcaactgtag
atgtgacgccgcttgcgtggaactgggcaactgctgcctggactaccaagagacatgcatcgagcccgag
cacatctggacctgcaacaagttcagatgcggcgagaagcggctgaccagatctctgtgcgcctgctctg
acgactgcaaggacaagggcgactgctgcatcaactactcctctgtgtgccagggcgagaagtcctgggt
tgaagaaccctgcgagtccatcaacgagcctcagtgtcctgccggcttcgagacacctcctactctgctg
ttctccctggatggcttcagagccgagtacctgcatacttggggaggcctgctgccagtgatctccaagc
tgaagaagtgcggcacctacaccaagaacatgaggcctgtgtaccctaccaagacattccccaaccacta
ctccatcgtgaccggcctgtatcctgagagccacggcatcatcgacaacaagatgtacgaccccaagatg
aacgcctccttcagcctgaagtccaaagagaagttcaaccccgagtggtataagggcgagcctatctggg
tcaccgctaagtaccagggactgaagtctggcaccttcttttggcctggctccgacgtggaaatcaacgg
catcttccccgacatctataagatgtacaacggctccgtgcctttcgaggaacgcattctggctgttctg
cagtggctgcagctgcctaaggatgagaggcctcacttctacaccctgtacctggaagaacctgactcct
ccggccactcttatggccctgtgtcctctgaagtgatcaaggccctgcagcgagtggacggaatggtcgg
aatgctgatggacggcctgaaagagctgaacctgcacagatgcctgaacctgatcctgatctccgaccac
ggcatggaacaggggagctgcaagaagtacatctacctgaacaagtacctgggcgacgtgaagaacatca
aagtgatctacggcccagccgccagactgaggccttctgatgtgcctgacaagtactactccttcaacta
cgagggaatcgcccggaacctgtcctgcagagagcctaaccagcacttcaagccctacctgaagcacttt
ctgcctaagcggctgcacttcgccaagtctgacagaatcgagcccctgaccttctatctggaccctcagt
ggcagctggccctgaatcctagcgagagaaagtactgtggctccggcttccacggctccgacaacgtgtt
ctctaatatgcaggccctgttcgtcggctacggccctggctttaaacacggcatcgaggccgacaccttc
gagaacatcgaggtgtacaatctgatgtgtgacctgctgaatctgacccctgctcctaacaacggcaccc
acggatctctgaaccatctgctgaagaatcccgtgtacacccctaagcaccccaaagaggttcaccctct
ggtccagtgtcctttcaccagaaatcctcgggacaacctgggctgctcttgcaacccttctatcctgcct
atcgaggactttcagacccagttcaacctgaccgtggccgaggaaaagatcatcaagcacgagacactgc
cctacggcagacctagagtgctgcagaaagagaacaccatctgcctgctgtcccagcaccagttcatgtc
cggctactcccaggacatcctgatgcctctgtggacctcctacaccgtggaccggaacgatagcttctcc
accgaggacttcagcaactgcctgtaccaggatttcagaatccctctgagccccgtgcacaagtgcagct
tctacaagaacaacaccaaggtgtcctacggcttcctgtctcctccacagctgaacaagaactccagcgg
catctactctgaggccctgctgaccaccaacatcgtgcccatgtaccagtccttccaagtgatctggcgg
tacttccacgacaccctgctgaggaagtacgccgaagaaagaaacggcgtgaacgtggtgtctggccccg
tgttcgacttcgactacgacggcagatgcgactctctggaaaacctgcggcagaaaagacgagtgatccg
gaatcaagagatcctgattcctacacacttctttatcgtgctgaccagctgcaaggatacctctcagacc
cctctgcactgcgagaatctggacaccctggccttcattctgcctcacagaaccgacaactccgagtcct
gtgtgcacggcaagcacgactcctcttgggtcgaagaactgctgatgctgcaccgggccagaatcaccga
tgtggaacacatcaccggcctgagcttctaccagcagcggaaagaacctgtgtccgatatcctgaagctg
aaaacccatctgccaaccttcagccaagaggacctgatcaacgacaagacccacacctgtcctccatgtc
ctgctccagaactgctcggaggcccctctgtgttcctgtttccacctaagccaaaggacacactgatgat
ctctcggacccctgaagtgacctgcgtggtggtggatgtgtctcacgaagatcccgaagtcaagttcaat
tggtacgtggacggcgtggaagtgcacaacgccaagaccaagcctagagaggaacagtacaactccacct
acagagtggtgtccgtgctgactgtgctgcaccaggattggctgaacggcaaagagtacaagtgcaaagt
gtccaacaaggctctgcccgctcctatcgaaaagaccatctccaaggctaagggccagcctcgggaacct
caggtttacaccctgcctccatctcgggaagagatgaccaagaaccaggtgtccctgacctgcctggtca
agggcttctacccttccgatatcgccgtggaatgggagtccaatggccagcctgagaacaactacaagac
aacccctcctgtgctggacagcgacggctcattcttcctgtactctaagctgacagtggacaagtcccgg
tggcagcaaggcaatgtgttttcctgctctgtgatgcacgaggccctccacaatcactacacccagaagt
ccctgtctctgtcccctggcaaatgatagctcgag
Legend-bold = start/stop codon; underlined = nucleotide
sequence of signal peptide.
SEQ ID NO: 79-Azurocidin-ENPP3-FC Nucleotide sequence
atgaccagactgaccgtgctggccctgctggccggcctgctggccagcagcagagccgccaagca
gggcagctgcagaaagaagtgcttcgacgccagcttcagaggcctggagaactgcagatgcgacgtggcc
tgcaaggacagaggcgactgctgctgggacttcgaggacacctgcgtggagagcaccagaatctggatgt
gcaacaagttcagatgcggcgagaccagactggaggccagcctgtgcagctgcagcgacgactgcctgca
gagaaaggactgctgcgccgactacaagagcgtgtgccagggcgagaccagctggctggaggagaactgc
gacaccgcccagcagagccagtgccccgagggcttcgacctgccccccgtgatcctgttcagcatggacg
gcttcagagccgagtacctgtacacctgggacaccctgatgcccaacatcaacaagctgaagacctgcgg
catccacagcaagtacatgagagccatgtaccccaccaagaccttccccaaccactacaccatcgtgacc
ggcctgtaccccgagagccacggcatcatcgacaacaacatgtacgacgtgaacctgaacaagaacttca
gcctgagcagcaaggagcagaacaaccccgcctggtggcacggccagcccatgaacctgaccgccatgta
ccagggcctgaaggccgccacctacttctggcccggcagcgaggtggccatcaacggcagcttccccagc
atctacatgccctacaacggcagcgtgcccttcgaggagagaatcagcaccctgctgaagtggctggacc
tgcccaaggccgagagacccagattctacaccatgtacttcgaggagcccgacagcagcggccacgccgg
cggccccgtgagcgccagagtgatcaaggccctgcaggtggtggaccacgccttcggcatgctgatggag
ggcctgaagcagagaaacctgcacaactgcgtgaacatcatcctgctggccgaccacggcatggaccaga
cctactgcaacaagatggagtacatgaccgactacttccccagaatcaacttcttctacatgtacgaggg
ccccgcccccagaatcagagcccacaacatcccccacgacttcttcagcttcaacagcgaggagatcgtg
agaaacctgagctgcagaaagcccgaccagcacttcaagccctacctgacccccgacctgcccaagagac
tgcactacgccaagaacgtgagaatcgacaaggtgcacctgttcgtggaccagcagtggctggccgtgag
aagcaagagcaacaccaactgcggcggcggcaaccacggctacaacaacgagttcagaagcatggaggcc
atcttcctggcccacggccccagcttcaaggagaagaccgaggtggagcccttcgagaacatcgaggtgt
acaacctgatgtgcgacctgctgagaatccagcccgcccccaacaacggcacccacggcagcctgaacca
cctgctgaaggtgcccttctacgagcccagccacgccgaggaggtgagcaagttcagcgtgtgcggcttc
gccaaccccctgcccaccgagagcctggactgcttctgcccccacctgcagaacagcacccagctggagc
aggtgaaccagatgctgaacctgacccaggaggagatcaccgccaccgtgaaggtgaacctgcccttcgg
cagacccagagtgctgcagaagaacgtggaccactgcctgctgtaccacagagagtacgtgagcggcttc
ggcaaggccatgagaatgcccatgtggagcagctacaccgtgccccagctgggcgacaccagccccctgc
cccccaccgtgcccgactgcctgagagccgacgtgagagtgccccccagcgagagccagaagtgcagctt
ctacctggccgacaagaacatcacccacggcttcctgtacccccccgccagcaacagaaccagcgacagc
cagtacgacgccctgatcaccagcaacctggtgcccatgtacgaggagttcagaaagatgtgggactact
tccacagcgtgctgctgatcaagcacgccaccgagagaaacggcgtgaacgtggtgagcggccccatctt
cgactacaactacgacggccacttcgacgcccccgacgagatcaccaagcacctggccaacaccgacgtg
cccatccccacccactacttcgtggtgctgaccagctgcaagaacaagagccacacccccgagaactgcc
ccggctggctggacgtgctgcccttcatcatcccccacagacccaccaacgtggagagctgccccgaggg
caagcccgaggccctgtgggtggaggagagattcaccgcccacatcgccagagtgagagacgtggagctg
ctgaccggcctggacttctaccaggacaaggtgcagcccgtgagcgagatcctgcagctgaagacctacc
tgcccaccttcgagaccaccatcgacaagacccacacctgccccccctgccccgcccccgagctgctggg
cggccccagcgtgttcctgttcccccccaagcccaaggacaccctgatgatcagcagaacccccgaggtg
acctgcgtggtggtggacgtgagccacgaggaccccgaggtgaagttcaactggtacgtggacggcgtgg
aggtgcacaacgccaagaccaagcccagagaggagcagtacaacagcacctacagagtggtgagcgtgct
gaccgtgctgcaccaggactggctgaacggcaaggagtacaagtgcaaggtgagcaacaaggccctgccc
gcccccatcgagaagaccatcagcaaggccaagggccagcccagagagccccaggtgtacaccctgcccc
ccagcagagaggagatgaccaagaaccaggtgagcctgacctgcctggtgaagggcttctaccccagcga
catcgccgtggagtgggagagcaacggccagcccgagaacaactacaagaccaccccccccgtgctggac
agcgacggcagcttcttcctgtacagcaagctgaccgtggacaagagcagatggcagcagggcaacgtgt
tcagctgcagcgtgatgcacgaggccctgcacaaccactacacccagaagagcctgagcctgagccccgg
caag

### Cloning and Expression of ENPP1 and ENPP3 Fusion Polypeptides

ENPP1, or an ENPP1 polypeptide, is prepared as described in US 2015/0359858 A1, which is incorporated herein in its entirety by reference. ENPP1 is a transmembrane protein localized to the cell surface with distinct intramembrane domains. In order to express ENPP1 as a soluble extracellular protein, the transmembrane domain of ENPP1 may be swapped for the transmembrane domain of ENPP2 or a signal peptide sequence such as Azurocidin, which results in the accumulation of soluble, recombinant ENPP1 in the extracellular fluid of the baculovirus cultures. Signal sequences of any other known proteins may be used to target the extracellular domain of ENPP1 for secretion as well, such as but not limited to the signal sequence of the immunoglobulin kappa and lambda light chain proteins. Further, the disclosure should not be construed to be limited to the polypeptides described herein, but also includes polypeptides comprising any enzymatically active truncation of the ENPP1 extracellular domain.

ENPP1 is made soluble by omitting the transmembrane domain. Human ENPP1 (SEQ ID NO:1) was modified to express a soluble, recombinant protein by replacing its transmembrane region (e.g., residues 77-98) with the corresponding subdomain of human ENPP2 (NCBI accession NP 00112433 5, e.g., residues 12-30) or Azurocidin signal sequence (SEQ ID 42).

The modified ENPP1 sequence was cloned into a modified pFastbac FIT vector possessing a TEV protease cleavage site followed by a C-terminus 9-F HS tag, and cloned and expressed in insect cells, and both proteins were expressed in a baculovirus system as described previously (Albright, et al., 2012, Blood 120:4432-4440; Saunders, et al., 2011, J. Biol. Chem. 18:994-1004; Saunders, et al., 2008, Mol. Cancer Ther. 7:3352-3362), resulting in the accumulation of soluble, recombinant protein in the extracellular fluid.

ENPP3 is poorly exported to the cell surface. Soluble ENPP3 polypeptide is constructed by replacing the signal sequence of ENPP3 with the native signal sequence of other ENPPs or Azurocidin or suitable signal sequences. Several examples of ENPP3 fusion constructs are disclosed in WO 2017/087936. Soluble ENPP3 constructs are prepared by using the signal export signal sequence of other ENPP enzymes, such as but not limited to ENPP7 and/or ENPP5. Soluble ENPP3 constructs are prepared using a signal sequence comprised of a combination of the signal sequences of ENPP1 and ENPP2 (“ENPP1-2-1” or “ENPP121” hereinafter). Signal sequences of any other known proteins may be used to target the extracellular domain of ENPP3 for secretion as well, such as but not limited to the signal sequence of the immunoglobulin kappa and lambda light chain proteins. Further, the disclosure should not be construed to be limited to the constructs described herein, but also includes constructs comprising any enzymatically active truncation of the ENPP3 extracellular domain.

In certain embodiments, the ENPP3 polypeptide is soluble. In some embodiments, the polypeptide of the disclosure includes an ENPP3 polypeptide that lacks the ENPP3 transmembrane domain. In another embodiment, the polypeptide of the disclosure includes an ENPP3 polypeptide wherein the ENPP3 transmembrane domain has been removed and replaced with the transmembrane domain of another polypeptide, such as, by way of non-limiting example, ENPP2, ENPPS or ENPP7 or Azurocidin signal sequence.

In some embodiments, the polypeptide of the disclosure comprises an IgG Fc domain. In certain embodiments, the polypeptide of the disclosure comprises an albumin domain. In other embodiments, the albumin domain is located at the C terminal region of the ENPP3 polypeptide. In yet other embodiments, the IgG Fc domain is located at the C terminal region of the ENPP3 polypeptide. In yet other embodiments, the presence of IgG Fc domain or albumin domain improves half-life, solubility, reduces immunogenicity and increases the activity of the ENPP3 polypeptide.

In certain embodiments, the polypeptide of the disclosure comprises a signal peptide resulting in the secretion of a precursor of the ENPP3 polypeptide, which undergoes proteolytic processing to yield the ENPP3 polypeptide. In other embodiments, the signal peptide is selected from the group consisting of signal peptides of ENPP2, ENPP5 and ENPP7. In yet other embodiments, the signal peptide is selected from the group consisting of SEQ ID NOs: 36-42.

In certain embodiments, the IgG Fc domain or the albumin domain is connected to the C terminal region of the ENPP3 polypeptide by a linker region. In other embodiments, the linker is selected from SEQ ID NOs: 43-75, where n is an integer ranging from 1-20.

### Production and Purification of ENPP1 and ENPP3 Fusion Polypeptides

To produce soluble, recombinant ENPP1 polypeptide for in vitro use, polynucleotide encoding the extracellular domain of ENPP1 (Human NPP1 (NCBI accession NP 006199)) was fused to the Fc domain of IgG (referred to as “ENPP1-Fc”) and was expressed in stable CHO cell lines. In some embodiments, ENPP1 polynucleotide encoding residues 96 to 925 of NCBI accession NP 006199 were fused to Fc domain to generate ENPP1 polypeptide.

Alternately the ENPP1 polypeptide can also be expressed from HEK293 cells, Baculovirus insect cell system or CHO cells or Yeast Pichia expression system using suitable vectors. The ENPP1 polypeptide can be produced in either adherent or suspension cells. Preferably the ENPP1 polypeptide is expressed in CHO cells. To establish stable cell lines the nucleic acid sequence encoding ENPP1 constructs are cloned into an appropriate vector for large scale protein production.

ENPP3 is produced by establishing stable transfections in either CHO or HEK293 mammalian cells. ENPP3 polynucleotide encoding ENPP3 (Human NPP3 (UniProtKB/Swiss-Prot: O14638.2) was fused to the Fc domain of IgG (referred to as “ENPP3-Fc”) and was expressed in stable CHO cell lines. In some embodiments, ENPP3 polynucleotide encoding residues 49-875 of UniProtKB/Swiss-Prot: O14638.2 was fused to Fc domain to generate ENPP3 polypeptide. The ENPP3 polypeptide can be produced in either adherent or suspension cells. To establish stable cell lines the nucleic acid sequence encoding NPP3 fusion polypeptides of the disclosure into an appropriate vector for large scale protein production. There are a variety of these vectors available from commercial sources and any of those can be used. ENPP3 polypeptides are produced following the protocols established in WO 2017/087936, the contents of which are hereby incorporated by reference in their entirety. ENPP1 polypeptides are produced following the protocols established in Albright, et al, 2015, Nat Commun. 6:10006, the contents of which are hereby incorporated by reference in their entirety.

A suitable plasmid containing the desired polypeptide constructs of ENPP1 or ENPP3 can be stably transfected into expression plasmid using established techniques such as electroporation or lipofectamine, and the cells can be grown under antibiotic selection to enhance for stably transfected cells. Clones of single, stably transfected cells are then established and screened for high expressing clones of the desired fusion protein. Screening of the single cell clones for ENPP1 or ENPP3 polypeptide expression can be accomplished in a high-throughput manner in 96 well plates using the synthetic enzymatic substrate pNP-TMP as previously described (Saunders, et al, 2008, Mol. Cancer Therap. 7(10):3352-62; Albright, et al, 2015, Nat Commun. 6:10006).

Upon identification of high expressing clones for ENPP3 or ENPP1 polypeptides through screening, protein production can be accomplished in shaking flasks or bio-reactors previously described for ENPP1 (Albright, et al, 2015, Nat Commun. 6:10006). Purification of ENPP3 or ENPP1 polypeptides can be accomplished using a combination of standard purification techniques known in the art. These techniques are well known in art and are selected from techniques such as column chromatograph, ultracentrifugation, filtration, and precipitation. Column chromatographic purification is accomplished using affinity chromatography such as protein-A and protein-G resins, metal affinity resins such as nickel or copper, hydrophobic exchange chromatography, and reverse-phase high-pressure chromatography (HPLC) using C8-C14 resins. Ion exchange may also be employed, such as anion and cation exchange chromatography using commercially available resins such as Q-sepharose (anion exchange) and SP-sepharose (cation exchange), blue sepharose resin and blue-sephadex resin, and hydroxyapatite resins. Size exclusion chromatography using commercially available S-75 and S200 Superdex resins can also be employed, as known in the art. Buffers used to solubilize the protein and provide the selection media for the above described chromatographic steps, are standard biological buffers known to practitioners of the art and science of protein chemistry.

Some examples of buffers that are used in preparation include citrate, phosphate, acetate, tris(hydroxymemyl)aminomethane, saline buffers, glycine-HCL buffers, Cacodylate buffers, and sodium barbital buffers, which are well known in art. Using a single techniques, or a series of techniques in combination, and the appropriate buffer systems purified ENPP3 and the crude starting material side by side on a Coomasie stained polyacrylamide gel after a single purification step. The ENPP3 protein can then be additionally purified using additional techniques and/or chromatographic steps as described above, to reach substantially higher purity such as −99% purity adjusted to the appropriate pH, one can purify the ENPP1 or ENPP3 polypeptides described to greater than 99% purity from crude material.

Following purification, ENPP1-Fc or ENPP3-Fc was dialyzed into PBS supplemented with Zn2+ and Mg2+(PBSplus) concentrated to between 5 and 7 mg/ml, and frozen at −80° C. in aliquots of 200-500 μl. Aliquots were thawed immediately prior to use and the specific activity of the solution was adjusted to 31.25 au/ml (or about 0.7 mg/ml depending on the preparation) by dilution in PBSplus.

### Dosage & Mode of Administration

In another embodiment, the hsNPP1 or hsNPP3 is administered in one or more doses containing about 1.0 mg/kg to about 5.0 mg/kg NPP1 or about 1.0 mg/kg to about 5.0 mg/kg NPP3 respectively. In another embodiment, the hsNPP1 or hsNPP3 is administered in one or more doses containing about 1.0 mg/kg to about 10.0 mg/kg NPP1 or about 1.0 mg/kg to about 10.0 mg/kg NPP3.

The time period between doses of the hsNPP1 or hsNPP3 is at least 2 days and can be longer, for example at least 3 days, at least 1 week, 2 weeks or 1 month. In one embodiment, the administration is weekly, bi-weekly, or monthly.

The recombinant hsNPP1 or hsNPP3 can be administered in any suitable way, such as intravenously, subcutaneously, or intraperitoneally.

The recombinant hsNPP1 or hsNPP3 can be administered in combination with one or more additional therapeutic agents. Exemplary therapeutic agents include, but are not limited to Bisphosphonate, Statins, Fibrates, Niacin, Aspirin, Clopidogrel, and warfarin.

In some embodiments, the recombinant hsNPP1 or hsNPP3 and additional therapeutic agent are administered separately and are administered concurrently or sequentially. In some embodiments, the recombinant hsNPP1 or hsNPP3 is administered prior to administration of the additional therapeutic agent. In some embodiments, the recombinant hsNPP1 or hsNPP3 is administered after administration of the additional therapeutic agent. In other embodiments, the recombinant hsNPP1 or hsNPP3 and additional therapeutic agent are administered together.

### Nucleic Acid Administration and Therapy

### Viral Vectors for In Vivo Expression of ENPP1 and ENPP3

The nucleic acids encoding the polypeptide(s) useful within the disclosure may be used in gene therapy protocols for the treatment of the diseases or disorders contemplated herein. The improved construct encoding the polypeptide(s) can be inserted into the appropriate gene therapy vector and administered to a patient to treat or prevent the diseases or disorder of interest.

Vectors, such as viral vectors, have been used in the prior art to introduce genes into a wide variety of different target cells. Typically, the vectors are exposed to the target cells so that transformation can take place in a sufficient proportion of the cells to provide a useful therapeutic or prophylactic effect from the expression of the desired polypeptide (e.g., a receptor). The transfected nucleic acid may be permanently incorporated into the genome of each of the targeted cells, providing long lasting effect, or alternatively the treatment may have to be repeated periodically. In certain embodiments, the (viral) vector transfects liver cells in vivo with genetic material encoding the polypeptide(s) of the disclosure.

A variety of vectors, both viral vectors and plasmid vectors are known in the art (see for example U.S. Pat. No. 5,252,479 and WO 93/07282). In particular, a number of viruses have been used as gene transfer vectors, including papovaviruses, such as SV40, vaccinia virus, herpes viruses including HSV and EBV, and retroviruses. Many gene therapy protocols in the prior art have employed disabled murine retroviruses. Several recently issued patents are directed to methods and compositions for performing gene therapy (see for example U.S. Pat. Nos. 6,168,916; 6,135,976; 5,965,541 and 6,129,705). Each of the foregoing patents is incorporated by reference in its entirety herein. Hence, genetic material such as a polynucleotide comprising an NPP1 or an NPP3 sequence can be introduced to a mammal in order to treat VSMC proliferation.

Certain modified viruses are often used as vectors to carry a coding sequence because after administration to a mammal, a virus infects a cell and expresses the encoded protein. Modified viruses useful according to the disclosure are derived from viruses which include, for example: parvovirus, picornavirus, pseudorabies virus, hepatitis virus A, B or C, papillomavirus, papovavirus (such as polyoma and SV40) or herpes virus (such as Epstein-Barr Virus, Varicella Zoster Virus, Cytomegalovirus, Herpes Zoster and Herpes Simplex Virus types 1 and 2), an RNA virus or a retrovirus, such as the Moloney murine leukemia virus or a lentivirus (i.e. derived from Human Immunodeficiency Virus, Feline Immunodeficiency Virus, equine infectious anemia virus, etc.). Among DNA viruses useful according to the disclosure are: Adeno-associated viruses adenoviruses, Alphaviruses, and Lentiviruses.

A viral vector is generally administered by injection, most often intravenously (by IV) directly into the body, or directly into a specific tissue, where it is taken up by individual cells. Alternately, a viral vector may be administered by contacting the viral vector ex vivo with a sample of the patient's cells, thereby allowing the viral vector to infect the cells, and cells containing the vector are then returned to the patient. Once the viral vector is delivered, the coding sequence expressed and results in a functioning protein. Generally, the infection and transduction of cells by viral vectors occur by a series of sequential events as follows: interaction of the viral capsid with receptors on the surface of the target cell, internalization by endocytosis, intracellular trafficking through the endocytic/proteasomal compartment, endosomal escape, nuclear import, virion uncoating, and viral DNA double-strand conversion that leads to the transcription and expression of the recombinant coding sequence interest. (Colella et al., Mol Ther Methods Clin Dev. 2017 Dec. 1; 8:87-104.).

Adeno-Associated Viral Vectors According to the Disclosure

AAV refers to viruses belonging to the genus Dependovirus of the Parvoviridae family. The AAV genome is approximately 4.7 kilobases long and is composed of linear single-stranded deoxyribonucleic acid (ssDNA) which may be either positive- or negative-sensed. The genome comprises inverted terminal repeats (ITRs) at both ends of the DNA strand, and two open reading frames (ORFs): rep and cap. The rep frame is made of four overlapping genes encoding non-structural replication (Rep) proteins required for the AAV life cycle. The cap frame contains overlapping nucleotide sequences of structural VP capsid proteins: VP1, VP2 and VP3, which interact together to form a capsid of an icosahedral symmetry.

The terminal 145 nucleotides are self-complementary and are organized so that an energetically stable intramolecular duplex forming a T-shaped hairpin may be formed. These hairpin structures function as an origin for viral DNA replication, serving as primers for the cellular DNA polymerase complex. Following wild type AAV infection in mammalian cells the rep genes (i.e. Rep78 and Rep52) are expressed from the P5 promoter and the P19 promoter, respectively, and both Rep proteins have a function in the replication of the viral genome. A splicing event in the rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV replicates in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia).

Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a patient's cells are infected with the resulting rAAV virions, the heterologous nucleic acid sequence enters and is expressed in the patient's cells.

Because the patient's cells lack the rep and cap genes, as well as the accessory function genes, the rAAV cannot further replicate and package their genomes. Moreover, without a source of 5 rep and cap genes, wtAAV cannot be formed in the patient's cells.

The AAV vector typically lacks rep and cap frames. Such AAV vectors can be replicated and packaged into infectious viral particles when present in a host cell that has been transfected with a vector encoding and expressing rep and cap gene products (i.e. AAV Rep and Cap proteins), and wherein the host cell has been transfected with a vector which encodes and expresses a protein from the adenovirus open reading frame E4orf6.

Delivery of a protein of interest to the cells of a mammal is accomplished by first generating an AAV vector comprising DNA encoding the protein of interest and then administering the vector to the mammal. Thus, the disclosure should be construed to include AAV vectors comprising DNA encoding the polypeptide(s) of interest. Once armed with the present disclosure, the generation of AAV vectors comprising DNA encoding this/these polypeptide(s)s will be apparent to the skilled artisan.

In one embodiment, the disclosure relates to an adeno-associated viral (AAV) expression vector comprising a sequence encoding mammal ENPP1 or mammal ENPP3, and upon administration to a mammal the vector expresses an ENPP1 or ENPP3 precursor in a cell, the precursor including an Azurocidin signal peptide fused at its carboxy terminus to the amino terminus of ENPP1 or ENPP3. The ENPP1 or ENPP3 precursor may include a stabilizing domain, such as an IgG Fc region or human albumin. Upon secretion of the precursor from the cell, the signal peptide is cleaved off and enzymatically active soluble mammal ENPP1 or ENPP3 is provided extracellularly.

An AAV expression vector may include an expression cassette comprising a transcriptional regulatory region operatively linked to a nucleotide sequence comprising a transcriptional regulatory region operatively linked to a recombinant nucleic acid sequence encoding a polypeptide comprising a Azurocidin signal peptide sequence and an ectonucleotide pyrophosphatase/phosphodiesterase (ENPP1) polypeptide sequence.

In some embodiments, the expression cassette comprises a promoter and enhancer, the Kozak sequence GCCACCATGG, a nucleotide sequence encoding mammal NPP1 protein or a nucleotide sequence encoding mammal NPP3 protein, other suitable regulatory elements and a polyadenylation signal.

In some embodiments, the AAV recombinant genome of the AAV vector according to the disclosure lacks the rep open reading frame and/or the cap open reading frame.

The AAV vector according to the disclosure comprises a capsid from any serotype. In general, the AAV serotypes have genomic sequences of significant homology at the amino acid and the nucleic acid levels, provide an identical set of genetic functions, and replicate and assemble through practically identical mechanisms. In particular, the AAV of the present disclosure may belong to the serotype 1 of AAV (AAV1), AAV2, AAV3 (including types 3A and 3B), AAV4, AAV5, AAV6, AAV7, AAV8, AAV9, AAV10, AAVrh10, AAV11, avian AAV, bovine AAV, canine AAV, equine AAV, or ovine AAV.

Examples of the sequences of the genome of the different AAV serotypes may be found in the literature or in public databases such as GenBank. For example, GenBank accession numbers NC 001401.2 (AAV2), NC 001829.1 (AAV4), NC 006152.1 (AAV5), AF028704.1 (AAV6), NC 006260.1 (AAV7), NC 006261.1 (AAV8), AX753250.1 (AAV9) and AX753362.1 (AAV10).

In some embodiments, the adeno-associated viral vector according to the disclosure comprises a capsid derived from a serotype selected from the group consisting of the AAV2, AAV5, AAV7, AAV8, AAV9, AAV10 and AAVrhl 0 serotypes. In another embodiment, the serotype of the AAV is AAV8. If the viral vector comprises sequences encoding the capsid proteins, these may be modified so as to comprise an exogenous sequence to direct the AAV to a particular cell type or types, or to increase the efficiency of delivery of the targeted vector to a cell, or to facilitate purification or detection of the AAV, or to reduce the host response.

In certain embodiments, the rAAV vector of the disclosure comprises several essential DNA elements. In certain embodiments, these DNA elements include at least two copies of an AAV ITR sequence, a promoter/enhancer element, a transcription termination signal, any necessary 5′ or 3′ untranslated regions which flank DNA encoding the protein of interest or a biologically active fragment thereof. The rAAV vector of the disclosure may also include a portion of an intron of the protein on interest. Also, optionally, the rAAV vector of the disclosure comprises DNA encoding a mutated polypeptide of interest.

In certain embodiments, the vector comprises a promoter/regulatory sequence that comprises a promiscuous promoter which is capable of driving expression of a heterologous gene to high levels in many different cell types. Such promoters include but are not limited to the cytomegalovirus (CMV) immediate early promoter/enhancer sequences, the Rous sarcoma virus promoter/enhancer sequences and the like. In certain embodiments, the promoter/regulatory sequence in the rAAV vector of the disclosure is the CMV immediate early promoter/enhancer. However, the promoter sequence used to drive expression of the heterologous gene may also be an inducible promoter, for example, but not limited to, a steroid inducible promoter, or may be a tissue specific promoter, such as, but not limited to, the skeletal a-actin promoter which is muscle tissue specific and the muscle creatine kinase promoter/enhancer, and the like.

In certain embodiments, the rAAV vector of the disclosure comprises a transcription termination signal. While any transcription termination signal may be included in the vector of the disclosure, in certain embodiments, the transcription termination signal is the SV40 transcription termination signal.

In certain embodiments, the rAAV vector of the disclosure comprises isolated DNA 5 encoding the polypeptide of interest, or a biologically active fragment of the polypeptide of interest. The disclosure should be construed to include any mammalian sequence of the polypeptide of interest, which is either known or unknown. Thus, the disclosure should be construed to include genes from mammals other than humans, which polypeptide functions in a substantially similar manner to the human polypeptide. Preferably, the nucleotide sequence comprising the gene encoding the polypeptide of interest is about 50% homologous, more preferably about 70% homologous, even more preferably about 80% homologous and most preferably about 90% homologous to the gene encoding the polypeptide of interest.

Further, the disclosure should be construed to include naturally occurring variants or recombinantly derived mutants of wild type protein sequences, which variants or mutants render the polypeptide encoded thereby either as therapeutically effective as full-length polypeptide, or even more therapeutically effective than full-length polypeptide in the gene therapy methods of the disclosure.

The disclosure should also be construed to include DNA encoding variants which retain the polypeptide's biological activity. Such variants include proteins or polypeptides which have been or may be modified using recombinant DNA technology, such that the protein or polypeptide possesses additional properties which enhance its suitability for use in the methods described herein, for example, but not limited to, variants conferring enhanced stability on the protein in plasma and enhanced specific activity of the protein. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The disclosure is not limited to the specific rAAV vector exemplified in the experimental examples; rather, the disclosure should be construed to include any suitable AAV vector, including, but not limited to, vectors based on AAV-1, AAV-3, AAV-4 and AAV-6, and the like. Also included in the disclosure is a method of treating a mammal having a disease or disorder in an amount effective to provide a therapeutic effect.

The method comprises administering to the mammal an rAAV vector encoding the polypeptide of interest. Preferably, the mammal is a human. Typically, the number of viral vector genomes/mammal which are administered in a single injection ranges from about 1×108 to about 5×1016. Preferably, the number of viral vector genomes/mammal which are administered in a single injection is from about 1×10¹⁰ to about 1×10¹⁵; more preferably, the number of viral vector genomes/mammal which are administered in a single injection is from about 5×10¹⁰ to about 5×10¹⁵; and, most preferably, the number of viral vector genomes which are administered to the mammal in a single injection is from about 5×10¹⁰ to about 5×10¹⁴.

When the method of the disclosure comprises multiple site simultaneous injections, or several multiple site injections comprising injections into different sites over a period of several hours (for example, from about less than one hour to about two or three hours) the total number of viral vector genomes administered may be identical, or a fraction thereof or a multiple thereof, to that recited in the single site injection method.

For administration of the rAAV vector of the disclosure in a single site injection, in certain embodiments a composition comprising the virus is injected directly into an organ of the subject (such as, but not limited to, the liver of the subject).

For administration to the mammal, the rAAV vector may be suspended in a pharmaceutically acceptable carrier, for example, HEPES buffered saline at a pH of about 7.8. Other useful pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey). The rAAV vector of the disclosure may also be provided in the form of a kit, the kit comprising, for example, a freeze-dried preparation of vector in a dried salts formulation, sterile water for suspension of the vector/salts composition and instructions for suspension of the vector and administration of the same to the mammal

The published application, US 2017/0290926—Smith et al., the contents of which are incorporated by reference in their entirety herein, describes in detail the process by which AAV vectors are generated, delivered and administered.

### RNA Based In Vivo Expression of ENPP1 and ENPP3 Polypeptides

The present disclosure provides compositions and methods for the production and delivery of recombinant double-stranded RNA molecules (dsRNA that encode ENPP1 or ENPP3 polypeptides described herein. The double stranded RNA particle (dsRP) can contain a dsRNA molecule enclosed in a capsid or coat protein. The dsRNA molecule can be a viral genome or portion of a genome, which can be derived from a wild-type viral genome. The RNA molecule can encode an RNA-dependent RNA polymerase (RDRP) and a polyprotein that forms at least part of a capsid or coat protein. The RNA molecule can also contain an RNA sub-sequence that encodes an ENPP1 or ENPP3 polypeptides that are translated by the cellular components of a host cell. When the dsRP is transfected into a host cell the sub-sequence can be translated by the cellular machinery of the host cell to produce the ENPP1 or ENPP3 polypeptides.

In another aspect the disclosure provides a method of producing a protein product in a host cell. The method includes transfecting a host cell with a dsRP having a recombinant double-stranded RNA molecule (dsRNA) and a capsid or coat protein. The RNA molecule can encode an RNA-dependent RNA polymerase and a polyprotein that forms at least part of the capsid or coat protein, and the dsRP can be able to replicate in the host cell. The RNA molecule has at least one RNA sub-sequence that encodes ENPP1 or ENPP3 polypeptides that is translated by cellular components of the host cell.

In another aspect the disclosure provides an RNA molecule translatable by a host cell. The RNA molecule can be any RNA molecule that encodes the ENPP1 or ENPP3 polypeptides described herein. In one embodiment the RNA molecule encodes an RNA-dependent RNA polymerase and a polyprotein that forms at least part of a capsid or coat protein of a dsRP and, optionally, can have at least one sub-sequence of RNA that encodes an additional protein product.

Production of dsRP

A dsRP of the disclosure can also be produced by presenting to a host cell a plasmid or other DNA molecule encoding a dsRP of the disclosure or encoding the genes of the dsRP. The plasmid or DNA molecule containing nucleotide sequences encoding desired protein such as ENPP1 or ENPP3 polypeptide is then transfected into the host cell and the host cell begins producing the dsRP of the disclosure. The dsRP can also be produced in the host cell by presenting to the host cell an RNA molecule encoding the genes of the dsRP. The RNA molecule can be (+)-strand RNA.

Once the dsRP of the disclosure has been presented to the host cell (or a plasmid encoding the genes of the dsRP of the disclosure, or an RNA molecule encoding the genes of the dsRP), the dsRP will be produced within the host cell using the cellular components of the host cell. The dsRP of the disclosure is therefore self-sustaining within the host cell and is propagated within the host cell. The host cell can be any suitable host cell such as, for example, a eukaryotic cell, a mammalian cell, a fungal cell, a bacterial cell, an insect cell, or a yeast cell. The host cell can propagate a recombinant dsRP after a recombinant dsRNA molecule of the disclosure or a DNA molecule encoding a dsRP of the disclosure is presented to and taken up by the host cell.

Methods of Producing a dsRNA Virus or dsRP

The disclosure also provides methods of producing a dsRP of the disclosure. A double-stranded or single-stranded RNA or DNA molecule can be presented to a host cell. The amplification of the dsRNA molecules in the host cell utilizes the natural production and assembly processes already present in many types of host cells (e.g., yeast). The disclosure can thus be applied by presenting to a host cell a single-stranded or double-stranded RNA or DNA molecule of the disclosure, which is taken up by the host cell and is utilized to produce the recombinant dsRP and protein or peptide encoded by the RNA sub-sequence using the host cell's cellular components. The disclosure can also be applied by providing to the host cell a linear or circular DNA molecule (e.g., a plasmid or vector) containing one or more sequences coding for an RNA-dependent RNA polymerase, a polyprotein that forms at least part of the capsid or coat protein of the dsRP, and a sub-sequence encoding the protein of interest such as ENPP1 or ENPP3 polypeptides as disclosed herein.

The presentation of a dsRNA or ssRNA molecule of the disclosure can be performed in any suitable way such as, for example, by presenting an RNA molecule of the disclosure directly to the host cell as “naked” or unmodified single-stranded or double-stranded RNA. The RNA molecule can be transfected (or transformed) into a yeast, bacterial, or mammalian host cell by any suitable method, for example by electroporation, exposure of the host cell to calcium phosphate, or by the production of liposomes that fuse with the cell membrane and deposit the viral sequence inside. It can also be performed by a specific mechanism of direct introduction of dsRNA from killer viruses or heterologous dsRNA into the host cell. This step can be optimized using a reporter system, such as red fluorescent protein (RFP), or by targeting a specific constitutive gene transcript within the host cell genome. This can be done by using a target with an obvious phenotype or by monitoring by quantitative reverse transcriptase PCR (RT-PCR).

In some embodiments a DNA molecule (e.g., a plasmid or other vector) that encodes an RNA molecule of the disclosure is introduced into the host cell. The DNA molecule can contain a sequence coding for the RNA molecule of a dsRP of the disclosure. The DNA molecule can code for an entire genome of the dsRP, or a portion thereof. The DNA molecule can further code for the at least one sub-sequence of RNA that produces the additional (heterologous) protein product. The DNA sequence can also code for gag protein or gag-pol protein, and as well as any necessary or desirable promoters or other sequences supporting the expression and purpose of the molecule. The DNA molecule can be a linear DNA, a circular DNA, a plasmid, a yeast artificial chromosome, or may take another form convenient for the specific application.

In one embodiment the DNA molecule can further comprise T7 ends for producing concatamers and hairpin structures, thus allowing for propagation of the virus or dsRP sequence in the host cell. The DNA molecule can be transfected or transformed into the host cell and then, using the host cellular machinery, transcribed and thus provide the dsRNA molecule having the at least one sub-sequence of RNA to the host cell. The host cell can then produce the encoded desired ENPP1 or ENPP3 polypeptide. The dsRNA can be packaged in the same manner that a wild-type virus would be, using the host cell's metabolic processes and machinery. The ENPP1 or ENPP3 polypeptide is also produced using the host cell's metabolic processes and cellular components.

The patent, U.S. Ser. No. 10/266,834 by Brown et al., the contents of which are incorporated by reference in their entirety herein, describes in detail the process by which dsRNA particles that encode polypeptides are generated, delivered and administered

### Pharmaceutical Compositions and Formulations

The disclosure provides pharmaceutical compositions comprising a polypeptide of the disclosure within the methods described herein. Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 500 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between about 0.1% and about 100% (w/w) active ingredient.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation. In certain embodiments, administration of the compound of the disclosure to a subject elevates the subject's plasma PPi to a level that is close to normal, where a normal level of PPi in mammals is 1-3 μM. “Close to normal” refers to 0 to 1.2 μM or 0-40% below or above normal, 30 nM to 0.9 μM or 1-30% 15 below or above normal, 0 to 0.6 μM or 0-20% below or above normal, or 0 to 0.3 μM or 0-10% below or above normal.

Administration of the compositions of the present disclosure to a patient, such as a mammal, such as a human, may be carried out using known procedures, at dosages and for periods of time effective to treat a disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. Dosage is determined based on the biological activity of the therapeutic compound which in turn depends on the half-life and the area under the plasma time of the therapeutic compound curve. The polypeptide according to the disclosure is administered at an appropriate time interval of every 2 days, or every 4 days, or every week or every month so as to achieve a continuous level of plasma PPi that is either close to the normal (1-3 μM) level or above (30-50% higher than) normal levels of PPi. Therapeutic dosage of the polypeptides of the disclosure may also be determined based on half-life or the rate at which the therapeutic polypeptide is cleared out of the body. The polypeptide according to the disclosure is administered at appropriate time intervals of either every 2 days, or every 4 days, every week or every month so as to achieve a constant level of enzymatic activity of ENPP1 or ENPP3 polypeptides.

For example, several divided doses may be administered daily, or the dose may be proportionally reduced as indicated by the exigencies of the therapeutic situation. A non-limiting example of an effective dose range for a therapeutic compound of the disclosure is from about and 50 mg/kg of body weight/per day. In certain embodiments, the effective dose range for a therapeutic compound of the disclosure is from about 50 ng to 500 ng/kg, preferably 100 ng to 300 ng/kg of bodyweight. One of ordinary skill in the art would be able to study the relevant factors and make the determination regarding the effective amount of the therapeutic compound without undue experimentation.

The compound can be administered to a patient as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the patient. Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. The frequency of administration of the various combination compositions of the disclosure varies from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

### Routes of Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

“Parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### EXAMPLES

The present disclosure is further exemplified by the following examples. The examples are for illustrative purpose only and are not intended, nor should they be construed as limiting the disclosure in any manner.

### Example 1: Efficacy of ENPP1 and ENPP1-Fc Fusion Protein in Mouse Aortic Allografts

Allograft vasculopathy remains one of the main complications hindering long-term graft survival, thus representing a major risk factor for mortality in patients subjected to solid organ transplantation. The aim of the example is to evaluate the efficacy of an ENPP1-Fc fusion protein or ENPP1 protein in a mouse model for aortic allografts. Therapeutic effects of the ENPP1 or ENPP1-Fc fusion protein are assessed with respect to the ability to inhibit stenosis after solid organ transplant.

Female DBA/2 (H-2^{d}) and C57BL/6J (H-2^{b}) mice ages of 5-6 weeks are used as donor and recipient mice respectively. (Bickerstaff et al, Murine renal allografts: spontaneous acceptance is associated with regulated T cell-mediated immunity, 2001, J. Immunol. 167, 4821-4827). Descending thoracic aortae of DBA/2 mice are transplanted into C57CL/6 mice in the infrarenal position, as already described. (Seppelt et al., Loss of Endothelial Barrier in Marfan Mice (mgR/mgR) Results in Severe Inflammation after Adenoviral Gene Therapy, 2016, PLoS ONE 11, e0148012.)

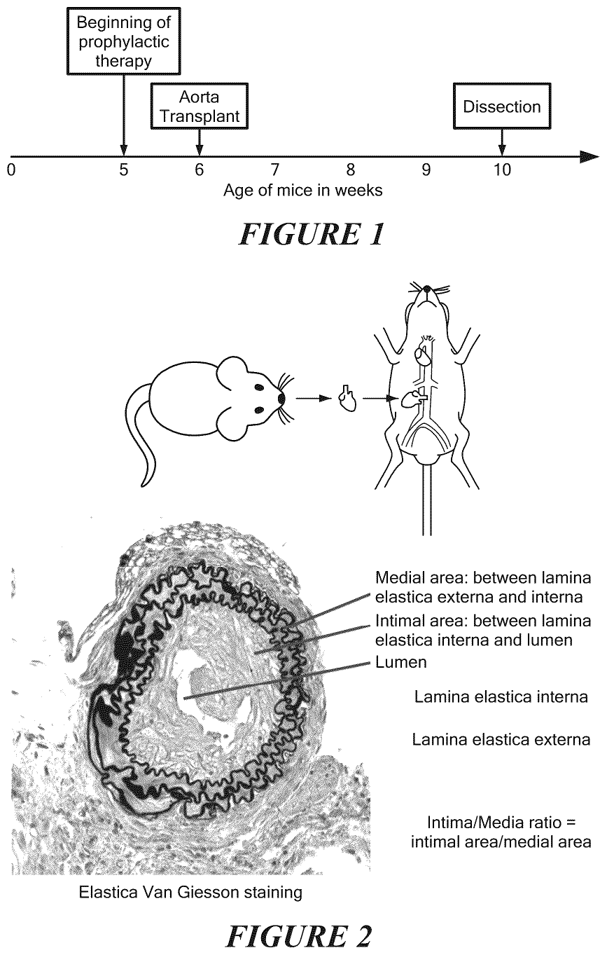

Donor mice are euthanized with CO₂. The thoracic cavity is opened, left ventricle is punctured, and the arterial circulatory system is perfused with 5 mL NaCl (4° C., 0.9%). The descending aorta is harvested and transplanted into the recipient mice to create the model for aortic allografts. Alternatively, the entire heart of the donor mice can be harvested and transplanted into the recipient mice as shown in FIG. 2 to create a solid organ transplant mouse model.

Recipient C57BL/6J mice are anesthetized by inhalation of 5% isoflurane. Novalgin (500 mg/mL; 200 mg/kg body weight) and Carprieve (50 mg/mL carprofen, 5 mg/kg body weight) are injected intraperitoneally. The abdominal cavity of recipient mice is opened and the infrarenal aorta was dissected. Titanium clips are applied, and the aorta was transected. Grafts are connected to recipient aorta with two end-to-end anastomoses (Prolene 11-0, nylon black, S&T, Neuhausen, Switzerland). After removal of the clips the graft was re-perfused. (Remes et al., Molecular Therapy: Methods & Clinical Development Vol. 15 Dec. 2019).

A control subset of recipient mice containing the transplanted aorta (n=5) is treated with tris buffered saline and the experimental subset of recipient mice with the transplanted aorta (n=5) is treated with ENPP1 or ENPP1-Fc to determine the effect of ENPP1 or ENPP1-Fc on vascular smooth muscle cell proliferation in the allografts. ENPP1 or ENPP1-Fc treatment (ENPP1 or ENPP1-Fc at 10 mg/kg body weight subcutaneously injected every day) is initiated after the aortic transplant in the experimental mice group and continued for 28 days until the transplanted aorta is harvested. Similarly, the control mice group are treated with Tris buffered saline, pH 7.4 after aortic transplant by subcutaneous injection every day continued for 28 days until the transplanted aorta is harvested. The arteries are then fixed with 4% paraformaldehyde in PBS for morphological analyses.

Serial sections (sections of 5 μm each) are collected. 5-μm thick frozen aortic sections (Microtom, HM 500 O) are randomly chosen from various intervals throughout the transplanted grafts and are stained by using Elastica van Gieson stain (Roth, Karlsruhe, Germany). ImageJ software is used to measure the circumference of the external elastic lamina, the internal elastic lamina and the luminal border. Afterward, ImageJ (Fiji version 1.51p, NIH, USA) is used to measure neointimal and medial areas with two investigators blinded toward the treatment regimen. The ratio of the two analyzed parameters is used as a measure of lumen obstruction. The medial area, the intimal area and the intima/media ratio (I/M ratio) were calculated.

Statistical analyses are performed using Student's t test (unpaired two-sample testing for means). Comparisons of multiple groups used one-way ANOVA, followed by the Bonferroni's post hoc test, are performed with GraphPad Prism software version 7. Probability values of p<0.05 are considered significant. Morphometric analysis shows that control non-treated mice developed vascular lesions and intense remodeling, accompanied by high degrees of vessel lumen obstruction.

In experimental mice treated with ENPP1 or ENPP1-Fc post transplantation, the degree of intimal hyperplasia is compared to control mice which received no ENPP1 or ENPP1-Fc. Quantitative analyses of sequential sections of transplanted aorta from untreated control mice are expected to exhibit significantly increased neointimal proliferation and this also is compared to ENPP1 or ENPP1-Fc treated mice at or after 28 days post-transplant. Control mice are expected to show thickening of arterial intima and this is compared to treated mice. Correspondingly, the I/M ratios of control and treated mice are compared.

### Example 2: Prophylactic Effect of ENPP1-Fc

The same experiment as described in Example 1 is modified to determine the prophylactic effect of ENPP1 or ENPP1-Fc in preventing or reducing allograft vasculopathy by administering ENPP1 or ENPP1-Fc to the experimental group one week prior to aortic transplantation, as shown in FIG. 1. Likewise, the control group is administered Tris buffered saline a week prior to the aortic transplant. The process is then repeated as above with the experimental group after the transplant being treated with 10 mg/kg dosage of ENPP1 or ENPP1-Fc and control group being treated with Tris buffered saline post-transplant. Morphological analysis is expected to show that the intimal area of experimental mice receiving subcutaneous ENPP1 or ENPP1-Fc is expected to be significantly reduced compared to control mice, whereas the medial area, between the external and internal lamina remains constant. The I/M ratio shows a statistically significant decrease in ENPP1 or ENPP1-Fc treated experimental mice compared to vehicle-treated control mice indicating that the prophylactic treatment of ENPP1 or ENPP1-Fc prior to aortic transplant exhibits a protective effect by lowering the level of VSMC proliferation.

### Example 3: Rat Model of Aortic Allograft

The same experiment as described in Example 1 can be performed using a rat model instead of a mouse model. A rat model for transplantation is described in Bogossian et al. (2016) Cardiovasc Ther 34(4):183. ENPP1 or ENPP1-Fc treated rats and control rats (receiving Tris buffer saline) having aortic allograft transplants are compared at 28 days after transplant surgery.

### Example 4: Efficacy of ENPP1 or ENPP1-Fc Fusion Protein in Cardiac Allograft Vasculopathy (CAV) in Swine Heart Transplant Model

The selection of donor-recipient pairs is based upon major histocompatibility complex incompatibility by mixed lymphocyte reaction (MLR). The stimulation index (SI) is calculated through the following formula: (mean cpm of allogeneic MLR)/(mean cpm of autologous MLR). The donor heart is heterotopically transplanted into the recipient swine abdomen by infrarenal allografting. The selected transplant donors and recipients are anesthetized using Zoletil (tiletamine plus zolazepam, 5 mg/kg), succinylcholine (1.1 mg/kg), and atropine (0.6 mg/kg,), and they are maintained under anesthesia using isoflurane (3%/1.5 L/min) administered through a ventilator after intubation. The recipient is placed in the left decubitus position, and vascular access was established for the administration of immunosuppressive drugs.

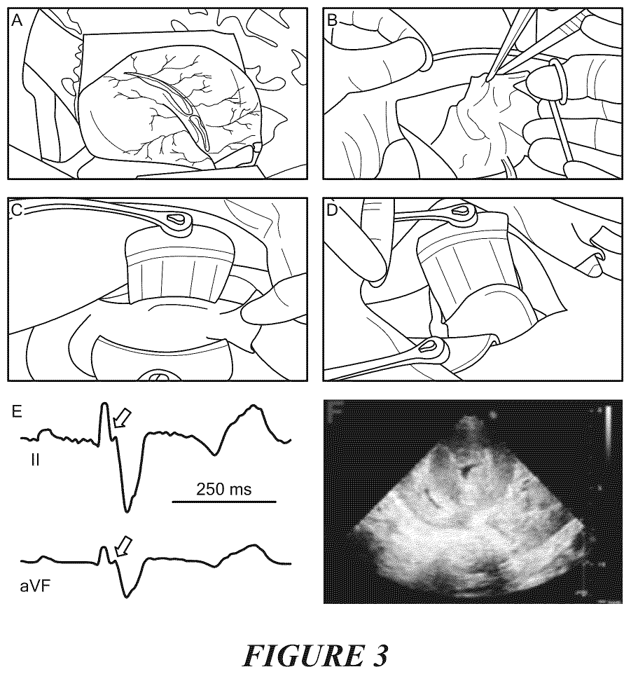

A right flank incision is created, and through a retroperitoneal approach, the infrarenal aorta and inferior vena cava are isolated (See FIG. 3). Next, the donor is heparinized (300 IU/kg intravenous injection (i.v.), and the donor heart is harvested after cardiac standstill is achieved using cold (4° C.) cardioplegic solution. An atrial septal defect was created in each donor heart, and the mitral valve is defunctionalized to minimize left ventricular atrophy and intracavitary thrombus formation. The recipient is heparinized (300 IU/kg i.v.), and the donor's pulmonary artery is anastomosed end-to-side to a 1 to 2 cm venotomy in the inferior vena cava with a continuous 5-0 polypropylene suture. Subsequently, the ascending aorta of the donor heart is anastomosed to the recipient's abdominal aorta in a similar manner, followed by the administration of protamine (1.5 mg/kg;) to stop bleeding. (Hsu et al., Transplantation. 2018 December; 102(12): 2002-2011.)

The beating rate of cardiac allograft was monitored daily through palpation, and electrocardiography is performed twice per week. When the beating rate of the allograft decreased, echocardiography is performed to assess systolic function. Follow-up is continued to the time of allograft arrest or the study end date (150 days).

A control subset of recipient pigs containing the transplanted heart (n=12) is treated with tris buffered saline and the experimental subset of recipient pigs with the transplanted heart (n=12) is treated with ENPP1 or ENPP1-Fc to determine the effect of ENPP1 on vascular smooth muscle cell proliferation in the solid organ transplants. ENPP1 or ENPP1-Fc treatment (ENPP1-Fc or ENPP1 at 10 mg/kg body weight subcutaneously injected every four days) is initiated after the heart transplant in the experimental pigs group and continued for 150 days until the transplanted heart is harvested. Similarly, the control pig group are treated with Tris buffered saline, pH 7.4 after heart transplant by intraperitoneal injection every 4 days is continued for 150 days until the transplanted heart is harvested.

Formalin-fixed cardiac specimens are embedded in paraffin, cross-sectioned, deparaffinized, rehydrated, and then subjected to hematoxylin and eosin (HE) or orcein staining. Intimal hyperplasia of the vascular grafts is examined using a Zeiss microscope and determined from computer images of orcein-stained cross sections. The area surrounded by the internal elastic lamina (IELA) and the luminal area (LA) are calculated using an image analysis program (Image J, Version 1.46r, NIH Image). The severity of intimal hyperplasia is calculated using the following formula: [(IELA−LA)/IELA]×100%. After calculation, the severity of intimal hyperplasia for each graft is evaluated in 3 randomly chosen fields per coronary section for 5 cross sections in a blinded manner, and the evaluated severity levels are averaged for statistical analysis.

Statistical analyses are performed using Student's t test (unpaired two-sample testing for means). Comparisons of multiple groups used one-way ANOVA, followed by the Bonferroni's post hoc test, are performed with GraphPad Prism software version 7. Probability values of p<0.05 are considered significant. Morphometric analysis shows that control non-treated pigs developed vascular lesions and intense remodeling, accompanied by high degrees of vessel lumen obstruction.

In experimental pigs treated with ENPP1 or ENPP1-Fc post transplantation, the degree of intimal hyperplasia is determined for control and ENPP1 or ENPP1-treated pigs by performing quantitative and qualitative analyses of sequential sections. Control pigs are expected to exhibit significantly increased neointimal proliferation at 150 days post-transplant. Control pigs are expected to show thickening of arterial intima and treated pigs are compared to control. Correspondingly, the I/M ratios of control and treated pigs are compared. Median survival time also is determined for the control and ENPP1 or ENPP1-treated groups. Graft survival time also is determined for control and ENPP1 or ENPP1-treated groups.

### Example 5: Efficacy of ENPP3 or ENPP3-Fc Fusion Protein in Mouse Aortic Allografts

Female DBA/2 (H-2^{d}) and C57BL/6J (H-2^{b}) mice ages of 5-6 weeks are used as donor and recipient mice respectively. (Bickerstaff et al, Murine renal allografts: spontaneous acceptance is associated with regulated T cell-mediated immunity, 2001, J. Immunol. 167, 4821-4827). Descending thoracic aortae of DBA/2 mice are transplanted into C57CL/6 mice in the infrarenal position, as already described. (Seppelt et al., Loss of Endothelial Barrier in Marfan Mice (mgR/mgR) Results in Severe Inflammation after Adenoviral Gene Therapy, 2016, PLoS ONE 11, e0148012.)

Donor mice are euthanized with CO₂. The thoracic cavity is opened, left ventricle is punctured, and the arterial circulatory system is perfused with 5 mL NaCl (4° C., 0.9%). The descending aorta is harvested and transplanted into the recipient mice to create the model for aortic allografts. Alternatively, the entire heart of the donor mice can be harvested and transplanted into the recipient mice as shown in FIG. 2 to create a solid organ transplant mouse model.

Recipient C57BL/6J mice are anesthetized by inhalation of 5% isoflurane. Novalgin (500 mg/mL; 200 mg/kg body weight) and Carprieve (50 mg/mL carprofen, 5 mg/kg body weight) are injected intraperitoneally. The abdominal cavity of recipient mice is opened and the infrarenal aorta was dissected. Titanium clips are applied, and the aorta was transected. Grafts are connected to recipient aorta with two end-to-end anastomoses (Prolene 11-0, nylon black, S&T, Neuhausen, Switzerland). After removal of the clips the graft was re-perfused. (Remes et al., Molecular Therapy: Methods & Clinical Development Vol. 15 Dec. 2019).

A control subset of recipient mice containing the transplanted aorta (n=5) is treated with tris buffered saline and the experimental subset of recipient mice with the transplanted aorta (n=5) is treated with ENPP3 or ENPP3-Fc to determine the effect of ENPP3-Fc on vascular smooth muscle cell proliferation in the allografts. ENPP3-Fc treatment (ENPP3-Fc at 10 mg/kg body weight subcutaneously injected every day) is initiated after the aortic transplant in the experimental mice group and continued for 28 days until the transplanted aorta is harvested. Similarly, the control mice group are treated with Tris buffered saline, pH 7.4 after aortic transplant by subcutaneous injection every day continued for 28 days until the transplanted aorta is harvested. The arteries are then fixed with 4% paraformaldehyde in PBS for morphological analyses.

Serial sections (sections of 5 μm each) are collected. 5-μm thick frozen aortic sections (Microtom, HM 500 O) are randomly chosen from various intervals throughout the transplanted grafts and are stained by using Elastica van Gieson stain (Roth, Karlsruhe, Germany). ImageJ software is used to measure the circumference of the external elastic lamina, the internal elastic lamina and the luminal border. Afterward, ImageJ (Fiji version 1.51p, NIH, USA) is used to measure neointimal and medial areas with two investigators blinded toward the treatment regimen. The ratio of the two analyzed parameters is used as a measure of lumen obstruction. The medial area, the intimal area and the intima/media ratio (UM ratio) were calculated.

Statistical analyses are performed using Student's t test (unpaired two-sample testing for means). Comparisons of multiple groups used one-way ANOVA, followed by the Bonferroni's post hoc test, are performed with GraphPad Prism software version 7. Probability values of p<0.05 are considered significant. Morphometric analysis shows that control non-treated mice developed vascular lesions and intense remodeling, accompanied by high degrees of vessel lumen obstruction.

In experimental mice treated with ENPP3 or ENPP3-Fc post transplantation, the degree of intimal hyperplasia is compared to control mice which received no ENPP3 or ENPP3-Fc. Quantitative analyses of sequential sections of transplanted aorta from untreated control mice are expected to exhibit significantly increased neointimal proliferation and this also is compared to ENPP3 or ENPP3-Fc treated mice at or after 28 days post-transplant. Control mice are expected to show thickening of arterial intima and this is compared to treated mice. Correspondingly, the TIM ratios of control and treated mice are compared.

### Example 6: Prophylactic Effect of ENPP3 or ENPP3-Fc

The same experiment as described in Example 5 is modified to determine the prophylactic effect of ENPP3 or ENPP3-Fc in preventing or reducing allograft vasculopathy by administering ENPP3 or ENPP3-Fc to the experimental group one week prior to aortic transplantation, as shown in FIG. 1. Likewise, the control group is administered Tris buffered saline a week prior to the aortic transplant. The process is then repeated as above with the experimental group after the transplant being treated with 10 mg/kg dosage of ENPP3 or ENPP3-Fc and control group being treated with Tris buffered saline post-transplant. Morphological analysis is expected to show that the intimal area of experimental mice receiving subcutaneous ENPP3 or ENPP3-Fc is expected to be significantly reduced compared to control mice, whereas the medial area, between the external and internal lamina remains constant. The FM ratio shows a statistically significant decrease in ENPP3 or ENPP3-Fc treated experimental mice compared to vehicle-treated control mice indicating that the prophylactic treatment of ENPP3 or ENPP3-Fc prior to aortic transplant exhibits a protective effect by lowering the level of VSMC proliferation.

### Example 7: Rat Model of Aortic Allograft

The same experiment as described in Example 5 can be performed using a rat model instead of a mouse model. A rat model for transplantation is described in Bogossian et al. (2016) Cardiovasc Ther 34(4):183. ENPP3 or ENPP3-Fc treated rats and control rats (receiving Tris buffer saline) having aortic allograft transplants are compared at 28 days after transplant surgery.

### Example 8: Efficacy of ENPP3-Fc Fusion Protein in Cardiac Allograft Vasculopathy (CAV) in Swine Heart Transplant Model

CAV remains the leading cause of allograft failure 1 year after transplantation. Cardiac allograft vasculopathy manifests as accelerated, diffuse coronary arteriosclerosis that has different pathogenesis than conventional native coronary artery disease (CAD). The efficacy of an ENPP3 or ENPP3-Fc fusion protein is evaluated in a large animal model of an organ transplant—specifically, heart transplant of domestic (Yorkshire) swine. Therapeutic effects of the ENPP3 or ENPP3-Fc fusion protein were assessed with respect to the ability to inhibit stenosis after heart transplant in Yorkshire swine.

The selection of donor-recipient pairs is based upon major histocompatibility complex incompatibility by mixed lymphocyte reaction (MLR). The stimulation index (SI) is calculated through the following formula: (mean cpm of allogeneic MLR)/(mean cpm of autologous MLR). The donor heart is heterotopically transplanted into the recipient swine abdomen by infrarenal allografting. The selected transplant donors and recipients are anesthetized using Zoletil (tiletamine plus zolazepam, 5 mg/kg), succinylcholine (1.1 mg/kg), and atropine (0.6 mg/kg,), and they are maintained under anesthesia using isoflurane (3%/1.5 L/min) administered through a ventilator after intubation. The recipient is placed in the left decubitus position, and vascular access was established for the administration of immunosuppressive drugs.

A right flank incision is created, and through a retroperitoneal approach, the infrarenal aorta and inferior vena cava are isolated (See FIG. 3). Next, the donor is heparinized (300 IU/kg intravenous injection (i.v.), and the donor heart is harvested after cardiac standstill is achieved using cold (4° C.) cardioplegic solution. An atrial septal defect was created in each donor heart, and the mitral valve is defunctionalized to minimize left ventricular atrophy and intracavitary thrombus formation. The recipient is heparinized (300 IU/kg i.v.), and the donor's pulmonary artery is anastomosed end-to-side to a 1 to 2 cm venotomy in the inferior vena cava with a continuous 5-0 polypropylene suture. Subsequently, the ascending aorta of the donor heart is anastomosed to the recipient's abdominal aorta in a similar manner, followed by the administration of protamine (1.5 mg/kg;) to stop bleeding. (Hsu et al., Transplantation. 2018 December; 102(12): 2002-2011.)

The beating rate of cardiac allograft was monitored daily through palpation, and electrocardiography is performed twice per week. When the beating rate of the allograft decreased, echocardiography is performed to assess systolic function. Follow-up is continued to the time of allograft arrest or the study end date (150 days).

A control subset of recipient pigs containing the transplanted heart (n=12) is treated with tris buffered saline and the experimental subset of recipient pigs with the transplanted heart (n=12) is treated with ENPP3 or ENPP3-Fc to determine the effect of ENPP3 polypeptides on vascular smooth muscle cell proliferation in the solid organ transplants. ENPP3 or ENPP3-Fc treatment (ENPP3 or ENPP3-Fc at 10 mg/kg body weight subcutaneously injected every four days) is initiated after the heart transplant in the experimental pigs group and continued for 150 days until the transplanted heart is harvested. Similarly, the control pig group are treated with Tris buffered saline, pH 7.4 after heart transplant by intraperitoneal injection every 4 days is continued for 150 days until the transplanted heart is harvested.

Formalin-fixed cardiac specimens are embedded in paraffin, cross-sectioned, deparaffinized, rehydrated, and then subjected to hematoxylin and eosin (HE) or orcein staining. Intimal hyperplasia of the vascular grafts is examined using a Zeiss microscope and determined from computer images of orcein-stained cross sections. The area surrounded by the internal elastic lamina (IELA) and the luminal area (LA) are calculated using an image analysis program (Image J, Version 1.46r, NIH Image). The severity of intimal hyperplasia is calculated using the following formula: [(IELA−LA)/IELA]×100%. After calculation, the severity of intimal hyperplasia for each graft is evaluated in 3 randomly chosen fields per coronary section for 5 cross sections in a blinded manner, and the evaluated severity levels are averaged for statistical analysis.

Statistical analyses are performed using Student's t test (unpaired two-sample testing for means). Comparisons of multiple groups used one-way ANOVA, followed by the Bonferroni's post hoc test, are performed with GraphPad Prism software version 7. Probability values of p<0.05 are considered significant. Morphometric analysis shows that control non-treated pigs developed vascular lesions and intense remodeling, accompanied by high degrees of vessel lumen obstruction.

In experimental pigs treated with ENPP3 or ENPP3-Fc post transplantation, the degree of intimal hyperplasia is determined for control and ENPP3-treated pigs by performing quantitative and qualitative analyses of sequential sections. Control pigs are expected to exhibit significantly increased neointimal proliferation at 150 days post-transplant. Control pigs are expected to show thickening of arterial intima and treated pigs are compared to control. Correspondingly, the TIM ratios of control and treated pigs are compared. Median survival time also is determined for the control and ENPP3-treated groups. Graft survival time also is determined for control and ENPP3-treated groups.

### Example 9: Efficacy of ENPP1-Fc Fusion Protein in In-Stent Restenosis Model

The efficacy of an ENPP1-Fc fusion protein was evaluated in large animal model of peripheral vascular injury—specifically, in-stent restenosis lesions in the peripheral vasculature of domestic (Yorkshire) swine. Therapeutic effects of the ENPP1-Fc fusion protein were assessed with respect to the ability to inhibit stenosis after angioplasty in previously injured and stented peripheral arteries of Yorkshire swine.

Four peripheral arterial sites were created for induction of neointimal response in each animal; one site was selected in each of four arteries (bilateral profunda and superficial femoral arteries).

All target sites were injured on Day 0 to create the in-stent restenosis model, 10 days prior to the first dose of ENPP1-Fc or a vehicle only control, and 14 days before repeat injury. The four peripheral artery sites were mapped using quantitative vascular angiography (QVA) in order to select the treatment site and correctly sized balloon and stent. The injury was created by overstretch of the artery with a standard angioplasty balloon catheter at a target 130% overstretch; three inflations were performed. Immediately following injury, a bare metal stent was deployed. Peripheral stents were self-expandable, targeting approximately a 120% overstretch.

ENPP1-Fc treatment occurred systemically starting on Day 10 and was dosed every 4 days subcutaneously until termination. On Day 14, all vessels were assessed by angiography and Optical Coherence Tomography (OCT). Then the previously injured and stented artery sites were subjected to a re-injury event consisting of overstretch of the artery with a single inflation of a standard angioplasty balloon catheter at the same pressure/diameter as the original pre-stent injury was done (130% of the baseline reference diameter). Following re-injury interventions, final post-procedural angiography and OCT were also recorded for select peripheral sites.

Four weeks following the re-injury event on Day 14, arteries underwent repeat imaging with angiography and endovascular imaging (OCT). The treated peripheral segments were explanted and stored in 10% neutral buffered formalin.

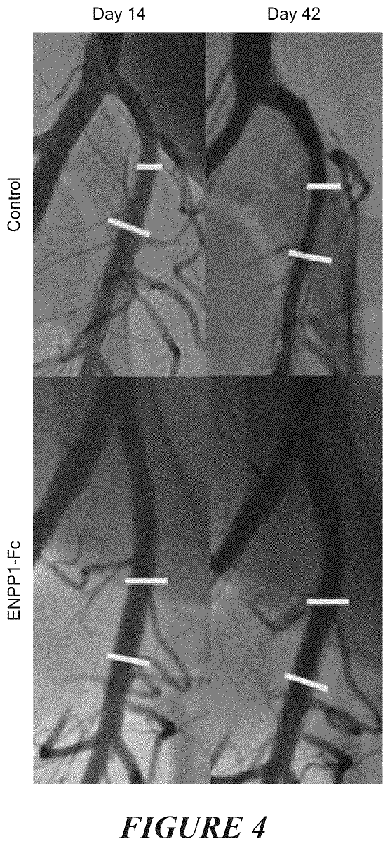

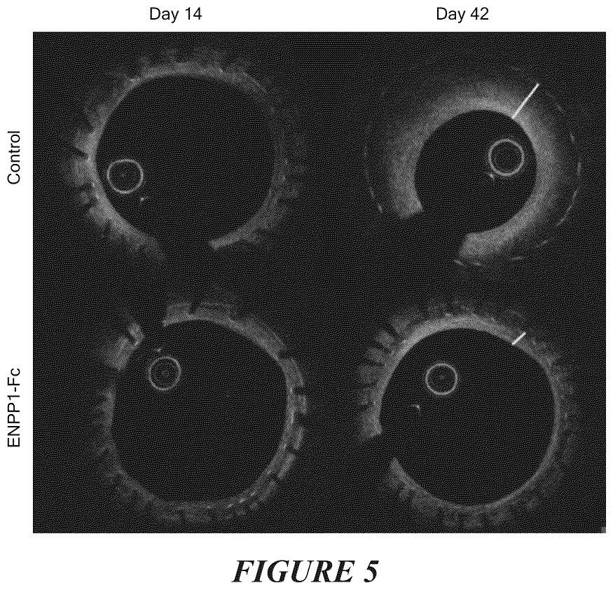

As shown in FIG. 4, angiography revealed a pronounced narrowing of the profunda at day 42 relative to the morphology of the vessel at day 14 in animals given the vehicle control. By contrast, in animals treated with ENPP1-Fc little visible change in profunda morphology was observed between day 14 and day 42. Similarly, as measured by OCT, pronounced intimal thickening was observed within the profunda at day 42 relative to the morphology of the vessel at day 14 in animals treated with the vehicle control. By contrast, little visible intimal thickening was observed between day 14 and day 42 in the profunda of animals treated with ENPP1-Fc (FIG. 5).

Tables 1 and 2 (below) summarizes the mean OCT values in all profunda arteries by treatment group.

TABLE 1

| | Day 14 Re-Injury, Profunda | | | | |
|---|---|---|---|---|---|
| | Lumen | Stent | Neointimal | Neointimal | % Area of |
| | Area (mm²) | Area (mm²) | Thickness (mm) | Area (mm²) | Stenosis |
| ENPP1-Fc | 12.10 ± 1.09 | 14.59 ± 1.24 | 0.19 ± 0.04 | 2.49 ± 0.52 | 17 ± 3 |
| Control | 10.82 ± 1.06 | 13.60 ± 0.82 | 0.23 ± 0.04 | 2.78 ± 0.40 | 21 ± 4 |

TABLE 2

| | Day 42 Termination, Profunda | | | | |
|---|---|---|---|---|---|
| | Lumen | Stent | Neointimal | Neointimal | % Area of |
| | Area (mm²) | Area (mm²) | Thickness (mm) | Area (mm²) | Stenosis |
| ENPP1-Fc | 12.95 ± 0.70 | 16.47 ± 0.89 | 0.27 ± 0.08 | 3.52 ± 1.07 | 21 ± 5 |
| Control | 9.51 ± 2.24 | 14.60 ± 1.40 | 0.45 ± 0.16 | 5.10 ± 1.34 | 35 ± 11 |

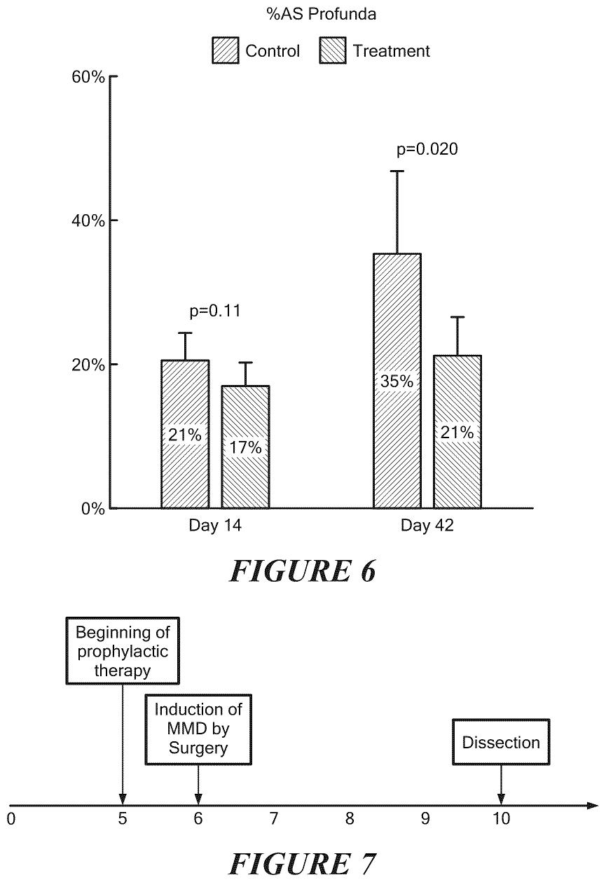

As set forth in the Table, the profunda arteries of animals treated with ENPP1-Fc had a higher lumen area at day 42 compared to the vehicle control group. The stent area was similar between both groups. Neointimal thickness and neointimal area were also reduced at day 42 in animals treated with ENPP1-Fc relative to the vehicle control animals. In additional, animals treated with ENPP1-Fc had a markedly lower % area of stenosis as compared to the vehicle control group (see FIG. 6). These data indicate that ENPP1 polypeptides are useful for, among other things, inhibiting the intimal thickening associated with injury of and/or to peripheral vessels:

### Example 10: Efficacy of ENPP3-Fc Fusion Protein in In-Stent Restenosis Model

The efficacy of an ENPP3-Fc fusion protein is evaluated in a large animal model of peripheral vascular injury—specifically, in-stent restenosis lesions in the peripheral vasculature of domestic (Yorkshire) swine. Therapeutic effects of the ENPP3-Fc fusion protein are assessed with respect to the ability to inhibit stenosis after angioplasty in previously injured and stented peripheral arteries of Yorkshire swine.

Four peripheral arterial sites are created for induction of neointimal response in each animal; one site is selected in each of four arteries (bilateral profunda and superficial femoral arteries).

All target sites are injured on Day 0 to create the in-stent restenosis model, 10 days prior to the first dose of ENPP3-Fc or a vehicle only control, and 14 days before repeat injury. The four peripheral artery sites are mapped using quantitative vascular angiography (QVA) in order to select the treatment site and correctly sized balloon and stent. The injury is created by overstretch of the artery with a standard angioplasty balloon catheter at a target 130% overstretch; three inflations are performed. Immediately following injury, a bare metal stent is deployed. Peripheral stents are self-expandable, targeting approximately a 120% overstretch.

ENPP3-Fc treatment will be systemically starting on Day 10 and dosed every 4 days subcutaneously until termination. On Day 14, all vessels are assessed by angiography and Optical Coherence Tomography (OCT). Then the previously injured and stented artery sites are subjected to a re-injury event consisting of overstretch of the artery with a single inflation of a standard angioplasty balloon catheter at the same pressure/diameter as the original pre-stent injury (130% of the baseline reference diameter). Following re-injury interventions, final post-procedural angiography and OCT are also recorded for select peripheral sites.

Four weeks following the re-injury event on Day 14, arteries will be subject to repeat imaging with angiography and endovascular imaging (OCT). The treated peripheral segments will be explanted and stored in 10% neutral buffered formalin.

### Example 11: Efficacy of ENPP1 or ENPP1-Fc Fusion Protein in MMD Mouse Model

Moyamoya is a cerebrovascular disorder characterized by progressive stenosis of the intracranial internal carotid arteries leading to both hemorrhagic and ischemic strokes Restriction of blood flow through the ICA leads to eventual development of new blood vessels resembling a “puff of smoke” (moyamoya in Japanese) in the subcortical region. The aim of the example is to evaluate the efficacy of an ENPP1-Fc fusion protein or ENPP1 for treatment in a mouse model for MMD. Therapeutic effects of the ENPP1-Fc fusion protein or ENPP1 are assessed with respect to the ability to inhibit vascular smooth muscle cell proliferation in MMD and reduce or prevent cerebral occlusions.

Generation of MMD Phenotype

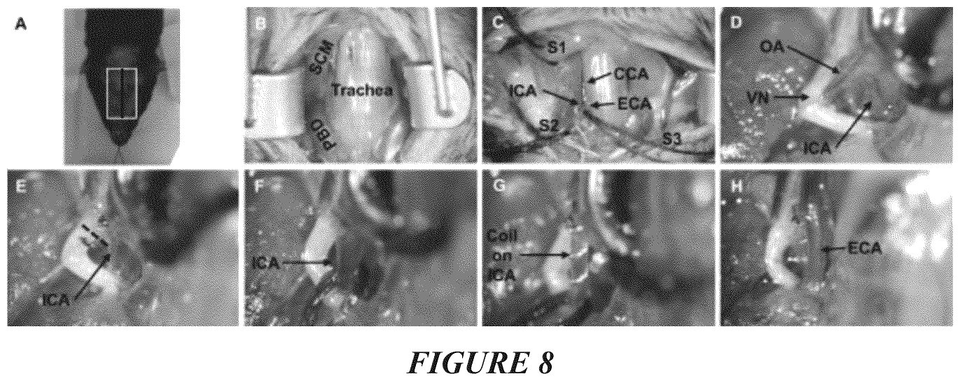

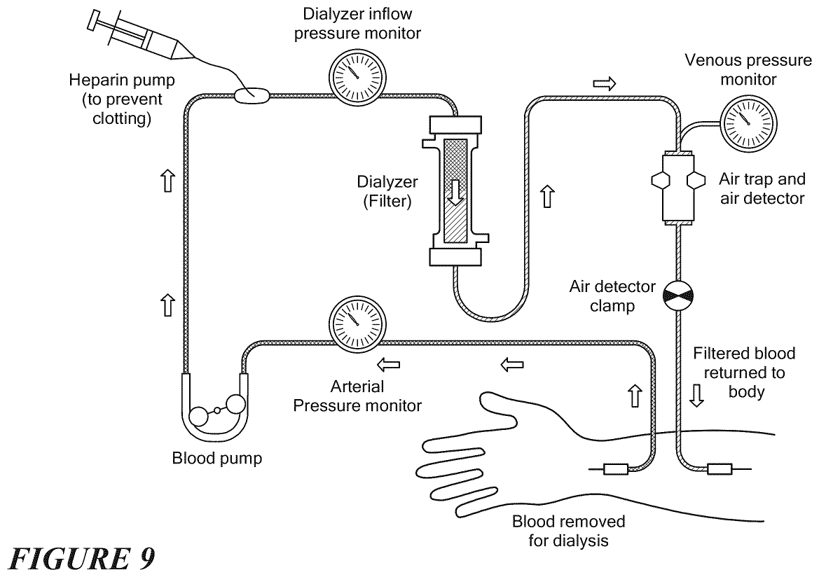

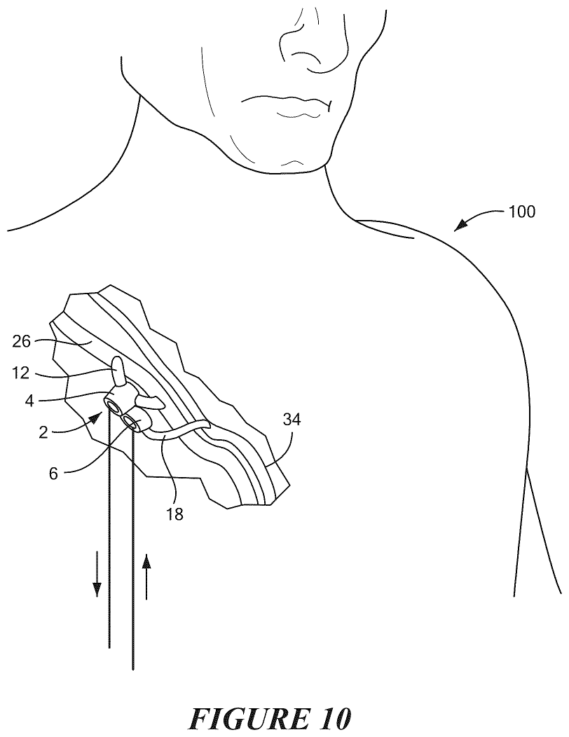

C57Bl/6 male mice (5-6 weeks old) obtained from Jackson Laboratories are anesthetized with a cocktail of ketamine and xylazine using a weight based ratio. Once the mice are anesthetized their cervical region are shaved, and the mouse is placed in the supine position with their head, forepaws and tail restrained (FIG. 8). With the mouse in the supine position, the shaved area is cleaned with alcohol and betadine. A midline incision is made from the angle of the mandible to the sternum exposing the trachea, common carotid artery (CCA) and bifurcation of the CCA into the internal carotid and external carotid artery (ICA/ECA). A retractor is used to hold the skin and separated salivary glands from impeding the surgical area. To increase the visual field, the sternocleidomastoid (SCM) muscle and the posterior belly of the digastric (PBD) muscle are exposed inferiorly and superiorly, respectively. The tip of a pair of curved forceps is gently placed under the SCM medial to lateral and one length of 4±0 suture was transferred underneath. The suture is looped around the SCM and secured using tape. This procedure is repeated with the PBD.

With the ICA isolated, the 6±0 suture is used as an anchor for coil placement. Fine tipped forceps are used to grasp the coil at one end and place it at an angle to the ICA so that the vessel inserts into the last rung of the coil. With the vessel in the last rung of the coil, the coil is inverted so that it is parallel to the ICA. Using the 6±0 suture, the vessel is gently rotated around the coil so that a length of vessel is placed in each rung of the coil. Vessel placement is assessed to ensure that it is not skipping a rung; if so, the vessel is uncoiled and repositioned until the coil completely encompassed the vessel. Thus, the MMD phenotype in both control and experimental subset of mice is induced by following the procedures discussed in Roberts et al. ((Roberts et al., Internal carotid artery stenosis: A novel surgical model for moyamoya syndrome, PLoS One. 2018; 13(1): e0191312.)

A control subset of MMD model mice (n=5) is treated with tris buffered saline and the experimental subset of MMD mice (n=5) is treated with ENPP1 or ENPP1-Fc post induction of MMD phenotype to determine the effect of ENPP1 or ENPP1-Fc on vascular smooth muscle cell proliferation and cerebral occlusions in the brain of the MMD mice. ENPP1 or ENPP1-Fc treatment (ENPP1 or ENPP1-Fc at 10 mg/kg body weight, subcutaneously injected every day) is initiated after the induction of MMD phenotype by surgery as described above in the experimental mice group and ENPP1 or ENPP1-Fc administration is continued for 28 days until the cerebral artery is harvested.

Similarly, the control mice group are treated with Tris buffered saline, pH 7.4 after the induction of MMD phenotype by surgery as described and Tris buffered saline is administered via subcutaneous injection every day and continued for 28 days until the cerebral artery is harvested. The arteries of both control and experimental group mice with MMD are then fixed with 4% paraformaldehyde in PBS for morphological analyses.

Visualization of Cerebrovasculature

To visualize the cerebrovasculature, all animals from each group are perfused with the fluorescent dye Di I (Li et al., Direct labeling and visualization of blood vessels with lipophilic carbocyanine dye Dd. Nat Protoc. 2008; 3(11):1703±8). Mice are subjected to a transcardial perfusion using a perfusion pump (set to 1 ml/min) to perfuse (room temperature) 5 ml of PBS, immediately followed by 10 ml of Di I working solution and then 10 ml of 10% buffered formalin. Brains are carefully removed from the skull ensuring that the Circle of Willis (CoW) remained intact.

The extracted brain is then post-fixed overnight at 4° C. with 10% buffered formalin. The brains are then transferred into PBS for long-term storage at 4° C. and protected from light. Fluorescently labelled brains were imaged using a 1×microscope (Nikon Eclipse E800/Nikon DS-Ril). Images of the cortical vasculature are taken to examine anastomoses and images of the CoW were used to measure vessel diameter. Image analysis is performed using Nikon NES Analysis software to measure vessel diameter (μm).

Diameter measurements are taken approximately 20 μm from the bifurcation of the supraclinoid internal carotid artery, M1 segment of the middle cerebral artery, and the A1 segment of the anterior cerebral artery. Anastomoses analysis is performed by counting the number of anastomoses (circle placed over each connection point on a magnified image) between the ACA and the MCA of both the ipsilateral and contralateral hemispheres. Diameters of the ICA, ACA and MCA vessels are examined by measuring the width of each vessel near the bifurcation point on both the ipsilateral and contralateral sides to determine if there was any difference in size between the experimental and control groups.

Morphological Analysis

Serial sections (sections of 5 μm each) of cerebral arteries such as MCA, ACA and ICA are collected for both control and experimental group. 5-μm thick frozen aortic sections (Microtome, HM 500 O) are stained by using Elastica van Gieson stain (Roth, Karlsruhe, Germany). ImageJ software is used to measure the circumference of the external elastic lamina, the internal elastic lamina and the luminal border. Afterward, ImageJ (Fiji version 1.51p, NIH, USA) is used to measure neointimal and medial areas with two investigators blinded toward the treatment regimen. The ratio of the two analyzed parameters is used as a measure of lumen obstruction. The medial area, the intimal area and the intima/media ratio (FM ratio) were calculated. (See FIG. 2).

Statistical analyses are performed using Student's t test (unpaired two-sample testing for means). Comparisons of multiple groups used one-way ANOVA, followed by the Bonferroni's post hoc test, are performed with GraphPad Prism software version 7. Probability values of p<0.05 are considered significant. Morphometric analysis shows that the control non-treated mice with MMD phenotype develop vascular lesions, occlusions and narrowing accompanied by high degrees of vessel lumen obstruction.

In experimental mice treated with ENPP1 or ENPP1-Fc post induction of MMD phenotype, the degree of intimal hyperplasia is compared to control mice which received no ENPP1 or ENPP1-Fc. Quantitative analyses of cerebral arteries from untreated control mice with MMD phenotype are expected to exhibit significantly increased neointimal proliferation and this also is compared to ENPP1 or ENPP1-Fc treated mice at or after 28 days post-surgery. Control mice are expected to exhibit thickening of arterial intima and this is compared to treated mice. Correspondingly, the I/M ratios of control and treated mice are also compared.

### Example 12: Prophylactic Effect of ENPP1 or ENPP1-Fc

The same experiment as described in Example 11 is modified to determine the prophylactic effect of ENPP1 or ENPP1-Fc in preventing or reducing vascular smooth muscle proliferation and cerebral occlusions by administering ENPP1 or ENPP1-Fc to the experimental group one week prior to induction of MMD phenotype, as shown in FIG. 7. Likewise, the control group is administered Tris buffered saline a week prior to induction of MMD phenotype. The process is then repeated as above with the experimental group after surgery being treated with 10 mg/kg dosage of ENPP1 or ENPP1-Fc and control group being treated with Tris buffered saline post-surgery.

Morphological analysis is expected to show that the intimal area of experimental mice with MMD phenotype receiving subcutaneous ENPP1 or ENPP1-Fc is expected to be significantly reduced compared to control mice, whereas the medial area, between the external and internal lamina remains constant. The I/M ratio is expected to decrease in ENPP1 or ENPP1-Fc treated experimental mice compared to vehicle-treated control mice. The prophylactic treatment of ENPP1 or ENPP1-Fc prior to induction of MMD phenotype is expected to confer protective effect by lowering the level of VSMC proliferation.

### Example 13: Efficacy of ENPP3 or ENPP3-Fc Fusion Protein in a Mouse Model of MMD

Moyamoya is a cerebrovascular disorder characterized by progressive stenosis of the intracranial internal carotid arteries leading to both hemorrhagic and ischemic strokes Restriction of blood flow through the ICA leads to eventual development of new blood vessels resembling a “puff of smoke” (moyamoya in Japanese) in the subcortical region. The aim of the example is to evaluate the efficacy of an ENPP3-Fc fusion protein or ENPP3 for treatment in a mouse model for MMD. Therapeutic effects of the ENPP3-Fc fusion protein or ENPP3 are assessed with respect to the ability to inhibit vascular smooth muscle cell proliferation in MMD and reduce or prevent cerebral occlusions.

Generation of MMD Phenotype

C57Bl/6 male mice (5-6 weeks old) obtained from Jackson Laboratories are anesthetized with a cocktail of ketamine and xylazine using a weight based ratio. Once the mice are anesthetized their cervical region are shaved, and the mouse is placed in the supine position with their head, forepaws and tail restrained (FIG. 8). With the mouse in the supine position, the shaved area is cleaned with alcohol and betadine. A midline incision is made from the angle of the mandible to the sternum exposing the trachea, common carotid artery (CCA) and bifurcation of the CCA into the internal carotid and external carotid artery (ICA/ECA). A retractor is used to hold the skin and separated salivary glands from impeding the surgical area. To increase the visual field, the sternocleidomastoid (SCM) muscle and the posterior belly of the digastric (PBD) muscle are exposed inferiorly and superiorly, respectively. The tip of a pair of curved forceps is gently placed under the SCM medial to lateral and one length of 4±0 suture was transferred underneath. The suture is looped around the SCM and secured using tape. This procedure is repeated with the PBD.

With the ICA isolated, the 6±0 suture is used as an anchor for coil placement. Fine tipped forceps are used to grasp the coil at one end and place it at an angle to the ICA so that the vessel inserts into the last rung of the coil. With the vessel in the last rung of the coil, the coil is inverted so that it is parallel to the ICA. Using the 6±0 suture, the vessel is gently rotated around the coil so that a length of vessel is placed in each rung of the coil. Vessel placement is assessed to ensure that it is not skipping a rung; if so, the vessel is uncoiled and repositioned until the coil completely encompassed the vessel. Thus, the MMD phenotype in both control and experimental subset of mice is induced by following the procedures discussed in Roberts et al. ((Roberts et al., Internal carotid artery stenosis: A novel surgical model for moyamoya syndrome, PLoS One. 2018; 13(1): e0191312.)

A control subset of MMD model mice (n=5) is treated with tris buffered saline and the experimental subset of MMD mice (n=5) is treated with ENPP3-Fc or ENPP3 post induction of MMD phenotype to determine the effect of ENPP3-Fc or ENPP3 on vascular smooth muscle cell proliferation and cerebral occlusions in the brain of the MMD mice. ENPP3-Fc treatment (ENPP3 or ENPP3-Fc at 10 mg/kg body weight, subcutaneously injected every day) is initiated after the induction of MMD phenotype by surgery as described above in the experimental mice group and ENPP3-Fc or ENPP3 administration is continued for 28 days until the cerebral artery is harvested.

Similarly, the control mice group are treated with Tris buffered saline, pH 7.4 after the induction of MMD phenotype by surgery as described and Tris buffered saline is administered via subcutaneous injection every day and continued for 28 days until the cerebral artery is harvested. The arteries of both control and experimental group mice with MMD are then fixed with 4% paraformaldehyde in PBS for morphological analyses.

Visualization of Cerebrovasculature

To visualize the cerebrovasculature, all animals from each group are perfused with the fluorescent dye Di I (Li et al., Direct labeling and visualization of blood vessels with lipophilic carbocyanine dye DiI. Nat Protoc. 2008; 3(11): 1703±8). Mice are subjected to a transcardial perfusion using a perfusion pump (set to 1 ml/min) to perfuse (room temperature) 5 ml of PBS, immediately followed by 10 ml of Di I working solution and then 10 ml of 10% buffered formalin. Brains are carefully removed from the skull ensuring that the Circle of Willis (CoW) remained intact.

The extracted brain is then post-fixed overnight at 4° C. with 10% buffered formalin. The brains are then transferred into PBS for long-term storage at 4° C. and protected from light. Fluorescently labelled brains were imaged using a 1×microscope (Nikon Eclipse E800/Nikon DS-Ril). Images of the cortical vasculature are taken to examine anastomoses and images of the CoW were used to measure vessel diameter. Image analysis is performed using Nikon NES Analysis software to measure vessel diameter (μm).

Diameter measurements are taken approximately 20 μm from the bifurcation of the supraclinoid internal carotid artery, M1 segment of the middle cerebral artery, and the A1 segment of the anterior cerebral artery. Anastomoses analysis is performed by counting the number of anastomoses (circle placed over each connection point on a magnified image) between the ACA and the MCA of both the ipsilateral and contralateral hemispheres. Diameters of the ICA, ACA and MCA vessels are examined by measuring the width of each vessel near the bifurcation point on both the ipsilateral and contralateral sides to determine if there was any difference in size between the experimental and control groups.

Measurements of the distal ICA and proximal ACA in control mice with MMD phenotype are expected to exhibit severe narrowing of vessel diameter post-surgery, and this is compared with the vessel diameter of ENPP3 or ENPP3-Fc treated mice with MMD phenotype.

Morphological Analysis

Serial sections (sections of 5 μm each) of cerebral arteries such as MCA, ACA and ICA are collected for both control and experimental group. 5-μm thick frozen aortic sections (Microtome, HM 500 O) are stained by using Elastica van Gieson stain (Roth, Karlsruhe, Germany). ImageJ software is used to measure the circumference of the external elastic lamina, the internal elastic lamina and the luminal border. Afterward, ImageJ (Fiji version 1.51p, NIH, USA) is used to measure neointimal and medial areas with two investigators blinded toward the treatment regimen. The ratio of the two analyzed parameters is used as a measure of lumen obstruction. The medial area, the intimal area and the intima/media ratio (FM ratio) were calculated. (See FIG. 2).

Statistical analyses are performed using Student's t test (unpaired two-sample testing for means). Comparisons of multiple groups used one-way ANOVA, followed by the Bonferroni's post hoc test, are performed with GraphPad Prism software version 7. Probability values of p<0.05 are considered significant. Morphometric analysis shows that the control non-treated mice with MMD phenotype develop vascular lesions, occlusions and narrowing accompanied by high degrees of vessel lumen obstruction.

In experimental mice treated with ENPP3 or ENPP3-Fc post induction of MMD phenotype, the degree of intimal hyperplasia is compared to control mice which received no ENPP3 or ENPP3-Fc. Quantitative analyses of cerebral arteries from untreated control mice with MMD phenotype are expected to exhibit significantly increased neointimal proliferation and this also is compared to ENPP3 or ENPP3-Fc treated mice at or after 28 days post-surgery. Control mice are expected to exhibit thickening of arterial intima and this is compared to treated mice. Correspondingly, the I/M ratios of control and treated mice are also compared.

### Example 14: Prophylactic Effect of ENPP3 or ENPP3-Fc

The same experiment as described in Example 13 is modified to determine the prophylactic effect of ENPP3 or ENPP3-Fc in preventing or reducing vascular smooth muscle proliferation and cerebral occlusions by administering ENPP3 or ENPP3-Fc to the experimental group one week prior to induction of MMD phenotype, as shown in FIG. 7. Likewise, the control group is administered Tris buffered saline a week prior to induction of MMD phenotype. The process is then repeated as above with the experimental group after surgery being treated with 10 mg/kg dosage of ENPP3 or ENPP3-Fc and control group being treated with Tris buffered saline post-surgery.

Morphological analysis is expected to show that the intimal area of experimental mice with MMD phenotype receiving subcutaneous ENPP3 or ENPP3-Fc is expected to be significantly reduced compared to control mice, whereas the medial area, between the external and internal lamina remains constant. The I/M ratio is expected to decrease in ENPP3 or ENPP3-Fc treated experimental mice compared to vehicle-treated control mice. The prophylactic treatment of ENPP3 or ENPP3-Fc prior to induction of MMD phenotype is expected to confer protective effect by lowering the level of VSMC proliferation.

### Example 15: Efficacy of ENPP1 or ENPP1-Fc Fusion Protein in a Mouse Model of AV Fistula

Failure

The efficacy of an ENPP1 or ENPP1-Fc fusion protein is evaluated in a mouse model of arterio-venous fistula failure as described in, e.g., Wong et al. (2014) J Vasc Surg 59:192-201. Unilateral AVFs are created between the external jugular vein and common carotid artery in male C57bl6 mice. The mice are divided into four cohorts: (1) mice that receive chronic subcutaneous treatment with an ENPP1-Fc fusion protein or ENPP1 prior to and after the AVF is created; (2) mice that receive a vehicle control treatment subcutaneously prior to and after the AVF is created; (3) mice that begin chronic subcutaneous treatment with an ENPP1-Fc fusion protein or ENPP1 following AVF creation; and (4) mice that receive a vehicle control treatment subcutaneously after the AVF creation.

The mice are followed over time and euthanized at various time points (such as one, two, and/or three weeks after AVF creation). Histological analysis is performed on sections of blood vessels at or promixal to the site of AVF.

It is anticipated that the extent of intimal hyperplasia in the AVF adjacent vessels of mice treated with ENPP1-Fc fusion protein will be markedly reduced as compared to those mice receiving the vehicle control.

### Example 16: Efficacy of ENPP3 or ENPP3-Fc Fusion Protein in a Mouse Model of AV Fistula Failure

The efficacy of an ENPP3-Fc fusion protein or ENPP3 is evaluated in a mouse model of arterio-venous fistula failure as described in, e.g., Wong et al. (2014) J Vasc Surg 59:192-201. Unilateral AVFs are created between the external jugular vein and common carotid artery in male C57bl6 mice. The mice are divided into four cohorts: (1) mice that receive chronic subcutaneous treatment with an ENPP3-Fc fusion protein or ENPP3 prior to and after the AVF is created; (2) mice that receive a vehicle control treatment subcutaneously prior to and after the AVF is created; (3) mice that begin chronic subcutaneous treatment with an ENPP3-Fc fusion protein or ENPP3 following AVF creation; and (4) mice that receive a vehicle control treatment subcutaneously after the AVF creation.

The mice are followed over time and euthanized at various time points (such as one, two, and/or three weeks after AVF creation). Histological analysis is performed on sections of blood vessels at or proximal to the site of AVF.

It is anticipated that the extent of intimal hyperplasia in the AVF adjacent vessels of mice treated with ENPP3-Fc fusion protein will be markedly reduced as compared to those mice receiving the vehicle control.

### Example 17: Treatment of a Human Cardiac Transplant Patient Suffering from Cardiac Allograft Vasculopathy

A human adult heart allograft recipient is identified by a medical practitioner as having CAV. The recipient administers or is administered chronically a pharmaceutical composition comprising a fusion protein comprising a soluble form of ENPP1 fused to a Fc region. Medical professionals monitor the recipient over time for cessation of unwanted intimal proliferation in one or more vessels of the allografted heart and/or partial or full resolution over time of vessel occlusion in the allografted heart. Treatment with the fusion protein is expected to halt or substantially reduce unwanted intimal proliferation in one or more vessels of the allografted heart and/or partially or fully resolve over time vessel occlusion in the allografted heart.

In another example, a pharmaceutical composition comprising a fusion protein comprising a soluble form of ENPP1 fused to a Fc region is chronically administered to the recipient of a cardiac allograft beginning at or around the time of transplantation to prevent, lessen the likelihood of occurrence of, or reduce the extent of unwanted intimal proliferation in one or more vessels of the allografted heart. Medical professionals monitor the recipient over time for the presence and/or level of unwanted intimal proliferation in one or more vessels of the allografted heart. Treatment with the fusion protein is expected to halt or substantially reduce unwanted intimal proliferation in one or more vessels of the allografted heart.

### Example 18: Treatment of a Human Suffering from MoyaMoya Disease

A human adult patient is identified by a medical practitioner as having Moyamoya disease. The recipient administers or is administered chronically a pharmaceutical composition comprising a fusion protein comprising a soluble form of ENPP1 fused to a Fc region. Medical professionals monitor the recipient over time for cessation of unwanted intimal proliferation in one or more vessels feeding the brain and/or partial or full resolution over time of the occlusion of such vessel or vessels. Treatment with the fusion protein is expected to halt or substantially reduce unwanted intimal proliferation in one or more vessels and/or partially or fully resolve over time vessel occlusion.

### Example 19: Treatment of a Dialysis Patient Who has Received a Hemodialysis Shunt

A pharmaceutical composition comprising a fusion protein comprising a soluble form of ENPP1 fused to a Fc region is chronically administered to a hemodialysis patient at or around the time that a hemodialysis shunt is placed in the subject to thereby prevent, lessen the likelihood of occurrence of, or reduce the extent of unwanted intimal proliferation in one or more vessels connected to or involved in the shunt. Medical professionals monitor the recipient over time for the presence and/or level of unwanted intimal proliferation in one or more of the vessels. Treatment with the fusion protein is expected to halt or substantially reduce unwanted intimal proliferation in one or more of the vessels.

### INCORPORATION BY REFERENCE

The disclosure of each and every U.S. and foreign patent and pending patent application and publication referred to herein is specifically incorporated herein by reference in its entirety, as are the contents of Sequence Listing and Figures.

### EQUIVALENTS

Those skilled in the art will recognize or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

### Other Embodiments

From the foregoing description, it will be apparent that variations and modifications may be made to the disclosure described herein to adopt it to various usages and conditions, including the use of different signal sequences to express functional variants of ENPP1 or ENPP3 or combinations thereof in different viral vectors having different promoters or enhancers or different cell types known in art to treat any diseases characterized by the presence of pathological calcification or ossification are within the scope according to the disclosure. Other embodiments according to the disclosure are within the following claims.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

Other embodiments are within the following claims.

1-155. (canceled)

156. A method for treating a subject at risk for developing Moyamoya disease or a subject afflicted with Moyamoya disease, the method comprising: administering to the subject an ENPP1 agent in an amount sufficient to thereby treat the subject.

157. The method of claim 156, wherein the ENPP1 agent comprises an ENPP1 polypeptide or a nucleic acid encoding an ENPP1 polypeptide or a viral vector comprising a nucleic acid encoding an ENPP1 polypeptide.

158. The method of claim 157, wherein the ENPP1 polypeptide comprises the extracellular domain of ENPP1.

159. The method of claim 157, wherein the ENPP1 polypeptide comprises amino acids 99 to 925 of SEQ ID NO: 1.

160. The method of claim 157, wherein the ENPP1 polypeptide comprises a heterologous protein and said heterologous protein increases the circulating half-life of the ENPP1 polypeptide in mammal.

161. The method of claim 160, wherein the heterologous protein is an Fc region of an immunoglobulin molecule or an albumin molecule.

162. The method of claim 160, wherein the heterologous protein is carboxy-terminal to the ENPP1 polypeptide.

163. The method of claim 160, wherein the ENPP1 agent comprises a linker and the linker separates the ENPP1 polypeptide and the heterologous protein.

164. The method of claim 160, wherein the ENPP1 agent is administered to the subject subcutaneously or intravenously.

165. The method of claim 160, wherein the ENPP1 agent comprises ENPP1 variants that retain enzymatic activity.

166. A method for inhibiting or preventing cerebral vascular occlusion or unwanted vascular smooth muscle cell proliferation in a subject at risk for developing Moyamoya disease or is afflicted with Moyamoya disease, the method comprising: administering to the subject an ENPP1 agent in an amount sufficient to thereby inhibit or prevent cerebral vascular occlusion or unwanted vascular smooth muscle cell proliferation in the subject.

167. The method of claim 166, wherein said subject is one who is expected to receive or who has received a surgical intervention as a treatment for Moyamoya disease.

168. The method of claim 166, wherein the subject bears the RNF213 R4810K mutation.

169. The method of claim 166, wherein said subject experiences stenosis, thrombosis, embolism and/or hemorrhage in the brain.

170. The method of claim 166, wherein the surgical intervention is a vascular bypass graft or cerebral revascularization.

171. The method of claim 170, wherein the ENPP1 agent or the ENPP3 agent is administered to the subject prior to the surgical intervention or concurrently with the surgical intervention or following the surgical intervention.

172. The method of claim 166, wherein the ENPP1 agent comprises an ENPP1 polypeptide or a nucleic acid encoding an ENPP1 polypeptide or a viral vector comprising a nucleic acid encoding an ENPP1 polypeptide.

173. The method of claim 172, wherein the ENPP1 polypeptide comprises the extracellular domain of ENPP1.

174. The method of claim 172, wherein the ENPP1 polypeptide comprises a heterologous protein and said heterologous protein increases the circulating half-life of the ENPP1 polypeptide in mammal.

175. The method of claim 174, wherein the heterologous protein is an Fc region of an immunoglobulin molecule or an albumin molecule.

176. The method of claim 174, wherein the ENPP1 agent comprises a linker and the linker separates the ENPP1 polypeptide and the heterologous protein.

177. The method of claim 174, wherein the ENPP1 agent is administered to the subject subcutaneously or intravenously.

178. The method of claim 174, wherein the ENPP1 agent comprises ENPP1 variants that retain enzymatic activity.

179. A method for reducing and/or preventing allograft vasculopathy in a subject having an allograft, the method comprising administering to the subject an effective amount of an ENPP1 agent to thereby reduce and/or prevent allograft vasculopathy in said subject.

180. The method of claim 179, wherein the ENPP1 agent is administered to the subject prior to the surgical intervention or concurrently with the surgical intervention or following the surgical intervention.

181. The method of claim 179, wherein the ENPP1 agent comprises an ENPP1 polypeptide or a nucleic acid encoding an ENPP1 polypeptide or viral vector comprising a nucleic acid encoding an ENPP1 polypeptide.

182. The method of claim 181, wherein the ENPP1 polypeptide comprises the extracellular domain of ENPP1.

183. The method of claim 181, wherein the ENPP1 polypeptide comprises a heterologous protein and said heterologous protein increases the circulating half-life of the ENPP1 polypeptide in mammal.

184. The method of claim 183, wherein the heterologous protein is an Fc region of an immunoglobulin molecule or an albumin molecule.

185. The method of claim 184, wherein the ENPP1 agent is administered to the subject subcutaneously or intravenously.

186. The method of claim 184, wherein the ENPP1 agent comprises ENPP1 variants that retain enzymatic activity.

187. A method for reducing and/or preventing progression of vascular smooth muscle cell proliferation in a subject's peripheral vessel at or around the site at which an arterio-venous dialysis shunt has been placed, the method comprising: administering to the subject an effective amount of an ENPP1 agent to thereby reduce and/or prevent progression of vascular smooth muscle cell proliferation in said peripheral vessel at or around the site the arterio-venous dialysis shunt has been placed.

188. The method of claim 187, wherein the agent is administered prior to, during and/or after said surgery or said shunt placement.

189. The method of claim 187, wherein the ENPP1 agent comprises an ENPP1 polypeptide or a nucleic acid encoding an ENPP1 polypeptide or a viral vector comprising a nucleic acid encoding an ENPP1 polypeptide.

190. The method of claim 187, wherein the ENPP1 polypeptide comprises a heterologous protein and said heterologous protein increases the circulating half-life of the ENPP1 polypeptide in mammal.

191. The method of claim 190, wherein the heterologous protein is an Fc region of an immunoglobulin molecule or an albumin molecule.


SEQUENCE LISTING (79 sequences)

SEQ ID NO: 1 - protein, 925 aa, synthetic construct
  REGION 1..925: note = ENPP1 Amino Acid Sequence - Wild Type
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 1077 aa, synthetic construct
  REGION 1..1077: note = Azurocidin-ENPP1-FC
  SIGNAL 19..20: note = Cleavage point of the signal sequence
  REGION 851..1077: note = MISC_FEATURE - Indicate Fc sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 1468 aa, synthetic construct
  REGION 1..1468: note = Azurocidin-ENPP1-Alb
  SIGNAL 19..20: note = Cleavage point of the signal sequence
  REGION 851..1468: note = MISC_FEATURE - Indicate Albumin sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 4 - protein, 1594 aa, synthetic construct
  REGION 1..1594: note = Azurocidin-ENPP1
  SIGNAL 19..20: note = Indicate cleavage point of signal sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 5 - protein, 888 aa, synthetic construct
  REGION 1..888: note = ENPP2 Amino Acid Sequence - Wild Type
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEAETRKFRGSRNENKENINGNFEPRKERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 6 - protein, 827 aa, synthetic construct
  REGION 1..827: note = Extracellular Domain of ENPP3
EKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 7 - protein, 875 aa, synthetic construct [sequence omitted]

SEQ ID NO: 8 - protein, 1073 aa, synthetic construct
  REGION 1..1073: note = Azurocidin-ENPP3-FC
  SIGNAL 19..20: note = Cleavage point of the signal sequence
  REGION 847..1073: note = MISC_FEATURE - Indicate Fc sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1464 aa, synthetic construct
  REGION 1..1464: note = Azurocidin-ENPP3-Albumin
  SIGNAL 19..20: note = Cleavage point of the signal sequence
  REGION 847..1464: note = MISC_FEATURE - Indicate Albumin sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 10 - protein, 846 aa, synthetic construct
  REGION 1..846: note = Azurocidin-ENPP3
  SIGNAL 19..20: note = Cleavage point of the signal sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 11 - protein, 453 aa, synthetic construct
  REGION 1..453: note = ENPP4 Amino Acid Sequence - Wild Type
MKLLVILLFSGLITGFRSDSSSSLPPKLLLVSFDGFRADYLKNYEFPHLQNFIKEGVLVEHVKNVFITKTFPNHYSIVTGLYEESHGIVANSMYDAVTKKHFSDSNDKDPFWWNEAVPIWVTNQLQENRSSAAAMWPGTDVPIHDTISSYFMNYNSSVSFEERLNNITMWLNNSNPPVTFATLYWEEPDASGHKYGPEDKENMSRVLKKIDDLIGDLVQRLKMLGLWENLNVIITSDHGMTQCSQDRLINLDSCIDHSYYTLIDLSPVAAILPKINRTEVYNKLKNCSPHMNVYLKEDIPNRFYYQHNDRIQPIILVADEGWTIVLNESSQKLGDHGYDNSLPSMHPFLAAHGPAFHKGYKHSTINIVDIYPMMCHILGLKPHPNNGTFGHTKCLLVDQWCINLPEAIAIVIGSLLVLTMLTCLIIIMQNRLSVPRPFSRLQLQEDDDDPLIG

SEQ ID NO: 12 - protein, 850 aa, synthetic construct
  REGION 1..850: note = ENPP51 Amino Acid Sequence
  SIGNAL 24..25: note = Cleavage point of the signal peptide sequence
MTSKFLLVSFILAALSLSTTFSLQPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQED

SEQ ID NO: 13 - protein, 1474 aa, synthetic construct
  REGION 1..1474: note = ENPP51 - ALB Amino Acid Sequence
  SIGNAL 24..25: note = Cleavage point of the signal sequence
  REGION 857..1474: note = MISC_FEATURE - Indicate Albumin sequence
MTSKFLLVSFILAALSLSTTFSLQPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQEDGGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 14 - protein, 1074 aa, synthetic construct
  REGION 1..1074: note = ENPP5-NPP3-Fc sequence
  SIGNAL 22..23: note = Cleavage point of the signal sequence
  REGION 848..1074: note = MISC_FEATURE - Indicate Albumin sequence
MTSKFLLVSFILAALSLSTTFSKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 1469 aa, synthetic construct
  REGION 1..1469: note = ENPP5-NPP3-Albumin sequence
  SIGNAL 22..23: note = Cleavage point of the signal peptide sequence
  REGION 862..1469: note = MISC_FEATURE - Indicate Albumin sequence
MTSKFLLVSFILAALSLSTTFSKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 16 - protein, 23 aa, synthetic construct
  REGION 1..23: note = ENPP5 Protein Export Signal Sequence
  SITE 23: note = MISC_FEATURE - X can be any amino acid
MTSKFLLVSFILAALSLSTTFSX

SEQ ID NO: 17 - protein, 1079 aa, synthetic construct
  REGION 1..1079: note = ENPP51-Fc
  SIGNAL 22..23: note = Cleavage point of the signal peptide sequence
  REGION 853..1079: note = MISC_FEATURE - Indicate Fc sequence
MTSKFLLVSFILAALSLSTTFSGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 1082 aa, synthetic construct
  REGION 1..1082: note = ENPP71-Fc Amino Acid Sequence
  SIGNAL 22..23: note = Cleavage point of the signall peptide sequence
  REGION 856..1082: note = MISC_FEATURE - Indicate Fc sequence
MRGPAVLLTVALATLLAPGAGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 849 aa, synthetic construct
  REGION 1..849: note = ENPP71 (lacking NPP1 N-Terminus GLK) Amino Acid Sequence
  SIGNAL 22..23: note = Cleavage point of the signal peptide sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 20 - protein, 1079 aa, synthetic construct
  REGION 1..1079: note = ENPP71 (lacking NPP1 N-Terminus GLK) - Fc Amino Acid Sequence
  SIGNAL 22..23: note = Cleavage point of the signal peptide sequence
  REGION 853..1079: note = MISC_FEATURE - Indicate Fc Sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 1474 aa, synthetic construct
  REGION 1..1474: note = - ENPP71 (lacking NPP1 N-Terminus GLK) - ALB Amino Acid Sequence
  SIGNAL 22..23: note = Cleavage point of the signal peptide sequence
  REGION 857..1474: note = MISC_FEATURE - Indicate Albumin sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 22 - protein, 1072 aa, synthetic construct
  REGION 1..1072: note = ENPP7-NPP3-Fc sequence
  SIGNAL 20..21: note = Cleavage point of the signal peptide sequence
  REGION 846..1072: note = MISC_FEATURE - Indicate Fc sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 1396 aa, synthetic construct
  REGION 1..1396: note = ENPP71-Albumin
  SIGNAL 23..24: note = Cleavage point of the signal peptide sequence
  REGION 857..1396: note = MISC_FEATURE - Indicate Fc sequence
MRGPAVLLTVALATLLAPGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDGGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTL

SEQ ID NO: 24 - protein, 1467 aa, synthetic construct
  REGION 1..1467: note = ENPP7-NPP3-Albumin
  SIGNAL 20..21: note = Cleavage point of the signal peptide sequence
  REGION 860..1467: note = MISC_FEATURE - Indicate Albumin sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 25 - protein, 1507 aa, synthetic construct
  REGION 1..1507: note = ENPP7-ENPP3-Albumin
  SIGNAL 20..21: note = Cleavage point of the signal peptide sequence
  REGION 900..1507: note = MISC_FEATURE - Indicate Albumin sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 26 - protein, 852 aa, synthetic construct
  REGION 1..852: note = ENPP71 Amino Acid Sequence
  SIGNAL 22..23: note = Cleavage point of the signal peptide sequence
MRGPAVLLTVALATLLAPGAGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 27 - protein, 925 aa, synthetic construct
  REGION 1..925: note = ENPP121 Amino Acid Sequence
  SIGNAL 92..93: note = Cleavage point of the signal peptide sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 28 - protein, 1155 aa, synthetic construct
  REGION 1..1155: note = ENPP121-Fc Amino Acid Sequence
  SIGNAL 92..93: note = Cleavage point of the signal peptide sequence
  REGION 929..1155: note = MISC_FEATURE - Indicate Fc sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 1550 aa, synthetic construct
  REGION 1..1550: note = ENPP121-ALB Amino Acid Sequence
  SIGNAL 92..93: note = Cleavage point of the signal peptide sequence
  REGION 933..1550: note = MISC_FEATURE - Indicate Albumin sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 30 - protein, 1147 aa, synthetic construct
  REGION 1..1147: note = ENPP121-NPP3-Fc sequence
  SIGNAL 95..96: note = Cleavage point of the signal peptide sequence
  REGION 921..1147: note = MISC_FEATURE - Indicate Fc sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 1542 aa, synthetic construct
  REGION 1..1542: note = ENPP121-NPP3-Albumin sequence
  SIGNAL 95..96: note = Cleavage point of the signal peptide sequence
  REGION 935..1542: note = MISC_FEATURE - Indicate Albumin sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 32 - protein, 98 aa, synthetic construct
  REGION 1..98: note = ENPP121GLK Protein Export Signal Sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLK

SEQ ID NO: 33 - protein, 623 aa, synthetic construct
  REGION 1..623: note = Albumin Sequence
GGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 34 - protein, 227 aa, synthetic construct
  REGION 1..227: note = Human IgG Fc domain, Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 618 aa, synthetic construct
  REGION 1..618: note = Albumin Sequence
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 36 - protein, 16 aa, synthetic construct
  REGION 1..16: note = ENPP2 Signal Peptide
LFTFAVGVNICLGFTA

SEQ ID NO: 37 - protein, 20 aa, synthetic construct
  REGION 1..20: note = Signal Sequence ENPP7
MRGPAVLLTVALATLLAPGA

SEQ ID NO: 38 - protein, 22 aa, synthetic construct
  REGION 1..22: note = Signal sequence ENPP7
MRGPAVLLTVALATLLAPGAGA

SEQ ID NO: 39 - protein, 95 aa, synthetic construct
  REGION 1..95: note = Signal Sequence ENPP1-2-1
MERDGCAGGGSRGGEGGRAPREGPAGNGRDRGRSHAAEAPGDPQAAASLLAPMDVGEEPLEKAARARTAKDPNTYKIISLFTFAVGVNICLGFTA

SEQ ID NO: 40 - protein, 17 aa, synthetic construct
  REGION 1..17: note = exENPP3
LLVIMSLGLGLGLGLRK

SEQ ID NO: 41 - protein, 22 aa, synthetic construct
  REGION 1..22: note = Signal Sequence ENPP5
MTSKFLLVSFILAALSLSTTFS

SEQ ID NO: 42 - protein, 19 aa, synthetic construct
  REGION 1..19: note = Signal Sequence - Azurocidin
MTRLTVLALLAGLLASSRA

SEQ ID NO: 43-46 - skipped (4 entries)

SEQ ID NO: 47 - protein, 15 aa, synthetic construct
  REGION 1..15: note = Linker
  REGION 1..15: note = MISC_FEATURE - Features section of the listing that any one or all of amino acids 1-15 can either be present or absent
RRRRRRRRRRRRRRR

SEQ ID NO: 48 - protein, 16 aa, synthetic construct
  REGION 1..16: note = Linker
DSSSEEKFLRRIGRFG

SEQ ID NO: 49 - protein, 12 aa, synthetic construct
  REGION 1..12: note = Linker
EEEEEEEPRGDT

SEQ ID NO: 50 - protein, 12 aa, synthetic construct
  REGION 1..12: note = Linker
APWHLSSQYSRT

SEQ ID NO: 51 - protein, 12 aa, synthetic construct
  REGION 1..12: note = Linker
STLPIPHEFSRE

SEQ ID NO: 52 - protein, 12 aa, synthetic construct
  REGION 1..12: note = Linker
VTKHLNQISQSY

SEQ ID NO: 53 - protein, 15 aa, synthetic construct
  REGION 1..15: note = Linker
  REGION 1..15: note = MISC_FEATURE - Features section of the listing that any one or all of amino acids 2-15 can either be present or absent
EEEEEEEEEEEEEEE

SEQ ID NO: 54 - skipped

SEQ ID NO: 55 - protein, 6 aa, synthetic construct
  REGION 1..6: note = Linker
GGSGGS

SEQ ID NO: 56 - protein, 7 aa, synthetic construct
  REGION 1..7: note = Linker
RSGSGGS

SEQ ID NO: 57 - protein, 15 aa, synthetic construct
  REGION 1..15: note = Linker
  REGION 1..15: note = MISC_FEATURE - Features section of the listing that any one or all of amino acids 2-15 can either be present or absent
DDDDDDDDDDDDDDD

SEQ ID NO: 58 - protein, 16 aa, synthetic construct
  REGION 1..16: note = Linker
LVIMSLGLGLGLGLRK

SEQ ID NO: 59 - protein, 15 aa, synthetic construct
  REGION 1..15: note = Linker
VIMSLGLGLGLGLRK

SEQ ID NO: 60 - protein, 14 aa, synthetic construct
  REGION 1..14: note = Linker
IMSLGLGLGLGLRK

SEQ ID NO: 61 - protein, 13 aa, synthetic construct
  REGION 1..13: note = Linker
MSLGLGLGLGLRK

SEQ ID NO: 62 - protein, 12 aa, synthetic construct
  REGION 1..12: note = Linker
SLGLGLGLGLRK

SEQ ID NO: 63 - protein, 11 aa, synthetic construct
  REGION 1..11: note = Linker
LGLGLGLGLRK

SEQ ID NO: 64 - protein, 10 aa, synthetic construct
  REGION 1..10: note = Linker
GLGLGLGLRK

SEQ ID NO: 65 - protein, 9 aa, synthetic construct
  REGION 1..9: note = Linker
LGLGLGLRK

SEQ ID NO: 66 - protein, 8 aa, synthetic construct
  REGION 1..8: note = Linker
GLGLGLRK

SEQ ID NO: 67 - protein, 7 aa, synthetic construct
  REGION 1..7: note = Linker
LGLGLRK

SEQ ID NO: 68 - protein, 6 aa, synthetic construct
  REGION 1..6: note = Linker
GLGLRK

SEQ ID NO: 69 - protein, 5 aa, synthetic construct
  REGION 1..5: note = Linker
LGLRK

SEQ ID NO: 70 - protein, 4 aa, synthetic construct
  REGION 1..4: note = Linker
GLRK

SEQ ID NO: 71-72 - skipped (2 entries)

SEQ ID NO: 73 - protein, 15 aa, synthetic construct
  REGION 1..15: note = Linker
  REGION 1..15: note = MISC_FEATURE - Features section of the listing that any one or all of amino acids 1-15 can either be present or absent
KKKKKKKKKKKKKKK

SEQ ID NO: 74 - protein, 15 aa, synthetic construct
  REGION 1..15: note = Linker
  REGION 1..15: note = MISC_FEATURE - Features section of the listing that any one or all of amino acids 2-15 can either be present or absent
DDDDDDDDDDDDDDD

SEQ ID NO: 75 - protein, 50 aa, synthetic construct
  REGION 1..50: note = Linker
  REGION 1..50: note = MISC_FEATURE - Amino acids 6-50 can either be present or absent in groups of 5(GGGGS) starting at position 6
GGGGSGGGGSGGGGSGGGGSGGGGSGGGGSGGGGSGGGGSGGGGSGGGGS

SEQ ID NO: 76 - DNA, 2625 bp, synthetic construct
  misc_feature 1..2625: note = ENPP3 Nucleotide sequence
  source 1..2625: mol_type = other DNA
atggaatctacgttgactttagcaacggaacaacctgttaagaagaacactcttaagaaatataaaatagcttgcattgttcttcttgctttgctggtgatcatgtcacttggattaggcctggggcttggactcaggaaactggaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaatgccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactatt

SEQ ID NO: 77 - DNA, 2775 bp, synthetic construct
  misc_feature 1..2775: note = ENPP1 Nucleotide sequence
  source 1..2775: mol_type = other DNA
atggaacgggacggctgtgccggcggaggatcaagaggcggagaaggcggcagagcccctagagaaggacctgccggcaacggcagagacagaggcagatctcatgccgccgaagcccctggcgatcctcaggctgctgcttctctgctggcccccatggatgtgggcgaggaacctctggaaaaggccgccagagccagaaccgccaaggaccccaacacctacaaggtgctgagcctggtgctgtccgtgtgcgtgctgaccaccatcctgggctgcatcttcggcctgaagcccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagat

SEQ ID NO: 78 - DNA, 3255 bp, synthetic construct
  misc_feature 1..3255: note = Azurocidin-ENPP1-FC Nucleotide sequence
  source 1..3255: mol_type = other DNA
ggtaccgccaccatgacaagactgacagtgctggctctgctggccggactgttggcctcttctagagctgctccttcctgcgccaaagaagtgaagtcctgcaagggcagatgcttcgagcggaccttcggcaactgtagatgtgacgccgcttgcgtggaactgggcaactgctgcctggactaccaagagacatgcatcgagcccgagcacatctggacctgcaacaagttcagatgcggcgagaagcggctgaccagatctctgtgcgcctgctctgacgactgcaaggacaagggcgactgctgcatcaactactcctctgtgtgccagggcgagaagtcctgggttgaagaaccctgcgagtccatcaacgagcctcagtgtcctgccggcttcgagacacctcctactctgctgttctccctggatggcttcagagccgagtacctgcatacttggggaggcctgctgccagtgatctccaagctgaagaagtgcggcacctacaccaagaacatgaggcctgtgtaccctaccaagacattccccaaccactactccatcgtgaccggcctgtatcctgagagccacggcatcatcgacaacaagatgtacgaccccaagatgaacgcctccttcagcctgaagtccaaagagaagttcaaccccgagtggtataagggcgagcctatctgggtcaccgctaagtaccagggactgaagtctggcaccttcttttggcctggctccgacgtggaaatcaacggcatcttccccgacatctataagatgtacaacggctccgtgcctttcgaggaacgcattctggctgttctgcagtggctgcagctgcctaaggatgagaggcctcacttctacaccctgtacctggaagaacctgactcctccggccactcttatggccctgtgtcctctgaagtgatcaaggccctgcagcgagtggacggaatggtcggaatgctgatggacggcctgaaagagctgaacctgcacagatgcctgaacctgatcctgatctccgaccacggcatggaacaggggagctgcaagaagtacatctacctgaacaagtacctgggcgacgtgaagaacatcaaagtgatctacggcccagccgccagactgaggccttctgatgtgcctgacaagtactactccttcaactacgagggaatcgcccggaacctgtcctgcagagagcctaaccagcacttcaagccctacctgaagcactttctgcctaagcggctgcacttcgccaagtctgacagaatcgagcccctgaccttctatctggaccctcagtggcagctggccctgaatcctagcgagagaaagtactgtggctccggcttccacggctccgacaacgtgttctctaatatgcaggccctgttcgtcggctacggccctggctttaaacacggcatcgaggccgacaccttcgagaacatcgaggtgtacaatctgatgtgtgacctgctgaatctgacccctgctcctaacaacggcacccacggatctctgaaccatctgctgaagaatcccgtgtacacccctaagcaccccaaagaggttcaccctctggtccagtgtcctttcaccagaaatcctcgggacaacctgggctgctcttgcaacccttctatcctgcctatcgaggactttcagacccagttcaacctgaccgtggccgaggaaaagatcatcaagcacgagacactgccctacggcagacctagagtgctgcagaaagagaacaccatctgcctgctgtcccagcaccagttcatgtccggctactcccaggacatcctgatgcctctgtggacctcctacaccgtggaccggaacgatagcttctccaccgaggacttcagcaactgcctgtaccaggatttcagaatccctctgagccccgtgcacaagtgcagcttctacaagaacaacaccaaggtgtcctacggcttcctgtctcctccacagctgaacaagaactccagcggcatctactctgaggccctgctgaccaccaacatcgtgcccatgtaccagtccttccaagtgatctggcggtacttccacgacaccctgctgaggaagtacgccgaagaaagaaacggcgtgaacgtggtgtctggccccgtgttcgacttcgactacgacggcagatgcgactctctggaaaacctgcggcagaaaagacgagtgatccggaatcaagagatcctgattcctacacacttctttatcgtgctgaccagctgcaaggatacctctcagacccctctgcactgcgagaatctggacaccctggccttcattctgcctcacagaaccgacaactccgagtcctgtgtgcacggcaagcacgactcctcttgggtcgaagaactgctgatgctgcaccgggccagaatcaccgatgtggaacacatcaccggcctgagcttctaccagcagcggaaagaacctgtgtccgatatcctgaagctgaaaacccatctgccaaccttcagccaagaggacctgatcaacgacaagacccacacctgtcctccatgtcctgctccagaactgctcggaggcccctctgtgttcctgtttccacctaagccaaaggacacactgatgatctctcggacccctgaagtgacctgcgtggtggtggatgtgtctcacgaagatcccgaagtcaagttcaattggtacgtggacggcgtggaagtgcacaacgccaagaccaagcctagagaggaacagtacaactccacctacagagtggtgtccgtgctgactgtgctgcaccaggattggctgaacggcaaagagtacaagtgcaaagtgtccaacaaggctctgcccgctcctatcgaaaagaccatctccaaggctaagggccagcctcgggaacctcaggtttacaccctgcctccatctcgggaagagatgaccaagaaccaggtgtccctgacctgcctggtcaagggcttctacccttccgatatcgccgtggaatgggagtccaatggccagcctgagaacaactacaagacaacccctcctgtgctggacagcgacggctcattcttcctgtactctaagctgacagtggacaagtcccggtggcagcaaggcaatgtgttttcctgctctgtgatgcacgaggccctccacaatcactacacccagaagtccctgtctctgtcccctggcaaatgatagctcgag

SEQ ID NO: 79 - DNA, 3219 bp, synthetic construct
  misc_feature 1..3219: note = Azurocidin-ENPP3-FC Nucleotide sequence
  source 1..3219: mol_type = other DNA
atgaccagactgaccgtgctggccctgctggccggcctgctggccagcagcagagccgccaagcagggcagctgcagaaagaagtgcttcgacgccagcttcagaggcctggagaactgcagatgcgacgtggcctgcaaggacagaggcgactgctgctgggacttcgaggacacctgcgtggagagcaccagaatctggatgtgcaacaagttcagatgcggcgagaccagactggaggccagcctgtgcagctgcagcgacgactgcctgcagagaaaggactgctgcgccgactacaagagcgtgtgccagggcgagaccagctggctggaggagaactgcgacaccgcccagcagagccagtgccccgagggcttcgacctgccccccgtgatcctgttcagcatggacggcttcagagccgagtacctgtacacctgggacaccctgatgcccaacatcaacaagctgaagacctgcggcatccacagcaagtacatgagagccatgtaccccaccaagaccttccccaaccactacaccatcgtgaccggcctgtaccccgagagccacggcatcatcgacaacaacatgtacgacgtgaacctgaacaagaacttcagcctgagcagcaaggagcagaacaaccccgcctggtggcacggccagcccatgaacctgaccgccatgtaccagggcctgaaggccgccacctacttctggcccggcagcgaggtggccatcaacggcagcttccccagcatctacatgccctacaacggcagcgtgcccttcgaggagagaatcagcaccctgctgaagtggctggacctgcccaaggccgagagacccagattctacaccatgtacttcgaggagcccgacagcagcggccacgccggcggccccgtgagcgccagagtgatcaaggccctgcaggtggtggaccacgccttcggcatgctgatggagggcctgaagcagagaaacctgcacaactgcgtgaacatcatcctgctggccgaccacggcatggaccagacctactgcaacaagatggagtacatgaccgactacttccccagaatcaacttcttctacatgtacgagggccccgcccccagaatcagagcccacaacatcccccacgacttcttcagcttcaacagcgaggagatcgtgagaaacctgagctgcagaaagcccgaccagcacttcaagccctacctgacccccgacctgcccaagagactgcactacgccaagaacgtgagaatcgacaaggtgcacctgttcgtggaccagcagtggctggccgtgagaagcaagagcaacaccaactgcggcggcggcaaccacggctacaacaacgagttcagaagcatggaggccatcttcctggcccacggccccagcttcaaggagaagaccgaggtggagcccttcgagaacatcgaggtgtacaacctgatgtgcgacctgctgagaatccagcccgcccccaacaacggcacccacggcagcctgaaccacctgctgaaggtgcccttctacgagcccagccacgccgaggaggtgagcaagttcagcgtgtgcggcttcgccaaccccctgcccaccgagagcctggactgcttctgcccccacctgcagaacagcacccagctggagcaggtgaaccagatgctgaacctgacccaggaggagatcaccgccaccgtgaaggtgaacctgcccttcggcagacccagagtgctgcagaagaacgtggaccactgcctgctgtaccacagagagtacgtgagcggcttcggcaaggccatgagaatgcccatgtggagcagctacaccgtgccccagctgggcgacaccagccccctgccccccaccgtgcccgactgcctgagagccgacgtgagagtgccccccagcgagagccagaagtgcagcttctacctggccgacaagaacatcacccacggcttcctgtacccccccgccagcaacagaaccagcgacagccagtacgacgccctgatcaccagcaacctggtgcccatgtacgaggagttcagaaagatgtgggactacttccacagcgtgctgctgatcaagcacgccaccgagagaaacggcgtgaacgtggtgagcggccccatcttcgactacaactacgacggccacttcgacgcccccgacgagatcaccaagcacctggccaacaccgacgtgcccatccccacccactacttcgtggtgctgaccagctgcaagaacaagagccacacccccgagaactgccccggctggctggacgtgctgcccttcatcatcccccacagacccaccaacgtggagagctgccccgagggcaagcccgaggccctgtgggtggaggagagattcaccgcccacatcgccagagtgagagacgtggagctgctgaccggcctggacttctaccaggacaaggtgcagcccgtgagcgagatcctgcagctgaagacctacctgcccaccttcgagaccaccatcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag